(19) United States
(12) Patent Application Publication
(10) Pub. No.: US 2025/0042956 A1
(43) Pub. Date: Feb. 6, 2025

(54) TOOLS FOR GENE SILENCING

(71) Applicant: The Regents of the University of California, Oakland, CA (US)

(72) Inventors: Steve E. JACOBSEN, Agoura Hills, CA (US); Ming WANG, Plano, TX (US)

(73) Assignee: The Regents of the University of California, Oakland, CA (US)

(21) Appl. No.: 18/715,424

(22) PCT Filed: Dec. 2, 2022
(86) PCT No.: PCT/US2022/080825

§ 371 Date: May 31, 2024

Related U.S. Application Data
(60) Provisional application No. 63/285,917, filed on Dec. 3, 2021.

(51) Int. Cl. C07K 14/415; C12N 15/82

(52) U.S. Cl. CPC C07K 14/415; C12N 15/8216

(57) ABSTRACT

The present disclosure relates to recombinant transcriptional repressor polypeptides that are capable of being targeted to specific loci, as well as methods of using these recombinant polypeptides for reducing expression of a target nucleic acid in plants.

### CROSS-REFERENCE TO RELATED APPLICATION

This application claims the benefit of U.S. Provisional Application No. 63/285,917, filed on Dec. 3, 2021, which is incorporated herein by reference in its entirety.

### REFERENCE TO AN ELECTRONIC SEQUENCE LISTING

The contents of the electronic sequence listing (262232002540SEQLIST.xml; Size: 311,130 bytes; and Date of Creation: Dec. 2, 2022) is herein incorporated by reference in its entirety.

### FIELD

The present disclosure relates to recombinant transcriptional repressor polypeptides that are capable of being targeted to specific loci, as well as methods of using these recombinant polypeptides for reducing expression of a target nucleic acid in plants.

### BACKGROUND

Epigenetic marks are enzyme-mediated chemical modifications of DNA and of its associated chromatin proteins. Although epigenetic marks do not alter the primary sequence of DNA, they do contain heritable information and play key roles in regulating genome function. Such modifications, including cytosine methylation, posttranslational modifications of histone tails and the histone core, and the positioning of nucleosomes (histone octamers wrapped with DNA), influence the transcriptional state and other functional aspects of chromatin. For example, methylation of DNA and certain residues on the histone H3 N-terminal tail, such as H3 lysine 9 (H3K9), are important for transcriptional gene silencing and the formation of heterochromatin. Such marks are essential for the silencing of nongenic sequences, including transposons, pseudogenes, repetitive sequences, and integrated viruses, that become deleterious to cells if expressed and hence activated. Epigenetic gene silencing is also important in developmental phenomena such as imprinting in both plants and mammals, as well as in cell differentiation and reprogramming. Having the ability to specifically control target gene silencing is thus of great interest.

Different pathways involved in epigenetic silencing have been previously described, and include histone deacetylation, H3K27 and H3K9 methylation, H3K4 demethylation, and DNA methylation of promoters. An avenue to achieve DNA methylation is via a phenomenon known as RNA-directed DNA methylation, where non-coding RNAs act to direct methylation of a DNA sequence. In plants, proteins generally do not link the recognition of a specific DNA sequence with the establishment of an epigenetic state. Thus, endogenous plant epigenetic regulators generally cannot be used for epigenetic silencing of specific genes or transgenes in plants.

Accordingly, a need exists for improved transcriptional repressors that are capable of being targeted to specific loci to reduce expression of targeted nucleic acids in plants.

### BRIEF SUMMARY

In one aspect, the present disclosure provides a method for producing a plant with reduced expression of a target nucleic acid, including: (a) providing a plant including a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide, and; (b) growing the plant under conditions whereby the recombinant polypeptide is expressed and targeted to the target nucleic acid, thereby reducing expression of the target nucleic acid to produce the plant with reduced expression of the target nucleic acid. In some embodiments the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments, the DNA binding domain includes a zinc finger. In some embodiments that may be combined with any of the preceding embodiments, expression of the target nucleic acid is reduced by at least 50% as compared to a corresponding control nucleic acid. In some embodiments that may be combined with any of the preceding embodiments, the recombinant polypeptide is encoded by a recombinant nucleic acid. In some embodiments that may be combined with any of the preceding embodiments, the transcriptional repressor polypeptide is a TRBIP1 polypeptide. In some embodiments, the TRBIP1 polypeptide includes an amino acid sequence having at least 80% identity to SEQ ID NO: 181. In some embodiments that may be combined with any of the preceding embodiments, the plant further includes a recombinant DNA methyltransferase polypeptide which is capable of being targeted to the target nucleic acid. In some embodiments, the DNA methyltransferase polypeptide is an MQ1 polypeptide. In some embodiments, the MQ1 polypeptide includes an amino acid sequence having at least 80% identity to SEQ ID NO: 212. In some embodiments that may be combined with any of the preceding embodiments, the method further includes: (c) crossing the plant with reduced expression of the target nucleic acid to a second plant to produce one or more F1 plants. In some embodiments, the method further includes: (d) selecting from the one or more F1 plants an F1 plant that (i) lacks the recombinant polypeptide, and (ii) has reduced expression of the target nucleic acid.

In another aspect, the present disclosure provides a recombinant nucleic acid including a plant promoter and which encodes a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. In some embodiments, the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments, the DNA binding domain includes a zinc finger.

In another aspect, the present disclosure provides an expression vector that includes a recombinant nucleic acid including a plant promoter and which encodes a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. In some embodiments, the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments, the DNA binding domain includes a zinc finger.

In another aspect, the present disclosure provides a plant cell or other host cell that includes an expression vector that includes a recombinant nucleic acid including a plant promoter and which encodes a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. In some embodiments, the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments, the DNA binding domain includes a zinc finger.

In another aspect, the present disclosure provides a plant including a plant cell that includes an expression vector that includes a recombinant nucleic acid including a plant promoter and which encodes a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. In some embodiments, the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments, the DNA binding domain includes a zinc finger.

In another aspect, the present disclosure provides a plant cell that includes a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide, wherein the plant cell includes a nucleic acid with reduced expression as compared to a corresponding control nucleic acid. In some embodiments, the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments that may be combined with any of the preceding embodiments, the DNA binding domain includes a zinc finger. In some embodiments that may be combined with any of the preceding embodiments, expression of the target nucleic acid is reduced by at least 50% as compared to a corresponding control nucleic acid. In some embodiments that may be combined with any of the preceding embodiments, the recombinant polypeptide is encoded by a recombinant nucleic acid.

In another aspect, the present disclosure provides a plant including a plant cell that includes a recombinant polypeptide including a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide, wherein the plant cell includes a nucleic acid with reduced expression as compared to a corresponding control nucleic acid. In some embodiments, the transcriptional repressor polypeptide includes an amino acid sequence having at least 80%, at least 85%, at least 90%, at least 95%, or 100% amino acid identity to any one of SEQ ID NO: 1-210. In some embodiments that may be combined with any of the preceding embodiments, the targeting domain includes a DNA-binding domain. In some embodiments that may be combined with any of the preceding embodiments, the DNA binding domain includes a zinc finger. In some embodiments that may be combined with any of the preceding embodiments, expression of the target nucleic acid is reduced by at least 50% as compared to a corresponding control nucleic acid. In some embodiments that may be combined with any of the preceding embodiments, the recombinant polypeptide is encoded by a recombinant nucleic acid.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E show the effector proteins obtained from zinc finger (ZF) target screening. FIG. 1A shows the list of effector proteins identified from ZF target screening, which were dependent (left) or independent (right) of DNA methylation. FIG. 1B shows the silencing fold (Log 10) of FWA gene of ZF fusion lines versus fwa, and the error bars indicate mean standard error. The asterisks indicate the p value calculated by t-test; *=p<0.05; **=p<0.01; ***=p<0.001. FIG. 1C shows the flowering time of fwa, Col-0, and four representative T2 ZF fusion lines. FIG. 1D shows the CG (red bars), CHG (green bars), and CHH (blue bars) DNA methylation levels over FWA promoter regions in fwa, Col-0, and representative T2 ZF fusion lines measured by bisulfite (BS)-PCR-seq. Pink vertical boxes indicate ZF binding sites. FIG. 1E shows the Observed/Expected values of up- (pink) and down-regulated (blue) differentially expressed genes (DEGs) over ZF off-target sites in ZF fusion lines, measured by Region Associated DEG (RAD) analysis. The asterisks indicate the p value calculated with hypergeometric test; *=p<0.05; **=p<0.01; ***=p<0.001; ****=p<0.0001.

FIGS. 2A-2H show target gene silencing by MOM1 complex and DNA methylation. FIG. 2A shows the flowering time of fwa, Col-0, MOM1-ZF, MOM2-ZF, PIAL1-ZF, and PIAL2-ZF representative T2 lines. FIG. 2B shows CG (red bars), CHG (green bars), and CHH (blue bars) DNA methylation levels over FWA promoter regions in fwa, Col-0, and representative T2 lines of MOM1-ZF, MOM2-ZF, PIAL1-ZF and PIAL2-ZF, measured by BS-PCR-seq. Pink vertical boxes indicate ZF binding sites. FIG. 2C illustrates metaplots showing relative CG, CHG, and CHH DNA methylation levels over ZF off-target sites in representative T2 lines of MOM1-ZF, MOM2-ZF, PIAL1-ZF, and PIAL2-ZF versus fwa measured by whole genome bisulfite sequencing (WGBS). FIG. 2D shows flowering time of fwa, Col-0, and T1 lines of PIAL2-ZF, MOM1-ZF and PHD1-ZF in fwa mutant as well as fwa introgressed mutants, including nrpd1, suvh2/9, morc6, dms3, drd1, rdm1, nrpe1, and drm1/2. FIG. 2E shows flowering time of MOM2-ZF (top panel) and PIAL1-ZF (bottom panel) in fwa introgressed morc6 mutant. FIG. 2F shows flowering time of MORC6-ZF in fwa introgressed aipp3, phd1, mom1, mom2, and pial1/2 mutants. FIG. 2G illustrates a Yeast Two Hybrid showing in vitro direct interactions between PIAL1 and PIAL2 with MORC6, MOM1 CMM2 domain, and MOM2, respectively. FIG. 2H shows the PIAL2 and MORC6 in vivo interaction shown by Co-immunoprecipitation (Co-IP) in MORC6-FLAG and PIAL2-Myc crossed lines.

FIGS. 3A-3G show the role of MOM1 complex in gene regulation and DNA methylation. FIG. 3A shows metaplots and heatmaps representing ChIP-seq signals of Pol V, MORC6-Myc, MOM1-Myc, PHD1-FLAG, AIPP3-FLAG, and PIAL2-Myc over Pol V peaks (n=10,868). FIG. 3B shows the distribution of MOM1 and Pol V ChIP-seq (see e.g., Liu, PLoS Genet., 2018) peaks over chromosomes based on the unsupervised clustering analysis. Cluster1 (n=12,526) represents the overlapped ChIP-seq peaks of MOM1 and Pol V, while Cluster 2 (n=1,788) represents MOM1 ChIP-seq peaks independent of Pol V. FIG. 3C shows ChIP-seq signals of Pol V (see e.g., Liu, PLoS Genet., 2018), MOM1, PIAL2, MORC4 (see e.g., Xue, Nat. Comm., 2021), MORC6, MORC7 (see e.g., Xue, Nat. Comm., 2021), PHD1, and AIPP3 over Cluster 1 and Cluster 2 ChTP-seq peaks of MOM1. FIG. 3D shows boxplots and heatmaps showing the variation of CG, CHG, and CHH DNA methylation in phd1-2, aipp3-1, mom2-2, mom1-3, morc6-3 and morchex mutants versus Col-0 wild type over hypo CHH hcDMRs of morchex mutant (n=520). FIG. 3E shows heatmap depicting the overlapping enrichment of CHH hcDMRs among aipp3-1, mom2-2, mom1-3 (see e.g., Han, Plant Cell., 2016), pial1/2 (see e.g., Han, Plant Cell., 2016), morc6-3 (see e.g., Stroud, Cell, 2013), and morchex (see e.g., Harris, PLoS Genet., 2016) mutants over morchex mutant hypo CHH hcDMRs (n=520) (see e.g., Harris, PLoS Genet., 2016). FIG. 3F shows a boxplot representing the expression level of genes that located within 1 kb distance to CHH hcDMRs (n=520) of morchex mutant in Col-0, phd1-2, aipp3-1, mom2-2, mom1-3, pial1/2, morc6-3, and morchex mutants. FIG. 3G shows flowering time of fwa, Col-0 controls, and Col-0 wild type, mom2-2, mom1-3, pial1/2, and nrpe1-11 mutants transformed with FWA transgene.

FIGS. 4A-4G show targeted gene silencing by telomere repeat-binding factors (TRBs), H3K27me3 deposition, and H3K4me3 demethylation. FIG. 4A shows flowering time of fwa, Col-0, and four representative T2 lines of MSI1-ZF, LHP1-ZF, and JMJ14-ZF. FIG. 4B shows CG (red bars), CHG (green bars), and CHH (blue bars) DNA methylation levels over FWA promoter regions in fwa, Col-0, and representative T2 lines of MSI1-ZF, LHP1-ZF, and JMJ14-ZF. Pink vertical boxes indicate ZF binding sites. FIG. 4C shows the Observed/Expected values of up- and down-regulated DEGs in of MSI1-ZF, LHP1-ZF, and JMJ14-ZF over ZF off-target sites (n=6,091), measured by RAD analysis. The asterisks indicate the p value calculated with a hypergeometric test; *=p<0.05; **=p<0.01; ***=p<0.001; ****=p<0.0001. FIG. 4D shows screenshots of H3K27me3 (top panel) and H3 (bottom panel) ChIP-seq signals over FWA region in fwa, TRB1-ZF, TRB2-ZF, ZF-TRB3, LHP1-ZF, MSI1-ZF, and ELF7-ZF. FLAG-ZF ChIP-seq indicate ZF binding site. FIG. 4E shows metaplots and heatmaps depicting the normalized H3K27me3 ChIP-seq signals over ZF off-targeting sites (n=6,091) and ZF off-targeting sites shuffle in the representative T2 lines of TRB1-ZF, TRB2-ZF, ZF-TRB3, LHP1-ZF, MSI1-ZF, and ELF7-ZF versus fwa, respectively. FIG. 4F shows screenshots of H3K4me3 (top panel) and H3 (bottom panel) ChIP-seq signals over FWA region in fwa, TRB1-ZF, TRB2-ZF, ZF-TRB3, JMJ14-ZF, and ELF7-ZF. The FLAG-ZF ChIP-seq signals indicate ZF binding site. FIG. 4G shows metaplots and heatmaps depicting the normalized H3K4me3 ChIP-seq signals in the representative T2 lines of TRB1-ZF, TRB2-ZF, ZF-TRB3, JMJ14-ZF, and ELF7-ZF over ZF off-targeting sites (n=6,091) and ZF off-targeting sites shuffle versus fwa, respectively.

FIG. 5A-5E show endogenous role of TRBs in H3K4me3 demethylation. FIG. 5A shows heatmap and metaplot showing TRB1-FLAG ChIP-seq peaks over JMJI4-FLAG ChIP-seq (n=4,041) peaks (left panel); TRB1-FLAG and JMJ14-FLAG ChIP-seq signals over TRB1-Cluster1 (n=892, middle panel) and TRB1-Cluster2 (n=6,710, right panel) respectively. FIG. 5B shows screenshots of TRB1-FLAG, TRB2-FLAG, TRB3-FLAG, JMJ14-FLAG, H3K27me3, and H3K4me3 ChIP-seq signals over a representative peak of TRB1-Cluster1 and TRB1-Cluster2, respectively. FIG. 5C shows metaplots and heatmaps indicating the H3K4me3 ChIP-seq signals in jmj14-1 and trb1/2/3 triple mutants versus Col-0 over JMJ14-FLAG ChIP-seq peaks (n=4,014) and JMJ14 peaks shuffle (left panel); over TRB1 Cluster1 peaks (n=892) and TRB1 Cluster1 peaks shuffle (middle panel); and TRB1 Cluster2 peaks (n=6,710) and TRB1 Cluster2 peaks shuffle (right panel). FIG. 5D illustrates a scatterplot showing the gene expression level in jmj14-1 mutant versus Col-0 (left panel) and trb1/2/3 triple mutant versus Col-0 (right panel). The red dots highlight JMJ14 binding genes and TRB1 binding genes in left and right panels, respectively. FIG. 5E illustrates a Venn diagram showing the overlap of all the up-regulated DEGs (left) and JMJ14-TRB1 co-bound DEGs (right) between trb1/2/3 triple mutant and jmj14-1 mutant in all genes (top panel) and TRB1-JMJ14 cobound genes (bottom panel).

FIGS. 6A-6G show targeted gene silencing by histone deacetylases (HDACs) and histone deacetylation. FIG. 6A shows screenshots of histone H3K9ac, H3K27ac, H4K16ac, and H3 ChIP-seq signals over FWA region in fwa, HD2A-ZF, HD2B-ZF, and HD2C-ZF. FIG. 6B shows heatmaps and metaplots representing the normalized H3K9ac (left panel), H3K27ac (middle panel), and H4K16ac (right panel) ChIP-seq signals over ZF off-target sites and ZF off-target sites shuffle in the representative T2 lines of HD2A-ZF, HD2B-ZF, and HD2C-ZF versus fwa. FIG. 6C shows the flowering time of fwa, Col-0, and four representative T2 lines of HDA6-ZF. FIG. 6D shows CG (red bars), CHG (green bars), and CHH (blue bars) DNA methylation levels over FWA promoter regions in fwa, Col-0, and representative T2 lines of HDA6-ZF, measured by BS-PCR-seq. Pink vertical boxes indicate ZF binding sites. FIG. 6E shows the Observed/Expected values of up- and down-regulated DEGs in HDA6-ZF over ZF off-target sites, measured by RAD analysis. The asterisks indicate the p value calculated with a hypergeometric test; *=p<0.05; **=p<0.01; ***=p<0.001; ****=p<0.0001. FIG. 6F shows screenshots of histone H3K9ac, H3K14ac, and H3 ChIP-seq signals over FWA region in fwa and HDA6-ZF. FIG. 6G shows heatmaps and metaplots representing the normalized H3K9ac (left panel) and H3K14ac (right panel) ChIP-seq signals over ZF off-target sites and ZF off-target sites shuffle in a HDA6-ZF T2 representative line versus fwa.

FIGS. 7A-7H show targeted gene silencing by ELF7 and Pol II transcription disruption. FIG. 7A shows screenshots of Pol II and H3 ChIP-seq signals over FWA region in fwa, ELF7-ZF, and HD2A-ZF. FIG. 7B shows heatmaps and metaplots representing normalized Pol II ChIP-seq signals in the representative T2 lines of ELF7-ZF (left panel) and HD2A-ZF (right panel) versus fwa, over ZF off-target sites (n=6,091) and ZF off-target sites shuffle, respectively. FIG. 7C shows a heatmap and metaplot showing normalized Pol II ChIP-seq signals over ELF7-FLAG ChIP-seq peaks (n=16,768) and ELF7 peaks shuffle in elf7-3 versus Col-0. FIG. 7D shows screenshots of ELF7-FLAG ChIP-seq signals, Pol II, and H3 ChIP-seq signals in Col-0 and elf7-3 mutants over two representative ELF7 targeting genes. FIG. 7E shows screenshots of Pol II ChTP-seq signals over FWA (top panel) and a representative ZF off-target gene (bottom panel) in fwa and a CPL2-ZF representative T2 transgenic line. FIG. 7F shows a heatmap and metaplot depicting normalized Pol II ChIP-seq signals over ZF off-target and ZF off-target sites shuffle in CPL2-ZF versus fwa. FIG. 7G shows a heatmap displaying Spearman correlation coefficients for all the down-regulated ZF targeting genes (distance≤100 bp, n=297) in HD2A-ZF, HD2B-ZF, HD2C-ZF, TRB1-F, TRB2-ZF, ZF-TRB3, ELF7-ZF, PHD1-ZF, and CPL2-ZF. Pairwise Spearman correlation values are shown in each box. FIG. 7H shows a boxplot showing intra TRB (TRB1-ZF, TRB2-ZF, and ZF-TRB3), intra HD2 (HD2A-ZF, HD2B-ZF, and HD2C-ZF) pathways, TRB-HD2 inter pathways Spearman correlation values displayed in FIG. 7G. The boxplot showing the median and the Tukey whiskers indicate 1.5 times the interquartile range from the 25th and 75th percentiles. The asterisks indicate the p value estimated by Wilcoxon rank-sum test; *=p<0.05 and **=p<0.01; n.s. represents no significance.

FIGS. 8A-8F show the ZF screening identified silencers. FIG. 8A shows a brief overview of RNA-directed DNA methylation (RdDM) pathway. FIG. 8B shows the flowering time of fwa, Col-0, and T1 lines of ZF fusions. FIG. 8C shows CG, CHG, and CHH DNA methylation levels over FWA promoter regions in fwa, Col-0, PHD1-ZF transgenic T2 line (+), PHD1-ZF segregant T2 line (−), ZF-SUVH2 transgenic T2 line (+), and ZF-SUVH2 segregant T2 line (−), measured by Bisulfite (BS)-PCR-seq. Pink vertical boxes indicate ZF binding sites. FIG. 8D shows the flowering time of ZF fusions T2 segregant lines (−) and transgenic lines (+). FIG. 8E illustrates a Western blot showing the protein expression levels of ZF fusion T2 transgenic lines with early flowering phenotype (left three samples; green line) and late flowering phenotype (right three samples; orange). FIG. 8F shows screenshots of RNA-seq signals in fwa and representative T2 transgenic lines of ZF fusions over two representative ZF off-target sites. The FLAG-ZF ChIP-seq indicates ZF binding sites.

FIGS. 9A-9F show targeted gene silencing by MOM1 complex and DNA methylation. FIG. 9A illustrates qRT-PCR showing the relative mRNA level of FWA gene in fwa, and four representative T2 lines of MOM1-ZF, MOM2-ZF, PIAL1-ZF, and PIAL2-ZF. Error bar indicates standard error of three technical replicates. FIG. 9B shows CG (red bars), CHG (green bars), and CHH (blue bars) DNA methylation levels over FWA promoter regions in Col-0, fwa, and the representative T2 transgenic lines (+) or segregant lines (−) of MOM1-ZF, MOM2-ZF, PIAL1-ZF, and PIAL2-ZF, measured by BS-PCR-seq. Pink vertical boxes indicate ZF binding sites. FIG. 9C illustrates screenshots of Whole Genome Bisulfite Sequencing (WGBS) showing CG (top panel), CHG (middle panel), and CHH (bottom panel) DNA methylation level over a representative ZF off-target site in fwa, and representative T2 lines of MOM1-ZF, MOM2-ZF, PIAL1-ZF, and PIAL2-ZF. FIG. 9D shows the flowering time of PHD1-ZF in fwa introgressed aipp3 and mom1 mutants; MOM1-ZF in fwa introgressed aipp3, phd1, mom2, and pial1/2 mutants; MOM2-ZF in fwa introgressed aipp3, phd1, mom1, and pial1/2 mutants; and PIAL2-ZF in fwa introgressed aipp3, phd1, mom1, and mom2 mutants. FIG. 9E shows the flowering time of MiniMOM1-ZF representative T2 lines. FIG. 9F shows CG, CHG, and CHH DNA methylation levels over FWA promoter regions in Col-0, fwa, and two representative Mini-MOM1-ZF T2 lines, measured by BS-PCR-seq. Pink vertical boxes indicate ZF binding sites.

FIGS. 10A-10F show the endogenous function of MOM1 complex in gene regulation. FIG. 10A shows screenshots of Pol V, MORC6-Myc, MOM1-Myc, PHD1-FLAG, AIPP3-FLAG, and PIAL2-Myc ChIP-seq signals and CG, CHG, and CHH DNA methylation level by WGBS over a representative RdDM site. FIG. 10B shows a metaplot and heatmap indicating H3K27me3 ChIP-seq signals in Col-0 over AIPP3-FLAG ChIP-seq (n=7,538) peaks. FIG. 10C shows screenshots of AIPP3-FLAG and H3K27me3 ChIP-seq signal over a representative site. FIG. 10D shows ChIP-seq signals of Pol V (see e.g., Liu, PLoS Genet., 2018), MOM1, PIAL2, MORC4 (see e.g., Xue, Nat. Comm., 2021), MORC6, and MORC7 (see e.g., Xue, Nat. Comm., 2021) over euchromatic transposable elements (TEs; H3K9me2 associated TEs) and heterochromatic TEs (non-H3K9me2 associated TEs).

FIG. 10E shows a heatmap showing the differentially expressed TEs (DE TEs, n=423) in three replicates of Col-0, phd1-2, phd1-3, aipp3-1, aipp3-2 mom1-2, mom1-3, mom2-1, mom2-2, pial1-2, pial2-1, pial1/2, morc6-3, and morchex mutants. FIG. 10F shows dotplots showing the DE bins (100 bp) over five Arabidopsis chromosomes in the mutants of phd1-2, phd1-3, aipp3-1, aipp3-2, mom1-2, mom1-3, mom2-1, mom2-2, pial1-2, pial2-1, pial1/2, morc6-3, and morchex versus Col-0 plants. The position of pericentromeric heterochromatin region of each chromosome is annotated at the bottom of each plot.

FIGS. 11A-11I show targeted silencing by TRBs, H3K27me3 deposition, and H3K4me3 demethylation. FIG. 11A shows the relative mRNA level of FWA in fwa, and three representative T2 lines of MSI1-ZF and LHP1-ZF, measured by qRT-PCR. Error bar indicates standard error of three technical replicates. FIG. 11B shows screenshots of RNA-seq signals in fwa, MSI1-ZF, and LHP1-ZF over a representative ZF off-target site. The FLAG-ZF ChIP-seq signals indicated ZF binding site. FIG. 11C shows screenshots of H3K27me3 and H3 ChIP-seq signals over a representative ZF off-target site in fwa and T2 lines of TRB1-ZF, TRB2-ZF, ZF-TRB3, LHP1-ZF, MSI1-ZF, and ELF7-ZF. FIG. 11D shows a Western blot showing the Co-immunoprecipitation (Co-IP) assay in JMJ14-FLAG (left panel) and TRB1-Myc F2 (right panel) crossed lines. FIG. 11E shows the relative mRNA level of FWA in fwa, and three representative T2 lines of JMJ14-ZF, measured by qRT-PCR. Error bar indicates standard error of three technical replicates. FIG. 11F shows screenshots of RNA-seq signals in fwa and JMJ14-ZF T2 line over a representative ZF off-target site. FLAG-ZF ChIP-seq signals indicated ZF binding site. FIG. 11G shows screenshots of H3K4me3 and H3 ChIP-seq signals in fwa and representative T2 transgenic lines of TRB1-ZF, TRB2-ZF, ZF-TRB3, JMJ14-ZF, and ELF7-ZF over a representative ZF off-target site. FIG. 11H shows screenshots of H3K27me3 and H3 ChIP-seq signals in fwa and JMJ14-ZF representative T2 lines over FWA. FIG. 1I shows a heatmap and metaplot showing the normalized H3K27me3 ChIP-seq signals over ZF off-target sites in JMJ14-ZF T2 lines versus fwa.

FIGS. 12A-12I show the endogenous role of TRBs in H3K27m3 deposition, H3K4me3 demethylation, and DNA methylation. FIG. 12A shows heatmaps and metaplots showing the ChIP-seq signals of TRB1-FLAG (left panel), TRB2-FLAG (middle panel), and TRB3-FLAG (right panel) over TRB1-FLAG ChIP-seq peaks (n=7,602). FIG. 12B shows a heatmap and metaplot showing H3K27me3 ChIP-seq signals in Col-0 over TRB1 ChIP-seq peaks. FIG. 12C shows pie charts indicating the annotation and percentage of TRB1-Cluster1 (left panel) or TRB1-Cluster2 (right panel) peaks over the whole genome. FIG. 12D shows the motif prediction by Homer showing the top 3 binding motifs of TRB1 (top panel), TRB2 (middle panel), and TRB3 (bottom panel). FIG. 12E illustrates the phenotype of trb1/2/3 triple mutants after 2- to 3-weeks growing in the MS medium (top panel), and the phenotype of the surviving trb1/2/3 triple mutant on soil, which were transferred from MS medium (bottom panel). FIG. 12F illustrates screenshots of ChIP-seq signals of TRB1-FLAG, TRB2-FLAG, TRB3-FLAG, and JMJ14-FLAG, as well as the normalized H3K27me3 and H3K4me3 ChIP-seq signals in Col-0, trb1/2/3 triple mutant, and jmj4-1 mutant, respectively. FIG. 12G shows metaplots and heatmaps showing the normalized H3K27me3 and H3K4me3 ChIP-seq signals in trb1/2/3 triple mutant versus Col-0, over H3K27me3 peaks (n=7,975) which were reduced in trb1/2/3 triple mutant. FIG. 12H shows CHH (left panel), CHG (middle panel), and CG (right panel) DNA methylation level in two replicates of Col-0 and trb1/2/3 triple mutants over RdDM sites, measured by WGBS. FIG. 12I shows screenshots of CG, CHG, and CHH DNA methylation level in Col-0 and trb1/2/3 triple mutants, respectively, over a representative RdDM site.

FIGS. 13A-13F show target gene silencing by HDACs and histone deacetylation. FIG. 13A shows a phylogenetic tree of four HD2 family proteins using ClustalW2. FIG. 13B shows screenshots of histone H3K9ac, H3K27ac, H4K16ac, and H3 ChIP-seq signals over two representative ZF off-target sites in fwa and T2 transgenic lines of HD2A-ZF, HD2B-ZF, and HD2C-ZF. FLAG-ZF ChIP-seq signal indicates ZF binding sites. FIG. 13C shows heatmaps and metaplots representing the FLAG ChIP-seq signals of HD2A-FLAG (left panel) and HD2C-FLAG (right panel) over HD2A peaks (n=3,645). FIG. 13D shows heatmaps and metaplots results of the normalized H3K9ac (left panel), H3K27ac (middle panel), and H4K16ac (right panel) ChIP-seq signals over HD2A-FLAG ChIP-seq peaks (n=3,645) and HD2A peaks shuffle in hd2a, hd2b, and hd2c mutants versus Col-0, respectively. FIG. 13E shows screenshots of HD2A-FLAG, HD2C-FLAG, histone H3K9ac, H3K27ac, H4K16ac, and H3 ChIP-seq signals in Col-0, hd2a, hd2b, and hd2c mutants over two representative HD2C binding sites. FIG. 13F illustrates FWA expression levels in fwa and three representative HDA6-ZF T2 lines by qRT-PCR. Error bar indicates standard error of three technical replicates.

FIGS. 14A-14H show the target gene silencing by ELF7 and Pol II transcription elongation. FIG. 14A shows screenshots of histone H3K36me2, H3K36me3, and H3 ChIP-seq signals over FWA in fwa and ELF7-ZF T2 lines. FIG. 14B shows heatmaps and metaplots depicting normalized H3K36me2 (left panel) and H3K36me3 (right panel) ChIP-seq signals over ZF off-target sites (n=6,091) and ZF off-target peaks shuffle in T2 transgenic lines of ELF7-ZF versus fwa. FIG. 14C shows physical phenotype of elf⁷-3 mutant, Col-0, and ELF7:ELF7-FLAG in elf7-3 mutant background. FIG. 14D shows screenshots of Pol II and H3 ChIP-seq signals over two representative ZF off-target binding sites in fwa and T2 transgenic lines of ELF7-ZF and HD2A-ZF. FIG. 14E shows heatmaps and metaplots showing Pol II, H3K36me2, and H3K36me3 ChIP-seq signals over ELF7-FLAG ChIP-seq peaks (n=16,768) in Col-0. FIG. 14F shows screenshots of ELF7-FLAG, Pol II, H3K36me2, and H3K36me3 ChIP-seq signals over four representative ELF7 binding genes. FIG. 14G shows of FLAG-ZF ChIP-seq and RNA-seq of fwa-1, HD2A-ZF, HD2B-ZF, HD2C-ZF, fwa-2, TRB1-ZF, TRB2-ZF, and ZF-TRB3 over representative down-regulated ZF targeting gene loci (distance≤100 bp, n=297), which were silenced in both HD2-ZFs and TRB-ZFs, HD2-ZFs only, and TRB-ZFs only, respectively. FIG. 14H shows heatmaps showing the expression level of down-regulated ZF targeting genes (distance≤100 bp, n=297) in HD2A-ZF, HD2B-ZF, HD2C-ZF, TRB1-ZF, TRB2-ZF, ZF-TRB3, ELF7-ZF, CPL2-ZF, and PHID1-ZF.

FIGS. 15A-15D show the leaf counts, FWA expression level and DNA methylation level over FWA promoter region in fwa and MBD2-ZF, ZF-SUVH7, SSRP1-ZF, SPT16-ZF, JMJ18-ZF, TRBIP1-ZF, TRBIP2-ZF and ASF1B-ZF. FIG. 15A shows the leaf number of fwa, Col-0, and T1 transgenic lines of MBD2-ZF, ZF-SUVH7, SSRP1-ZF, SPT16-ZF, JMJ18-ZF, TRBIP1-ZF, TRBIP2-ZF and ASF1B-ZF. FIG. 15B shows relative mRNA level of FWA in fwa, and three representative T1 lines of MBD2-ZF, ZF-SUVH7, SSRP1-ZF, SPT16-ZF, JMJ18-ZF, TRBIP1-ZF, TRBIP2-ZF and ASF1B-ZF. Error bar indicates standard error of three technical replicates. FIG. 15C shows CG (red bars), CHG (green bars), and CHH (blue bars) DNA methylation levels over FWA promoter regions in fwa, Col-0, and representative T1 lines of MBD2-ZF, ZF-SUVH7, SPT16-ZF, JMJ18-ZF, TRBIP1-ZF and ASF1B-ZF, measured by BS-PCR-seq. Pink vertical boxes indicate ZF binding sites. FIG. 15D shows relative DNA methylation level over FWA promoter region in fwa, Col-0 and three representative SSRP1-ZF and TRBIP2 T1 lines. Error bar indicates standard error of three technical replicates.

FIGS. 16A-16B show RNA-seq results for fwa compared to transgenic lines of ELF7-ZF, CPL2-ZF, MSI1-ZF, JMJ14-ZF, and LHP1-ZF over the endogenous gene of each respective transgene. 3′ UTRs of each zinc finger line were not included in the transgene. FIG. 16A shows screenshots of RNA-seq signals in two independent lines each of fwa and transgenic ELF7-ZF over the endogenous ELF7 locus. FIG. 16B shows screenshots of RNA-seq signals in two independent lines each of fwa and transgenic CPL2-ZF over the endogenous CPL2 locus. FIG. 16C shows screenshots of RNA-seq signals in two independent lines each of fwa and transgenic MSI1-ZF over the endogenous MSI1 locus. FIG. 16D shows screenshots of RNA-seq signals in two independent lines each of fwa and transgenic JMJ14-ZF over the endogenous JMJ14 locus. FIG. 16E shows screenshots of RNA-seq signals in two independent lines each of fwa and transgenic LHP1-ZF over the endogenous LHP1 locus.

FIGS. 17A-17D show ChIP-seq results for H3K27me3, H3K4me3, RNA Polymerase II, H3K9ac, H3K27ac, H3K27ac, H4K16ac, and H3 in fwa and negative control EYFP-ZF T2 transgenic plants. FIG. 17A shows screenshots of H3K27me3, H3K4me3, RNA Polymerase II, and H3 ChIP-seq signals over the FWA locus in fwa and T2 transgenic lines of EYFP-ZF. The FLAG-ZF ChIP-seq signals indicate ZF binding sites. FIG. 17B shows heatmaps and metaplots showing the normalized H3K27me3, H3K4me3, and RNA Polymerase II ChIP-seq signals over ZF off-target sites (n=6,091) and shuffle sites in EYFP-ZF T2 transgenic line versus fwa. FIG. 17C shows screen shots of H3K9ac, H3K14ac, H3K27ac, H4K16ac, and H3 ChIP-seq signals over the FWA locus in fwa and T2 transgenic lines of EYFP-ZF. FIG. 17D shows heatmaps and metaplots showing the normalized H3K9ac, H3K14ac, H3K27ac, H4K16ac ChIP-seq signals over ZF off-target sites (n=6,091) and shuffle sites in EYFP-ZF T2 transgenic line versus fwa.

FIGS. 18A-18D show removal of H3K4me3 and histone acetylation ChIP-seq signal over regions with high levels of pre-existing ChIP-seq signal. FIG. 18A shows heatmaps and metaplots showing the normalized H3K4me3 ChIP-seq signal over 3 clusters of zinc finger off-target sites in JMJ14-ZF and ELF7-ZF transgenic lines versus fwa (left panel). H3K4me3-cluster1, H3K4me3-cluster2, and H3K4me3-cluster3 represent high, medium, and low levels of pre-existing H3K4me3 ChIP-seq signal, respectively (right panel). FIG. 18B shows heatmaps and metaplots showing the normalized H3K9ac ChIP-seq signal over three clusters of zinc finger off-target sites in HD2A-ZF, HD2B-ZF, and HD2C-ZF transgenic lines versus fwa (left panel). H3K9ac-cluster1, H3K9ac-cluster2, and H3K9ac-cluster3 represent high, medium, and low levels of pre-existing H3K9ac ChIP-seq signal, respectively (right panel). FIG. 18C shows heatmaps and metaplots showing the normalized H3K27ac ChIP-seq signal over three clusters of zinc finger off-target sites in HD2A-ZF, HD2B-ZF, and HD2C-ZF transgenic lines versus fwa (left panel). H3K27ac-cluster1, H3K27ac-cluster2, and H3K27ac-cluster3 represent high, medium, and low levels of pre-existing H3K27ac ChIP-seq signal, respectively (right panel). FIG. 18D shows heatmaps and metaplots showing the normalized H4K16ac ChIP-seq signal over three clusters of zinc finger off-target sites in HD2A-ZF, HD2B-ZF, and HD2C-ZF transgenic lines versus fwa (left panel). H4K16ac-cluster1, H4K16ac-cluster2, and H4K16ac-cluster3 represent high, medium, and low levels of pre-existing H4K16ac ChIP-seq signal, respectively (right panel).

FIGS. 19A-19F show target gene silencing by MSI1-ZF, HDA6-ZF, and histone deacetylation. FIG. 19A shows screenshots of histone H3K9ac, H3K14ac, and H3 ChIP-seq signals over the FWA locus in fwa, HDA6-ZF, and MSI1-ZF. FIG. 19B shows heatmaps and metaplots showing the normalized H3K9ac (left two columns) and H3K14ac (right two columns) ChIP-seq signal over zinc finger off-target sites and zinc finger off-target sites shuffle in HDA6-ZF (first and third columns) and MSI1-ZF (second and fourth columns) T2 representative lines versus fwa. FIG. 19C shows heatmaps and metaplots showing normalized H3K9ac ChIP-seq signal over three clusters of zinc finger off-target sites in HDA6-ZF (left panel, first column) and MSI1-ZF (left panel, second column) versus fwa. H3K9ac-cluster1 (dark blue in metaplot, top heatmap), H3K9ac-cluster2 (light blue in metaplot, middle heatmap), and H3K9ac-cluster3 (yellow in metaplot, bottom heatmap) represent high, medium, and low levels of pre-existing H3K9ac ChIP-seq signal, respectively (right panel). FIG. 19D shows heatmaps and metaplots showing normalized H3K14ac ChIP-seq signal over three clusters of zinc finger off-target sites in HDA6-ZF (left panel, first column) and MSI1-ZF (left panel, second column) versus fwa. H3K14ac-cluster1 (dark blue in metaplot, top heatmap), H3K14ac-cluster2 (light blue in metaplot, middle heatmap), and H3K14ac-cluster3 (yellow in metaplot, bottom heatmap) represent high, medium, and low levels of pre-existing H3K14ac ChIP-seq signal, respectively (right panel). FIG. 19E shows screenshots indicating H3K9ac, H3K14ac, and H3 ChIP-seq signal over two representative zinc finger off-target sites in fwa and T2 transgenic lines of HDA6-ZF and MSI1-ZF.

FIGS. 20A-20D show a new machine learning model for predicting and providing the importance of varied chromatin features. FIG. 20A shows screenshots of FLAG-ZF ChIP-seq and RNA-seq in fwa, ChIP-seq signals of H3K4me3, H3K9ac, H3K14ac, H4K16ac, H3K27me3, and RNA Polymerase II in fwa, ATAC-seq signals in fwa, and CG, CHG, and CHH DNA methylation levels in fwa over two representative zinc finger targeting genes. FIG. 20B shows screenshots displaying RNA-seq levels of a representative zinc finger off-target gene AT3G13470 in fwa-1, HD2A-ZF, HD2B-ZF, HD2C-ZF, ELF7-ZF, fwa-2, LHP1-ZF, and CPL2-ZF. FLAG-ZF ChIP-seq signal indicates the zinc finger binding site. FIG. 20C shows a bar chart indicating the accuracy of cross validation in each ZF transgenic line, for CPL2-ZF, HD2A-ZF, HD2B-ZF, HD2C-ZF, HDA6-ZF, LHP1-ZF, MSI1-ZF, ZF-SUVH2, JMJ14-ZF, JMJ18-ZF, and ELF7-ZF. FIG. 20D shows line point charts displaying the variable importance of different chromatin features of ZF transgenic lines and inputs for machine learning. The chromatin features are expression level, ATAC-seq signal, H3K4me3 ChIP-seq signal, CG methylation level, GC content, H3K27me3 ChIP-seq signal, H3K9ac ChIP-seq signal, CHH methylation level, RNA Polymerase II ChIP-seq signal, CHH number, H3K27ac ChIP-seq signal, CHG number, CHG methylation number, H4K16ac ChIP-seq signal, and CG number. The ZF transgenic lines are HD2A-ZF (first row left), HD2B-ZF (first row right), HD2C-ZF (second row left), HDA6-ZF (second row right), JMJ14-ZF (third row left), JMJ18-ZF (third row right), LHP1-ZF (fourth row left), MSI1-ZF (fourth row right), ZF-SUVH2 (fifth row left), CPL2-ZF (fifth row right), and ELF7-ZF (bottom row left).

FIGS. 21A-21E show that some effectors also trigger gene silencing in SunTag system T1 and T2 plants. FIG. 21A shows Western blots showing the expression levels of dCas9 in SunTag-JMJ14 (top), SunTag-LHP1 (middle), and SunTag-ELF7 (bottom) in fwa (left) and fwa rdr6 (right) backgrounds. FIG. 21B shows dot plots displaying flowering time as measured by leaf number of fwa rdr6, Col-O, and the SunTag T1 transgenic lines SunTag-Ctrl, SunTag-JMJ14, SunTag-LHP1, SunTag-ELF7, SunTag-HD2C, SunTag-SUVH2, and SunTag-CPL2. FIG. 21C shows RT-qPCR results showing the relative mRNA level of FWA in fwa rdr6, Col-O, and the SunTag T1 transgenic lines SunTag-Ctrl, SunTag-JMJ14, SunTag-LHP1, SunTag-ELF7, SunTag-HD2C, SunTag-SUVH2, and SunTag-CPL2. Error bars indicate the mean standard error of three technical replicates of each sample. FIG. 21D shows dot plots displaying leaf numbers of Col-O, fwa rdr6, and the SunTag T2 transgenic lines SunTag-Ctrl, SunTag-JMJ14, SunTag-LHP1, SunTag-ELF7, and SunTag-HD2C. FIG. 21E shows qRT-PCR results showing the relative mRNA level of FWA in fwa rdr6 and the SunTag T2 transgenic lines SunTag-Ctrl, SunTag-JMJ14, SunTag-LHP1, SunTag-ELF7, and SunTag-HD2C. Error bars indicate the mean standard error of three technical replicates of each sample.

FIGS. 22A-22J show that TRB proteins interact and colocalize with JMJ14 over gene body regions. FIG. 22A shows a Western blot showing a Co-Immunoprecipitation (Co-IP) assay in F2 lines of Myc-JMJ14 crossed with each of FLAG-TRB3 (second column), FLAG-TRB2 (third column), and FLAG-TRB1 (fourth column). Myc-JMJ14 (first column) serves as a negative control for the anti-FLAG Western (left), a positive control for the anti-Myc Western of the input sample (top right), and a negative control for the anti-Myc FLAG-IP sample (bottom right). FIG. 22B shows a screenshot showing FLAG ChIP-seq signal in FLAG-TRB1 (top row), FLAG-TRB2 (second row), FLAG-TRB3 (third row) and FLAG-JMJ14 (bottom row) T2 lines over a representative co-targeted gene. FIG. 22C shows metaplots and heatmaps depicting the ChIP-seq signals of FLAG-TRB1 (first column), FLAG-TRB2 (middle column), and FLAG-TRB3 (right column) over FLAG-JMJ14 peaks (n=4,014) and shuffle peaks. FIG. 22D shows motif prediction results by Homer showing the top three binding motifs of TRB1 (top), TRB2 (middle), and TRB3 (bottom). FIG. 22E shows motif prediction results by Homer showing that JMJ14, TRB1, TRB2, and TRB3 are all predicted to target the CTTGnnnnnCAAG motif. FIG. 22F shows heatmaps and metaplots representing FLAG-JMJ14 (left), FLAG-TRB1 (middle) and H3K4me3 (right) ChIP-seq signals over TRB1 Cluster 1 (n=743) and Cluster 2 (n=4,822) peak proximal genes. FIG. 22G shows heatmaps and metaplots showing ChIP-seq signals of FLAG-TRB1 (left) and FLAG-JMJ14 (right) over FLAG-TRB1 Cluster 1 (n=892; blue in metaplot, top heatmap) and Cluster 2 (n=6,710; orange in metaplot, bottom heatmap) peaks. FIG. 22H shows heatmaps and metaplots showing ChIP-seq signals of H3K4me3 over FLAG-TRB1 Cluster 1 (n=892; blue in metaplot, top heatmap) and Cluster 2 (n=6,710; orange in metaplot, bottom heatmap) peaks. FIG. 22I shows a screenshot displaying the ChIP-seq signals of FLAG-TRB1 (first row), FLAG-TRB2 (second row), FLAG-TRB3 (third row), FLAG-JMJ14 (fourth row), H3K4me3 (fifth row), and H3K27me3 (bottom row) over a representative locus containing FLAG-TRB1 Cluster 1 and Cluster 2 peaks, indicated above the screenshot. FIG. 22J shows pie charts depicting the annotations of the FLAG-TRB1 Cluster 1 peaks (top panel) and Cluster 2 peaks (bottom panel). The annotation categories are promoter, 5′ UTR, 3′ UTR, 1st Exon, Other Exon, 1st Intron, Other Intron, Downstream (<=300), and Distal Intergenic.

FIGS. 23A-23D show upregulation of H3K4me3 ChIP-seq signals over JMJ14 and TRB co-targeting regions in both the trb1/2/3 triple mutant and jmj14-1 mutant. FIG. 23A shows metaplots and heatmaps showing the normalized H3K4me3 ChIP-seq levels of jmj14-1 and trb1/2/3 mutants versus Col-0 wild type plants, over FLAG-JMJ14 peaks and shuffle peaks (n=4,014). FIG. 23B shows metaplots and heatmaps showing H3K4me3 ChIP-seq signals of jmj14-1 (left) and trb1/2/3 (right) mutants versus Col-0 wild type plants over FLAG-TRB1 Cluster 1 peaks (teal in metaplot, top heatmap) and Cluster 1 shuffle peaks (orange in metaplot, bottom heatmap) (n=892). FIG. 23C shows metaplots and heatmaps showing H3K4me3 ChIP-seq signals of jmj14-1 (left) and trb1/2/3 (right) mutants versus Col-0 wild type plants over FLAG-TRB1 Cluster 2 peaks (teal in metaplot, top heatmap) and Cluster 2 shuffle peaks (orange in metaplot, bottom heatmap) (n=6,710). FIG. 23D shows screenshots displaying the FLAG ChIP-seq signals (blue or pink) of FLAG-TRB1 (first blue row), FLAG-TRB2 (second blue row), FLAG-TRB3 (third blue row), and FLAG-JMJ14 (pink row), the histone H3K4me3 ChIP-seq signals (orange) of Col-0 (first orange row), trb1/2/3 mutant (second orange row), and jmj14-1 mutant (third orange row), and the H3K27me3 ChIP-seq signals (teal) of Col-O (first teal row) and trb1/2/3 (second teal row) over two representative loci. The locus on the left has representative Cluster 1 peaks, annotated at the top. The locus on the right has a representative Cluster 2 peak, annotated at the top.

FIGS. 24A-24C show reduced H3K27me3 ChIP-seq signals in the trb1/2/3 mutant. FIG. 24A shows a bar chart indicating the number of regions with up- or down-regulated H3K27me3 ChIP-seq signals in trb1/2/3 mutant versus Col-O wild type plants. FIG. 24B shows a screenshot showing FLAG ChIP-seq signals (blue) in FLAG-TRB1 (first blue row), FLAG-TRB2 (second blue row), and FLAG-TRB3 (third blue row), and the H3K27me3 ChIP-seq signals (green) in Col-O (first green row) and trb1/2/3 mutant (second green row), over a representative region. FIG. 24C shows metaplots and heatmaps representing H3K27me3 ChIP-seq signals in trb1/2/3 mutant versus Col-O wildtype over FLAG-TRB1 peaks (blue in metaplot, top heatmap) and shuffle peaks (pink in metaplot, bottom heatmap) (left panel, n=7,602), FLAG-TRB2 peaks (blue in metaplot, top heatmap) and shuffle peaks (pink in metaplot, bottom heatmap) (middle panel, n=4,425), and FLAG-TRB3 peaks (blue in metaplot, top heatmap) and shuffle peaks (pink in metaplot, bottom heatmap) (right panel, n=2,440).

FIGS. 25A-25D show that up-regulated differentially expressed genes (DEGs) in trb1/2/3 mutant and jmj14-1 mutant were highly overlapped. FIG. 25A shows scatterplots showing the gene expression level in jmj14-1 (y-axis) versus Col-O (x-axis) (left panel), and in trb1/2/3 (y-axis) versus Col-O (x-axis) (right panel). Red dots highlight genes bound by JMJ14 (left panel) or TRB1 (right panel). FIG. 25B shows box plots depicting the expression level of FLAG-JMJ14 bound and up-regulated DEGs (blue, n=240), FLAG-TRB1 bound and up-regulated DEGs (green, n=325), and non-TRB1 nor JMJ14 bound genes (pink, n=12,948), in Col-O (first column per color), trb1/2/3 mutant (second column per color), and jmj14-1 mutant (third column per color). FIG. 25C shows a screenshot depicting the FLAG ChIP-seq signals (blue or pink) of FLAG-TRB1 (first blue row), FLAG-TRB2 (second blue row), FLAG-TRB3 (third blue row), and FLAG-JMJ14 (pink row), the histone H3K4me3 ChIP-seq signals (orange) of Col-O (first orange row), trb1/2/3 mutant (second orange row), and jmj14-1 mutant (third orange row), and the RNA-seq signals (green) of Col-O (first two green rows), trb1/2/3 triple mutant (third and fourth green rows), and jmj14-1 mutant (fifth and sixth green rows). FIG. 25D shows Venn diagrams showing the overlap (red) of all up-regulated DEGs (left panel) or JMJ14-TRB1 co-bound up-regulated DEGs (right panel) between the trb1/2/3 triple mutant (pink) and the jmj14-1 mutant (blue).

FIGS. 26A-26D show that up-regulated DEGs are associated with H3K4me3 induction. FIG. 26A shows a bar chart that indicates the number of up- (blue) and down-regulated (orange) DEGs in jmj14-1 mutant (left) and trb1/2/3 mutant (right), versus Col-0. FIG. 26B shows density of DEGs in jmj14-1 mutant (upper panel) and trb1/2/3 mutant (lower panel) over TRB1 and JMJ14 co-bound sites (blue) or non-binding sites (red). FIG. 26C shows metaplots and heatmaps representing normalized H3K4me3 ChIP-seq signals in jmj14-1 mutant versus Col-0 over jmj14-1 mutant up-regulated DEGs (blue in metaplot, top heatmap) or shuffle sites (pink in metaplot, bottom heatmap) (left, n=485), and in trb1/2/3 mutant versus Col-0 over trb1/2/3 mutant up-regulated DEGs (blue in metaplot, top heatmap) or shuffle sites (pink in metaplot, bottom heatmap) (right, n=1,688). FIG. 26D shows boxplots showing the log 2 value of Shannon Entropy of JMJ14 bound and up-regulated DEGs in jmj14-1 mutant (blue, n=240), TRB1 bound and up-regulated DEGs in trb1/2/3 mutant (yellow, n=325), and control genes not bound by JMJ14 or TRB (red, n=12,948).

FIGS. 27A-27F show methylation-independent silencing of FWA and early flowering phenotype in TRBIP1/2-ZF transgenic lines. FIG. 27A shows dot plots of flowering time measured by leaf number of fwa (first row), Col-0 (second row), four representative T2 lines of TRBIP1-ZF (third through sixth rows), and four representative T2 lines of TRBIP2-ZF (seventh through tenth rows). FIG. 27B shows RT-PCR results indicating the relative mRNA levels of FWA in fwa (purple), four representative lines of TRBIP1-ZF (blue), and four representative lines of TRBIP2-ZF (orange). FIG. 27C shows BS-PCR results indicating the DNA methylation level at the FWA promoter region in fwa (top row), Col-0 (second row), TRBIP1-ZF transgenic plant (third row), and TRBIP2-ZF transgenic plant (bottom row). FIG. 27D shows a screenshot of FLAG-ZF ChIP-seq (top row), H3K27me3 ChIP-seq (teal), H3K4me3 ChIP-seq (blue), and H3 ChIP-seq (red) in fwa (second, fourth, and sixth rows) and TRBIP1-ZF T2 transgenic plants (third, fifth, and seventh rows). FIG. 27E shows metaplots and heatmaps showing histone H3K4me3 ChIP-seq signal over zinc finger off target sites (light blue in metaplot, top heatmap) and shuffle sites (pink in metaplot, bottom heatmap) in TRBIP1-ZF versus fwa (n=6,091). FIG. 27F shows metaplots and heatmaps showing histone H3K27me3 ChIP-seq signal over zinc finger off target sites (light blue in metaplot, top heatmap) and shuffle sites (pink in metaplot, bottom heatmap) in TRBIP1-ZF versus fwa (n=6,091).

FIGS. 28A-28E show that co-targeting of TRBIP1 with MQ1 by the straight fusion method triggered synergistic earlier flowering than dCas9-MQ1 and TRBIP1-dCas9. FIG. 28A shows designs of TRBIP1-dCas9-MQ1 (top), TRBIP1-dCas9 (middle), and dCas9-MQ1 (bottom), using Xten and SV40 as linkers between TRBIP1 and dCas9, and between dCas9 and MQ1, respectively. FIG. 28B shows dot plots showing flowering times measured by leaf number of Col-0 (top row), fwa rdr6 (second row), dCas9-MQ1 T1 (third row), TRBIP1-dCas9 T1 (fourth row), and TRBIP1-dCas9-MQ1 T1 (bottom row). dCas9-MQ1 T1 (third row), TRBIP1-dCas9 T1 (fourth row), and TRBIP1-dCas9-MQ1 T1 (bottom row) are all in an fwa rdr6 background. FIG. 28C shows McrBC-qPCR results showing the relative DNA methylation level of fwa×rdr6 (first two columns), Col-O (third and fourth columns), 16 T1 transgenic lines of TRBIP1-dCas9-MQ1 (fifth through twentieth columns), 15 T1 transgenic lines of dCas9-MQ1 (twenty-first through thirty-fourth columns), and 12 T1 transgenic lines of TRBIP1-dCas9 (thirty-fifth through forty-sixth columns) in fwa rdr6 background. FIG. 28D shows an anti-FLAG Western blot (top) showing the protein expression level of T1 transgenic lines of FLAG-tagged TRBIP1-dCas9-MQ1 (red-labelled columns), FLAG-tagged dCas9-MQ1 (blue-labelled columns), and FLAG-tagged TRBIP1-dCas9 (pink-labelled columns). A negative control lane is indicated in green. Ponceau staining in shown for control (bottom). FIG. 28E shows RT-qPCR results showing the relative FWA expression levels of fwa×rdr6 (first through fourth columns), 12 T1 transgenic lines of TRBIP1-dCas9-MQ1 (fifth through sixteenth columns), 11 T1 transgenic lines of dCas9-MQ1 (seventeenth through twenty-seventh columns), and 12 T1 transgenic lines of TRBIP1-dCas9 (twenty-eighth through thirty-ninth columns) in an fwa rdr6 background.

FIGS. 29A-29E show the results of co-targeting TRB3, JMJ14, and MSI1 with MQ1 by the straight fusion method. FIG. 29A shows designs of TRB3-dCas9-MQ1 (left) and TRB3-dCas9 (right), using Xten and SV40 as linkers between TRB3 and dCas9, and between dCas9 and MQ1, respectively. FIG. 29B shows designs of JMJ14-dCas9-MQ1 (left) and JMJ14-dCas9 (right), using Xten and SV40 as linkers between JMJ14 and dCas9, and between dCas9 and MQ1, respectively. FIG. 29C shows designs of MSI1-dCas9-MQ1 (left) and MSI1-dCas9 (right), using Xten and SV40 as linkers between MSI1 and dCas9, and between dCas9 and MQ1, respectively. FIG. 29D shows McrBC-qPCR results showing the relative DNA methylation level of fwa×rdr6 (first two columns), Col-O (third and fourth columns), 8 T1 transgenic lines of TRB3-dCas9-MQ1 (fifth through twelfth columns), 8 T1 transgenic lines of TRB3-dCas9 (thirteenth through twentieth columns), 8 T1 transgenic lines of JMJ14-dCas9-MQ1 (twenty-first through twenty-eighth columns), 8 T1 transgenic lines of JMJ14-dCas9 (twenty-ninth through thirty-sixth columns), 8 T1 transgenic lines of MSI1-dCas9-MQ1 (thirty-seventh through forty-fourth columns), and 8 T1 transgenic lines of MSI1-dCas9 (forty-fifth through fifty-second columns) in fwa rdr6 background. FIG. 29E shows RT-qPCR results showing the relative FWA expression levels of fwa×rdr6 (first through third columns), 4 T1 transgenic lines of JMJ14-dCas9-MQ1 (fourth through seventh columns), 4 T1 transgenic lines of JMJ14-dCas9 (eighth through eleventh columns), 4 T1 transgenic lines of MSI1-dCas9-MQ1 (twelfth through fifteenth columns), 4 T1 transgenic lines of MSI1-dCas9 (sixteenth through nineteenth columns), 4 T1 transgenic lines of TRB3-dCas9-MQ1 (twentieth through twenty-third columns), and 4 T1 transgenic lines of TRB3-dCas9 (twenty-fourth through twenty-seventh columns) in an fwa rdr6 background.

FIGS. 30A-30D show that co-targeting TRBIP1 with MQ1 by the SunTag method triggered synergistic earlier flowering than SunTag-MQ1 and SunTag-TRBIP1. FIG. 30A shows designs of SunTag-MQ1-TRBIP1 (top), SunTag-TRBIP1 (middle), and SunTag-MQ1 (bottom). FIG. 30B shows dot plots depicting flowering time measured by leaf number of fwa rdr6 (first row), Col-0 (second row), T1 transgenic SunTag-TRBIP1 (third row), T1 transgenic SunTag-MQ1 (fourth row), T1 transgenic SunTag-MQ1-TRBIP1 (fifth row), T1 transgenic SunTag-MQ1-TRB3 (sixth row), T1 transgenic SunTag-MQ1-JMJ14 (seventh row), and T1 transgenic SunTag-MQ1-MSI1 (bottom row), where all SunTag lines are in an fwa rdr6 background. FIG. 30C shows McrBC-qPCR results showing the relative DNA methylation levels of fwa×rdr6 (first and second columns), Col-0 (third and fourth columns), 16 representative T1 transgenic lines of SunTag-MQ1-TRBIP1 (fifth through twentieth columns), and 16 representative T1 transgenic lines of SunTag-MQ1 (twenty-first through thirty-sixth columns), where all SunTag lines are in an fwa rdr6 background. FIG. 30D shows RT-qPCR results showing the relative FWA expression level of fwa×rdr6 (last two columns), 6 representative T1 transgenic lines of SunTag-MQ1 (first through sixth columns), and 6 representative T1 transgenic lines of SunTag-MQ1-TRBIP1 (seventh through twelfth columns), where all SunTag lines are in an fwa rdr6 background.

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### General Terms

The terminology used herein is for the purpose of describing particular embodiments and is not intended to be limiting.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “isolated” and “purified” as used herein refers to a material that is removed from at least one component with which it is naturally associated (e.g., removed from its original environment). The term “isolated,” when used in reference to an isolated protein, refers to a protein that has been removed from the culture medium of the host cell that expressed the protein. As such an isolated protein is free of extraneous or unwanted compounds (e.g., nucleic acids, native bacterial or other proteins, etc.).

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### Overview

The following description is presented to enable a person of ordinary skill in the art to make and use the various embodiments. Descriptions of specific devices, techniques, methods, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown.

The present disclosure relates to recombinant transcriptional repressor polypeptides that are capable of being targeted to specific loci, as well as methods of using these recombinant polypeptides for reducing expression of a target nucleic acid in plants. The present disclosure is based, at least in part, on Applicant's discovery that various polypeptides may be targeted to specific loci and that, once targeted, can facilitate a reduction in expression of the target nucleic acids. Accordingly, the present disclosure provides methods for targeting a recombinant polypeptide to a target nucleic acid, where the recombinant polypeptide contains a transcriptional repressor polypeptide and a targeting domain, and where the transcriptional repressor polypeptide is at least one of a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. Once targeted to the target nucleic acid, the recombinant polypeptide facilitates reduced expression of the target nucleic acid. Also provided are nucleic acids encoding the recombinant polypeptides, expression vectors containing nucleic acids that encode the recombinant polypeptides, plant cells containing the recombinant polypeptides, plants containing the recombinant polypeptides, and plants having reduced expression of a target nucleic acid as a consequence of having the recombinant polypeptides targeted to the target nucleic acid.

Each one of the aforementioned recombinant polypeptides may be expressed in a host cell individually or in various combinations to act to reduce expression of a target nucleic acid.

### Recombinant Polypeptides

Certain aspects of the present disclosure relate to recombinant polypeptides containing a transcriptional repressor polypeptide and a targeting domain. These recombinant polypeptides may be targeted to a target nucleic acid to facilitate reduced expression of the target nucleic acid. In addition to recombinant polypeptides containing a transcriptional repressor polypeptide and a targeting domain, these polypeptides may contain other features as described herein and as well be apparent to one of skill in the art. Other amino acid and/or polypeptide sequence features of the recombinant polypeptides may be used to provide additional functionality and/or features to the recombinant polypeptide including e.g. subcellular localization, downstream detection, etc. as will be readily apparent to one of skill in the art.

As used herein, a “polypeptide” is an amino acid sequence including a plurality of consecutive polymerized amino acid residues (e.g., at least about 15 consecutive polymerized amino acid residues). “Polypeptide” refers to an amino acid sequence, oligopeptide, peptide, protein, or portions thereof, and the terms “polypeptide” and “protein” are used interchangeably.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

A “recombinant” polypeptide, protein, or enzyme of the present disclosure may be a polypeptide, protein, or enzyme that may be encoded by e.g. a “recombinant nucleic acid” or “heterologous nucleic acid” or “recombinant polynucleotide.”

Recombinant polypeptides of the present disclosure that are composed of individual polypeptide domains may be described based on the individual polypeptide domains of the overall recombinant polypeptide. A domain in such a recombinant polypeptide refers to the particular stretches of contiguous amino acid sequences with a particular function or activity. For example, for a recombinant polypeptide that is a fusion of a transcriptional repressor polypeptide and a targeting domain, the contiguous amino acids that encode the transcriptional repressor polypeptide may be described as the “transcriptional repressor domain” in the overall recombinant polypeptide, and the contiguous amino acids that encode the targeting domain may be described as the “targeting domain” in the over recombinant polypeptide. Individual domains in an overall recombinant polypeptide may also be referred to as units of the recombinant polypeptide. Recombinant polypeptides that are composed of individual polypeptide domains may also be referred to as fusion polypeptides.

Fusion polypeptides of the present disclosure may contain an individual polypeptide domain that is in various N-terminal or C-terminal orientations relative to other individual polypeptide domains present in the fusion polypeptide. Fusion of individual polypeptide domains in fusion polypeptides may also be direct or indirect fusions. Direct fusions of individual polypeptide domains refer to direct fusion of the coding sequences of each respective individual polypeptide domain. In embodiments where the fusion is indirect, a linker domain or other contiguous amino acid sequence may separate the coding sequences of two individual polypeptide domains in a fusion polypeptide.

Polypeptides of the present disclosure may be detecting using antibodies. Techniques for detecting polypeptides using antibodies include, for example, enzyme linked immunosorbent assays (ELISAs), Western blots, immunoprecipitations, and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### Linkers

Various linkers may be used in the construction of recombinant polypeptides as described herein. In general, linkers are short peptides that separate the different domains in a multi-domain protein. They may play an important role in fusion proteins, affecting the crosstalk between the different domains, the yield of protein production, and the stability and/or the activity of the fusion proteins. Linkers are generally classified into 2 major categories: flexible or rigid. Flexible linkers are typically used when the fused domains require a certain degree of movement or interaction, and these linkers are usually composed of small amino acids such as, for example, glycine (G), serine (S) or proline (P).

The certain degree of movement between domains allowed by flexible linkers is an advantage in some fusion proteins. However, it has been reported that flexible linkers can sometimes reduce protein activity due to an inefficient separation of the two domains. In this case, rigid linkers may be used since they enforce a fixed distance between domains and promote their independent functions. A thorough description of several linkers has been provided in Chen X et al., 2013, Advanced Drug Delivery Reviews 65 (2013) 1357-1369).

Various linkers may be used in, for example, the construction of recombinant polypeptides as described herein. Linkers may be used to separate the coding sequences of a transcriptional repressor polypeptide and a targeting domain. For example, a variety of wiggly/flexible linkers, stiff/rigid linkers, short linkers, and long linkers may be used as described herein. Various linkers as described herein may be used in the construction of recombinant polypeptides as described herein.

A variety of shorter or longer linker regions are known in the art, for example corresponding to a series of glycine residues, a series of adjacent glycine-serine dipeptides, a series of adjacent glycine-glycine-serine tripeptides, or known linkers from other proteins. A flexible linker may include, for example, the amino acid sequence: SSGPPPGTG (SEQ ID NO: 213) and variants thereof. A rigid linker may include, for example, the amino acid sequence: AEAAAKEAAAKA (SEQ ID NO: 214) and variants thereof. The XTEN linker, SGSETPGTSESATPES (SEQ ID NO: 215) and variants thereof, described in Guiling et et al, 2014 (Nature Biotechnology 32, 577-582), may also be used.

### Nuclear Localization Signals (NLS)

Recombinant polypeptides of the present disclosure may contain one or more nuclear localization signals (NLS). Nuclear localization signals may also be referred to as nuclear localization sequences, domains, peptides, or other terms readily apparent to those of skill in the art. Nuclear localization signals are a translocation sequence that, when present in a polypeptide, direct that polypeptide to localize to the nucleus of a eukaryotic cell.

Various nuclear localization signals may be used in recombinant polypeptides of the present disclosure. For example, one or more SV40-type NLS or one or more REX NLS may be used in recombinant polypeptides. Recombinant polypeptides may also contain two or more tandem copies of a nuclear localization signal. For example, recombinant polypeptides may contain at least two, at least three, at least for, at least five, at least six, at least seven, at least eight, at least nine, or at least ten copies, either tandem or not, of a nuclear localization signal.

### Tags, Reporters, and Other Features

Recombinant polypeptides of the present disclosure may contain one or more tags that allow for e.g. purification and/or detection of the recombinant polypeptide. Various tags may be used herein and are well-known to those of skill in the art. Exemplary tags may include HA, GST, FLAG, MBP, etc., and multiple copies of one or more tags may be present in a recombinant polypeptide.

Recombinant polypeptides of the present disclosure may contain one or more reporters that allow for e.g. visualization and/or detection of the recombinant polypeptide. A reporter polypeptide encodes a protein that may be readily detectable due to its biochemical characteristics such as, for example, enzymatic activity or chemifluorescent features. Reporter polypeptides may be detected in a number of ways depending on the characteristics of the particular reporter. For example, a reporter polypeptide may be detected by its ability to generate a detectable signal (e.g. fluorescence), by its ability to form a detectable product, etc. Various reporters may be used herein and are well-known to those of skill in the art. Exemplary reporters may include GFP, GUS, mCherry, luciferase, etc., and multiple copies of one or more tags may be present in a recombinant polypeptide.

Recombinant polypeptides of the present disclosure may contain one or more polypeptide domains that serve a particular purpose depending on the particular goal/need. Recombinant polypeptides may contain translocation sequences that target the polypeptide to a particular cellular compartment or area. Suitable features will be readily apparent to those of skill in the art.

### Transcriptional Repressor Polypeptides

Certain aspects of the present disclosure relate to recombinant polypeptides containing a transcriptional repressor polypeptide and a targeting domain. Transcriptional repressor polypeptides may include, for example, a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. Recombinant polypeptides that contain a transcriptional repressor polypeptide may also be referred to as recombinant transcriptional repressor polypeptides (e.g. a recombinant polypeptide that contains a PHD1 polypeptide as the transcriptional repressor polypeptide may be referred to as a “recombinant PHD1 polypeptide”).

### PHD1 Polypeptides

Certain aspects of the present disclosure relate to PHD1 polypeptides. Recombinant PHD1 polypeptides of the present disclosure may be capable of being targeted to a specific nucleic acid sequence on a target nucleic acid and may be used in reducing the expression of a target nucleic acid, such as a gene, in plants.

PHD1 proteins are known in the art. PHD1 is a PHD finger containing protein, which is a subunit of MOM1 complex. The MOM1 complex include (MORPHEUS MOLECULE 1) MOM1, MOM2, ASI1-IMMUNOPRECIPITATED PROTEIN 3 (AIPP3), PROTEIN INHIBITOR OF ACTIVATED STAT LIKE 1, PIAL1, and PIAL2. The MOM1 complex has been shown to interact with and co-localize with MORC6 over some RNA-directed DNA methylation (RdDM) sites to maintain the DNA methylation. As described herein, PHD1-ZF triggered DNA methylation over FWA promoter region and some ZF off-target sites and led to gene silencing, which in turn restored early flowering phenotype in fwa epiallele.

In some embodiments, a PHD1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length PHD1 polypeptide. In some embodiments, PHD1 polypeptides include sequences with one or more amino acids removed from the consecutive amino acid sequence of an endogenous or wild-type full-length PHD1 polypeptide. In some embodiments, PHD1 polypeptides may include sequences with one or more amino acids replaced/substituted with an amino acid different from an endogenous or wild-type amino acid present at a given amino acid position in a consecutive amino acid sequence of an endogenous or wild-type full-length PHD1 polypeptide. In some embodiments, PHD1 polypeptides may include sequences with one or more amino acids added to an otherwise consecutive amino acid sequence of an endogenous or wild-type full-length PHD1 polypeptide.

Suitable PHD1 polypeptides may be identified from monocot and dicot plants. Examples of suitable PHD1 polypeptides may include, for example, those listed in Table 1, homologs thereof, and orthologs thereof.

TABLE 1
PHD1 Polypeptides

| Organism | Gene Name | SEQ ID NO: |
|---|---|---|
| Arabidopsis thaliana | NP_001117432.1 | 1 |
| Glycine max | XP_006589501.1 | 2 |
| Zea mays | AQK39401.1 | 3 |
| Oryza sativa | KAF2906777.1 | 4 |
| Sorghum bicolor | KAG0520158.1 | 5 |
| Cucumis sativus | XP_011659124.1 | 6 |
| Vitis vinifera | XP_010664973.1 | 7 |
| Gossypium hirsutum | XP_040942147.1 | 8 |
| Citrus sinensis | XP_024952436.1 | 9 |
| Populus trichocarpa | XP_002307485.2 | 10 |

In some embodiments, a PHD1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 1, 2, 3, 4, 5, 6, 7, 8, 9, and/or 10.

### PIAL1 Polypeptides

Certain aspects of the present disclosure relate to PIAL1 polypeptides. Recombinant PIAL1 polypeptides of the present disclosure may be capable of being targeted to a specific nucleic acid sequence on a target nucleic acid and may be used in reducing the expression of a target nucleic acid, such as a gene, in plants.

PIAL1 proteins are known in the art. PROTEIN INHIBITOR OF ACTIVATED STAT LIKE1 (PIAL1) PIAL1 and PIAL2 are PIAS-type small ubiquitin-related modifier (SUMO) E3 ligase like proteins, which are involved in the promotion of SUMO chain formation and are required for optimal activity of SCE1 (see e.g., Tomanov, Plant Cell, 2014). PIAL1 and PIAL2 contain SP-RING domains, SIM domains, and IND domains. RING and SIM domains are required for SUMO activity of PIAL1 and PIAL2, while IND domain is required for their interaction with MOM1. PIAL1, PIAL2, and MOM1 form a complex and mediate transcriptional silencing over heterochromatin regions. The silencing activity is independent of SUMO E3 ligase activity (see e.g., Han, Plant Cell, 2016). As described herein, PIAL1-ZF triggered DNA methylation over FWA promoter region and some ZF off-target sites and led to gene silencing, which in turn restored early flowering phenotype in fwa epiallele.

In some embodiments, a PIAL1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length PIAL1 polypeptide. In some embodiments, PIAL1 polypeptides include sequences with one or more amino acids removed from the consecutive amino acid sequence of an endogenous or wild-type full-length PIAL1 polypeptide. In some embodiments, PIAL1 polypeptides may include sequences with one or more amino acids replaced/substituted with an amino acid different from an endogenous or wild-type amino acid present at a given amino acid position in a consecutive amino acid sequence of an endogenous or wild-type full-length PIAL1 polypeptide. In some embodiments, PIAL1 polypeptides may include sequences with one or more amino acids added to an otherwise consecutive amino acid sequence of an endogenous or wild-type full-length PIAL1 polypeptide.

Suitable PIAL1 polypeptides may be identified from monocot and dicot plants. Examples of suitable PIAL1 polypeptides may include, for example, those listed in Table 2, homologs thereof, and orthologs thereof.

TABLE 2
PIAL1 Polypeptides

| Organism | Gene Name | SEQ ID NO: |
|---|---|---|
| Arabidopsis thaliana | NP_172366.3 | 11 |
| Glycine max | XP_014632565.1 | 12 |
| Zea mays | NP_001307038.1 | 13 |
| Oryza sativa | XP_015643308.1 | 14 |
| Sorghum bicolor | XP_021305330.1 | 15 |
| Cucumis sativus | XP_011654715.1 | 16 |
| Vitis vinifera | XP_010658435.1 | 17 |
| Gossypium hirsutum | XP_040930210.1 | 18 |
| Citrus sinensis | XP_006476486.1 | 19 |
| Populus trichocarpa | XP_024453412.1 | 20 |

In some embodiments, a PIAL1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 11, 12, 13, 14, 15, 16, 17, 18, 19, and/or 20.

### PIAL2 Polypeptides

Certain aspects of the present disclosure relate to PIAL2 polypeptides. Recombinant PIAL2 polypeptides of the present disclosure may be capable of being targeted to a specific nucleic acid sequence on a target nucleic acid and may be used in reducing the expression of a target nucleic acid, such as a gene, in plants.

PIAL2 proteins are known in the art. PIAL2 proteins are functionally redundant with PIAL1 proteins. However, PIAL2 proteins are more dominant than PIAL1 in heterochromatic transcriptional silencing (see e.g., Han, Plant Cell, 2016). PIAL2 proteins contain RING domains, IND domains, and SIM domains. PIAL2 proteins also possess SUMO E3 ligase activity (see e.g., Han, Plant Cell, 2016). PIAL2 directly interacts with MORC6, and the loss of function pial1/2 double mutant has been shown to reduce CHH DNA methylation regions that significantly overlap with morc6 mutant. PIAL1, PIAL2, and MOM1 complex also participate in the maintenance of heterochromatic TE stability, as well as in FWA transgene silencing. As described herein, PIAL2-ZF triggered DNA methylation over FWA promoter region and some ZF off-target sites and led to gene silencing, which in turn restored early flowering phenotype in fwa epiallele.

In some embodiments, a PIAL2 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length PIAL2 polypeptide. In some embodiments, PIAL2 polypeptides include sequences with one or more amino acids removed from the consecutive amino acid sequence of an endogenous or wild-type full-length PIAL2 polypeptide. In some embodiments, PIAL2 polypeptides may include sequences with one or more amino acids replaced/substituted with an amino acid different from an endogenous or wild-type amino acid present at a given amino acid position in a consecutive amino acid sequence of an endogenous or wild-type full-length PIAL2 polypeptide. In some embodiments, PIAL2 polypeptides may include sequences with one or more amino acids added to an otherwise consecutive amino acid sequence of an endogenous or wild-type full-length PIAL2 polypeptide.

Suitable PIAL2 polypeptides may be identified from monocot and dicot plants. Examples of suitable PIAL2 polypeptides may include, for example, those listed in Table 3, homologs thereof, and orthologs thereof.

TABLE 3
PIAL2 Polypeptides

| Organism | Gene Name | SEQ ID NO: |
|---|---|---|
| Arabidopsis thaliana | NP_198973.3 | 21 |
| Glycine max | XP_006573790.1 | 22 |
| Zea mays | NP_001307038.1 | 23 |
| Oryza sativa | XP_015643308.1 | 24 |
| Sorghum bicolor | XP_021305330.1 | 25 |
| Cucumis sativus | XP_011654714.1 | 26 |
| Vitis vinifera | XP_019071879.1 | 27 |
| Gossypium hirsutum | XP_040955207.1 | 28 |
| Citrus sinensis | XP_006476486.1 | 29 |
| Populus trichocarpa | XP_024453412.1 | 30 |

In some embodiments, a PIAL2 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 21, 22, 23, 24, 25, 26, 27, 28, 29, and/or 30.

### TRB1 Polypeptides

Certain aspects of the present disclosure relate to TRB1 polypeptides. Recombinant TRB1 polypeptides of the present disclosure may be capable of being targeted to a specific nucleic acid sequence on a target nucleic acid and may be used in reducing the expression of a target nucleic acid, such as a gene, in plants.

TRB1 proteins are known in the art. Arabidopsis has three Telomeric Repeat Binding factors, TRB1, TRB2, and TRB3, which are close homologs and are functionally redundant. The double mutants of any of TRB1, TRB2, and TRB3 have no physical phenotype, while the triple mutant shows strong dwarf and infertile phenotypes. TRB proteins are known by their role in telomere binding and protection. TRB proteins bind double-stranded telomeric DNA repeats through the N-terminal Myb domain (see e.g., Schrumpfova, Plant Mol. Biol., 2014). TRB proteins are also known to recruit Polycomb Repressive Complex 2 (PRC2) for deposition of the repressive histone mark H3K27me3 (see e.g., Bloomer, PNAS, 2020; Zhou, Nat. Genet., 2018). TRB proteins haven also been shown to form multiple functionally redundant protein complexes with PWWP proteins, EPCR1/2 proteins, and ARID proteins, which are named PEAT complexes. These PEAT complexes are also involved in heterochromatin silencing (see e.g., Tan, EMBO, 2018). However, TRB proteins have been shown to localize over the entirety of the genome and are not limited to telomeric regions. As a Myb transcription factor, TRBs bind promoter regions and regulate gene expression. In addition, TRB proteins interact with JMJ14 proteins and its interacting proteins NAC050 and NAC052, which in turn regulate the demethylation of positive histone mark H3K4me3. As described herein, TRB1-ZF triggered gene silencing of FWA and some other ZF off-target genes without adding DNA methylation, and FWA repression restored early flowering phenotype in fwa epiallele. Histone H3K27me3 deposition and H3K4me3 demethylation were observed over FWA and some ZF off-target sites in TRB1-ZF.

In some embodiments, a TRB1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length TRB1 polypeptide. In some embodiments, TRB1 polypeptides include sequences with one or more amino acids removed from the consecutive amino acid sequence of an endogenous or wild-type full-length TRB1 polypeptide. In some embodiments, TRB1 polypeptides may include sequences with one or more amino acids replaced/substituted with an amino acid different from an endogenous or wild-type amino acid present at a given amino acid position in a consecutive amino acid sequence of an endogenous or wild-type full-length TRB1 polypeptide. In some embodiments, TRB1 polypeptides may include sequences with one or more amino acids added to an otherwise consecutive amino acid sequence of an endogenous or wild-type full-length TRB1 polypeptide.

Suitable TRB1 polypeptides may be identified from monocot and dicot plants. Examples of suitable TRB1 polypeptides may include, for example, those listed in Table 4, homologs thereof, and orthologs thereof.

TABLE 4
TRB1 Polypeptides

| Organism | Gene Name | SEQ ID NO: |
|---|---|---|
| Arabidopsis thaliana | NP_564559.1 | 31 |
| Glycine max | XP_003546290.1 | 32 |
| Zea mays | ACG38321.1 | 33 |
| Oryza sativa | XP_015613057.1 | 34 |
| Sorghum bicolor | XP_002457295.1 | 35 |
| Cucumis sativus | XP_031739750.1 | 36 |
| Vitis vinifera | XP_002266866.2 | 37 |
| Gossypium hirsutum | XP_016691598.2 | 38 |
| Citrus sinensis | XP_006486912.1 | 39 |
| Populus trichocarpa | XP_024464338.1 | 40 |

In some embodiments, a TRB1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 31, 32, 33, 34, 35, 36, 37, 38, 39, and/or 40.

### TRB2 Polypeptides

Certain aspects of the present disclosure relate to TRB2 polypeptides. Recombinant TRB2 polypeptides of the present disclosure may be capable of being targeted to a specific nucleic acid sequence on a target nucleic acid and may be used in reducing the expression of a target nucleic acid, such as a gene, in plants.

TRB2 proteins are known in the art. TRB2 proteins are shown to be a close homolog of TRB1 and TRB3. In addition, TRB2 protein function is redundant with TRB1 and TRB3, as described in TRB1 part above. As described herein, TRB2-ZF triggered gene silencing of FWA and some other ZF off-target genes without adding DNA methylation, and FWA repression restored early flowering phenotype in fwa epiallele. Histone H3K27me3 deposition and H3K4me3 demethylation were observed over FWA and some ZF off-target sites in TRB2-ZF.

In some embodiments, a TRB2 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length TRB2 polypeptide. In some embodiments, TRB2 polypeptides include sequences with one or more amino acids removed from the consecutive amino acid sequence of an endogenous or wild-type full-length TRB2 polypeptide. In some embodiments, TRB2 polypeptides may include sequences with one or more amino acids replaced/substituted with an amino acid different from an endogenous or wild-type amino acid present at a given amino acid position in a consecutive amino acid sequence of an endogenous or wild-type full-length TRB2 polypeptide. In some embodiments, TRB2 polypeptides may include sequences with one or more amino acids added to an otherwise consecutive amino acid sequence of an endogenous or wild-type full-length TRB2 polypeptide.

Suitable TRB2 polypeptides may be identified from monocot and dicot plants. Examples of suitable TRB2 polypeptides may include, for example, those listed in Table 5, homologs thereof, and orthologs thereof.

TABLE 5
TRB2 Polypeptides

| Organism | Gene Name | SEQ ID NO: |
|---|---|---|
| Arabidopsis thaliana | AAL73441.1 | 41 |
| Glycine max | XP_003517350.1 | 42 |
| Zea mays | NP_001141858.1 | 43 |
| Oryza sativa | XP_015613057.1 | 44 |
| Sorghum bicolor | XP_002455864.1 | 45 |
| Cucumis sativus | XP_011657903.1 | 46 |
| Vitis vinifera | XP_002266866.2 | 47 |
| Gossypium hirsutum | XP_016697329.2 | 48 |
| Citrus sinensis | XP_006466449.1 | 49 |
| Populus trichocarpa | XP_002320656.2 | 50 |

In some embodiments, a TRB2 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 41, 42, 43, 44, 45, 46, 47, 48, 49, and/or 50.

### TRB3 Polypeptides

Certain aspects of the present disclosure relate to TRB3 polypeptides. Recombinant TRB3 polypeptides of the present disclosure may be capable of being targeted to a specific nucleic acid sequence on a target nucleic acid and may be used in reducing the expression of a target nucleic acid, such as a gene, in plants.

TRB3 proteins are known in the art. TRB3 protein are shown to be a close homolog of TRB1 and TRB2. In addition, TRB3 protein function is redundant with TRB1 and TRB2, as described in TRB1 part above. As described herein, ZF-TRB3 triggered gene silencing of FWA and some other ZF off-target genes without adding DNA methylation, and FWA repression restored early flowering phenotype in fwa epiallele. Histone H3K27me3 deposition and H3K4me3 demethylation were observed over FWA and some ZF off-target sites in ZF-TRB3.

In some embodiments, a TRB3 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length TRB3 polypeptide. In some embodiments, TRB3 polypeptides include sequences with one or more amino acids removed from the consecutive amino acid sequence of an endogenous or wild-type full-length TRB3 polypeptide. In some embodiments, TRB3 polypeptides may include sequences with one or more amino acids replaced/substituted with an amino acid different from an endogenous or wild-type amino acid present at a given amino acid position in a consecutive amino acid sequence of an endogenous or wild-type full-length TRB3 polypeptide. In some embodiments, TRB3 polypeptides may include sequences with one or more amino acids added to an otherwise consecutive amino acid sequence of an endogenous or wild-type full-length TRB3 polypeptide.

Suitable TRB3 polypeptides may be identified from monocot and dicot plants. Examples of suitable TRB3 polypeptides may include, for example, those listed in Table 6, homologs thereof, and orthologs thereof.

TABLE 6
TRB3 Polypeptides

| Organism | Gene Name | SEQ ID NO: |
|---|---|---|
| Arabidopsis thaliana | NP_001326904.1 | 51 |
| Glycine max | XP_003517350.1 | 52 |
| Zea mays | ONM40899.1 | 53 |
| Oryza sativa | XP_015613057.1 | 54 |
| Sorghum bicolor | XP_002455864.1 | 55 |
| Cucumis sativus | XP_011657903.1 | 56 |
| Vitis vinifera | XP_002266866.2 | 57 |
| Gossypium hirsutum | XP_016697329.2 | 58 |
| Citrus sinensis | XP_024947953.1 | 59 |
| Populus trichocarpa | XP_002320656.2 | 60 |

In some embodiments, a TRB3 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 51, 52, 53, 54, 55, 56, 57, 58, 59, and/or 60.

### MSI1 Polypeptides

Certain aspects of the present disclosure relate to MSI1 polypeptides. Recombinant MSI1 polypeptides of the present disclosure may be capable of being targeted to a specific nucleic acid sequence on a target nucleic acid and may be used in reducing the expression of a target nucleic acid, such as a gene, in plants.

MSI1 proteins are known in the art. MULTICOPY SUPRESSOR OF IRA1 (MSI1) is a histone binding WD-40 domain containing protein, which is one of the core subunits of PRC2 complex, which is a homolog of Drosophila p55 protein. The PRC2 complex is involved in the deposition of the negative histone mark H3K27me3 for the maintenance of transcriptional repression. Additionally, MSI1 can bind to histone H4. MSI1 has been shown to interact with chromatin assembly factor CAF-1 and histone deacetylases, suggesting a role in nucleosome assembly (see e.g., Hennig, Trends Cell Biol., 2005). As described herein, MSI1-ZF triggered gene silencing of FWA and some other ZF off-target genes without adding DNA methylation, and FWA repression restored early flowering phenotype in fwa epiallele. Histone H3K27me3 deposition was observed over FWA and some ZF off-target sites in MSI1-ZF.

In some embodiments, a MSI1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length MSI1 polypeptide. In some embodiments, MSI1 polypeptides include sequences with one or more amino acids removed from the consecutive amino acid sequence of an endogenous or wild-type full-length MSI1 polypeptide. In some embodiments, MSI1 polypeptides may include sequences with one or more amino acids replaced/substituted with an amino acid different from an endogenous or wild-type amino acid present at a given amino acid position in a consecutive amino acid sequence of an endogenous or wild-type full-length MSI1 polypeptide. In some embodiments, MSI1 polypeptides may include sequences with one or more amino acids added to an otherwise consecutive amino acid sequence of an endogenous or wild-type full-length MSI1 polypeptide.

Suitable MSI1 polypeptides may be identified from monocot and dicot plants. Examples of suitable MSI1 polypeptides may include, for example, those listed in Table 7, homologs thereof, and orthologs thereof.

TABLE 7
MSI1 Polypeptides

| Organism | Gene Name | SEQ ID NO: |
|---|---|---|
| Arabidopsis thaliana | NP_200631.1 | 61 |
| Glycine max | NP_001237595.2 | 62 |
| Zea mays | NP_001105556.1 | 63 |
| Oryza sativa | XP_015632366.1 | 64 |
| Sorghum bicolor | XP_002464182.1 | 65 |
| Cucumis sativus | XP_004133950.1 | 66 |
| Vitis vinifera | XP_002265142.1 | 67 |
| Gossypium hirsutum | XP_016705767.2 | 68 |
| Citrus sinensis | XP_006478521.1 | 69 |
| Populus trichocarpa | XP_002320581.1 | 70 |

In some embodiments, a MSI1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 61, 62, 63, 64, 65, 66, 67, 68, 69, and/or 70.

### LHP1 Polypeptides

Certain aspects of the present disclosure relate to LHP1 polypeptides. Recombinant LHP1 polypeptides of the present disclosure may be capable of being targeted to a specific nucleic acid sequence on a target nucleic acid and may be used in reducing the expression of a target nucleic acid, such as a gene, in plants.

LHP1 proteins are known in the art. LIKE HETEROCHROMATIN PROTEIN (LHP)1 is the Arabidopsis homologue of HP1, which is a component of PRC1. HP1 proteins are characterized by the presence of a chromodomain and a chromo-shadow domain. These chromodomains bind to repressive histone mark H3K27me3 throughout the genome (see e.g., Turck, Plant Mol. Biol., 2007). LHP1 is required for epigenetic gene silencing of several flowering regulating genes controlled by PRC2, such as FLOWERING LOCUS T (FT), FLOWERING LOCUS C (FLC), AGAMOUS (AG), and APETALA 3 (AP3) (see e.g., Turck, Plant Mol. Biol., 2007). LHP1 has also been found to interact with MSI1. Loss of function mutant lhp1 has been shown to obtain similar changes in gene expression and defective phenotype, suggesting that LHP1 might recruit PRC2 complex to the H3K27me3 sites in a positive feedback loop (see e.g., Derkacheva, EMBO, 2013). As described herein, LHP1-ZF triggered gene silencing of FWA and some other ZF off-target genes without adding DNA methylation, and FWA repression restored early flowering phenotype in fwa epiallele. Histone H3K27me3 deposition was observed over FWA and some ZF off-target sites in LHP1-ZF.

In some embodiments, a LHP1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length LHP1 polypeptide. In some embodiments, LHP1 polypeptides include sequences with one or more amino acids removed from the consecutive amino acid sequence of an endogenous or wild-type full-length LHP1 polypeptide. In some embodiments, LHP1 polypeptides may include sequences with one or more amino acids replaced/substituted with an amino acid different from an endogenous or wild-type amino acid present at a given amino acid position in a consecutive amino acid sequence of an endogenous or wild-type full-length LHP1 polypeptide. In some embodiments, LHP1 polypeptides may include sequences with one or more amino acids added to an otherwise consecutive amino acid sequence of an endogenous or wild-type full-length LHP1 polypeptide.

Suitable LHP1 polypeptides may be identified from monocot and dicot plants. Examples of suitable LHP1 polypeptides may include, for example, those listed in Table 8, homologs thereof, and orthologs thereof.

TABLE 8
LHP1 Polypeptides

| Organism | Gene Name | SEQ ID NO: |
|---|---|---|
| Arabidopsis thaliana | NP_197271.1 | 71 |
| Glycine max | XP_003548606.1 | 72 |
| Zea mays | AQK66310.1 | 73 |
| Oryza sativa | XP_015614736.1 | 74 |
| Sorghum bicolor | XP_002489226.1 | 75 |
| Cucumis sativus | XP_004145010.1 | 76 |
| Vitis vinifera | XP_002273726.1 | 77 |
| Gossypium hirsutum | XP_016754644.1 | 78 |
| Citrus sinensis | XP_006484756.1 | 79 |
| Populus trichocarpa | XP_002325456.1 | 80 |

In some embodiments, a LHP1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 71, 72, 73, 74, 75, 76, 77, 78, 79, and/or 80.

### HD2A Polypeptides

Certain aspects of the present disclosure relate to HD2A polypeptides. Recombinant HD2A polypeptides of the present disclosure may be capable of being targeted to a specific nucleic acid sequence on a target nucleic acid and may be used in reducing the expression of a target nucleic acid, such as a gene, in plants.

HD2A proteins are known in the art. HD2 proteins are plant specific HD-tuin type histone deacetylases (HDACs), and include HD2A, HD2B, HD2C, and HD2D. IP-MS data has shown that these HD2 proteins form a complex. Additionally, it has been shown that HD2 proteins can interact with other types of HDAC proteins, such as HDA6 and HDA19, suggesting that these HDAC proteins might functionally associate with each other for histone deacetylation (see e.g., Luo, Plant Signal. Behav., 2012b). In addition, it has been shown that HD2A, HD2B, and HD2C tethered to a GAL4 DNA binding domain provides target gene silencing activity. HD2 proteins have a conserved amino terminal EFWG region, which is important for gene silencing activity (see e.g., Wu, Plant Mol. Biol., 2003; Zhou, Plant Mol. Biol., 2004). This observation is consistent with data that showed that ZF108 fused with HD2A, HD2B, and HD2C led to silencing of FWA and ZF off-target gene. HD2A contains a single C2H2 type zinc finger domain, which may be involved in DNA-binding or protein-protein interactions. HD2A is also known to be required for deacetylation of histone H3K9, and thus is involved in rRNA dosage control and nucleolar dominance (see e.g., Lawrence, Mol. Cell, 2004). ChIP-seq data has also shown that H3K9 acetylation level is significantly increased over HD2A-targeting sites in hd2a, hd2b, and hd2c mutants. As described herein, HD2A-ZF triggered gene silencing of FWA and some other ZF off-target genes without adding DNA methylation, and restored early flowering phenotype in fwa epiallele. Histone H3K9, H3K27 and H4K16 deacetylation was observed over FWA and some ZF off-target sites in HD2A-ZF.

In some embodiments, a HD2A polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length HD2A polypeptide. In some embodiments, HD2A polypeptides include sequences with one or more amino acids removed from the consecutive amino acid sequence of an endogenous or wild-type full-length HD2A polypeptide. In some embodiments, HD2A polypeptides may include sequences with one or more amino acids replaced/substituted with an amino acid different from an endogenous or wild-type amino acid present at a given amino acid position in a consecutive amino acid sequence of an endogenous or wild-type full-length HD2A polypeptide. In some embodiments, HD2A polypeptides may include sequences with one or more amino acids added to an otherwise consecutive amino acid sequence of an endogenous or wild-type full-length HD2A polypeptide.

Suitable HD2A polypeptides may be identified from monocot and dicot plants. Examples of suitable HD2A polypeptides may include, for example, those listed in Table 9, homologs thereof, and orthologs thereof.

TABLE 9
HD2A Polypeptides

| Organism | Gene Name | SEQ ID NO: |
|---|---|---|
| Arabidopsis thaliana | NP_566872.1 | 81 |
| Glycine max | NP_001235884.1 | 82 |
| Zea mays | XP_008655442.1 | 83 |
| Oryza sativa | AAW57802.1 | 84 |
| Sorghum bicolor | XP_002458956.1 | 85 |
| Cucumis sativus | XP_004145780.1 | 86 |
| Vitis vinifera | XP_010654035.1 | 87 |
| Gossypium hirsutum | XP_016671982.1 | 88 |
| Citrus sinensis | XP_006484979.1 | 89 |
| Populus trichocarpa | XP_002313645.2 | 90 |

In some embodiments, a HD2A polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 81, 82, 83, 84, 85, 86, 87, 88, 89, and/or 90.

### HD2B Polypeptides

Certain aspects of the present disclosure relate to HD2B polypeptides. Recombinant HD2B polypeptides of the present disclosure may be capable of being targeted to a specific nucleic acid sequence on a target nucleic acid and may be used in reducing the expression of a target nucleic acid, such as a gene, in plants.

HD2B proteins are known in the art. HD2B proteins are another member of HD2 protein family, as described in HD2A part above. HD2B proteins contain a conserved amino acid region (EFWG) that is important for its role in targeting gene silencing. In addition, HD2B also contains glutamate-rich regions. It has also been shown that suppression of HD2B is important for seed dormancy (see e.g., Yano, Plant Mol. Biol., 2013). As described herein, HD2B-ZF triggered gene silencing of FWA and some other ZF off-target genes without adding DNA methylation, and restored early flowering phenotype in fwa epiallele. Histone H3K9, H3K27 and H4K16 deacetylation was observed over FWA and some ZF off-target sites in HD2B-ZF.

In some embodiments, a HD2B polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length HD2B polypeptide. In some embodiments, HD2B polypeptides include sequences with one or more amino acids removed from the consecutive amino acid sequence of an endogenous or wild-type full-length HD2B polypeptide. In some embodiments, HD2B polypeptides may include sequences with one or more amino acids replaced/substituted with an amino acid different from an endogenous or wild-type amino acid present at a given amino acid position in a consecutive amino acid sequence of an endogenous or wild-type full-length HD2B polypeptide. In some embodiments, HD2B polypeptides may include sequences with one or more amino acids added to an otherwise consecutive amino acid sequence of an endogenous or wild-type full-length HD2B polypeptide.

Suitable HD2B polypeptides may be identified from monocot and dicot plants. Examples of suitable HD2B polypeptides may include, for example, those listed in Table 10, homologs thereof, and orthologs thereof.

TABLE 10
HD2B Polypeptides

| Organism | Gene Name | SEQ ID NO: |
|---|---|---|
| Arabidopsis thaliana | NP_851056.1 | 91 |
| Glycine max | NP_001241996.1 | 92 |
| Zea mays | NP_001334129.1 | 93 |
| Oryza sativa | AAF70196.1 | 94 |
| Sorghum bicolor | XP_002441441.1 | 95 |
| Cucumis sativus | XP_004145780.1 | 96 |
| Vitis vinifera | XP_002270966.2 | 97 |
| Gossypium hirsutum | XP_016750931.1 | 98 |
| Citrus sinensis | XP_006484979.1 | 99 |
| Populus trichocarpa | XP_006381322.1 | 100 |

In some embodiments, a HD2B polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 91, 92, 93, 94, 95, 96, 97, 98, 99, and/or 100.

### HD2C Polypeptides

Certain aspects of the present disclosure relate to HD2C polypeptides. Recombinant HD2C polypeptides of the present disclosure may be capable of being targeted to a specific nucleic acid sequence on a target nucleic acid and may be used in reducing the expression of a target nucleic acid, such as a gene, in plants.

HD2C proteins are known in the art. HD2C protein belongs to the HD2 protein family, as described above. HD2C proteins contain a conserved EFWG region that is important for its silencing activity. Moreover, HD2C proteins also contain a zinc finger domain at the C-terminal and an aspartate-rich region in the middle. It has been shown that HD2C proteins mediate histone H4K16 deacetylation, which in turn modulates rRNA at the transcriptional level. In addition, HD2C proteins directly bind to pre-rRNAs and small nucleolar RNAs to regulate rRNA methylation at the posttranscriptional level (see e.g., Chen, Plant Cell, 2018). It has also been shown that hd2c mutant decrease tolerance to ABA and salt as HD2C interacts with HDA6 to repress the expression of ABA responsive genes, ABI1 and ABI2, probably through histone deacetylation at H3K9 and H3K14 (see e.g., Luo, J. Exp. Bot., 2012a). As described herein, HD2C-ZF triggered gene silencing of FWA and some other ZF off-target genes without adding DNA methylation, and restored early flowering phenotype in fwa epiallele. Histone H3K9, H3K27 and H4K16 deacetylation was observed over FWA and some ZF off-target sites in HD2C-ZF.

In some embodiments, a HD2C polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length HD2C polypeptide. In some embodiments, HD2C polypeptides include sequences with one or more amino acids removed from the consecutive amino acid sequence of an endogenous or wild-type full-length HD2C polypeptide. In some embodiments, HD2C polypeptides may include sequences with one or more amino acids replaced/substituted with an amino acid different from an endogenous or wild-type amino acid present at a given amino acid position in a consecutive amino acid sequence of an endogenous or wild-type full-length HD2C polypeptide. In some embodiments, HD2C polypeptides may include sequences with one or more amino acids added to an otherwise consecutive amino acid sequence of an endogenous or wild-type full-length HD2C polypeptide.

Suitable HD2C polypeptides may be identified from monocot and dicot plants. Examples of suitable HD2C polypeptides may include, for example, those listed in Table 11, homologs thereof, and orthologs thereof.

TABLE 11
HD2C Polypeptides

| Organism | Gene Name | SEQ ID NO: |
|---|---|---|
| Arabidopsis thaliana | NP_195994.3 | 101 |
| Glycine max | NP_001240859.1 | 102 |
| Zea mays | AQK95837.1 | 103 |
| Oryza sativa | XP_015639422.1 | 104 |
| Sorghum bicolor | XP_021305986.1 | 105 |
| Cucumis sativus | XP_004137868.1 | 106 |
| Vitis vinifera | XP_010654035.1 | 107 |
| Gossypium hirsutum | XP_016671982.1 | 108 |
| Citrus sinensis | XP_006484979.1 | 109 |
| Populus trichocarpa | ABK93977.1 | 110 |

In some embodiments, a HD2C polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 101, 102, 103, 104, 105, 106, 107, 108, 109, and/or 110.

### ELF7 Polypeptides

Certain aspects of the present disclosure relate to ELF7 polypeptides. Recombinant ELF7 polypeptides of the present disclosure may be capable of being targeted to a specific nucleic acid sequence on a target nucleic acid and may be used in reducing the expression of a target nucleic acid, such as a gene, in plants.

ELF7 proteins are known in the art. EARLY FLOWERING 7 (ELF7) is a homolog of RNA polymerase II (Pol II) Associated Factor 1 (PAF1), which contains a Paf1 domain. ELF7, together with VERNALIZATION INDEPENDENCE 4 (VIP4), VIP5, VIP6 (ELF8) and CDC73 form the Arabidopsis PAF1 complex (PAF1C). PAF1C associates with Pol II transcript elongation complex, suggesting that PAF1C might contribute to the transcription elongation step (see e.g., Antosz, Plant Cell, 2017). This is consistent with data suggesting that ELF7 interacts with Pol II. As described herein, it has also been shown that ELF7-ZF is able to hold Pol II at ZF targeting sites, which leads to target gene silencing by inhibiting Pol II transcription elongation. Additionally, it has been shown that the elf⁷ mutant displays an early flowering phenotype, which is probably caused by FLC suppression. ELF7 might promote FLC expression through H3K4me3 accumulation (see e.g., He, Genes Dev., 2004).

In some embodiments, a ELF7 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length ELF7 polypeptide. In some embodiments, ELF7 polypeptides include sequences with one or more amino acids removed from the consecutive amino acid sequence of an endogenous or wild-type full-length ELF7 polypeptide. In some embodiments, ELF7 polypeptides may include sequences with one or more amino acids replaced/substituted with an amino acid different from an endogenous or wild-type amino acid present at a given amino acid position in a consecutive amino acid sequence of an endogenous or wild-type full-length ELF7 polypeptide. In some embodiments, ELF7 polypeptides may include sequences with one or more amino acids added to an otherwise consecutive amino acid sequence of an endogenous or wild-type full-length ELF7 polypeptide.

Suitable ELF7 polypeptides may be identified from monocot and dicot plants. Examples of suitable ELF7 polypeptides may include, for example, those listed in Table 12, homologs thereof, and orthologs thereof.

TABLE 12
ELF7 Polypeptides

| Organism | Gene Name | SEQ ID NO: |
|---|---|---|
| Arabidopsis thaliana | NP_178091.2 | 111 |
| Glycine max | XP_040873128.1 | 112 |
| Zea mays | AQK50750.1 | 113 |
| Oryza sativa | XP_015650443.1 | 114 |
| Sorghum bicolor | XP_002445079.1 | 115 |
| Cucumis sativus | XP_004141783.2 | 116 |
| Vitis vinifera | XP_002278075.3 | 117 |
| Gossypium hirsutum | XP_040932278.1 | 118 |
| Citrus sinensis | XP_024957203.1 | 119 |
| Populus trichocarpa | XP_002303312.3 | 120 |

In some embodiments, a ELF7 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 111, 112, 113, 114, 115, 116, 117, 118, 119, and/or 120.

### CPL2 Polypeptides

Certain aspects of the present disclosure relate to CPL2 polypeptides. Recombinant CPL2 polypeptides of the present disclosure may be capable of being targeted to a specific nucleic acid sequence on a target nucleic acid and may be used in reducing the expression of a target nucleic acid, such as a gene, in plants.

CPL2 proteins are known in the art. CARBOXYL-TERMINAL DOMAIN (CTD) PHOSPHATASE-LIKE 2 (CPL2) is a CTD phosphatase that dephosphorylates the Ser5-PO4 of Pol II. CPL2 proteins are members of haloacid dehydrogenase (HAD) like superfamily, which includes enzymes such as phosphatases, phosphonatases, P-type ATPases, among others. CPL2 proteins have a FCP1 homology domain, which is required for phosphatase enzyme activity. CPL2 also has a double-stranded RNA-binding domain (dsRBD) to allow DNA binding. It has also been shown that CPL2 forms a complex with AIPP3, PHD2, and PHD3, which serves as a reader of H3K27me3 and unmodified H3K4 (see e.g., Qian, J. Integr. Plant Biol., 2021; Zhang, Nat. Comm., 2020). As described herein, CPL2-ZF triggers gene silencing of FWA as well as ZF targeting sites via Pol II Ser5 dephosphorylation.

In some embodiments, a CPL2 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length CPL2 polypeptide. In some embodiments, CPL2 polypeptides include sequences with one or more amino acids removed from the consecutive amino acid sequence of an endogenous or wild-type full-length CPL2 polypeptide. In some embodiments, CPL2 polypeptides may include sequences with one or more amino acids replaced/substituted with an amino acid different from an endogenous or wild-type amino acid present at a given amino acid position in a consecutive amino acid sequence of an endogenous or wild-type full-length CPL2 polypeptide. In some embodiments, CPL2 polypeptides may include sequences with one or more amino acids added to an otherwise consecutive amino acid sequence of an endogenous or wild-type full-length CPL2 polypeptide.

Suitable CPL2 polypeptides may be identified from monocot and dicot plants. Examples of suitable CPL2 polypeptides may include, for example, those listed in Table 13, homologs thereof, and orthologs thereof.

TABLE 13
CPL2 Polypeptides

| Organism | Gene Name | SEQ ID NO: |
|---|---|---|
| Arabidopsis thaliana | NP_001190199.1 | 121 |
| Glycine max | KAG4377735.1 | 122 |
| Zea mays | AQK73018.1 | 123 |
| Oryza sativa | XP_025878125.1 | 124 |
| Sorghum bicolor | XP_002452510.1 | 125 |
| Cucumis sativus | XP_004147918.1 | 126 |
| Vitis vinifera | XP_010654171.1 | 127 |
| Gossypium hirsutum | XP_016671865.1 | 128 |
| Citrus sinensis | XP_006481008.1 | 129 |
| Populus trichocarpa | XP_006373980.2 | 130 |

In some embodiments, a CPL2 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 121, 122, 123, 124, 125, 126, 127, 128, 129, and/or 130.

### MBD2 Polypeptides

Certain aspects of the present disclosure relate to MBD2 polypeptides. Recombinant MBD2 polypeptides of the present disclosure may be capable of being targeted to a specific nucleic acid sequence on a target nucleic acid and may be used in reducing the expression of a target nucleic acid, such as a gene, in plants.

MBD2 proteins are known in the art. METHYL-CpG-BINDING DOMAIN PROTEIN 2 (MBD2) is a homolog of human MBD proteins that serve as CG methyl readers. Arabidopsis MBD2 proteins contain a CW-type zinc finger and an MBD binding domain. As described herein, it has been shown that MBD2-ZF triggers gene silencing of FWA and restored early flowering phenotype in a DNA-methylation independent manner. However, the endogenous function of MBD2 is still unknown.

In some embodiments, a MBD2 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length MBD2 polypeptide. In some embodiments, MBD2 polypeptides include sequences with one or more amino acids removed from the consecutive amino acid sequence of an endogenous or wild-type full-length MBD2 polypeptide. In some embodiments, MBD2 polypeptides may include sequences with one or more amino acids replaced/substituted with an amino acid different from an endogenous or wild-type amino acid present at a given amino acid position in a consecutive amino acid sequence of an endogenous or wild-type full-length MBD2 polypeptide. In some embodiments, MBD2 polypeptides may include sequences with one or more amino acids added to an otherwise consecutive amino acid sequence of an endogenous or wild-type full-length MBD2 polypeptide.

Suitable MBD2 polypeptides may be identified from monocot and dicot plants. Examples of suitable MBD2 polypeptides may include, for example, those listed in Table 14, homologs thereof, and orthologs thereof.

TABLE 14
MBD2 Polypeptides

| Organism | Gene Name | SEQ ID NO: |
|---|---|---|
| Arabidopsis thaliana | NP_001190421.1 | 131 |
| Glycine max | XP_006583579.1 | 132 |
| Zea mays | NP_001105176.1 | 133 |
| Oryza sativa | XP_015611936.1 | 134 |
| Sorghum bicolor | XP_021308486.1 | 135 |
| Cucumis sativus | XP_004150390.1 | 136 |
| Vitis vinifera | XP_002263085.1 | 137 |
| Gossypium hirsutum | XP_016690230.1 | 138 |
| Citrus sinensis | XP_006474318.1 | 139 |
| Populus trichocarpa | XP_006372215.2 | 140 |

In some embodiments, a MBD2 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 131, 132, 133, 134, 135, 136, 137, 138, 139, and/or 140.

### SUVH7 Polypeptides

Certain aspects of the present disclosure relate to SUVH7 polypeptides. Recombinant SUVH7 polypeptides of the present disclosure may be capable of being targeted to a specific nucleic acid sequence on a target nucleic acid and may be used in reducing the expression of a target nucleic acid, such as a gene, in plants.

SUVH7 proteins are known in the art. SU(VAR)3-9 HOMOLOG 7 (SUVH7) encodes a SET domain protein. SUVH7 has an AT-hook motif, pre-SET domain, SET domain, post-SET domain, and SRA domain. Arabidopsis has 10 SUVH genes, some of which were known as histone methyltransferases, such as SUVH4, SUVH5, and SUVH6 (see e.g., Jackson, Plant Physiol., 2002). The other SUVHs, for example, SUVH1 and SUVH3 are known as DNA methylation readers (see e.g., Harris, Science, 2018). As described herein, it has been shown that ZF-SUVH7 triggers FWA gene silencing and restored early flowering phenotype in a DNA-methylation independent way. However, the endogenous function of SUVH7 is still unknown.

In some embodiments, a SUVH7 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length SUVH7 polypeptide. In some embodiments, SUVH7 polypeptides include sequences with one or more amino acids removed from the consecutive amino acid sequence of an endogenous or wild-type full-length SUVH7 polypeptide. In some embodiments, SUVH7 polypeptides may include sequences with one or more amino acids replaced/substituted with an amino acid different from an endogenous or wild-type amino acid present at a given amino acid position in a consecutive amino acid sequence of an endogenous or wild-type full-length SUVH7 polypeptide. In some embodiments, SUVH7 polypeptides may include sequences with one or more amino acids added to an otherwise consecutive amino acid sequence of an endogenous or wild-type full-length SUVH7 polypeptide.

Suitable SUVH7 polypeptides may be identified from monocot and dicot plants. Examples of suitable SUVH7 polypeptides may include, for example, those listed in Table 15, homologs thereof, and orthologs thereof.

TABLE 15
SUVH7 Polypeptides

| Organism | Gene Name | SEQ ID NO: |
|---|---|---|
| Arabidopsis thaliana | NP_564036.1 | 141 |
| Glycine max | XP_003546685.1 | 142 |
| Zea mays | PWZ13563.1 | 143 |
| Oryza sativa | XP_015616962.1 | 144 |
| Sorghum bicolor | XP_002459560.1 | 145 |
| Cucumis sativus | XP_004144645.1 | 146 |
| Vitis vinifera | XP_002273935.1 | 147 |
| Gossypium hirsutum | XP_040965752.1 | 148 |
| Citrus sinensis | XP_006471267.1 | 149 |
| Populus trichocarpa | XP_024453196.1 | 150 |

In some embodiments, a SUVH7 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 141, 142, 143, 144, 145, 146, 147, 148, 149, and/or 150.

### SSRP1 Polypeptides

Certain aspects of the present disclosure relate to SSRP1 polypeptides. Recombinant SSRP1 polypeptides of the present disclosure may be capable of being targeted to a specific nucleic acid sequence on a target nucleic acid and may be used in reducing the expression of a target nucleic acid, such as a gene, in plants.

SSRP1 proteins are known in the art. Structure Specific Recognition Protein 1 (SSRP1) protein is a component of histone chaperone (Facilitates Chromatin Transcription) FACT complex. SSRP1 proteins contain an N-terminal domain (NT/D), which is required for heterodimerization with another FACT subunit, SPT16. SSRP1 proteins also have a middle domain, acidic region, HMG-box domain (HMG), and a nuclear localization signal. FACT complex interacts with core histones and DNA, and it associates with Pol II transcription elongation complex (see e.g., Antosz, Plant Cell, 2017), suggesting its role in Pol II transcription elongation. FACT complex has been known to regulate nucleosome dynamics, which is important to maintain chromatin structure and prevent cryptic transcription. FACT complex contributes to the transition of seeds from dormancy to germination, and the transition of plants from vegetative to reproductive state by regulating the expression of DOG1 and FLC. FACT complex associates with DNA demethylase DEMETER to facilitate DNA demethylation and gene imprinting during reproduction (see e.g., Frost, PNAS, 2018).

In some embodiments, a SSRP1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length SSRP1 polypeptide. In some embodiments, SSRP1 polypeptides include sequences with one or more amino acids removed from the consecutive amino acid sequence of an endogenous or wild-type full-length SSRP1 polypeptide. In some embodiments, SSRP1 polypeptides may include sequences with one or more amino acids replaced/substituted with an amino acid different from an endogenous or wild-type amino acid present at a given amino acid position in a consecutive amino acid sequence of an endogenous or wild-type full-length SSRP1 polypeptide. In some embodiments, SSRP1 polypeptides may include sequences with one or more amino acids added to an otherwise consecutive amino acid sequence of an endogenous or wild-type full-length SSRP1 polypeptide.

Suitable SSRP1 polypeptides may be identified from monocot and dicot plants. Examples of suitable SSRP1 polypeptides may include, for example, those listed in Table 16, homologs thereof, and orthologs thereof.

TABLE 16
SSRP1 Polypeptides

| Organism | Gene Name | SEQ ID NO: |
|---|---|---|
| Arabidopsis thaliana | NP_189515.1 | 151 |
| Glycine max | XP_003517023.1 | 152 |
| Zea mays | NP_001334293.1 | 153 |
| Oryza sativa | XP_015617055.1 | 154 |
| Sorghum bicolor | XP_002457217.1 | 155 |
| Cucumis sativus | XP_004147459.1 | 156 |
| Vitis vinifera | XP_002282538.1 | 157 |
| Gossypium hirsutum | XP_040933411.1 | 158 |
| Citrus sinensis | XP_006482545.1 | 159 |
| Populus trichocarpa | XP_024444051.1 | 160 |

In some embodiments, a SSRP1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 151, 152, 153, 154, 155, 156, 157, 158, 159, and/or 160.

### SPT16 Polypeptides

Certain aspects of the present disclosure relate to SPT16 polypeptides. Recombinant SPT16 polypeptides of the present disclosure may be capable of being targeted to a specific nucleic acid sequence on a target nucleic acid and may be used in reducing the expression of a target nucleic acid, such as a gene, in plants.

SPT16 proteins are known in the art. Suppressor of Ty16 (SPT16) is a FACT subunit that forms a heterodimer with SSRP1. SPT16 proteins, as described above, have an N-terminal domain (NT/D), dimerization domain, middle domain, and C-terminal domain. FACT complex plays a critical role in regulating nucleosome dynamics and Pol II transcription elongation, as described above. As described herein, it was shown that both SSRP1-ZF and SPT16-ZF can trigger FWA gene silencing and restored early flowering phenotype of fwa in a DNA-methylation independent manner.

In some embodiments, a SPT16 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length SPT16 polypeptide. In some embodiments, SPT16 polypeptides include sequences with one or more amino acids removed from the consecutive amino acid sequence of an endogenous or wild-type full-length SPT16 polypeptide. In some embodiments, SPT16 polypeptides may include sequences with one or more amino acids replaced/substituted with an amino acid different from an endogenous or wild-type amino acid present at a given amino acid position in a consecutive amino acid sequence of an endogenous or wild-type full-length SPT16 polypeptide. In some embodiments, SPT16 polypeptides may include sequences with one or more amino acids added to an otherwise consecutive amino acid sequence of an endogenous or wild-type full-length SPT16 polypeptide.

Suitable SPT16 polypeptides may be identified from monocot and dicot plants. Examples of suitable SPT16 polypeptides may include, for example, those listed in Table 17, homologs thereof, and orthologs thereof.

TABLE 17
SPT16 Polypeptides

| Organism | Gene Name | SEQ ID NO: |
|---|---|---|
| Arabidopsis thaliana | NP_001329034.1 | 161 |
| Glycine max | XP_003552890.1 | 162 |
| Zea mays | PWZ21428.1 | 163 |
| Oryza sativa | XP_015635636.1 | 164 |
| Sorghum bicolor | XP_002466155.1 | 165 |
| Cucumis sativus | XP_011658313.1 | 166 |
| Vitis vinifera | RVW22100.1 | 167 |
| Gossypium hirsutum | XP_016665922.1 | 168 |
| Citrus sinensis | XP_006480296.1 | 169 |
| Populus trichocarpa | XP_002318930.2 | 170 |

In some embodiments, a SPT16 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 161, 162, 163, 164, 165, 166, 167, 168, 169, and/or 170.

### JMJ18 Polypeptides

Certain aspects of the present disclosure relate to JMJ18 polypeptides. Recombinant JMJ18 polypeptides of the present disclosure may be capable of being targeted to a specific nucleic acid sequence on a target nucleic acid and may be used in reducing the expression of a target nucleic acid, such as a gene, in plants.

JMJ18 proteins are known in the art. JUMONJI DOMAIN-CONTAINING PROTEIN 18 (JMJ18) is a JmjC domain containing protein that act as a histone H3K4 demethylases. JMJ18 proteins also contain a C5HC2-type zinc finger, JmjN domain, “FY-rich” domain N-terminal (FYRN), and “FY-rich” domain C-terminal (FYRC). JMJ18 has been shown to repress FLC expression though histone demethylation of H3K4me2 and H3K4me3 (see e.g., Yang, Plant Mol. Biol., 2012). As described herein, it was shown that JMJ18-ZF triggers FWA silencing and restored early flowering phenotype of fwa in a DNA-methylation independent manner.

In some embodiments, a JMJ18 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length JMJ18 polypeptide. In some embodiments, JMJ18 polypeptides include sequences with one or more amino acids removed from the consecutive amino acid sequence of an endogenous or wild-type full-length JMJ18 polypeptide. In some embodiments, JMJ18 polypeptides may include sequences with one or more amino acids replaced/substituted with an amino acid different from an endogenous or wild-type amino acid present at a given amino acid position in a consecutive amino acid sequence of an endogenous or wild-type full-length JMJ18 polypeptide. In some embodiments, JMJ18 polypeptides may include sequences with one or more amino acids added to an otherwise consecutive amino acid sequence of an endogenous or wild-type full-length JMJ18 polypeptide.

Suitable JMJ18 polypeptides may be identified from monocot and dicot plants. Examples of suitable JMJ18 polypeptides may include, for example, those listed in Table 18, homologs thereof, and orthologs thereof.

TABLE 18
JMJ18 Polypeptides

| Organism | Gene Name | SEQ ID NO: |
|---|---|---|
| Arabidopsis thaliana | NP_001185118.1 | 171 |
| Glycine max | XP_006580234.1 | 172 |
| Zea mays | NP_001352824.1 | 173 |
| Oryza sativa | XP_015640426.2 | 174 |
| Sorghum bicolor | XP_021305129.1 | 175 |
| Cucumis sativus | XP_011659340.1 | 176 |
| Vitis vinifera | XP_010655858.1 | 177 |
| Gossypium hirsutum | XP_016684356.2 | 178 |
| Citrus sinensis | XP_006468391.1 | 179 |
| Populus trichocarpa | XP_024454603.1 | 180 |

In some embodiments, a JMJ18 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 171, 172, 173, 174, 175, 176, 177, 178, 179, and/or 180.

### TRBIP1 Polypeptides

Certain aspects of the present disclosure relate to TRBIP1 polypeptides. Recombinant TRBIP1 polypeptides of the present disclosure may be capable of being targeted to a specific nucleic acid sequence on a target nucleic acid and may be used in reducing the expression of a target nucleic acid, such as a gene, in plants.

TRBIP1 proteins are known in the art. TRB Interacting Protein 1 (TRBIP1 AT4G35510) interacts with TRB proteins. As described herein, it was shown that TRBIP1-ZF triggers gene silencing in FWA in a DNA-methylation independent manner. Additionally, TRBI1 proteins are annotated as PHD finger-like proteins in The Arabidopsis Information Resource (TAIR) database. However, the endogenous function of TRBIP1 proteins have not been elucidated.

In some embodiments, a TRBIP1 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length TRBIP1 polypeptide. In some embodiments, TRBIP1 polypeptides include sequences with one or more amino acids removed from the consecutive amino acid sequence of an endogenous or wild-type full-length TRBIP1 polypeptide. In some embodiments, TRBIP1 polypeptides may include sequences with one or more amino acids replaced/substituted with an amino acid different from an endogenous or wild-type amino acid present at a given amino acid position in a consecutive amino acid sequence of an endogenous or wild-type full-length TRBIP1 polypeptide. In some embodiments, TRBIP1 polypeptides may include sequences with one or more amino acids added to an otherwise consecutive amino acid sequence of an endogenous or wild-type full-length TRBIP1 polypeptide.

Suitable TRBIP1 polypeptides may be identified from monocot and dicot plants. Examples of suitable TRBIP1 polypeptides may include, for example, those listed in Table 19, homologs thereof, and orthologs thereof.

TABLE 19
TRBIP1 Polypeptides

| Organism | Gene Name | SEQ ID NO: |
|---|---|---|
| Arabidopsis thaliana | NP_195276.3 | 181 |
| Glycine max | XP_003517132.1 | 182 |
| Setaria italica | XP_004966523.1 | 183 |
| Spinacia oleracea | XP_021839485.1 | 184 |
| Sorghum bicolor | KAG0516341.1 | 185 |
| Cucumis sativus | XP_011650244.1 | 186 |
| Vitis vinifera | XP_002277317.1 | 187 |
| Gossypium hirsutum | XP_016726278.1 | 188 |
| Citrus sinensis | XP_006467090.1 | 189 |
| Populus trichocarpa | XP_002306433.1 | 190 |

In some embodiments, a TRBIP1 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 181, 182, 183, 184, 185, 186, 187, 188, 189, and/or 190.

### TRBIP2 Polypeptides

Certain aspects of the present disclosure relate to TRBIP2 polypeptides. Recombinant TRBIP2 polypeptides of the present disclosure may be capable of being targeted to a specific nucleic acid sequence on a target nucleic acid and may be used in reducing the expression of a target nucleic acid, such as a gene, in plants.

TRBIP2 proteins are known in the art. TRB Interacting Protein 2 (TRBIP2 AT2G17540) interacts with TRB proteins and is a close homolog of TRBIP1. Both TRBIP1 and TRBIP2 are plant specific proteins, but their endogenous function has not been studied yet. As described herein, it was shown that TRBIP2 proteins can trigger the silencing of FWA and ZF-target genes in a DNA-methylation independent manner.

In some embodiments, a TRBIP2 polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length TRBIP2 polypeptide. In some embodiments, TRBIP2 polypeptides include sequences with one or more amino acids removed from the consecutive amino acid sequence of an endogenous or wild-type full-length TRBIP2 polypeptide. In some embodiments, TRBIP2 polypeptides may include sequences with one or more amino acids replaced/substituted with an amino acid different from an endogenous or wild-type amino acid present at a given amino acid position in a consecutive amino acid sequence of an endogenous or wild-type full-length TRBIP2 polypeptide. In some embodiments, TRBIP2 polypeptides may include sequences with one or more amino acids added to an otherwise consecutive amino acid sequence of an endogenous or wild-type full-length TRBIP2 polypeptide.

Suitable TRBIP2 polypeptides may be identified from monocot and dicot plants. Examples of suitable TRBIP2 polypeptides may include, for example, those listed in Table 20, homologs thereof, and orthologs thereof.

TABLE 20
TRBIP2 Polypeptides

| Organism | Gene Name | SEQ ID NO: |
|---|---|---|
| Arabidopsis thaliana | NP_001118339.1 | 191 |
| Glycine max | XP_003517132.1 | 192 |
| Theobroma cacao | EOX90646.1 | 193 |
| Spinacia oleracea | XP_021839485.1 | 194 |
| Cajanus cajan | KYP34966.1 | 195 |
| Cucumis sativus | XP_011650244.1 | 196 |
| Vitis vinifera | XP_002277317.1 | 197 |
| Gossypium hirsutum | XP_016692244.1 | 198 |
| Citrus sinensis | XP_006467090.1 | 199 |
| Populus trichocarpa | XP_002306433.1 | 200 |

In some embodiments, a TRBIP2 polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 191, 192, 193, 194, 195, 196, 197, 198, 199, and/or 200.

### ASF1B Polypeptides

Certain aspects of the present disclosure relate to ASF1B polypeptides. Recombinant ASF1B polypeptides of the present disclosure may be capable of being targeted to a specific nucleic acid sequence on a target nucleic acid and may be used in reducing the expression of a target nucleic acid, such as a gene, in plants.

ASF1B proteins are known in the art. ANTI-SILENCING FUNCTION 1B (ASF1B) is a histone H3/H4 chaperone and is a homolog of ASF1A. ASF1 proteins belong to ASF1-like superfamily, which are involved in chromatin assembly and disassembly. In yeast and metazoans, ASF1 proteins facilitate replication-dependent H3.1 deposition and replication-independent H3.3 deposition. In Arabidopsis, ASF1 proteins associate with acetylated histone H3 and H4, as well as the HAM superfamily histone acetyltransferases, which contribute to cell cycle control and DNA repair (see e.g., Lario, Plant Phys., 2013). As described herein, ASF1B-ZF inhibited FWA expression and restored early flowering phenotype in a DNA methylation independent manner.

In some embodiments, a ASF1B polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, or 241 or more consecutive amino acids of an endogenous or wild-type full-length ASF1B polypeptide. In some embodiments, ASF1B polypeptides include sequences with one or more amino acids removed from the consecutive amino acid sequence of an endogenous or wild-type full-length ASF1B polypeptide. In some embodiments, ASF1B polypeptides may include sequences with one or more amino acids replaced/substituted with an amino acid different from an endogenous or wild-type amino acid present at a given amino acid position in a consecutive amino acid sequence of an endogenous or wild-type full-length ASF1B polypeptide. In some embodiments, ASF1B polypeptides may include sequences with one or more amino acids added to an otherwise consecutive amino acid sequence of an endogenous or wild-type full-length ASF1B polypeptide.

Suitable ASF1B polypeptides may be identified from monocot and dicot plants. Examples of suitable ASF1B polypeptides may include, for example, those listed in Table 21, homologs thereof, and orthologs thereof.

TABLE 21
ASF1B Polypeptides

| Organism | Gene Name | SEQ ID NO: |
|---|---|---|
| Arabidopsis thaliana | NP_198627.1 | 201 |
| Glycine max | NP_001235672.1 | 202 |
| Zea mays | XP_008656015.1 | 203 |
| Oryza sativa | XP_015639753.1 | 204 |
| Sorghum bicolor | XP_002440209.1 | 205 |
| Cucumis sativus | XP_004150672.1 | 206 |
| Vitis vinifera | XP_002283949.1 | 207 |
| Gossypium hirsutum | XP_016696772.1 | 208 |
| Citrus sinensis | XP_006468797.1 | 209 |
| Populus trichocarpa | XP_024454731.1 | 210 |

In some embodiments, a ASF1B polypeptide of the present disclosure has an amino acid sequence with at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 201, 202, 203, 204, 205, 206, 207, 208, 209, and/or 210.

### Co-Targeting

Certain aspects of the present disclosure relate to co-targeting a target nucleic acid with 1) one or more of a transcriptional repressor polypeptide (e.g. a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide) and 2) a DNA methyltransferase polypeptide (e.g. an MQ1 polypeptide). An exemplary MQ1 polypeptide is set forth in SEQ ID NO: 212. Other suitable DNA methyltransferases for use in co-targeting a target nucleic acid as described in the present disclosure are well-known to those of skill in the art.

Co-targeting a target nucleic acid with 1) one or more of a transcriptional repressor polypeptide and 2) a DNA methyltransferase polypeptide may result in increased efficiency of methylation and/or reduced expression of the target nucleic acid. In some embodiments involving co-targeting of the target nucleic acid, the transcriptional repressor polypeptide is a TRBIP1 polypeptide and the DNA methyltransferase polypeptide is an MQ1 polypeptide.

In embodiments involving co-targeting a target nucleic acid with 1) one or more of a transcriptional repressor polypeptide (e.g. a TRBIP1 polypeptide) and 2) a DNA methyltransferase polypeptide (e.g. an MQ1 polypeptide), the target nucleic acid may experience an increase in DNA methylation of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, or about 300% or more as compared to a corresponding control (e.g. a nucleic acid targeted with only a transcriptional repressor polypeptide as described herein).

In embodiments involving co-targeting a target nucleic acid with 1) one or more of a transcriptional repressor polypeptide (e.g. a TRBIP1 polypeptide) and 2) a DNA methyltransferase polypeptide (e.g. an MQ1 polypeptide), the target nucleic acid may experience a decrease in transcriptional expression of about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a corresponding control (e.g. a nucleic acid targeted with only a transcriptional repressor polypeptide as described herein).

### Targeting Domains

Certain aspects of the present disclosure relate to recombinant polypeptides that contain a targeting domain and are capable of being targeted to a target nucleic acid. A targeting domain generally refers to a polypeptide or amino acid sequence that is able to facilitate or is involved in facilitating, either directly or indirectly, targeting of a recombinant polypeptide to a target nucleic acid sequence. For example, the targeting domain may directly confer the specific targeting functionality of the recombinant polypeptide to the target nucleic acid, or the targeting domain may be associated with or interact with another agent that confers the specific targeting functionality of the recombinant polypeptide to the target nucleic acid. In some embodiments, the targeting domain may associate with a DNA-binding polypeptide that is able to be targeted to a target nucleic acid. Suitable targeting domains for use in the present disclosure are described herein and will be readily apparent to one of skill in the art.

### DNA-Binding Domains

In some embodiments, the targeting domain is or may include a DNA-binding domain or have DNA-binding activity. In some embodiments, this DNA-binding activity is achieved through a heterologous DNA-binding domain (e.g. binds with a sequence affinity other than that of a DNA-binding domain that may be present in the endogenous protein). In some embodiments, recombinant polypeptides of the present disclosure contain a DNA-binding domain. Recombinant polypeptides of the present disclosure may contain one DNA binding domain or they may contain more than one DNA-binding domain. Heterologous DNA-binding domains may be recombinantly fused to a transcriptional repressor polypeptide of the present disclosure such that the transcriptional repressor is then targeted to a specific nucleic acid sequence and can facilitate reduced expression and/or silencing of the specific nucleic acid.

In some embodiments, the DNA-binding domain is a zinc finger domain. A zinc finger domain generally refers to a DNA-binding protein domain that contains zinc fingers, which are small protein structural motifs that can coordinate one or more zinc ions to help stabilize their protein folding. Zinc fingers were first identified as DNA-binding motifs (Miller et al., 1985), and numerous other variations of them have been characterized. Recent progress has been made that allows the engineering of DNA-binding proteins that specifically recognize any desired DNA sequence. For example, it was shown that a three-finger zinc finger protein could be constructed to block the expression of a human oncogene that was transformed into a mouse cell line (Choo and Klug, 1994).

Zinc fingers can generally be classified into several different structural families and typically function as interaction modules that bind DNA, RNA, proteins, or small molecules. Suitable zinc finger domains of the present disclosure may contain two, three, four, five, six, seven, eight, or nine zinc fingers. Examples of suitable zinc finger domains may include, for example, Cys2His2 (C2H2) zinc finger domains, C-x8-C-x5-C-x3-H (CCCH) zinc finger domains, multi-cysteine zinc finger domains, and zinc binuclear cluster domains.

In some embodiments, the DNA-binding domain binds a specific nucleic acid sequence. For example, the DNA-binding domain may bind a sequence that is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, or a high number of nucleotides in length.

In some embodiments, a recombinant polypeptide of the present disclosure further contains two N-terminal CCCH zinc finger domains. In some embodiments, the zinc finger domain is an engineered zinc finger array, such as a C2H2 zinc finger array. Engineered arrays of C2H2 zinc fingers can be used to create DNA-binding proteins capable of targeting desired genomic DNA sequences. Methods of engineering zinc finger arrays are well known in the art, and include, for example, combining smaller zinc fingers of known specificity. An exemplary zinc finger is ZF108 which targets the FWA locus of Arabidopsis and whose amino acid sequence is presented in SEQ ID NO: 211.

In some embodiments, recombinant polypeptides of the present disclosure may contain a DNA-binding domain other than a zinc finger domain. Examples of such DNA-binding domains may include, for example, TAL (transcription activator-like) effector targeting domains, helix-turn-helix family DNA-binding domains, basic domains, ribbon-helix-helix domains, TBP (TATA-box binding protein) domains, barrel dimer domains, RHB domains (real homology domain), BAH (bromo-adjacent homology) domains, SANT domains, Chromodomains, Tudor domains, Bromodomains, PHD domains (plant homeo domain), WD40 domains, and MBD domains (methyl-CpG-binding domain).

In some embodiments, the DNA-binding domain is a TAL effector targeting domain. TAL effectors generally refer to secreted bacterial proteins, such as those secreted by Xanthomonas or Ralstonia bacteria when infecting various plant species. Generally, TAL effectors are capable of binding promoter sequences in the host plant, and activate the expression of plant genes that aid in bacterial infection. TAL effectors recognize plant DNA sequences through a central repeat targeting domain that contains a variable number of approximately 34 amino acid repeats. Moreover, TAL effector targeting domains can be engineered to target specific DNA sequences. Methods of modifying TAL effector targeting domains are well known in the art, and described in Bogdanove and Voytas, Science. 2011 Sep. 30; 333(6051):1843-6.

Other DNA-binding domains for use in the methods and compositions of the present disclosure will be readily apparent to one of skill in the art, in view of the present disclosure.

### RNA-Guided DNA-Binding Proteins and Systems

In some embodiments, the targeting domain is or may include an RNA-guided DNA binding protein. For example, the targeting domain may be an RNA-guided DNA binding protein (e.g. Cas9, Cas12, etc.) and employ a CRISPR-based targeting system to target a recombinant polypeptide to a target nucleic acid.

CRISPR systems naturally use small base-pairing guide RNAs to target and cleave foreign DNA elements in a sequence-specific manner (Wiedenheft et al., 2012). There are diverse CRISPR systems in different organisms that may be used to target proteins of the present disclosure to a target nucleic acid. One of the simplest systems is the type II CRISPR system from Streptococcus pyogenes. Only a single gene encoding the CAS9 protein and two RNAs, a mature CRISPR RNA (crRNA) and a partially complementary trans-acting RNA (tracrRNA), are necessary and sufficient for RNA-guided silencing of foreign DNAs (Jinek et al., 2012). Maturation of crRNA requires tracrRNA and RNase III (Deltcheva et al., 2011). However, this requirement can be bypassed by using an engineered small guide RNA (gRNA) containing a designed hairpin that mimics the tracrRNA-crRNA complex (Jinek et al., 2012). Base pairing between the gRNA and target DNA normally causes double-strand breaks (DSBs) due to the endonuclease activity of CAS9.

It is known that the endonuclease domains of the CAS9 protein can be mutated to create a programmable RNA-dependent DNA-binding protein (dCAS9) (Qi et al., 2013). The fact that duplex gRNA-dCAS9 binds target sequences without endonuclease activity has been used to tether regulatory proteins, such as transcriptional activators or repressors, to promoter regions in order to modify gene expression (Gilbert et al., 2013), and CAS9 transcriptional activators have been used for target specificity screening and paired nickases for cooperative genome engineering (Mali et al., 2013, Nature Biotechnology 31:833-838). Thus, dCAS9 may be used as a modular RNA-guided platform to recruit different proteins to DNA in a highly specific manner. One of skill in the art would recognize other RNA-guided DNA binding protein/RNA complexes that can be used equivalently to CRISPR-CAS9.

Various CAS proteins suitable for use in the methods and compositions of the present disclosure are known in the art and described herein. In some embodiments, the CAS polypeptide may be a Cas9 polypeptide having an amino acid sequence that has at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 216.

Targeting using CRISPR-based systems may be beneficial over other genome targeting techniques in certain instances. For example, one need only change the guide RNAs in order to target fusion proteins to a new genomic location, or even multiple locations simultaneously. In addition, guide RNAs can be extended to include sites for binding to proteins, such as the MS2 protein, which can be fused to proteins of interest. Variations of CRISPR-based targeting may also be used herein (e.g. a SunTag system) to facilitate targeting of a recombinant polypeptides to a target nucleic acid, as will be readily apparent to one of skill in the art.

Suitable CRISPR-based targeting systems and variations thereof are well-known in the art and may be used in the embodiments of the present disclosure in view of the guidance provided herein. For example, WO2018/136783 describes a SunTag-based targeting system for use in plants. WO2018/136783 is incorporated herein by reference in its entirety.

SunTag-based targeting in the context of the present disclosure may involve the recruitment of multiple copies of a transcriptional repressor polypeptide to a target nucleic acid in plants via CRISPR-based targeting. In certain aspects, this specific targeting involves the use of a system that includes (1) a nuclease-deficient CAS9 (dCAS9) polypeptide that is recombinantly fused to a multimerized epitope, (2) a transcriptional repressor polypeptide that is recombinantly fused to an affinity polypeptide, and (3) a guide RNA (gRNA). In this aspect, the dCAS9 portion of the dCAS9-multimerized epitope fusion protein is involved with targeting a target nucleic acid as directed by the guide RNA. The multimerized epitope portion of the dCAS9-multimerized epitope fusion protein is involved with binding to the affinity polypeptide (which is recombinantly fused to a transcriptional repressor). The affinity polypeptide portion of the transcriptional repressor-affinity polypeptide fusion protein is involved with binding to the multimerized epitope so that the transcriptional repressor can be in association with dCAS9. The transcriptional repressor portion of the transcriptional repressor-affinity polypeptide fusion protein is involved with repressing transcription of a target nucleic acid once the complex has been targeted to a target nucleic acid via the guide RNA.

As described above, certain aspects of the present disclosure involve CRISPR-based targeting of a target nucleic acid, which may involve use of a CRISPR-CAS9 targeting system. CRISPR-CAS9 systems may involve the use of a CRISPR RNA (crRNA), a trans-activating CRISPR RNA (tracrRNA), and a CAS9 protein. The crRNA and tracrRNA aid in directing the CAS9 protein to a target nucleic acid sequence, and these RNA molecules can be specifically engineered to target specific nucleic acid sequences. In particular, certain aspects of the present disclosure involve the use of a single guide RNA (gRNA) that reconstitutes the function of the crRNA and the tracrRNA. Further, certain aspects of the present disclosure involve a CAS9 protein that does not exhibit DNA cleavage activity (dCAS9). As disclosed herein, gRNA molecules may be used to direct a dCAS9 protein to a target nucleic acid sequence.

Certain aspects of the present disclosure involving SunTag-based targeting relate to recombinant polypeptides that contain an affinity polypeptide. Affinity polypeptides of the present disclosure may bind to one or more epitopes (e.g. a multimerized epitope). In some embodiments, an affinity polypeptide is present in a recombinant polypeptide that contains a transcriptional repressor polypeptide and an affinity polypeptide.

A variety of affinity polypeptides are known in the art and may be used herein. Generally, the affinity polypeptide should be stable in the conditions present in the intracellular environment of a plant cell. Additionally, the affinity polypeptide should specifically bind to its corresponding epitope with minimal cross-reactivity. The affinity polypeptide may be an antibody such as, for example, an scFv. The antibody may be optimized for stability in the plant intracellular environment. When a GCN4 epitope is used in the methods described herein, a suitable affinity polypeptide that is an antibody may contain an anti-GCN4 scFv domain. Other exemplary affinity polypeptides include, for example, proteins with SH2 domains or the domain itself, 14-3-3 proteins, proteins with SH3 domains or the domain itself, the Alpha-Syntrophin PDZ protein interaction domain, the PDZ signal sequence, or proteins from plants which can recognize AGO hook motifs (e.g. AGO4 from Arabidopsis thaliana).

Certain aspects of the present disclosure involving SunTag-based targeting relate to recombinant polypeptides that contain an epitope or a multimerized epitope. Epitopes of the present disclosure may bind to an affinity polypeptide. In some embodiments, an epitope or multimerized epitope is present in a recombinant polypeptide that contains a dCAS9 polypeptide.

Epitopes of the present disclosure may be used for recruiting affinity polypeptides (and any polypeptides they may be recombinantly fused to) to a dCAS9 polypeptide. In embodiments where a dCAS9 polypeptide is fused to an epitope or a multimerized epitope, the dCAS9 polypeptide may be fused to one copy of an epitope, multiple copies of an epitope, more than one different epitope, or multiple copies of more than one different epitope as further described herein.

A variety of epitopes and multimerized epitopes are known in the art and may be used herein. In general, the epitope or multimerized epitope may be any polypeptide sequence that is specifically recognized by an affinity polypeptide of the present disclosure. Exemplary epitopes may include a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, a VSV-G epitope, and a GCN4 epitope. Other exemplary amino acid sequences that may serve as epitopes and multimerized epitopes include, for example, phosphorylated tyrosines in specific sequence contexts recognized by SH2 domains, characteristic consensus sequences containing phosphoserines recognized by 14-3-3 proteins, proline rich peptide motifs recognized by SH3 domains, the PDZ protein interaction domain or the PDZ signal sequence, and the AGO hook motif from plants.

Epitopes described herein may also be multimerized. Multimerized epitopes may include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 or more copies of an epitope.

Multimerized epitopes may be present as tandem copies of an epitope, or each individual epitope may be separated from another epitope in the multimerized epitope by a linker or other amino acid sequence. Suitable linker regions are known in the art and are described herein. The linker may be configured to allow the binding of affinity polypeptides to adjacent epitopes without, or without substantial, steric hindrance. Linker sequences may also be configured to provide an unstructured or linear region of the polypeptide to which they are recombinantly fused. The linker sequence may comprise e.g. one or more glycines and/or serines. The linker sequences may be e.g. at least 2, at least 3, at least 4, at least 5, at least 6, at least 7, at least 8, at least 9, or at least 10 or more amino acids in length.

### Recombinant Nucleic Acids

Certain aspects of the present disclosure relate to recombinant nucleic acids encoding recombinant polypeptides.

As used herein, the terms “polynucleotide,” “nucleic acid,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications. As used herein, the symbols for nucleotides and polynucleotides are those recommended by the IUPAC-IUB Commission of Biochemical Nomenclature.

In one aspect, the present disclosure provides recombinant nucleic acids that encode recombinant polypeptides that contain e.g. a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide. In some embodiments, the recombinant nucleic acid encodes a recombinant polypeptide that contains a transcriptional repressor polypeptide that has an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to one or more of SEQ ID NO: 1-210.

Sequences of the polynucleotides of the present disclosure may be prepared by various suitable methods known in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteucci et al., (1980) Tetrahedron Lett 21:719-722; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those of ordinary skill in the art, such as utilization of polymerase chain reactions (PCR; e.g., U.S. Pat. No. 4,683,195).

The nucleic acids employed in the methods and compositions described herein may be codon optimized relative to a parental template for expression in a particular host cell. Cells differ in their usage of particular codons, and codon bias corresponds to relative abundance of particular tRNAs in a given cell type. By altering codons in a sequence so that they are tailored to match with the relative abundance of corresponding tRNAs, it is possible to increase expression of a product (e.g. a polypeptide) from a nucleic acid. Similarly, it is possible to decrease expression by deliberately choosing codons corresponding to rare tRNAs. Thus, codon optimization/deoptimization can provide control over nucleic acid expression in a particular cell type (e.g. bacterial cell, plant cell, mammalian cell, etc.). Methods of codon optimizing a nucleic acid for tailored expression in a particular cell type are well-known to those of skill in the art.

### Methods of Identifying Sequence Similarity

Various methods are known to those of skill in the art for identifying similar (e.g. homologs, orthologs, paralogs, etc.) polypeptide and/or polynucleotide sequences, including phylogenetic methods, sequence similarity analysis, and hybridization methods.

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou and Nei, Mol. Biol. & Evo. 4:406-425 (1987)). Homologous sequences may also be identified by a reciprocal BLAST strategy. Evolutionary distances may be computed using the Poisson correction method (Zuckerkandl and Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e. by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes). Groups of similar genes can also be identified with pair-wise BLAST analysis (Feng and Doolittle, J. Mol. Evol. 25: 351-360 (1987)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount, Bioinformatics: Sequence and Genome Analysis Cold Spring Harbor Laboratory Press, Cold Spring Harbor, N.Y., page 543 (2001)).

To find sequences that are homologous to a reference sequence, BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used.

Methods for the alignment of sequences and for the analysis of similarity and identity of polypeptide and polynucleotide sequences are well-known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g. charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer assisted methods. As noted below, a variety of computer programs for performing sequence alignment are available, or can be produced by one of skill.

The determination of percent sequence identity and/or similarity between any two sequences can be accomplished using a mathematical algorithm. Examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul, Proc. Natl. Acad. Sci. USA 87:2264-2268 (1990), modified as in Karlin and Altschul, Proc. Natl. Acad. Sci. USA 90:5873-5877 (1993).

Computer implementations of these mathematical algorithms can be utilized for comparison of sequences to determine sequence identity and/or similarity. Such implementations include, for example: CLUSTAL in the PC/Gene program (available from Intelligenetics, Mountain View, Calif.); the AlignX program, version10.3.0 (Invitrogen, Carlsbad, CA) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. Gene 73:237-244 (1988); Higgins et al. CABIOS 5:151-153 (1989); Corpet et al., Nucleic Acids Res. 16:10881-90 (1988); Huang et al. CABIOS 8:155-65 (1992); and Pearson et al., Meth. Mol. Biol. 24:307-331 (1994). The BLAST programs of Altschul et al. J. Mol. Biol. 215:403-410 (1990) are based on the algorithm of Karlin and Altschul (1990) supra.

Polynucleotides homologous to a reference sequence can be identified by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor, N.Y. (“Sambrook”) (1989); Berger and Kimmel, Guide to Molecular Cloning Techniques, Methods in Enzymology, vol. 152 Academic Press, Inc., San Diego, Calif (“Berger and Kimmel”) (1987); and Anderson and Young, “Quantitative Filter Hybridisation.” In: Hames and Higgins, ed., Nucleic Acid Hybridisation, A Practical Approach. Oxford, TRL Press, 73-111 (1985)).

Encompassed by the disclosure are polynucleotide sequences that are capable of hybridizing to the disclosed polynucleotide sequences and fragments thereof under various conditions of stringency (see, for example, Wahl and Berger, Methods Enzymol. 152: 399-407 (1987); and Kimmel, Methods Enzymo. 152: 507-511, (1987)). Full length cDNA, homologs, orthologs, and paralogs of polynucleotides of the present disclosure may be identified and isolated using well-known polynucleotide hybridization methods.

### Target Nucleic Acids and Sequences

Recombinant polypeptides of the present disclosure may be targeted to specific target nucleic acid acids to reduce expression of the target nucleic acid.

Certain aspects of the present disclosure relate to target sites on target nucleic acids. A target site generally refers to a location of a target nucleic acid that is targeted by a recombinant polypeptide of the present disclosure (e.g. a nucleotide sequence of a target nucleic acid that can be bound by a targeting agent, such as e.g. a DNA-binding domain, in a recombinant polypeptide). In some embodiments, the target site may include both the nucleotide sequence targeted as well as at least 5, at least 10, at least 15, at least 20, at least 25, at least 30, at least 35, at least 40, at least 45, or at least 50 nucleotides or more on the 3′ side, the 5′ side, or both the 3′ and 5′ side of the nucleotide sequence in the target nucleic acid that is targeted. In some embodiments, the target site may contain at least 30, at least 40, at least 50, at least 60, at least 70, at least 80, at least 90, at least 100, at least 125, at least 150, at least 175, or at least 200 or more nucleotides.

In some embodiments, a recombinant polypeptide is targeted to a particular locus. A locus generally refers to a specific position on a chromosome or other nucleic acid molecule. A locus may contain, for example, a polynucleotide that encodes a protein or an RNA. A locus may also contain, for example, a non-coding RNA, a gene, a promoter, a 5′ untranslated region (UTR), an exon, an intron, a 3′ UTR, or combinations thereof. In some embodiments, a locus may contain a coding region for a gene.

In some embodiments, a recombinant polypeptide is targeted to a gene. A gene generally refers to a polynucleotide that can produce a functional unit (for example, a protein or a noncoding RNA molecule). A gene may contain a promoter, an enhancer sequence, a leader sequence, a transcriptional start site, a transcriptional stop site, a polyadenylation site, one or more exons, one or more introns, a 5′ UTR, a 3′ UTR, or combinations thereof. A gene sequence may contain a polynucleotide sequence encoding a promoter, an enhancer sequence, a leader sequence, a transcriptional start site, a transcriptional stop site, a polyadenylation site, one or more exons, one or more introns, a 5′ UTR, a 3′ UTR, or combinations thereof.

The target nucleic acid sequence may be located within the coding region of a target gene or upstream or downstream thereof. Moreover, the target nucleic acid sequence may reside endogenously in a target gene or may be inserted into the gene, e.g., heterologous, for example, using techniques such as homologous recombination. For example, a target gene of the present disclosure can be operably linked to a control region, such as a promoter, that contains a sequence that can be recognized by a targeting agent (e.g. a DNA-binding domain) or other factor in association with a targeting agent (e.g. a guide RNA) such that a recombinant polypeptide may be targeted to that sequence.

The target nucleic acid sequence may be located in a region of chromatin. In some embodiments, the target nucleic acid sequence may be in a region of open chromatin or similar region of DNA that is generally accessible to transcriptional machinery. Regions of open chromatin may be characterized by nucleosome depletion, nucleosome disruption, accessibility to transcriptional machinery, and/or a transcriptionally active state. Regions of open chromatin will be readily understood and identifiable by one of skill in the art.

Target genes or nucleic acid regions to be targeted for reduced expression by a recombinant polypeptide of the present disclosure will be readily apparent to those of skill in the art depending on the particular application and/or purpose. For example, genes with particular agricultural importance may be targeted for reduced expression according to the methods of the present disclosure. Exemplary genes to be targeted for reduced expression may include, for example, those involved in light perception (e.g. PHYB, etc.), those involved in the circadian clock (e.g. CCA1, LHY, etc.), those involved in flowering time (e.g. CO, FT, etc.), those involved in meristem size (e.g. WUS, CLV3, etc.), those involved in plant architecture (S, SP, TFL1, SFT, etc.) and genes involved in embryogenesis, chromatin structure, stress response, growth and development, etc.

In some embodiments, the target nucleic acid is endogenous to the plant where the expression of one or more genes is to be reduced according to the methods described herein. In some embodiments, the target nucleic acid is a transgene of interest that has been inserted into a plant. Suitable target nucleic acids will be readily apparent to one of skill in the art depending on the particular need or outcome. The target nucleic acid sequence may be in e.g. a region of euchromatin (e.g. highly expressed gene), or the target nucleic acid sequence may be in a region of heterochromatin (e.g. centromere DNA).

In some embodiments, the target nucleic acid may be in a region of repressive chromatin. Repressive chromatin generally refers to regions of chromatin where transcription is repressed or otherwise generally transcriptionally inactive. Exemplary regions of repressive chromatin include, for example, regions with repressive DNA methylation, compact chromatin, and/or no transcription).

### Recombinant Expression

Recombinant nucleic acids and/or recombinant polypeptides of the present disclosure may be present in host cells (e.g. plant cells). In some embodiments, recombinant nucleic acids are present in an expression vector and may encode a recombinant polypeptide, and the expression vector may be present in host cells (e.g. plant cells). In some embodiments, recombinant nucleic acids and/or recombinant polypeptides are present in host cells (e.g. plant cells) via direct introduction into the cell (e.g. via RNPs).

In some embodiments, the genes encoding the recombinant polypeptides in the plant cell may be heterologous to the plant cell. In certain embodiments, the plant cell does not naturally produce one or more polypeptides of the present disclosure, and contains heterologous nucleic acid constructs capable of expressing one or more genes necessary for producing those molecules. In certain embodiments, the plant cell does not naturally produce one or more polypeptides of the present disclosure, and is provided the one or more polypeptides through exogenous delivery of the polypeptides directly to the plant cell without the need to express a recombinant nucleic acid encoding the recombinant polypeptide in the plant cell.

Recombinant polypeptides of the present disclosure may be introduced into host cells (e.g. plant cells) via any suitable methods known in the art. For example, a recombinant polypeptide can be exogenously added to plant cells and the plant cells are maintained under conditions such that the recombinant polypeptide is targeted to one or more target nucleic acids to reduce expression of the target nucleic acids in the plant cells. Alternatively, a recombinant nucleic acid encoding a recombinant polypeptide of the present disclosure can be expressed in plant cells and the plant cells are maintained under conditions such that the recombinant polypeptide is targeted to one or more target nucleic acids to reduce expression of the target nucleic acids in the plant cells. Additionally, in some embodiments, a recombinant polypeptide of the present disclosure may be transiently expressed in a plant via viral infection of the plant, or by introducing a recombinant polypeptide-encoding RNA into a plant to facilitate reduced expression of a target nucleic acid of interest. Methods of introducing recombinant proteins via viral infection or via the introduction of RNAs into plants are well known in the art. For example, Tobacco rattle virus (TRV) has been successfully used to introduce zinc finger nucleases in plants to cause genome modification (“Nontransgenic Genome Modification in Plant Cells”, Plant Physiology 154:1079-1087 (2010)). TRV and other appropriate viruses may be used herein to facilitate editing in plants cells.

In some embodiments, a recombinant polypeptide and a guide RNA may be exogenously and directly supplied to a plant cell as a ribonucleoprotein (RNP) complex. This particular form of delivery is useful for facilitating transgene-free editing in plants. Modified guide RNAs which are resistant to nuclease digestion could also be used in this approach. Transgene-free callus from plants cells provided with an RNP could be used to regenerate whole plants.

A recombinant nucleic acid encoding a recombinant polypeptide of the present disclosure can be expressed in a plant with any suitable plant expression vector. Typical vectors useful for expression of recombinant nucleic acids in higher plants are well known in the art and include, for example, vectors derived from the tumor-inducing (Ti) plasmid of Agrobacterium tumefaciens (e.g., see Rogers et al., Meth. in Enzymol. (1987) 153:253-277). These vectors are plant integrating vectors in that on transformation, the vectors integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, CA).

In addition to regulatory domains, recombinant polypeptides of the present disclosure can be expressed as a fusion protein that is coupled to, for example, a maltose binding protein (“MBP”), glutathione S transferase (GST), hexahistidine, c-myc, or the FLAG epitope for ease of purification, monitoring expression, or monitoring cellular and subcellular localization.

Moreover, a recombinant nucleic acid encoding a recombinant polypeptide of the present disclosure can be modified to improve expression of the recombinant protein in plants by using codon preference/codon optimization to target preferential expression in plant cells. When the recombinant nucleic acid is prepared or altered synthetically, advantage can be taken of known codon preferences of the intended plant host where the nucleic acid is to be expressed. For example, recombinant nucleic acids of the present disclosure can be modified to account for the specific codon preferences and GC content preferences of monocotyledons and dicotyledons, as these preferences have been shown to differ (Murray et al., Nucl. Acids Res. (1989) 17: 477-498).

The present disclosure further provides expression vectors encoding recombinant polypeptides of the present disclosure. A nucleic acid sequence coding for the desired recombinant nucleic acid of the present disclosure can be used to construct a recombinant expression vector which can be introduced into the desired host cell. A recombinant expression vector will typically contain a nucleic acid encoding a recombinant protein of the present disclosure, operably linked to transcriptional initiation regulatory sequences which will direct the transcription of the nucleic acid in the intended host cell, such as tissues of a transformed plant.

Recombinant nucleic acids e.g. encoding recombinant polypeptides of the present disclosure may be expressed on multiple expression vectors or they may be expressed on a single expression vector. For example, plant expression vectors may include (1) a cloned gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

In some embodiments, expression of a nucleic acid of the present disclosure may be driven (in operable linkage) with a promoter (e.g. a promoter functional in plants or a plant-specific promoter). A promoter generally refers to a DNA sequence that contains an RNA polymerase binding site, transcription start site, and/or TATA box and assists or promotes the transcription and expression of an associated transcribable polynucleotide sequence such as, for example, a gene. A plant promoter, or functional fragment thereof, can be employed to e.g. control the expression of a recombinant nucleic acid of the present disclosure in regenerated plants. The selection of the promoter used in expression vectors will determine the spatial and temporal expression pattern of the recombinant nucleic acid in the modified plant, e.g., the nucleic acid encoding the recombinant polypeptide of the present disclosure is only expressed in the desired tissue or at a certain time in plant development or growth. Certain promoters will express recombinant nucleic acids in all plant tissues and are active under most environmental conditions and states of development or cell differentiation (i.e., constitutive promoters). Other promoters will express recombinant nucleic acids in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the recombinant nucleic acid under various inducing conditions.

Examples of suitable constitutive promoters may include, for example, the core promoter of the Rsyn7, the core CaMV 35S promoter (Odell et al., Nature (1985) 313:810-812), CaMV 19S (Lawton et al., 1987), rice actin (Wang et al., 1992; U.S. Pat. No. 5,641,876; and McElroy et al., Plant Cell (1985) 2:163-171); ubiquitin (Christensen et al., Plant Mol. Biol. (1989)12:619-632; and Christensen et al., Plant Mol. Biol. (1992) 18:675-689), pEMU (Last et al., Theor. Appl. Genet. (1991) 81:581-588), MAS (Velten et al., EMBO J. (1984) 3:2723-2730), nos (Ebert et al., 1987), Adh (Walker et al., 1987), the P- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skilled artisans, and constitutive promoters described in, for example, U.S. Pat. Nos. 5,608,149; 5,608,144; 5,604,121; 5,569,597; 5,466,785; 5,399,680; 5,268,463; and 5,608,142.

Recombinant nucleic acids of the present disclosure may be expressed using an RNA Polymerase III (Pol III) promoter such as, for example, the U6 promoter or the H1 promoter (eLife 2013 2:e00471). For example, an approach in plants has been described using three different Pol III promoters from three different Arabidopsis U6 genes, and their corresponding gene terminators (BMC Plant Biology 2014 14:327). One skilled in the art would readily understand that many additional Pol III promoters could be utilized to, for example, simultaneously express many guide RNAs to many different locations in the genome simultaneously. The use of different Pol III promoters for each gRNA expression cassette may be desirable to reduce the chances of natural gene silencing that can occur when multiple copies of identical sequences are expressed in plants.

Recombinant nucleic acids of the present disclosure may be expressed using an RNA Polymerase II (Pol II) promoter such as, for example, the CmYLCV promoter and the 35S promote. Use of a Pol II promoter to drive expression of nucleic acids (e.g. guide RNA expression) may provide additional flexibility for controlling the strength/degree of expression and may provide the possibility of tissue-specific expression. One skilled in the art would recognize appropriate Pol II promoters for use in the methods and compositions of the present disclosure.

Examples of suitable tissue specific promoters may include, for example, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992), the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990), the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the a-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a recombinant nucleic acid of the present disclosure in a specific tissue or may be otherwise under more precise environmental or developmental control. Such promoters are referred to here as “inducible” promoters. Environmental conditions that may affect transcription by inducible promoters include, for example, pathogen attack, anaerobic conditions, or the presence of light. Examples of inducible promoters include, for example, the AdhI promoter which is inducible by hypoxia or cold stress, the Hsp70 promoter which is inducible by heat stress, and the PPDK promoter which is inducible by light. Examples of promoters under developmental control include, for example, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue specific or tissue specific promoter may be used to control the expression of various recombinant polypeptides of the present disclosure.

The recombinant nucleic acids of the present disclosure and/or a vector housing a recombinant nucleic acid of the present disclosure, may also contain a regulatory sequence that serves as a 3′ terminator sequence. A terminator sequence generally refers to a nucleic acid sequence that marks the end of a gene or transcribable nucleic acid during transcription. One of skill in the art would readily recognize a variety of terminators that may be used in the recombinant nucleic acids of the present disclosure. For example, a recombinant nucleic acid of the present disclosure may contain a 3′ NOS terminator. In some embodiments, recombinant nucleic acids of the present disclosure contain a transcriptional termination site. Transcription termination sites may include, for example, OCS terminators, rbcS-E9 terminators, NOS terminators, HSP18.2 terminators, and poly-T terminators.

Recombinant nucleic acids of the present disclosure may include one or more introns. Introns may be included in e.g. recombinant nucleic acids being expressed on a vector in a host cell. The inclusion of one of more introns in a recombinant nucleic acid to be expressed may be particularly helpful to increase expression in plant cells.

Recombinant nucleic acids of the present disclosure may also contain selectable markers. A selectable marker can be used to assist in the selection of transformed cells or tissue due to the presence of a selection agent, such as an antibiotic or herbicide, where the selectable marker gene provides tolerance or resistance to the selection agent. Thus, the selection agent can bias or favor the survival, development, growth, proliferation, etc., of transformed cells expressing the selectable marker gene. Selectable marker genes may include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or Cp4-EPSPS). Selectable marker genes which provide an ability to visually screen for transformants may also be used such as, for example, luciferase or green fluorescent protein (GFP), or a gene expressing a beta glucuronidase or uidA gene (GUS) for which various chromogenic substrates are known. In some embodiments, a nucleic acid molecule provided herein contains a selectable marker gene selected from the group consisting of nptll, aph IV, aadA, aac3, aacC4, bar, pat, DMO, EPSPS, aroA, luciferase, GFP, and GUS.

### Plants and Plant Cells

Certain aspects of the present disclosure relate to plants and plant cells that contain recombinant polypeptides that are targeted to one or more target nucleic acids in the plant/plant cell in order to reduce expression of the target nucleic acid.

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, for example, whole plants, plant cells, plant organs, e.g., leafs, stems, roots, meristems, plant seeds, protoplasts, callus, cell cultures, and any groups of plant cells organized into structural and/or functional units.

Various plant cells may be used in the present disclosure so long as they remain viable after being transformed or otherwise modified to express recombinant nucleic acids or house recombinant polypeptides. Preferably, the plant cell is not adversely affected by the transduction of the necessary nucleic acid sequences, the subsequent expression of the proteins or the resulting intermediates.

As disclosed herein, a broad range of plant types may be modified to incorporate recombinant polypeptides and/or polynucleotides of the present disclosure. Suitable plants that may be modified include both monocotyledonous (monocot) plants and dicotyledonous (dicot) plants.

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Examples of suitable conifer plants may include, for example, loblolly pine (Pinus taeda), slash pine (Pinus elliotii), ponderosa pine (Pinus ponderosa), lodgepole pine (Pinus contorta), Monterey pine (Pinus radiata), Douglas-fir (Pseudotsuga menziesii), Western hemlock (Isuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago s sp.), orchard grass, tall fescue, perennial ryegrass, creeping bent grass, and redtop.

Examples of suitable crop plants and model plants may include, for example, Arabidopsis, corn, rice, alfalfa, sunflower, canola, soybean, cotton, peanut, sorghum, wheat, tobacco, and lemna.

The plants and plant cells of the present disclosure may be genetically modified in that recombinant nucleic acids have been introduced into the plants, and as such the genetically modified plants and/or plant cells do not occur in nature. A suitable plant of the present disclosure is e.g. one capable of expressing one or more nucleic acid constructs encoding one or more recombinant proteins.

As used herein, the terms “transgenic plant” and “genetically modified plant” are used interchangeably and refer to a plant which contains within its genome a recombinant nucleic acid. Generally, the recombinant nucleic acid is stably integrated within the genome such that the polynucleotide is passed on to successive generations. However, in certain embodiments, the recombinant nucleic acid is transiently expressed in the plant. The recombinant nucleic acid may be integrated into the genome alone or as part of a recombinant expression cassette. “Transgenic” is used herein to include any cell, cell line, callus, tissue, plant part or plant, the genotype of which has been altered by the presence of exogenous nucleic acid including those transgenics initially so altered as well as those created by sexual crosses or asexual propagation from the initial transgenic.

Plant transformation protocols as well as protocols for introducing recombinant nucleic acids of the present disclosure into plants may vary depending on the type of plant or plant cell, e.g., monocot or dicot, targeted for transformation. Suitable methods of introducing recombinant nucleic acids of the present disclosure into plant cells and subsequent insertion into the plant genome include, for example, microinjection (Crossway et al., Biotechniques (1986) 4:320-334), electroporation (Riggs et al., Proc. Natl. Acad Sci. USA (1986) 83:5602-5606), Agrobacterium-mediated transformation (U.S. Pat. No. 5,563,055), direct gene transfer (Paszkowski et al., EMBO J. (1984) 3:2717-2722), and ballistic particle acceleration (U.S. Pat. No. 4,945,050; Tomes et al. (1995). “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al., Biotechnology (1988) 6:923-926).

Additionally, recombinant polypeptides of the present disclosure can be targeted to a specific organelle within a plant cell. Targeting can be achieved by providing the recombinant protein with an appropriate targeting peptide sequence. Examples of such targeting peptides include, for example, secretory signal peptides (for secretion or cell wall or membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al., J Biol Chem (2000) 10:1074; and Luque and Correas, J Cell Sci (2000) 113:2485-2495).

Modified plants may be grown in accordance with conventional methods (e.g., see McCormick et al., Plant Cell. Reports (1986) 81-84.). These plants may then be grown, and pollinated with either the same transformed strain or different strains, with the resulting hybrid having the desired phenotypic characteristic. Two or more generations may be grown to ensure that the subject phenotypic characteristic is stably maintained and inherited and then seeds harvested to ensure the desired phenotype or other property has been achieved.

The present disclosure also provides plants derived from plants having reduced expression of a target nucleic acid as a consequence of the methods of the present disclosure. A plant having reduced expression of a target nucleic acid as a consequence of the methods of the present disclosure may be crossed with itself or with another plant to produce an F1 plant. In some embodiments, one or more of the resulting F1 plants may also have reduced expression of a target nucleic acid. Accordingly, in some embodiments, provided are progeny plants that are the progeny (either directly or indirectly) of plants having reduced expression of a targeted nucleic acid as a consequence of the methods of the present disclosure. These progeny plants may also have reduced expression of a target nucleic acid. Progeny plants may also have an altered or modified phenotype as compared to a corresponding control plant.

Further provided are methods of screening plants derived from plants having reduced expression of a target nucleic acid as a consequence of the methods of the present disclosure. In some embodiments, the derived plants (e.g. F1 or F2 plants resulting from or derived from crossing the plant having reduced expression of a target nucleic acid as a consequence of the methods of the present disclosure with another plant) can be selected from a population of derived plants. For example, provided are methods of selecting one or more of the derived plants that (i) lack recombinant nucleic acids, and (ii) have reduced expression of a target nucleic acid. Because the reduced expression of the target nucleic acid may be heritable, progeny plants as described herein do not necessarily need to contain a recombinant polypeptide in order to maintain the reduced expression of the target nucleic acid.

### Methods of Reducing Expression of a Target Nucleic Acid

Growing and/or cultivation conditions sufficient for the recombinant polypeptides and/or polynucleotides of the present disclosure to be expressed and/or maintained in the plant/plant cell and to be targeted to and reduce expression of one or more target nucleic acids of the present disclosure are well known in the art and include any suitable growing conditions disclosed herein. Typically, the plant is grown under conditions sufficient to express a recombinant polypeptide of the present disclosure, and for the expressed recombinant polypeptides to be localized to the nucleus of cells of the plant in order to be targeted to and reduce expression of the target nucleic acids (if those target nucleic acids are present in the nucleus). Generally, the conditions sufficient for the expression of the recombinant polypeptide (if being encoded from a recombinant nucleic acid) will depend on the promoter used to control the expression of the recombinant polypeptide. For example, if an inducible promoter is utilized, expression of the recombinant polypeptide in a plant will require that the plant to be grown in the presence of the inducer.

### Growth Conditions

As noted above, growing conditions sufficient for the recombinant polypeptides of the present disclosure to be expressed and/or maintained in the plant and to be targeted to one or more target nucleic acids to reduce expression of the one or more target nucleic acids may vary depending on a number of factors (e.g. species of plant, use of inducible promoter, etc.). Suitable growing conditions may include, for example, ambient environmental conditions, standard laboratory conditions, standard greenhouse conditions, growth in long days under standard environmental conditions (e.g. 16 hours of light, 8 hours of dark), growth in 12 hour light: 12 hour dark day/night cycles, etc.

Various time frames may be used to observe reduced expression of a target nucleic acid according to the methods of the present disclosure. Plants and/or plant cells may be observed/assayed for reduced expression of a target nucleic acid after, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or more after being cultivated/grown in conditions sufficient for a recombinant polypeptide to facilitate reducing expression of a target nucleic acid.

### Reduced Expression of a Target Nucleic Acid

A target nucleic acid of the present disclosure may have its expression reduced/decreased/downregulated as compared to a corresponding control nucleic acid. A target nucleic acid of the present disclosure in a plant cell housing recombinant polypeptides of the present disclosure may have its expression decreased/downregulated by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to a corresponding control. Various controls will be readily apparent to one of skill in the art. For example, a control may be a corresponding plant or plant cell that does not contain recombinant polypeptides of the present disclosure (e.g. wild-type plant or plant cell).

A target nucleic acid may have its expression reduced/decreased/downregulated at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 40-fold, at least about 50-fold, at least about 75-fold, at least about 100-fold, at least about 150-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 1,250-fold, at least about 1,500-fold, at least about 1,750-fold, at least about 2,000-fold, at least about 2,500-fold, at least about 3,000-fold, at least about 3,500-fold, at least about 4,000-fold, at least about 4,500-fold, at least about 5,000-fold, at least about 5,500-fold, at least about 6,000-fold, at least about 6,500-fold, at least about 7,000-fold, at least about 7,500-fold, at least about 8,000-fold, at least about 8,500-fold, at least about 9,000-fold, at least about 9,500-fold, at least about 10,000-fold, at least about 12,000-fold, at least about 14,00-fold, at least about 16,000-fold, at least about 18,000-fold, or at least about 20,000-fold or more as compared to a corresponding control nucleic acid. As stated above, various controls will be readily apparent to one of skill in the art. For example, a control nucleic acid may be a corresponding nucleic acid from a plant or plant cell that does not contain a nucleic acid encoding a recombinant polypeptide of the present disclosure.

Comparisons in the present disclosure may also be in reference to corresponding control plants/plant cells. Various control plants will be readily apparent to one of skill in the art. For example, a control plant or plant cell may be a plant or plant cell that does not contain a recombinant polypeptide (e.g. a wild-type plant) of the present disclosure.

Methods of probing the expression level of a nucleic acid are well-known to those of skill in the art. For example, qRT-PCR analysis may be used to determine the expression level of a population of nucleic acids isolated from a nucleic acid-containing sample (e.g. plants, plant tissues, or plant cells).

In some embodiments, recombinant polypeptides of the present disclosure may facilitate an epigenetic change or other chromatin modification at the target nucleic acid that does not involve a change to the actual nucleic acid nucleotide sequence. Such epigenetic changes and/or chromatin modifications at the target nucleic acid may include, for example, increased DNA methylation, H3K27me3 deposition, H3K4me3 removal/demethylation, and histone deacetylation (e.g. H3K9, H3K14, H3K27, and H4K16 deacetylation). Target nucleic acids of the present disclosure may exhibit one or more of increased DNA methylation, H3K27me3 deposition, H3K4me3 removal/demethylation, and histone deacetylation (e.g. H3K9, H3K14, H3K27, and H4K16 deacetylation) at a level or frequency that is at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% higher as compared to a corresponding control nucleic acid. Various controls will be readily apparent to one of skill in the art. For example, a control nucleic acid may be a corresponding nucleic acid from a plant or plant cell that does not contain recombinant polypeptides of the present disclosure (e.g. wild-type plant or plant cell).

In some embodiments, recombinant polypeptides of the present disclosure may interfere with transcription of the target nucleic acid. Such interference may include, e.g. interference with RNA Polymerase II transcription elongation and RNA Polymerase II Serine 5 (Ser-5) dephosphorylation. Target nucleic acids of the present disclosure may exhibit one or more of interference with RNA Polymerase II transcription elongation and RNA Polymerase II Serine 5 (Ser-5) dephosphorylation at a level or frequency that is at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% higher as compared to a corresponding control nucleic acid. Various controls will be readily apparent to one of skill in the art. For example, a control nucleic acid may be a corresponding nucleic acid from a plant or plant cell that does not contain recombinant polypeptides of the present disclosure (e.g. wild-type plant or plant cell).

### Kits

Certain aspects of the present disclosure relate to an article of manufacture or kit comprising a polynucleotide, vector, cell, and/or composition described herein. In some embodiments, the kit further comprises a packed insert comprising instructions for the use of the polynucleotide, vector, cell, and/or composition. In some embodiments, the article of manufacture or kit further comprises one or more buffer, e.g., for storing, transferring, or otherwise using the polynucleotide, vector, cell, and/or composition. In some embodiments, the kit further comprises one or more containers for storing the polynucleotide, vector, cell, and/or composition.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

### EXAMPLES

The following examples are offered to illustrate provided embodiments and are not intended to limit the scope of the present disclosure.

### Example 1: A Gain of Function Screen for Regulators of Gene Silencing in Arabidopsis

### Summary

The Examples provided herein illustrate Applicant's discovery of diverse mechanisms of gene repression in Arabidopsis. Gene silencing is critical in many developmental contexts in plants, yet the mechanisms by which gene repression occurs are not completely understood. A gain of function screen was performed for proteins involved in gene silencing by fusing 260 putative Arabidopsis chromatin proteins with an artificial zinc finger domain, ZF108, designed to bind the FWA promoter region and selecting for the restoration of silencing in the fwa epigenetic background in which FWA has lost DNA methylation and is overexpressed. This screen uncovered many candidate gene silencers, including a RING/FYVE/PHD superfamily protein PHD1, three telomere repeat-binding factors (TRBs), three HD2 type histone deacetylases, the PAF1C subunit Early Flowering 7 (ELF7), and the carboxyl-terminal domain phosphatase-like 2 (CPL2). Comprehensive mechanistic investigations indicated that these silencers suppressed gene expression either through the establishment of DNA methylation, or via DNA methylation-independent processes including the establishment of histone H3K27me3 deposition, H3K4me3 demethylation, H3K9, H3K14, H3K27, and H4K16 deacetylation, or inhibition of RNA Polymerase II transcription elongation or Ser-5 dephosphorylation. These results provide a more comprehensive understanding of epigenetic regulatory pathways and provide an armament of tools for targeted manipulation of gene expression.

### Introduction

Transcriptional gene regulation is a fundamental biological process that controls the on or off states of gene expression, and involves DNA methylation, histone modification and chromatin remodeling (see e.g., Feng, Science, 2010). In plants, DNA methylation is generally linked to transcriptional gene silencing. For example, the Arabidopsis FWA gene is normally DNA methylated and silenced in all tissues, except in the developing endosperm where it is demethylated and imprinted (see e.g., Kinoshita, Science, 2004). Stable fwa epialleles have been discovered that have permanently lost this DNA methylation, resulting in heritable overexpression of FWA that causes a delay in flowering time (see e.g., Soppe, Mol. Cell, 2000).

Artificial zinc fingers are DNA binding domains that can be designed to bind a specific sequence and guide effector fusion proteins to specific loci (see e.g., Segal, PNAS, 1999). For example, artificial zinc finger 108 (hereafter ZF) was designed to bind the Arabidopsis FWA promoter in the region that is normally methylated in Col-0 wild type plants (see e.g., Johnson, Nature, 2014Johnson et al., 2014). When ZF was fused with the RdDM component SUVH9 and transformed into fwa epiallele containing plants, FWA DNA methylation and suppression and the early flowering phenotype were restored (see e.g., Johnson, Nature, 2014). It was later shown that ZF fusions with the RdDM related factors SAWADEE homeodomain homolog 1 (SHH1), NUCLEAR RNA POLYMERASE D 1 (NRPD1), RNA-dependent RNA polymerase 2 (RDR2), Microrchidia (MORC1), MORC6, MORC7, RNA-DIRECTED DNA METHYLATION 1 (RDM1) and DEFECTIVE IN MERISTEM SILENCING 3 (DMS3) also caused FWA silencing and methylation (see e.g., Gallego-Bartolome, Cell, 2019; Xue, Nat. Comm., 2021).

In this Example, a gain of function approach was utilized to screen a panel of 260 putative Arabidopsis chromatin proteins by fusing each to ZF, transforming these into the unmethylated fwa epiallele background, and screening for fusions capable of inducing an early flowering phenotype indicative of silencing FWA.

### Materials and Methods

### Plant Materials and Growth Conditions

All the plants used in the Examples were in the Arabidopsis thaliana Col-0 ecotype, grown under long-day conditions (16h light and 8h dark). The T-DNA insertion lines used in this study included aipp3-1 (GABI_058D11), aipp3-2 (SAIL_1246_E10), mom1-2 (SAIL_610_GO1), mom1-3 (SALK_141293), mom2-1 (WiscDsLox364H07), mom2-2 (SAIL_548_H02), pial1 (CS358389)pial2 (SALK_043892) double mutant, trb1-2 (SALK_001540) trb2 (CS882628) trb3 (SALK_134645) triple mutant, jmj4-1 (SALK_135712), hd2a (GK-355H03), hd2b (SAIL_1247_A02), hd2c (SALK_129799), elf7-3 (SALK_019433), morc6-3 (GABI_599B06), and morchex consisting of morc1-2 (SAIL_893_B06), morc2-1 (SALK_072774C), morc4-1 (GK-249F08), morc5-1 (SALK_049050C), morc6-3 (GABI_599B06), and morc7-1 (SALK_051729). Moreover, three phd1 mutant alleles were generated using a YAO promoter driven CRISPR/Cas9 system (see e.g., Yan, Mol. Plant, 2015). phd1-2 contained a single nucleotide T insertion and phd1-3 contained a 12-nucleotide deletion and an 18-nucleotide duplication, both of which led to early termination of the protein at amino acid 53 located within the PHD domain. phd1-4 contained a single nucleotide T insertion causing early termination of the protein at amino acid 85 just past the PHD domain. The fwa background RdDM mutants, including nrpd1-4 (SALK_083051), suvh2 (SALK_079574) suvh9 (SALK_048033), morc6-3 (GABI_599B06), rdm1-4 (EMS, (see e.g., Gao, Nature, 2010)), drd1-6 (EMS, (see e.g., Kanno, Nat. Methods, 2004)), dms3-4 (SALK_125019C), nrpe1-1 (EMS), and drm1-2 (SALK_031705) drm2-2 (SALK_150863) were described in (see e.g., Gallego-Bartolome, Cell, 2019). The other fwa background mutants in MOM1 complex were phd1-2, aipp3-1 (GABI_058D11), mom1-3 (SALK_141293), mom2-2 (SAIL_548_H02), and pial1-2 (CS358389) pial2-1 (SALK_043892), which were generated by following the method described in (Johnson et al., 2014). All the transgenic plants were generated by Agrobacterium (AGLO strain) mediated floral dipping.

### Molecular Cloning

The pMDC123 is a Gateway-compatible binary destination vector that includes a plant UBQ10 promoter, followed by an N-terminal ZF and 3×FLAG epitope tag, a Gateway cassette, and an OCS terminator. The list of selected effectors from Arabidopsis Gene ORFeome Collection in the pENTR/D-TOPO vectors were all cloned into pMDC123 destination vector via LR reaction using Gateway LR Clonase II (Invitrogen).

This pEG302 is also a Gateway-compatible binary destination vector, which consists of a gateway cassette, followed by a C-terminal 3×FLAG epitope tag, a ZF, a Biotin Ligase Recognition Peptide (BLRP), and an OCS terminator. The sequence of native promoter (1.5 kb upstream from the 5′UTR or until the next gene annotation) and genomic DNA (No stop codon) of the effectors were cloned into pENTR D-TOPO vectors (Invitrogen), which were used to deliver the genomic DNA sequences of these effectors into the destination vector using Gateway LR Clonase II (Invitrogen).

This pEG302 destination vector contains a gateway cassette, followed by a C-terminal 3×FLAG epitope tag, a BLRP, and an OCS terminator. The cloning method is the same as pEG302-EFFECTOR (gDNA)-3×FLAG-ZF.

This pEG302 destination vector contains a gateway cassette, followed by a C-terminal 9XMYC, a BLRP, and an OCS terminator. The cloning method is the same as pEG302-EFFECTOR (gDNA)-3×FLAG-ZF.

### BS-PCR-Seq

The leaf tissue from 4- to 5-week-old Col-0 wild type, fwa, and the representative T2 ZF lines showing early flowering phenotype were collected to perform Bisulfite PCR at FWA promoter regions. CTAB-based method was used to extract DNA and the EpiTect Bisulfite kit (QIAGEN) was used for DNA conversion. The converted DNA was used as a template to amplify three different regions over promoter and 5′ transcribed regions of FWA, including Region 1 (chr4: 13038143-13038272), Region 2 (chr4: 13038356-13038499) and Region3 (chr4: 13038568-13038695). Pfu Turbo Cx (Agilent), dNTP (Takara Bio), and the primers designed for the above-mentioned FWA regions were used to perform PCR reactions. Three different PCR products from three regions of each sample were pooled and purified with AMPure beads (Beckman Coulter). The purified PCR products were used to construct libraries by the Kapa DNA Hyper Kit (Roche) together with TruSeq DNA UD indexes for Illumina (Illumina), and the libraries were sequenced on Illumina iSeq 100.

BS-PCR-seq data analysis used the pipeline described in Gallego-Bartolome, Cell, 2019. The raw pair-end sequencing reads of each sample were combined, and aligned to both strands of reference genome TAIR10 using BSMAP (v.2.90) (see e.g., Xi, BMC Bioinform., 2009), and the alignment allowed up to 2 mismatches and 1 best hit. The reads with less than 20 reads coverage of cytosines and the reads with more than 3 consecutives methylated CHH sites were removed. The methylation level of each cytosine was calculated using the ratio of C/(C+T), and only the methylation data within the designed FWA regions was kept making a plot using customized R scripts.

### ChIP-Seq

A previous protocol for ChIP-seq was followed with minor modifications (see e.g., Johnson, Nature, 2014). Briefly, a total of 2-4 grams of leaves (histone ChIP-seq) or unopened flower buds (FLAG ChIP-seq) were collected, of which the leaf tissue of 4- to 5-week-old Col-0 wild type, hd2 mutants, elf mutants, fwa, and all the ZF lines showing early flowering phenotype were used for histone and Pol II Ser5 ChIP-seq; the 2-week-old seedlings of Col-0 wild type, jmj14-1 mutant, and trb1/2/3 triple mutant were used for H3K4me3 and H3 ChIP-seq; the inflorescences tissue of FLAG or Myc T2 transgenic lines were used for FLAG or MYC ChIP-seq. The plant materials were ground with liquid nitrogen and fixed with 1% formaldehyde containing nuclei isolation buffering for 10 minutes before adding fresh-made glycine to terminate the crosslinking reaction. The nuclei were isolated and disrupted by SDS, and the chromatin was sheared via Bioruptor Plus (Diagenode) and immunoprecipitated with antibody at 4° C. overnight. Next, the magnetic Protein A and Protein G Dynabeads (Invitrogen) were added and incubated at 4° C. for 2 hours. After washing and elution, the reverse crosslinking was done at 65° C. overnight. Then the protein-DNA complex was treated with Protease K (Invitrogen) at 45° C. for 4 hours, and the DNA was purified and precipitated with 3M Sodium Acetate (Invitrogen), GlycoBlue (Invitrogen) and Ethanol at −20° C. overnight. The precipitated DNA was directly used for library construction using the Ovation Ultra Low System V2 kit (NuGEN), and the libraries were sequenced on Illumina NovaSeq 6000 or HiSeq 4000 instruments.

The ChIP-seq raw reads were trimmed using trim_galore (https://world wide web.bioinformatics.babraham.ac.uk/projects/trim_galore/) and then aligned to TAIR10 genome using bowtie version 1.1.2 (see e.g., Langmead, Genome Biol., 2009), which allowed one unique mapping site 0 mismatch. The Samtools version 1.9 (see e.g., Li, Bioinformatics, 2009) was used to remove the duplicated reads, and together with deeptools version 3.1.3 (see e.g., Ramirez, Nucleic Acids Res., 2016) to generate tracks using RPKM for the normalization. The peaks were called using MACS2 version 2.1.1. (see e.g., Zhang, 2008), and the peaks that were frequently existed in previous FLAG ChIP-seq of Col-0 were removed.

For FLAG-ZF ChIP-seq, the FLAG ChIP-seq was performed in the unopened flower buds of FLAG-TRB1-ZF T2 transgenic plants and fwa plants. The peaks were called by FLAG-TRB1-ZF against fwa, and the peaks with 4 folds or higher signal enrichment were kept as the ZF off-target sites, while the other FLAG ChIP-seq used signal enrichment of 2 folds or higher for the following analysis.

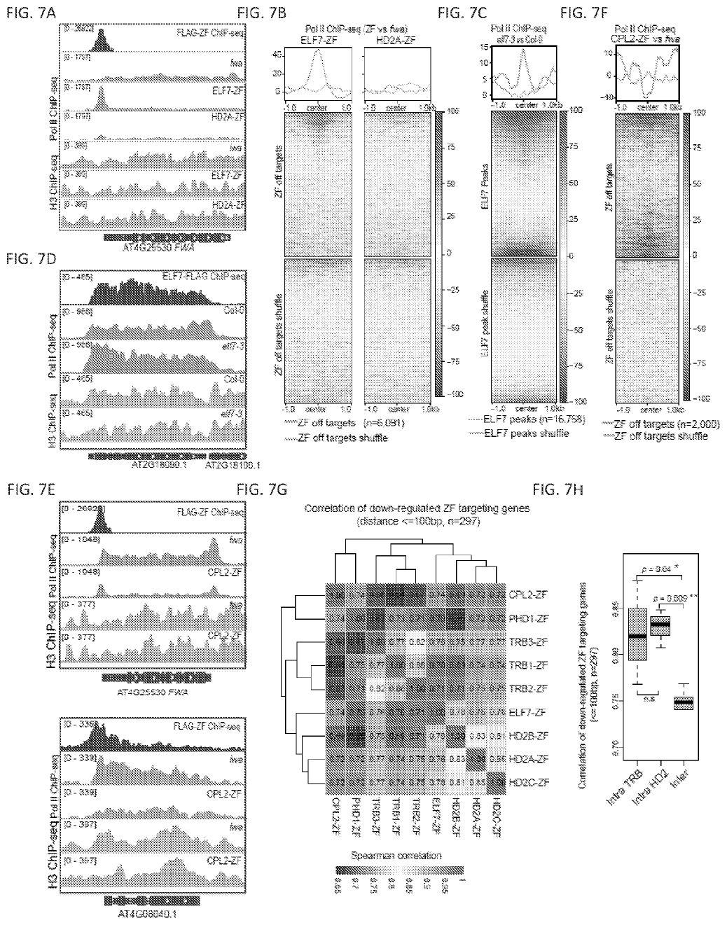

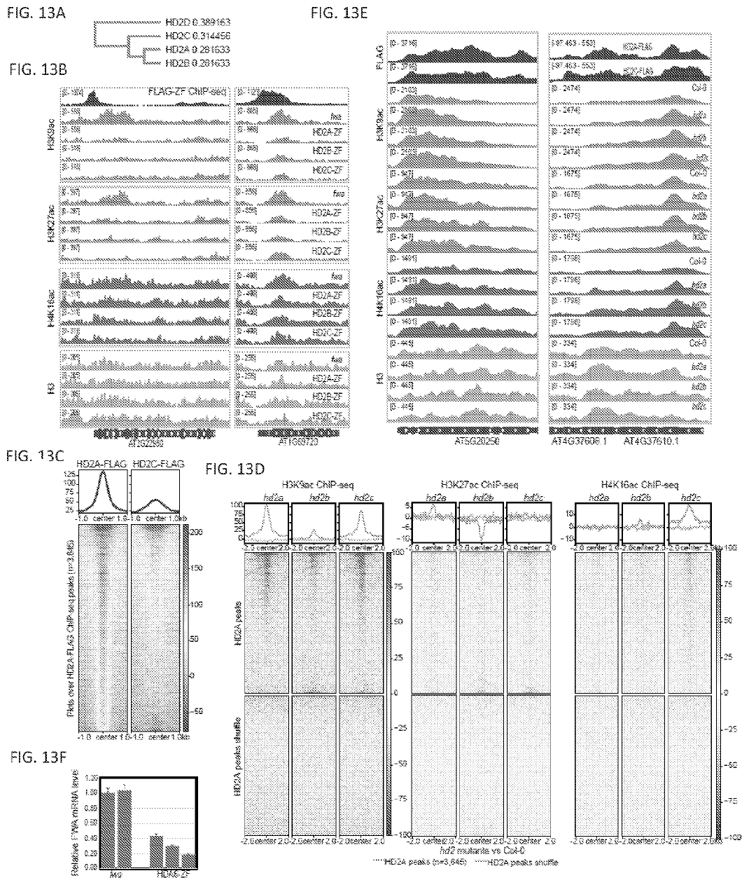

For the comparison of Histone and Pol II (Histone/Pol II) enrichment over ZF off-target sites between ZF lines and fwa, the Histone/Pol II ChIP-seq of each sample including both ZF lines and fwa were normalized with their respective H3 ChIP-seq first by using bigwigCompare, and then the normalized Histone/Pol II ChIP-seq of ZF lines were further normalized to fwa by using bigwigCompare, which were then used to make the metaplot over ZF off-target peaks and random shuffle peaks. This method was also applied in the comparison of H3K9ac, H3K27ac, and H4K16ac ChIP-seq enrichments among Col-0 wild type, hd2a, hd2b, and hd2c mutants over HD2A peaks and shuffle (FIG. 13D), and Pol II ChIP-seq enrichments between Col-0 wild type and elf⁷-3 mutant over ELF7 peaks and shuffle (FIG. 7C).

### RNA-Seq

One leaf tissue of 4- to 5-week-old plants with similar age from fwa and early flowering ZF transgenic lines; 2-week-old seedlings of Col-0 wild type, jmj4-1, trb1/2/3 triple mutant, phd1-2, phd1-4, aipp3-1, aipp3-2, mom1-2, mom1-3, mom2-1, mom2-2, pial1, pial2-1, and pial1/2 double mutants were collected for RNA extraction using Direct-zol RNA MiniPrep kit (Zymo Research). 1 g of total RNA was used to prepare the libraries for RNA-seq following TruSeq Stranded mRNA kit (Illumina), and the libraries were sequenced on Illumina NovaSeq 6000 or HiSeq 4000 instruments.

The RNA-seq raw reads were aligned to TAIR10 genome using bowtie2 (see e.g., Langmead, Nat. Methods, 2012) and the expression levels were calculated with rsem-calculate-expression from RSEM with default settings (see e.g., Li, BMC Bioinform., 2011). The RNA-seq tracks were generated using Samtools version 1.9 (see e.g., Li, Bioinformatics, 2009) and normalized with RPKM using bamCoverage from deeptools version 3.1.3 (see e.g., Ramirez, Nucleic Acids Res., 2016). The differentially expressed genes (DEGs) were called using customized scripts of run_DE_analysis.pl from Trinity version 2.8.5 (see e.g., Grabherr, Nat. Biotech., 2011). log 2 FC≥1 and FDR<0.05 were used as a cut off.

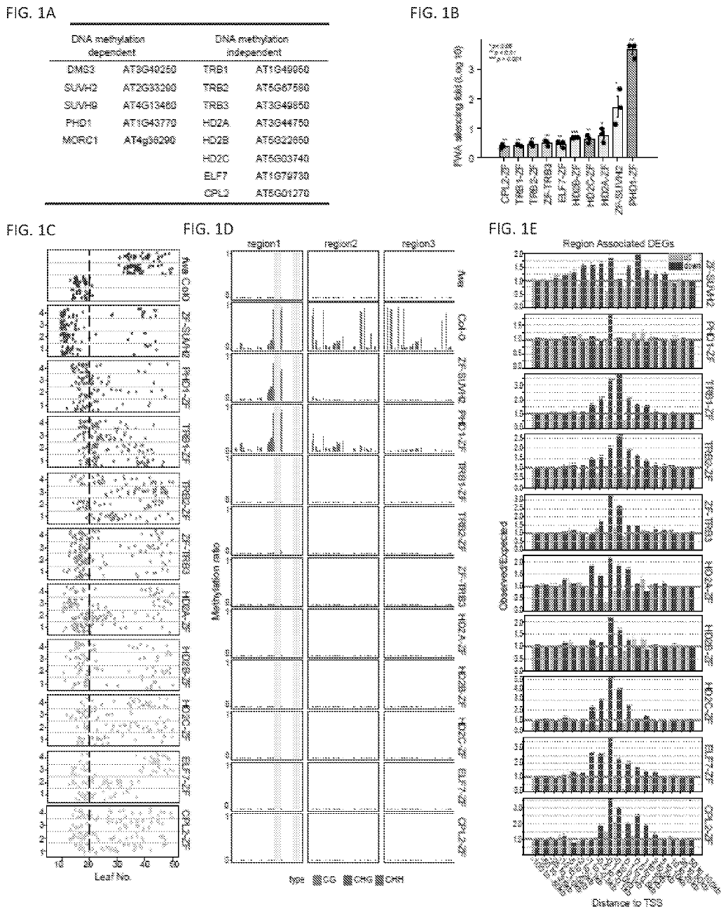

For the silencing fold in FIG. 1B, the RPKMs of FWA gene coding region of each ZF lines and fwa were calculated, and log 10 value of ZF/fwa were presented.

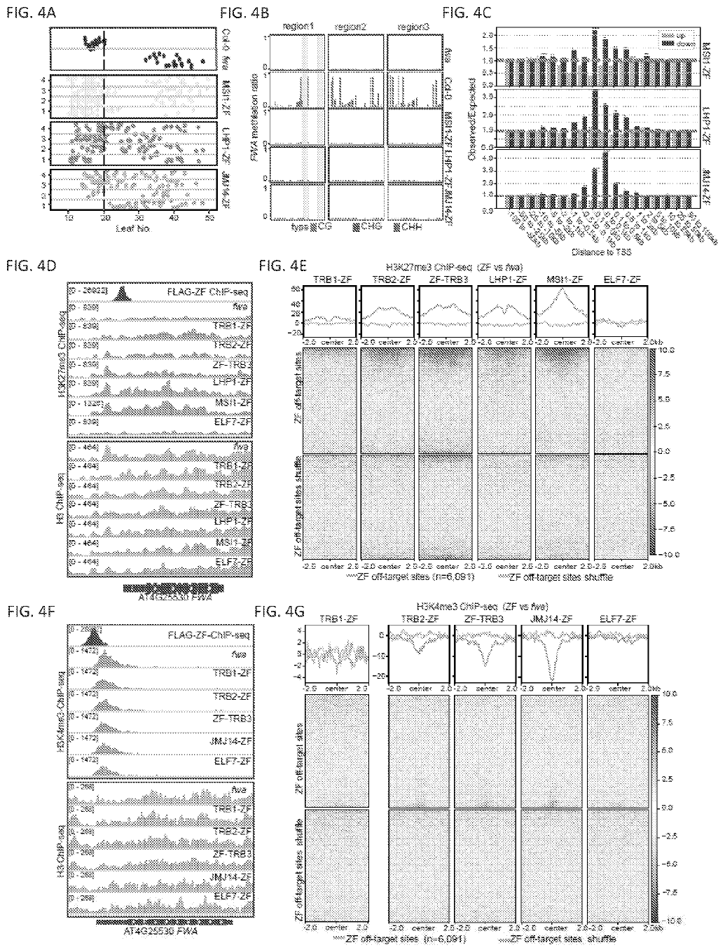

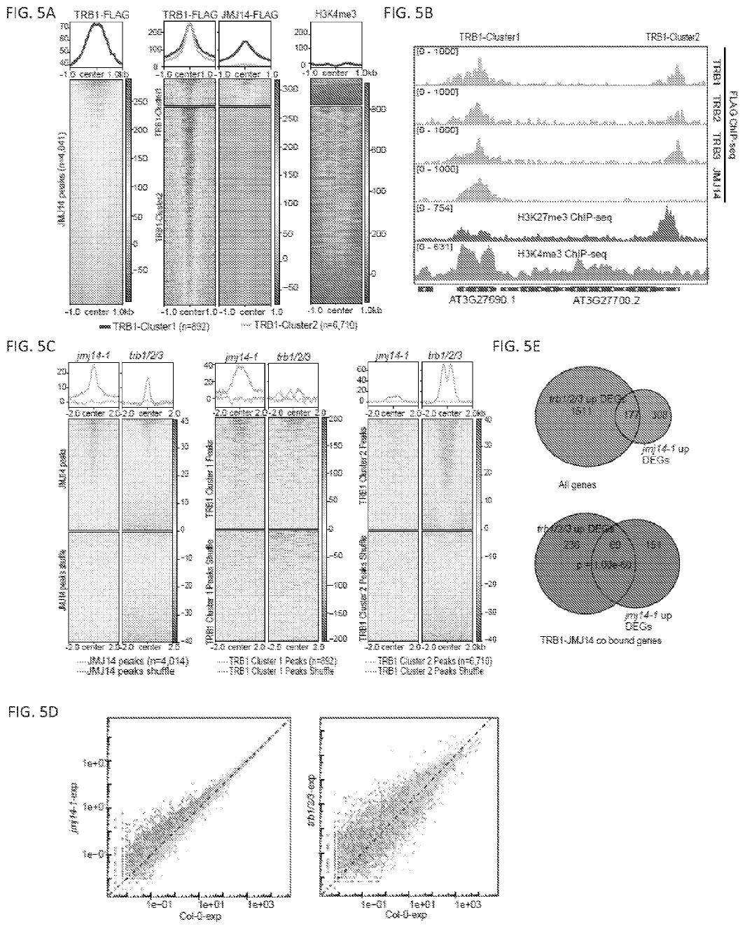

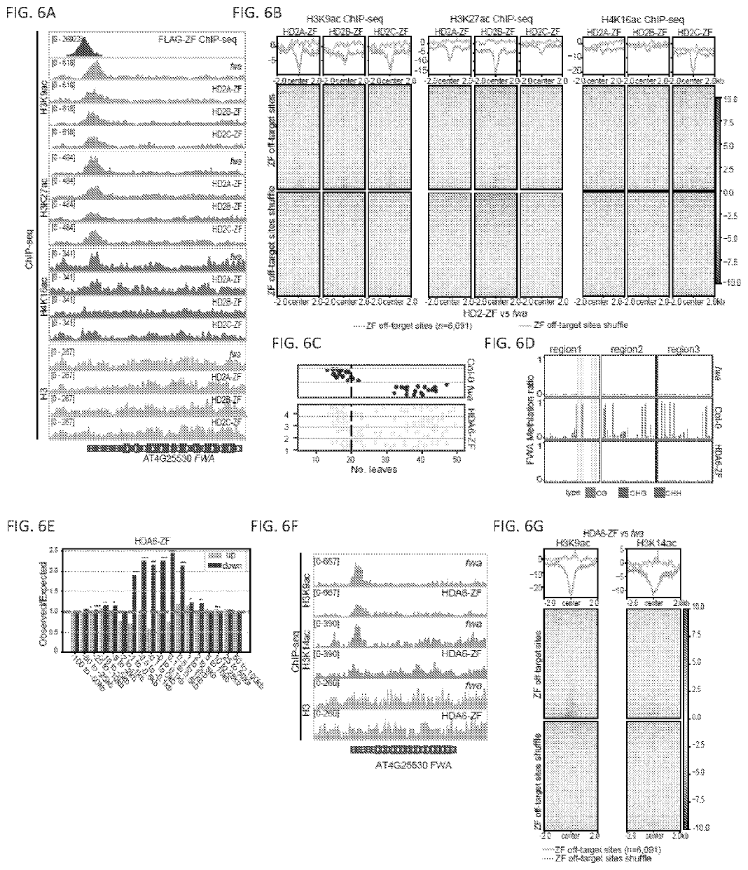

The method of region associated DEGs analysis in FIG. 1E, FIG. 4B, and FIG. 6D were described at (see e.g., Guo, Bioinformatics, 2021). We used the FLAG-ZF ChIP-seq peaks (n=6,091) as input of the favorite regions, and up- and down-regulated DEGs of ZF lines versus fwa were used respectively as the inputs of DEGs.

### Flowering Time Measurement

The flowering times were measured by the leaf counts, and each dot in the dot plots represented the leaf number of individual plants. Plants with 20 or less leaves are considered as early flowering.

### Down-Regulated ZF Targeting Genes

Down-regulated ZF targeting genes were extracted from the files that generated by RAD analysis, which contains the list of up or down regulated genes and distance to the ZF targeting sites. The down-regulated genes (ZFs versus fwa) within 100 bp of ZF target sites were selected as the down-regulated ZF targeting genes of each ZF lines.

### Results

To screen for regulators of gene silencing, the native Arabidopsis gene FWA was utilized as a reporter. FWA is a homeodomain-containing transcription factor gene that causes a late flowering phenotype when overexpressed. In Col-0 wild type plants, FWA is completely silenced by DNA methylation. In fwa epialleles, which have permanently lost this DNA methylation, FWA misexpression causes late flowering (see e.g., Soppe, Mol. Cell, 2000). In order to find putative gene silencing regulators, the Arabidopsis ORFeome collections (see e.g., Pruneda-Paz, Cell Reports, 2014; Yamada, Science, 2003) were searched for proteins with chromatin related annotations including chromatin, chromo, silencing, methylation, SET, histone, bromo, tudor, transcription, and PHD. 260 of these putative Arabidopsis chromatin proteins were cloned into a destination vector driven by a UBQ10 promoter and fused with an N-terminal zinc finger (ZF) designed to bind the FWA promoter (see e.g., Johnson, Nature, 2014). These fusions were individually transformed into fwa plants to screen for gene regulators that triggered FWA silencing and restored an early flowering phenotype.

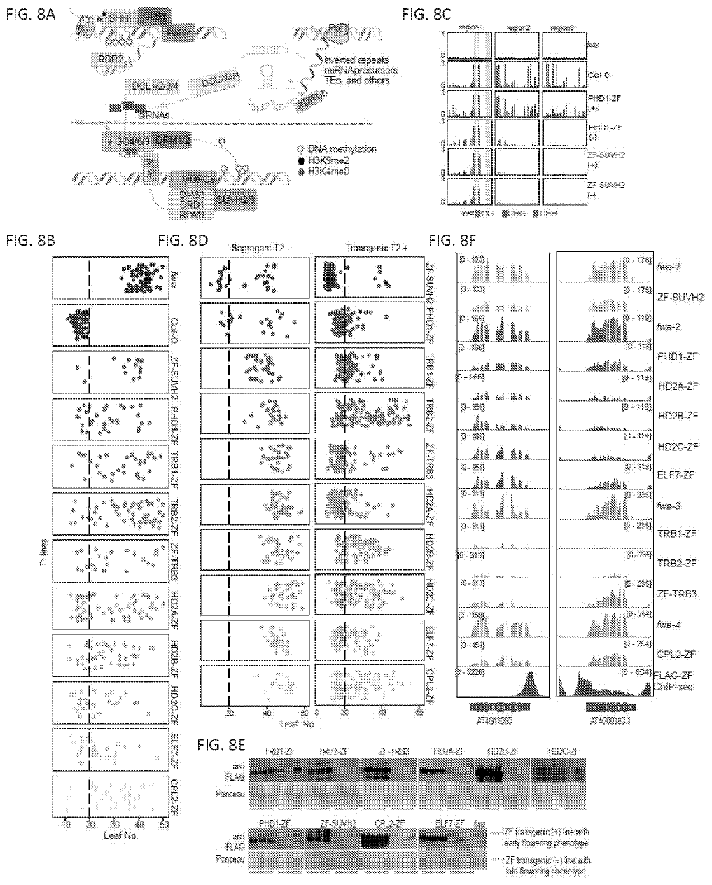

The screen identified a number of effector proteins that successfully restored the early flowering phenotype of the fwa epiallele (FIG. 1A). For some of these effectors, PHD1, TRB1, TRB2, HD2A, HD2B, HD2C, ELF7, and CPL2, only a small number of T1 lines displayed early flowering, suggesting that FWA silencing by these factors was inefficient. C-terminal zinc finger (ZF) fusions with these effector proteins driven by their native promoter in a native genomic DNA sequence context were created, which significantly increased the percentage of early flowering T1 lines (FIG. 8B), suggesting that the fusion of ZF can influence the functionality of some effectors.

Among these effector proteins, DMS3, SUVH2, SUVH9, and MORC1 are known players in the RNA-directed DNA methylation (RdDM) pathway, and previous studies have shown that DMS3-ZF, SUVH9-ZF, and MORC1-ZF could successfully restore an early flowering phenotype to fwa plants by establishment of DNA methylation at FWA promoter regions (see e.g., Gallego-Bartolome, Cell, 2019; Johnson, Nature, 2014). SUVH2 is a close homolog of SUVH9 that functions in RdDM (see e.g., Johnson, Nature, 2014), and as expected ZF-SUVH2 silenced FWA expression and restored the early flowering phenotype by adding DNA methylation to the promoter (FIGS. 1B-1D and FIGS. 8A-8B).

A gene not previously implicated in the RdDM pathway that repressed FWA expression, restored early flowering, and installed FWA promoter DNA methylation was also identified (FIGS. 1B-1D and FIGS. 8A-8B). This gene encodes a RING/FYVE/PHD zinc finger superfamily protein, previously named PHD1 (see e.g., Han, Plant Cell., 2016). PHD1-ZF repressed FWA expression even more strongly than ZF-SUVH2 (FIG. 1B).

Several gene regulators that silenced FWA in a DNA methylation independent manner (FIGS. 1A-1D), including three telomere repeat-binding factors (TRB1, TRB2 and TRB3), three HD2 type histone deacetylases (HD2A, HD2B and HD2C), a PAF1 homolog ELF7, and the Pol II CTD Ser5 phosphatase CPL2 (FIG. 1A) were also identified. The ZF fusions of these proteins restored an early flowering phenotype to the fwa epiallele to a similar level as in wild type Col-0 plants (FIG. 1C), even though the FWA silencing was much lower than ZF-SUVH2 and PHD1-ZF (FIG. 1B), which suggests that FWA expression only needs to be suppressed below a threshold level in order to have a strong effect on flowering time.

To test inheritability of the DNA methylation in ZF-SUVH2 and PHD1-ZF, bisulfite amplicon sequencing analysis (BS-PCR-seq) was performed to evaluate DNA methylation at FWA promoter regions in T2 lines that still contained the transgene and in lines that had segregated away the transgene (null segregants). DNA methylation at FWA by ZF-SUVH2 and PHD1-ZF was observed in both transgenic and null segregant T2 lines, showing that DNA methylation established by these fusion proteins was heritable in the absence of the transgene (FIG. 8C), as has been shown for other fusion proteins that target methylation to FWA (see e.g., Gallego-Bartolome, Cell, 2019; Johnson, Nature, 2014). The heritability of the early flowering phenotype in these lines was investigated and, as expected, the early flowering phenotype was also inherited in many null segregant plants in the T2 population (FIG. 8D).

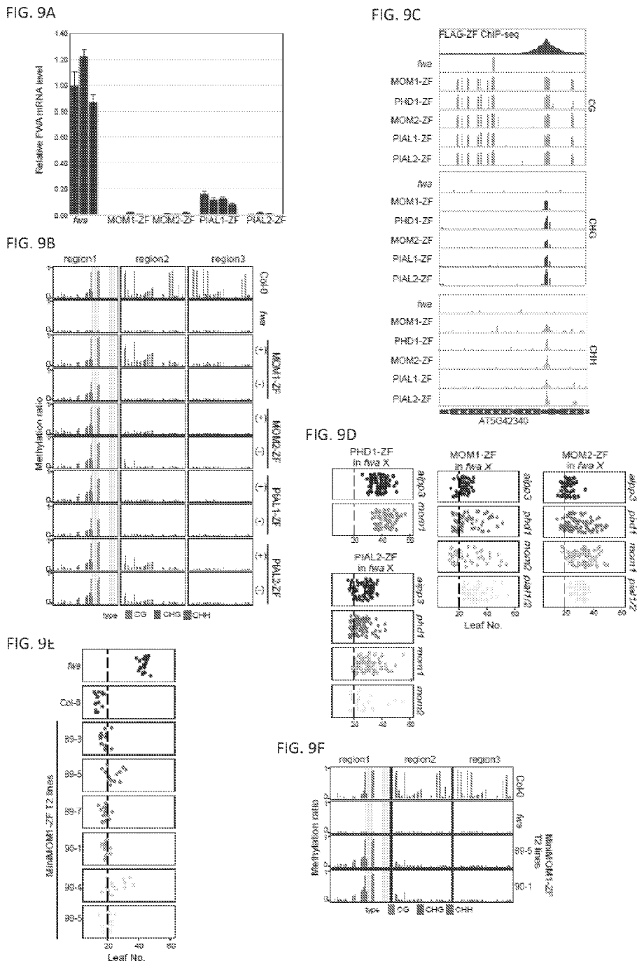

The heritability of the flowering time phenotypes for the ZF fusions that were not associated with FWA DNA methylation were similarly analyzed. In T2 plants that inherited the fusion protein transgenes, the early flowering phenotype was usually maintained (FIG. 9B). However, in all null segregant plants, the flowering time reverted to the typical late flowering phenotype of fwa plants (FIG. 8D), showing that the persistent presence of the fusion protein transgenes was needed for FWA silencing. Within the population of transgene containing T2 plants, a wide variation in flowering time was observed (FIG. 8D). This was likely due to differences in the expression level of the fusion proteins, as plants with high levels of transgene expression tended to have an early flowering phenotype, while plants with low protein expression levels tended to have a late flowering phenotype, as observed by Western blotting (FIG. 8E).

Although ZF was designed to bind the FWA promoter, it also binds to thousands of off-target sites throughout the genome (see e.g., Gallego-Bartolome, Cell, 2019). To explore whether the different ZF fusion proteins could regulate other genes near these off-target binding, RNA-seq data was analyzed. Genes near 6,091 ZF ChIP-seq peaks that had changed at least four-fold relative to fwa non-transgenic controls were analyzed. The differentially expressed genes (DEGs) near ZF off-target peaks were analyzed using Region Associated DEG (RAD) analysis (see e.g., Guo, Bioinformatics, 2021). This analysis showed that all the ZF fusions showed a significantly higher number of downregulated DEGs than upregulated DEGs when the ZF peak was within one kilobase of the start site of the gene (FIG. 1E and FIG. 8F). RNA expression levels for two representative genes downregulated by the ZF fusions are shown in FIG. 8F. These results show that all the identified fusions are able to repress other genes in addition to FWA.

### Example 2: Targeted Gene Silencing by DNA Methylation

Arabidopsis de novo DNA methylation requires the RNA-directed DNA methylation (RdDM) pathway, which can be divided into two main arms (see e.g., Gallego-Bartolome, Cell, 2019; Law, Nat. Rev. Genet., 2010; Matzke, Nat. Rev. Genet., 2014). The upstream arm consists of the biogenesis of 24 nucleotide small interfering RNAs (siRNAs), in a process that is initiated by transcription by RNA polymerase IV (Pol IV). Pol IV transcripts are made double stranded by RNA-dependent RNA polymerase 2 (RDR2), diced into siRNAs by Dicer-like 3 (DCL3), DLC2, and DCL4, and loaded into ARGONAUTE4 (AGO4), AGO6 and AGO9 (see e.g., Blevins, eLife, 2015; Zhai, Cell, 2015; Zilberman, Science, 2003). The downstream arm of the RdDM pathway consists of the synthesis of non-coding RNAs by RNA polymerase V (Pol V), which serve as scaffolds upon which AGO/siRNA complexes bind and then direct the DOMAINS REARRANGED METHYLASE2 (DRM2) to methylate target DNA (see e.g., Liu, Nat. Plants, 2018; Zhong, Cell, 2014). Pol V is recruited to chromatin by the DDR complex consisting of DMS3, DRD1, and RDM1, which is in turn recruited by methylated DNA via the methyl binding domain proteins SU(VAR)3-9 homolog 2 (SUVH2) and SUVH9 (see e.g., Johnson, Nature, 2014; Liu, PLoS Genet., 2014; Wongpalee, Nat. Comm., 2019) (FIG. 8A). RdDM pathway components also associate with the MORC proteins, which are loaded onto chromatin at RdDM sites, are retained at these sites in RdDM mutant backgrounds, and are needed at some sites for the efficiency of RdDM maintenance (see e.g., Jing, Mol. Plant, 2016; Liu, PLoS Genet., 2016; Xue, Nat. Comm., 2021). Once established, DNA methylation is maintained in a CG context by the DNA METHYLTRANSFERASE 1 (MET1), in the CHG (where H=A, T, or C) context mainly by CHROMOMETHYLASE 3 (CMT3), and in the CHH context by both CMT2 and the RdDM pathway (see e.g., Du, Mol. Cell Bio., 2015). In this Example, a novel PHD domain protein, PHD1, as well several of its interactors, MORPHEUS MOLECULE 1 (MOM1), MOM2, PROTEIN INHIBITOR OF ACTIVATED STAT LIKE1 (PIAL1), and PIAL2, were found to trigger FWA silencing via establishment of DNA methylation.

### Materials and Methods

### IP-MS

The method of IP-MS used in this Example has been described in a recent paper (see e.g., Xue, Nat. Comm., 2021). Ten grams of unopened floral buds from Col-0 wild type and FLAG-tag transgenic plants, including PHD1, MOM1, AIPP3, MOM2, MORC6, PIAL2, TRB1, TRB2, TRB3, JMJ14, HD2A, HD2B, and ELF7 were collected and ground into fine powder with liquid nitrogen. These samples were resuspended with 25 mL IP buffer and homogenized until lump-free by dounce homogenizer. The lysate was filtered through Miracloth and incubated with 250 μL anti-FLAG M2 magnetic beads (Sigma) at 4° C. for 2 hours. The magnetic beads were washed with IP buffer and eluted with TBS containing 250 μg/mL 3×FLAG peptides. The eluted proteins were precipitated with trichloroacetic acid (Sigma) and subject to MS analyses as described previously (see e.g., Xue, Nat. Comm., 2021).

### BS-PCR-seq

Unless stated otherwise, BS-PCR-seq was performed as in Example 1.

### WGBS

The leaf tissue of 4- to 5-week-old Arabidopsis Col-0 wild type, phd1-2, phd1-3, mom2-1, mom2-2, aipp3-1,fwa, and ZF transgenic lines (PHD1-ZF, MOM1-ZF, MOM2-ZF, PIAL1-ZF, and PIAL2-ZF) with early flowering phenotype were collected for DNA extraction using DNeasy Plant Mini Kit (QIAGEN). A total of 500 ng DNA was sheared with Covaris S2 (Covaris) into around 200 bp at 4° C. The DNA fragments were used to perform end repair reaction using the Kapa Hyper Prep kit (Roche), and together with Illumina TruSeq DNA sgl Index Set A/B (Illumina) to perform adapter ligation. The ligation products were purified with AMPure beads (Beckman Coulter), and then converted with EpiTect Bisulfite kit (QIAGEN). The converted ligation products were used as templates, together with the primers from the Kapa Hyper Prep kit (Roche) and MyTaq Master mix (Bioline) to perform PCR. The PCR products were purified with AMPure beads (Beckman Coulter) and sequenced by Illumina NovaSeq 6000 instrument.

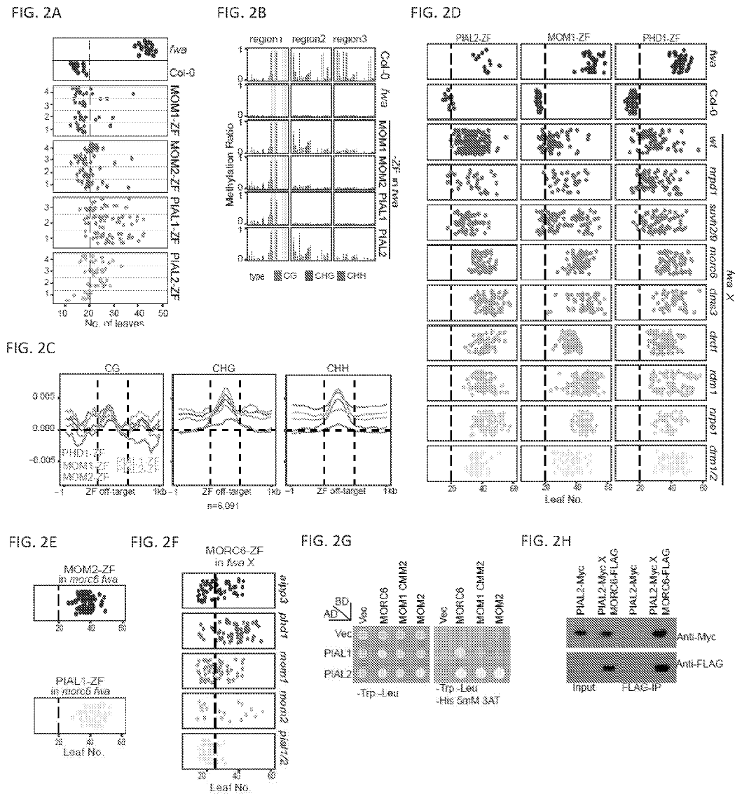

The WGBS data analysis has been described in Gallego-Bartolome, Cell, 2019 with minor modifications. The WGBS raw reads were aligned to both strands of reference genome TAIR10 using BSMAP (v.2.74) (see e.g., Xi, BMC Bioinform., 2009), and the alignment allowed up to 2 mismatches and 1 best hit. The reads with more than 3 consecutives methylated CHH sites were removed, and the methylation level was calculated with the ratio of C/(C+T). For FIG. 2C, the methylation levels at 1 kb flanking regions of ZF off target sites in PHD1-ZF, MOM1-ZF, MOM2-ZF, PIAL1-ZF, and PIAL2-ZF were subtracted by the methylation level of fwa and plotted with R package pheatmap.

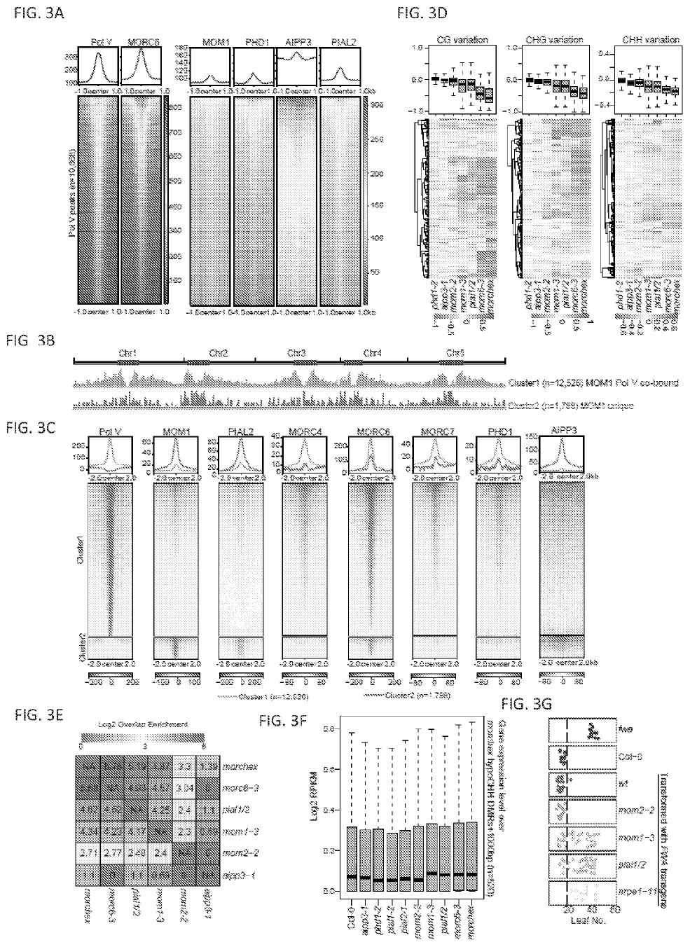

For FIG. 3D, the hcDMRs (p<0.01, >33 supported controls) of Col-0 wild type, aipp3-1, phd1-2, mom1-2, mom2-1, pial1 pial2, morc6, and morchex mutants were called using a previous method (see e.g., Zhang, PNAS, 2018), which were then used to generate the heat map using R package pheatmap [R. Kolde, Pheatmap: pretty heatmaps]. Y2H

The cDNA sequences of PIAL1, PIAL2, MOM2, MORC6, and MOM1 CMM2 domain (aa1660-aa1860, (see e.g., Han, Plant Cell Rep., 2016)) were first cloned into gateway entry vectors followed by LR reaction with pGBKT7-GW (Addgene 61703) and pGADT7-GW (Addgene 61702) destination vectors. Pairs of plasmid DNA for the desired protein interaction to be tested were co-transformed into the yeast strain AH109. Combinations of the empty pGBKT7-GW or pGADT7-GW vectors and the plasmids of desired proteins were used for transformation of yeast cells to test for self-activation. Transformed yeast cells were plated on synthetic dropout medium without Trp and Leu (SD-TL) and incubated for 2-3 days to allow for the growth of positive colonies carrying both plasmids. Three yeast colonies of each tested protein interaction pairs were picked and mixed in 150 μl 1×TE solution, and 3 μl of the 1×TE solution with the yeast cells were blotted on synthetic dropout medium without Trp, Leu, and His (SD-TLH) and with 5 mM 3-amino-1,2,4-triazole (3AT) to inhibit background growth. Growth of yeast on SD-TLH with 5 mM 3AT medium after 2-3 days of incubation indicates the interaction between the GAL4-AD fusion protein and the GAL4-BD fusion protein.

### Co-IP

The Co-IP assays in this study were performed by following previous protocol (see e.g., Wang, Science, 2016) with some modifications. 2 grams of 2-week-old seedling tissue were collected from Arabidopsis transgenic lines: JMJ14-FLAG×TRB1-Myc and TRB1-Myc; MORC6-FLAG×PIAL2-Myc and PIAL2-Myc. The samples were ground into fine powder with liquid nitrogen, resuspended with 10 mL IP buffer, and incubate 20 minutes at 4° C. The lysate was centrifuged and filtered through Miracloth for two times, and the supernatant was incubated with 30 μL anti-FLAG M2 Affinity Gel (Millipore) for 2 hours at 4° C. The anti-FLAG beads were washed with IP buffer for a total of 5 times, and the protein was eluted with 40 μL elution buffer (IP buffer containing 100 μg/mL 3×FLAG peptide as final concentration). The supernatant was mixed with 5×SDS loading buffer and subject to Western blot.

### Molecular Cloning

This pYAO-hSpCAS9-PHD1-K.O vector was used to generate PHD1 mutant alleles in Arabidopsis Col-0 background and the cloning method has been described in (see e.g., Yan, Mol. Plant, 2015). We first selected two guide RNAs by using the CRISPR-PLANT website at: https://world wide web.genome.arizona.edu/crispr/CRISPRsearch.html. The AtU6-26-sgRNA construct were used to perform overlapping PCR to combine PHD1 gRNAs with AtU6-26 small nuclear RNA promoter, which were then inserted into the SpeI digested pYAO:hSpCas9 construct by In-fusion (Takara Bio).

### Results

In Arabidopsis, de novo DNA methylation requires the RNA-directed DNA methylation (RdDM) pathway. Previous work demonstrated that directly tethering RdDM components with ZF can trigger de novo DNA methylation of the FWA promoter (see e.g., Gallego-Bartolome, Cell, 2019; Johnson, Nature, 2014). Since PHD1 is a novel factor that has not been implicated in DNA methylation control, the mechanism by which the PHD1-ZF fusion triggered DNA methylation at FWA was investigated. To identify PHD1 interacting proteins, a pPHD1:PHD1-FLAG transgenic line in the background of aphd1 loss of function mutant was generated and used for Immunoprecipitation-Mass Spectrometry (IP-MS) analysis. PHD1 pulled down a large number of peptides of MOM1 and a bromo-adjacent homology (BAH) domain-containing protein ASI1-IMMUNOPRECIPITATED PROTEIN 3 (AIPP3) (see e.g., Duan, PNAS, 2017), as well as a smaller number of peptides of MOM2, PIAL1, and PIAL2. A recent study that reported IP-MS on PHD1 also detected AIPP3 and MOM1 peptides, but not MOM2, PIAL1, or PIAL2 (see e.g., Qian, J. Integr. Plant Biol., 2021). An earlier study that performed IP-MS on MOM1 identified peptides of AIPP3 and PHD1, as well as PIAL2 (see e.g., Han, Plant Cell Rep., 2016). A similar study that performed an IP-MS analysis on epitope tagged MOM1 and AIPP3 found that both pulled down each other as well as PHD1, MOM2, PIAL1, and PIAL2. MOM1 encodes a SWI2/SNF2 related protein that is required for the silencing of DNA methylated genes and transposable elements (TEs) by a poorly understood mechanism. However, mom1 mutations have very little effect on the maintenance of DNA methylation, showing that they mainly work downstream of DNA methylation (see e.g., Amedeo, Nature, 2000). It was therefore surprising that MOM1 complex components might be involved in the establishment of DNA methylation.

To further study the possible role of PHD1 complex proteins in DNA methylation targeting, ZF fusion proteins with MOM1, MOM2, PIAL1, PIAL2 and AIPP3 were created and transformed into the fwa epiallele. Interestingly, each of these proteins, except AIPP3, restored the early flowering phenotype, significantly repressed FWA expression, and induced DNA methylation at the FWA promoter region (FIGS. 2A-2B and FIG. 9A). PIAL2 was more efficient at triggering FWA silencing than PIAL1, which is consistent with the observation that, while PIAL1 and 2 are similar genes that act in a genetically redundant fashion, the pial2 single mutant displayed a stronger phenotype than the pial1 single mutant (see e.g., Han, Plant Cell., 2016). To test the heritability of DNA methylation in these ZF fusions, BS-PCR-seq was performed in the T2 transgenic and null segregant lines. Indeed, the FWA methylation was inherited in both cases (FIG. 9B). Whole genome bisulfite sequencing (WGBS) showed that PHD1-ZF, MOM1-ZF, MOM2-ZF, PIAL1-ZF, and PIAL2-ZF also enhanced DNA methylation at ZF off-target sites (FIG. 2C and FIG. 9C).

To investigate which step in the RdDM pathway might be involved in the targeting of FWA methylation by these factors, PIAL2-ZF, MOM1-ZF, and PHD1-ZF were transformed into fwa backgrounds in which RdDM mutations had been introgressed, including nrpd1, suvh2/9, dms3, drd1, rdm1, nrpe1, and drm1/2 (see e.g., Gallego-Bartolome, Cell, 2019). PIAL2-ZF, MOM1-ZF, and PHD1-ZF were still capable of triggering an early flowering phenotype in nrpd1 (the largest subunit of Pol IV), suggesting that siRNA biogenesis was not needed for methylation targeting (FIG. 2D and FIG. 8A). These fusions were also capable of triggering silencing in the suvh2/9 mutant background (FIG. 2D), showing that the SUVH2 and SUVH9 factors that normally recruit the DDR complex and Pol V to chromatin were not needed for silencing (FIG. 8A). However, PIAL2-ZF, MOM1-ZF and PHD1-ZF silencing activity was blocked by DDR component mutations (dms3, drd1, and rdm1) as well as by mutations in the largest subunit of Pol V (nrpe1) and the DRM de novo methyltransferases (drm1/2) (FIG. 2D). These results place the action of PIAL2-ZF, MOM1-ZF, and PHD1-ZF upstream of the DDR complex, but downstream of SUVH2 and SUVH9 (FIG. 8A).

Interestingly, the RdDM factor MORC6 was previously shown to have identical behavior in these assays, with MORC6-ZF capable of triggering FWA methylation in wild type, nrpd1, and suvh2/9, but not in dms3, drd1, rdm1, nrpe1, or drm1/2 (see e.g., Gallego-Bartolome, Cell, 2019). Furthermore, like mom1 mutations, morc6 mutations show very little effect on the maintenance of CHH at RdDM sites (see e.g., Amedeo, Nature, 2000; Moissiard, PLoS Genet., 2012; Xue, Nat. Comm., 2021). These similarities prompted the testing of the targeting of PIAL2-ZF, MOM1-ZF, PHD1-ZF, MOM2-ZF, and PIAL1-ZF in the morc6 fwa genetic background. Interestingly, these ZF fusions failed to trigger FWA silencing in morc6 (FIGS. 2D-2E), suggesting that the MOM1 complex acts upstream of MORC6. To further confirm this order of action MORC6-ZF was transformed into fwa backgrounds in which the phd1-2, mom1-3, aipp3-1, and pial1/2 double mutants had been introgressed. MORC6-ZF successfully targeted silencing of FWA in all backgrounds (FIG. 2F), confirming that MORC6 acts downstream of the PHD1-MOM1 complex in the targeting of FWA silencing.

To dissect the hierarchy of action of MOM1 complex components, MOM1-ZF and PHD1-ZF were initially transformed into mom1 fwa and phd1-2 fwa mutant backgrounds (FIG. 9D). MOM1-ZF was able to trigger early flowering in phd1-2 fwa, positioning MOM1 downstream of PHD1 (FIG. 9D). Consistent with this order of action, the mom1 mutant blocked PHD1-ZF activity (FIG. 9D). PHD1-ZF activity was also blocked in the aipp3 fwa mutant, positioning PHD1 upstream of AIPP3 as well (FIG. 9D). These results were consistent with the IP-MS result showing that the MOM1-PHD1 interaction was abolished in the aipp3-1 mutant. To further dissect the hierarchy, PIAL2-ZF was transformed into aipp3 fwa, phd1 fwa, mom2 fwa and mom1 fwa mutant backgrounds. FIG. 9D shows that PIAL2-ZF triggered an early flowering phenotype in all mutant backgrounds, suggesting that PIAL2 might act at the most downstream position within the MOM1 complex. MOM1-ZF was also transformed into aipp3 fwa, mom2 fwa and pial1/2 fwa. MOM1-ZF was also able to trigger early flowering in all these mutant backgrounds (FIG. 9D), suggesting that MOM1 acts at a step parallel with PIAL1/2 in targeting DNA methylation. MOM1-ZF showed a lower efficiency of triggering early flowering in the pial1/2 fwa mutant compared to the other mutants (FIG. 9D), suggesting that PIAL1/2 is required for the full functionality of MOM1-ZF. Additionally, MOM2-ZF was transformed into aipp3 fwa, phd1 fwa, mom1 fwa, and pial1/2 fwa, and like MOM1-ZF and PIAL2-ZF, MOM2-ZF was able to trigger early flowering in all the mutants with lower efficiency (FIG. 9D), suggesting that MOM2 also acts with MOM1 and PIAL2 in a very downstream step in triggering methylation.

A small domain of MOM1 called the CMM2 domain has been shown to retain some MOM1 function in silencing downstream of DNA methylation (see e.g., Nishimura, PLoS Genet., 2012). A ZF fusion with the CMM2 domain together with a nuclear localization signal (called miniMOM1) (see e.g., Caikovski PLoS Genet., 2008) was efficient at targeting FWA silencing and methylation (FIGS. 9E-9F). IP-MS was performed with a miniMOM1-FLAG line and peptides for MOM2, PIAL1, and PIAL2, but not for AIPP3 or PHD1, were found. These results further suggest that AIPP3 and PHD1 are dispensable for the targeting of methylation to FWA, which is consistent with MOM1, PIAL1/PIAL2, and MOM2 acting as the most downstream factors in the MOM1 complex.

Given that PIAL1/PIAL2, MOM1, and MOM2 appeared to be the most downstream critical components of the MOM1 complex required for triggering FWA methylation and that ZF fusions of these proteins failed to trigger methylation in a morc6 mutant, at least one of these components might physically interact with MORC6. Indeed, PIAL2 was able to interact with MORC6 in a yeast two hybrid assay (FIG. 2G). This interaction was confirmed by co-IP, with MORC6-FLAG interacting with PIAL2-Myc (FIG. 2H). While there could certainly be other important interactions, these results suggest that the MOM1 complex likely recruits MORC6 in part via a physical interaction between PIAL2 and MORC6. MORC6 then triggers FWA methylation via its interaction with the RdDM machinery (see e.g., Xue, Nat. Comm., 2021).

### Example 3: Endogenous Functions of MOM1 Complex Components

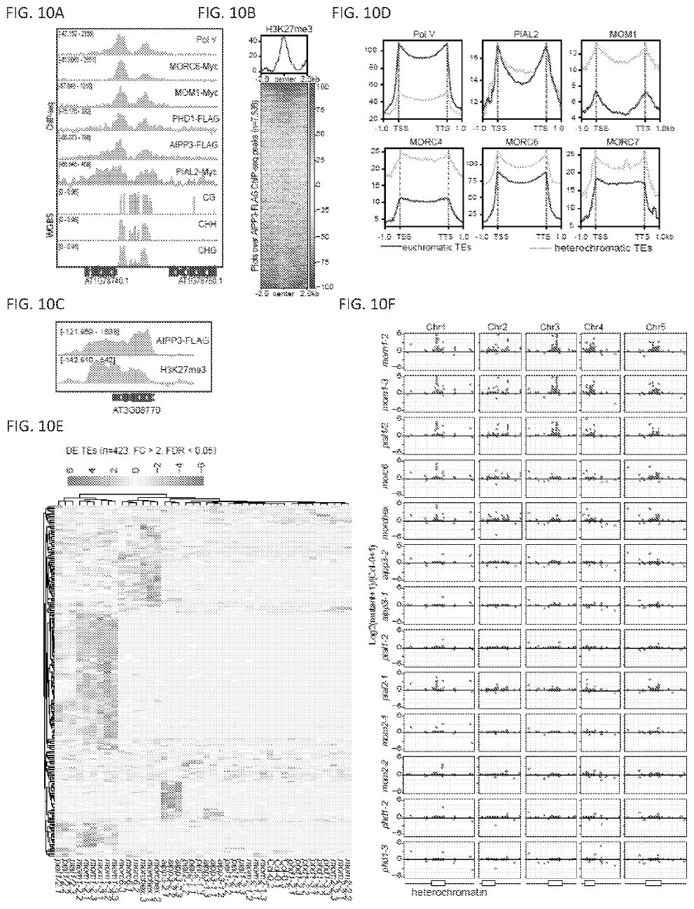

To study the endogenous function of MOM1 complex components and their possible role in DNA methylation control, chromatin immunoprecipitation sequencing (ChIP-seq) was performed in FLAG or MYC tagged lines. MOM1, PHD1, AIPP3, MORC6, and PIAL2 were all highly colocalized with Pol V at RdDM sites (FIG. 3A and FIG. 10A), suggesting an endogenous role for the MOM1 complex in RdDM. AIPP3 was also present at H3K27me3 sites (FIGS. 10B-10C), consistent with recent studies showing that AIPP3 also acts in a separate complex that reads the H3K27me3 mark (see e.g., Qian, J. Integr. Plant Biol., 2021; Zhang, Nat. Comm., 2020). MOM1 and PIAL2, as well as MORC6, MORC4, and MORC7 (see e.g., Xue, Nat. Comm., 2021), also showed broad localization over pericentromeric regions not necessarily overlapping with RdDM sites (FIGS. 3B-3C), consistent with mom1, pial1/2, and morc mutant RNA-seq data showing upregulation of heterochromatin in these regions (see e.g., Han, Plant Cell Rep., 2016; Harris, PLoS Genet., 2016; Moissiard, Science, 2012; Xue, Nat. Comm., 2021). Consistent with this pattern, MOM1, MORCs, and to a lesser extent PIAL2 were more highly enriched on heterochromatic transposable elements (TEs) with high levels of H3K9me2 and abundant in pericentromeric regions as compared to euchromatic TEs with lower H3K9me2 levels mostly present in the chromosome arms, which is the opposite pattern to that of Pol V ChIP-seq (see e.g., Liu, PLoS Genet., 2018) (FIG. 10D). Thus, while MOM1 and PIAL2 show strong localization to RdDM sites, they and the MORC proteins are also present at other DNA methylated sites, consistent with the hypothesis that MORC proteins and MOM1 complex components play dual roles at both RdDM sites and in deep pericentromeric heterochromatin where they are involved in TE repression downstream of DNA methylation (see e.g., Amedeo, Nature, 2000; Moissiard, PLoS Genet., 2012).

To further explore the functions of MOM1 complex components, RNA-seq was performed in Col-0, phd1-2, phd1-3, mom1-1, mom1-3, aipp3-1, aipp3-2, mom2-1, mom2-2, pial1-1, pial2-1, morc6-3, pial1/2, and morc1/2/4/5/6/7 hextuple (morchex) mutants (see e.g., Harris, PLoS Genet., 2016; Xue, Nat. Comm., 2021). The upregulated differentially expressed TEs (DE-TEs) in the morc6 and morchex mutants (see e.g., Harris, PLoS Genet., 2016) showed a prominent overlap with those of the mom1-2, mom1-3, and pial1/2 mutants (FIG. 10E). These results are consistent with the overlapping patterns of genomic localization between MOM1, PIAL2, and MORCs (FIG. 3A and FIG. 10A). The phd1, aipp3, and mom2 mutants on the other hand showed little change in expression at these same sites (FIG. 10E), suggesting that these factors are less important for this silencing function. The mom1 and pial1/2 mutants also showed broader transcriptional upregulation in pericentromeric regions (FIG. 10F), as previously reported (see e.g., Han, Plant Cell Rep., 2016), and to a higher extent than that seen in the morc mutants.

Whole-genome bisulfite sequencing (WGBS) was performed in phd1-2, phd1-3, aipp3-1, and mom2-2, analyzed with previously published WGBS data from the morc6-3, morchex, mom1-3 and pial1/2 mutants (see e.g., Han, Plant Cell Rep., 2016; Harris, PLoS Genet., 2016; Stroud, Cell, 2013), and followed by analysis using the High-Confidence Differentially Methylated Regions (hcDMRs) pipeline (see e.g., Zhang, PNAS, 2018). We observed a small number of CHH hcDMRs in mom1-3, mom2-2, and pial1/2 double mutant, which significantly overlapped with those of morc6 and morchex at RdDM sites (FIGS. 3D-3E). This is consistent with an earlier analysis that showed a strong overlap of mom1 hypomethylated DMRs with those of the morchex mutant (see e.g., Zhang, PNAS, 2018). On the other hand, the aipp3-1 mutant only shared 1 out of 13 hypo CHH-hcDMRs with the morc6 mutant (FIG. 3E), and neither of the phd1 mutant alleles tested showed any hypo CHH-hcDMRs. These DNA methylation results are consistent with the RNA-seq results showing that the genes located within 1 kb of the 520 CHH hcDMR regions previously found in the morchex mutant were upregulated in mom1-3, pial1/2, morc6 and morchex mutants, but not in phd1-2, aipp3-1, pial1-2, pial2-1, or mom2-2 mutants (FIG. 3F). These results show that MOM1/PIAL1/PIAL2, along with MORCs, are required for the maintenance of CHH methylation and gene silencing at a small subset of RdDM sites, while AIPP3, PHD1, and MOM2 seem to have a minimal role in this process.

The MORC genes have previously been shown to play a role in the initial establishment of DNA methylation at the FWA locus. Studies have shown that when an extra copy of FWA is introduced into Arabidopsis plants via Agrobacterium-mediated transformation, it is very efficiently methylated and silenced in the wild type background. However, this methylation and silencing is blocked in RdDM mutants, leading to overexpression and a late flowering phenotype (see e.g., Cao and Jacobsen, Curr. Biol., 2002). FWA silencing in this assay was also shown to be partially blocked in the morchex mutant (see e.g., Xue et al., Nat. Comm., 2021). To test whether MOM1 complex components are involved in this process mom1-3, mom2-2, and pial1/2 mutants were transformed with FWA. Similar to morc6, the mom1-3 and pial1/2 mutants caused a late flowering phenotype in most transgenic lines, whereas the mom2-2 mutant showed almost no effect (FIG. 3G). Together, these results show that in addition to their previously recognized role in silencing downstream of DNA methylation, MOM1 and PIAL1/PIAL2 also act in methylation control at RdDM sites together with MORCs.

### Example 4: Targeted Gene Silencing by TRBs, LHP1, MSI1, and H3K7Me3 Deposition

In addition to DNA methylation, histone modifications also contribute to gene silencing. For example, Arabidopsis FLOWERING LOCUS C (FLC) is repressed by the subunits of Polycomb Repressive Complex 2 (PRC2), including CURLY LEAF (CLF), EMBRYONIC FLOWER 2 (EMF2), and FERTILIZATION INDEPENDENT ENDOSPERM (FIE). The PRC2 complex is conserved in plants and animals and acts to deposit histone H3K27 trimethylation at specific loci (see e.g., Jiang, PLoS, 2008; Mozgova, Annu. Rev. Plant Biol., 2015). Another component of the PRC2 complex MULTICOPY SUPPRESSOR OF IRA1 (MSI1) also interacts with the Arabidopsis PRC1 component LIKE HETEROCHROMATIN PROTEIN 1 (LHP1) (see e.g., Derkacheva, EMBO, 2013; Mylne, PNAS, 2006; Zhang, Nat. Struct. Mol. Biol., 2007), which is important for H3K27me3 mediated gene silencing.

In this Example, a number of proteins capable of silencing FWA in a DNA methylation independent manner, including TELOMERE REPEAT BINDING FACTOR (TRB)1, 2, and 3, LHP1, and MSI1 were found. The fusion proteins also triggered silencing at many other genes whose promoters were nearby ZF off-target DNA sequences.

### Results

TRB1, TRB2, and TRB3 were among the ZF fusions that caused repression of FWA without affecting DNA methylation. Arabidopsis TRB proteins are well known for their role in telomere binding and protection (see e.g., Schrumpfova, Plant J., 2014). In addition, recent studies have shown that TRBs can recruit Polycomb Repressive Complex 2 (PRC2) for deposition of the repressive histone mark H3K27me3 (see e.g., Bloomer, PNAS, 2020; Zhou, Nat. Genet., 2018). It therefore seemed possible that TRB1-ZF, TRB2-ZF, and ZF-TRB3 (TRB-ZFs) may trigger FWA silencing by targeting the deposition of H3K27me3.

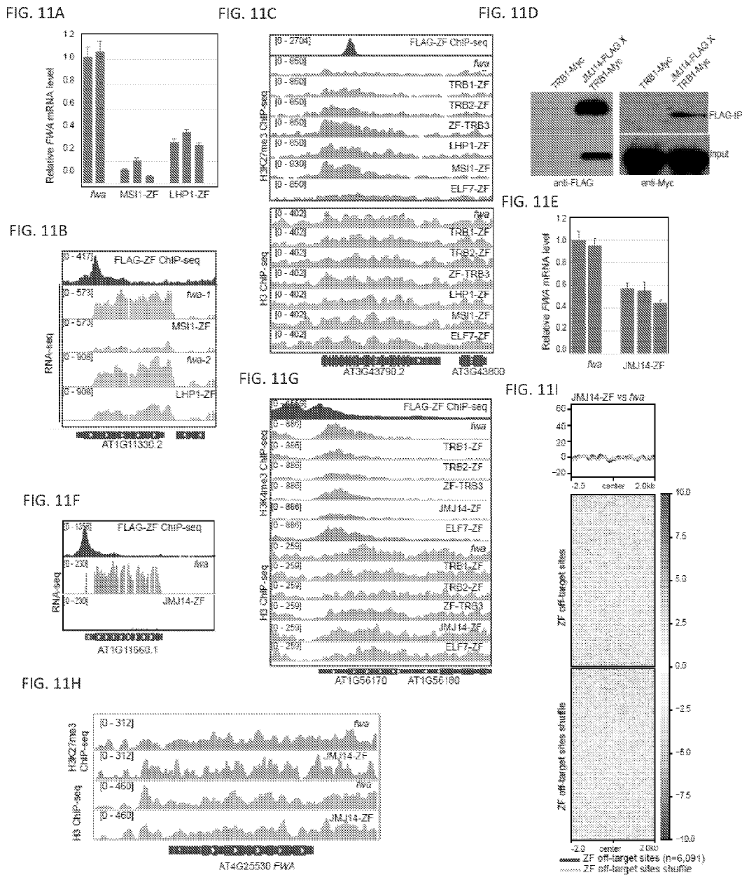

To test whether depositing H3K27me3 at FWA could lead to gene silencing and an early flowering phenotype, ZF was fused with several subunits of the PRC2 complex including FIS2, MSI1, VRN2, EMF2, and CLF, as well as with LHP1 (a component of the PRC1 complex), none of which were included in the initial screen, and transformed these fusion constructs into the fwa background. Both MSI1-ZF and LHP1-ZF caused a very early flowering phenotype (FIG. 4A). Consistent with the results of TRB-ZFs, neither MSI1-ZF nor LHP1-ZF triggered DNA methylation at FWA (FIG. 4B), though they caused a significant level of FWA repression (FIG. 11A). In addition, RAD analysis indicated that, similar to TRB-ZFs (FIG. 1E), MSI1-ZF and LHP1-ZF showed more downregulated DEGs than upregulated DEGs near ZF off-target sites, showing that they could target the silencing of additional genes (FIG. 4C and FIG. 11B).

To verify the deposition of H3K27me3 at FWA and ZF off-target sites H3K27me3 and H3 ChIP-seq were performed in TRB-ZFs, MSI1-ZF, and LHP1-ZF plants. Indeed, H3K27me3 ChIP-seq signals were higher at FWA in TRB-ZFs, LHP1-ZF, and MSI1-ZF when compared to fwa control plants (FIG. 4D). We also observed H3K27me3 ChIP enrichment in TRB-ZFs, LHP1-ZF, and MSI1-ZF when plotting over 6,091 ZF off-target sites (FIG. 4E and FIG. 11C). Together, these results suggest that tethering TRBs, MSI1, and LHP1 to FWA and other target genes can cause gene silencing associated with H3K27me3 deposition.

### Example 5: Targeted Gene Silencing by TRBs, JMJ14, and H3K4Me3 Removal

In this Example, a number of proteins capable of silencing FWA in a DNA methylation independent manner, including TELOMERE REPEAT BINDING FACTOR (TRB)1, 2, and 3, and JMJ14 were found. The fusion proteins also triggered silencing at many other genes whose promoters were nearby ZF off-target DNA sequences.

### Results

To further characterize TRB1/2/3 directed gene silencing, pTRB:TRB-FLAG transgenes in their respective mutant background were generated and IP-MS to identify TRB interacting proteins was performed. Consistent with previous studies (see e.g., Bloomer, PNAS, 2020; Zhou, Nat. Genet., 2018), TRBs pulled down LHP1 and several PRC2 subunits. Interestingly, peptides of the Arabidopsis histone H3K4me3 demethylase JMJ14 as well as its two known NAC domain interactors, NAC050 and NAC052 (see e.g., Ning, PLoS Genet., 2015; Zhang, Nat. Struct. Mol. Biol., 2015), were also pulled down by TRB-FLAG, suggesting that TRBs may recruit the JMJ14-NAC050-NAC052 complex to remove H3K4me3, contributing to gene silencing. To verify the interaction of TRBs and JMJ14, IP-MS was performed in pJMJ14:JMJ14-FLAG transgenic plants, which pulled down several peptides of TRB1. To further confirm the interaction, a co-IP experiment was performed in pJMJ14:JMJ14-FLAG and pTRB1:TRB1-Myc crossed lines, where JMJ14 indeed pulled down TRB1 (FIG. 11D).

To test whether directly targeting JMJ14 to the FWA promoter region could trigger silencing, JMJ14 was fused with ZF. JMJ14-ZF successfully triggered an early flowering phenotype and silenced FWA in a DNA methylation independent manner (FIGS. 4A-4B and FIG. 11E). RAD analysis of JMJ14-ZF RNA-seq data identified more downregulated than upregulated DEGs near ZF off-target sites, suggesting that the silencing activity of JMJ14 can also act at other loci (FIG. 4C and FIG. 11F).

H3K4me3 and H3 ChIP-seq was performed in TRB-ZFs, JMJ14-ZF and an fwa control. Both TRB-ZFs and JMJ14-ZF caused a reduction of H3K4me3 at the FWA locus (FIG. 4F). However, in the ZF off-target regions, JMJ14-ZF showed a much stronger H3K4me3 reduction than the other ZF lines (FIG. 4G and FIG. 11G). In addition, unlike TRB-ZFs, MSI1-ZF and LHP1-ZF (FIGS. 4D-4E), JMJ14-ZF did not show accumulation of H3K27me3 at FWA (FIG. 11H), nor at ZF off-target regions (FIG. 11I). Thus, silencing by JMJ14 is likely acting directly via removal of H3K4me3 rather than by accumulation of H3K27me3, a mark which can act antagonistically with H3K4me3 (see e.g., Piunti, Science, 2016; Voigt, Genes Dev., 2013; Yang, Nat. Genet., 2018).

### Example 6: Endogenous Function of TRBs-JMJ14 in Gene Regulation

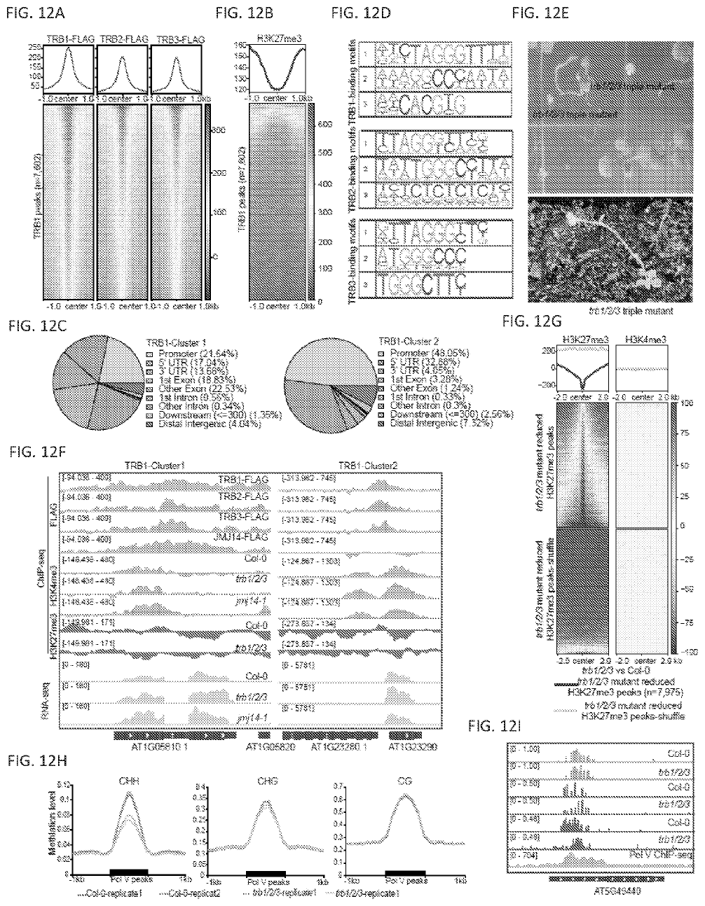

To study the natural function of TRBs-JMJ14, ChIP-seq was performed utilizing pTRB:TRB-FLAG and pJMJ14:JMJ14-FLAG transgenic lines to examine target sites throughout the genome. Consistent with their reported genetic redundancy (see e.g., Kuchar, FEBS Lett., 2004; Zhou, Nat. Genet., 2018), TRB1, TRB2 and TRB3 were highly co-localized throughout the genome (FIG. 12A). TRBs have been reported to recruit the PRC2 complex for H3K27me3 deposition (see e.g., Bloomer, PNAS, 2020; Zhou, Nat. Genet., 2018), and an enrichment of H3K27me3 signals flanking TRB peaks was observed (FIG. 12B), with TRB peaks mainly located at promoter regions and H3K27me3 distributed over gene bodies. Also, consistent with the observed TRB-JMJ14 interaction, an overlap between JMJ14 and TRB peaks was observed (FIG. 5A). However, there was a large fraction of TRB1 peaks that did not overlap with JMJ14 peaks (FIG. 5A). Therefore, the TRB peaks were sorted into two clusters: peaks that overlapped JMJ14 peaks (Cluster 1) and peaks that did not (Cluster 2) (FIGS. 5A-5B).

Interestingly, more than 80% of the peaks in Cluster 2 were located at promoter and 5′ UTR regions, compared to less than 40% of those in Cluster 1 (FIG. 12C). On the other hand, 40% of TRB Cluster 1 peaks were located in exons compared to less than 5% of those in Cluster 2 (FIG. 12C). These data suggest that the colocalization of TRBs and JMJ14 occurs most often in gene bodies, but not as often at promoters. Previous work has shown that the peaks of TRB1 ChIP-seq are enriched in the telobox motif (see e.g., Zhou, Plant Cell Rep., 2016). Consistent with this result, motif prediction using TRB binding sites showed an enrichment for sequences similar to the Arabidopsis telomere repeat sequence TTTAGGG, not only in TRB1 ChIP-seq, but also in TRB2 and TRB3 ChIP-seq datasets (FIG. 12D). The overlap of H3K4me3 ChIP-seq signals with TRB and JMJ14 peaks was also examined. Cluster 1 TRB peaks that also colocalized with JMJ14 showed very low levels of H3K4me3, consistent with the demethylation activity of JMJ14. Cluster 2 peaks showed much higher levels of H3K4me3, especially at the flanks of TRB peak centers (FIGS. 5A-5B). This is likely because TRB peaks were most often in promoter regions while H3K4me3 is usually located near the start of transcription and in the 5′ transcribed regions of genes (FIGS. 5A-5B and FIG. 12C).

TRB1, TRB2 and TRB3 are redundant homologs, and the single and double mutants of any combination show no morphological phenotype (see e.g., Zhou, Nat. Genet., 2018). A trb1/2/3 triple mutant was also reported to be viable (see e.g., Zhou, Nat. Genet., 2018). T-DNA insertion mutant lines were utilized to create a trb1/2/3 triple mutant, which exhibited a much stronger morphological defect than previously reported. trb1/2/3 seedlings were very small and yellowish when grown on solid media, and when transplanted onto soil, they often failed to survive and were completely infertile (FIG. 12E). The previously described triple mutant utilized either a Ws-2 background T-DNA insertion allele (trb2-1) or a CRISPR/Cas9 generated trb2 allele (trb2-2) and a different T-DNA insertion allele of trb3-2 which was likely not a null allele (see e.g., Zhou, Nat. Genet., 2018). ChIP-seq analysis also showed that our trb1/2/3 allele had a stronger impact on H3K27me3 levels, with 7,975 decreased and 2,184 increased H3K27me3 regions compared to 730 decreased and 609 increased regions in the previously reported trb1/2/3 mutant (see e.g., Zhou, Nat. Genet., 2018).

To investigate the function of TRBs and JMJ14 in H3K4me3 control, H3K4me3 ChIP-seq was performed in trb1/2/3 and jmj14-1 mutants. H3K4me3 ChIP-seq signals were strongly increased at JMJ14 binding sites in both the trb1/2/3 triple mutant and the jmj14-1 mutant (FIG. 5C and FIG. 12F). Interestingly, H3K4me3 ChIP-seq signals were also increased in the trb1/2/3 triple mutant at TRB1 Cluster 2 peaks, which did not overlap with JMJ14 peaks (FIG. 5C and FIG. 12F), suggesting that TRBs likely influence the level of H3K4me3 in both a JMJ14-dependent and -independent manner. Although it seemed possible this could be due to the loss of H3K27me3 in trb1/2/3, especially because H3K4me3 and H3K27me3 have been shown to be in some cases mutually antagonistic (see e.g., Geisler and Paro, Development, 2015; Piunti, Science, 2016; Qian, Nat. Comm., 2018; Rothbart, B B A, 2014), there were a much greater number of regions of increased H3K4me3 (16,634) than regions decreased in H3K27me3 (7,975) in the trb1/2/3 mutant. In addition, the majority of H3K27me3 reduced regions in trb1/2/3 did not gain H3K4me3 ChIP-seq signals (FIG. 12G). These data suggest that the induction of H3K4me3 in trb1/2/3 mutant cannot be explained by changes in H3K27me3 levels.

To uncover genes that are regulated by TRBs and JMJ14, an RNA-seq was performed in the trb1/2/3 triple mutant and compared to jmj14-1 mutant plants or Col-0 wild type. Both mutants exhibited many DEGs, the majority of which were upregulated (FIG. 5D). If TRBs and JMJ14 coordinately regulate gene expression, a common set of DEGs in the mutants is expected. Indeed, the DEGs of the trb1/2/3 triple mutant and the jmj14-1 mutant significantly overlapped with a total of 177 out of 485 upregulated DEGs in the jmj14-1 mutant also upregulated in the trb1/2/3 triple mutant (FIG. 5E). This overlap was even greater when only including genes that were bound by both TRB1 and JMJ14 (FIG. 5E).

Previous reports showed that the jmj14-1 mutant exhibits a mild reduction in CHH DNA methylation at sites of RdDM (see e.g., Deleris, EMBO Rep., 2010; Greenberg, PLoS Genet., 2013). Because of the interaction between TRBs and JMJ14, WGBS was performed in the trb1/2/3 triple mutant. Indeed, a reduction of CHH DNA methylation at RdDM sites in trb1/2/3 was observed (FIGS. 12H-12I).

### Example 7: Targeted Gene Silencing by HDACs and Histone Deacetylation

Gene silencing can be achieved not only by adding repressive histone marks, but also by removing activating histone marks. For example, histone H3K4me2 and H3K4me3 are correlated with gene activity, and erasing H3K4me2 and H3K4me3 through H3K4 demethylases, such as JUMONJI 14 (JMJ14), can lead to gene repression (see e.g., Lu, Cell Res., 2010). Histone acetylation is another positive histone modification mark, which corresponds to active gene expression. Arabidopsis contains a large number of histone deacetylases (HDACs) that act to remove histone acetylation, including 18 members that are phylogenetically classified into three groups, such as Reduced Potassium Dependency-3/Histone Deacetylase-1 type (RPD3/HDA1), Histone Deacetylase-2 type (HD2), and Silent Information Regulator-2 (SIR2)-like type (see e.g., Hollender, J. Integr. Plant Biol., 2008). HDA6 is one of the most well studied HDACs, and the hda6 mutant shows upregulation of FLC and its homologs MAF4 and MAF5 along with increased H3 acetylation (see e.g., Yu, Plant Physiol., 2011). HD2 is a family of plant-specific HDACs, which has 4 members, HD2A, HD2B, HD2C, and HD2D (see e.g., Chen, Plant Cell, 2018). In this Example, HDACs were tethered with ZF to study gene silencing of FWA.

### Results

Three members of a family of histone deacetylases (HDACs), HD2A, HD2B, and HD2C were found to cause silencing of FWA when tethered with ZF but did not target DNA methylation (FIGS. 1B-1D). A fourth more distantly related member of this family called HD2D, did not cause silencing of FWA (FIG. 13A). It was previously shown that Arabidopsis HD2A is required for H3K9 deacetylation and rRNA gene silencing (see e.g., Lawrence, Mol. Cell, 2004), and that HD2C mediates H4K16 deacetylation and is involved in ribosome biogenesis (see e.g., Chen, J. Exp. Bot., 2018), suggesting that HD2 family members can deacetylate multiple sites. H3K9ac, H4K16ac, H3K27ac and H3 patterns were profiled by ChIP-seq in HD2A-ZF, HD2B-ZF, and HD2C-ZF plants. H3K9ac, H4K16ac and H3K27ac at FWA were moderately reduced in HD2A-ZF, HD2B-ZF, and HD2C-ZF plants (FIG. 6A). H3K9ac, H3K27ac, and H4K16ac ChIP-seq signals were plotted over ZF off-target sites. A reduction in HD2A-ZF, HD2B-ZF, and HD2C-ZF plants was observed (FIG. 6B and FIG. 13B). These results show that HD2A, HD2B, and HD2C can repress gene expression and reduce histone acetylation at FWA and other genes.

To identify interactors of HD2 family proteins, pHD2A:HD2A-FLAG and pHD2B:HD2B-FLAG transgenic lines in their respective mutant backgrounds were generated and IP-MS was performed. Both HD2A and HD2B pulled down many peptides of all four HD2 proteins, suggesting that this protein family forms a complex. This is consistent with a previously published IP-MS analysis of HD2C showing an interaction with HD2A, B, and D (see e.g., Chen, J. Exp. Bot., 2018). pHD2C:HD2C-FLAG was also generated and a ChIP-seq analysis was performed for HD2A, B, and C. HD2A and HD2C were highly overlapping across the genome (FIG. 13C), while HD2B ChIP-seq signals were not above background. The role of HD2A, HD2B, and HD2C in histone deacetylation using H3K9ac, H4K16ac and H3K27ac ChIP-seq in hd2a, hd2b, and hd2c T-DNA insertion mutant plants was explored. As previously reported for an hd2c mutant (see e.g., Chen, J. Exp. Bot., 2018), H4K16ac ChIP-seq signals were greatly increased over HD2A target sites in hd2c mutants, but not in hd2a or hd2b mutants (FIGS. 13D-13E) (see e.g., Colville, Plant Cell Rep., 2011). In addition, H3K9ac was increased in all three hd2 mutants at HD2A target sites, while H3K27ac showed only small variations in the three mutants (FIGS. 13D-13E). These results confirm that the HD2 family proteins are involved in histone deacetylation.

HDA6 was previously reported to interact with HD2A, HD2C, and HD2D in co-IP and bimolecular fluorescence complementation (BiFC) experiments (see e.g., Chen, J. Exp. Bot., 2010; Luo, Plant Singal. Behav., 2012). ZF fusions of HDA6 as well as other histone deacetylase family members HDA9, HDA15, and HDA19 were tested for silencing activity. HDA6-ZF, but not the other fusions, triggered an early flowering phenotype and FWA silencing (FIG. 6C and FIG. 13F) and this silencing was not accompanied by FWA DNA methylation (FIG. 6D). RNA-seq in HDA6-ZF was performed and more downregulated DEGs than upregulated DEGs at ZF off-target sites using RAD analysis were found (FIG. 6E), showing that HDA6 can act as a negative gene regulator.

HDA6 has been reported to deacetylate several substrates including K9, K14, K18, K23, and K27 of the H3 histone tail and K5, K8, and K12 of the H4 histone tail (see e.g., To, BBA, 2011), with H3K9ac and H3K14ac confirmed in multiple studies (see e.g., Earley, Genes Dev., 2006; Lin, Genome Res., 2020). H3K9ac, H3K14ac, and H3 ChIP-seq in HDA6-ZF was performed. Indeed, both H3K9ac and H3K14ac ChIP-seq signals were reduced at FWA as well as at ZF off-target sites in HDA6-ZF plants (FIGS. 6F-6G), suggesting that HDA6 represses target gene expression at least partially via histone H3K9 and H3K14 deacetylation. Together these results demonstrate that a variety of different histone deacetylase proteins of different classes can be harnessed for targeted gene silencing.

### Example 8: FWA Silencing Targeted by ELF7-ZF and Interference with Pol II Elongation

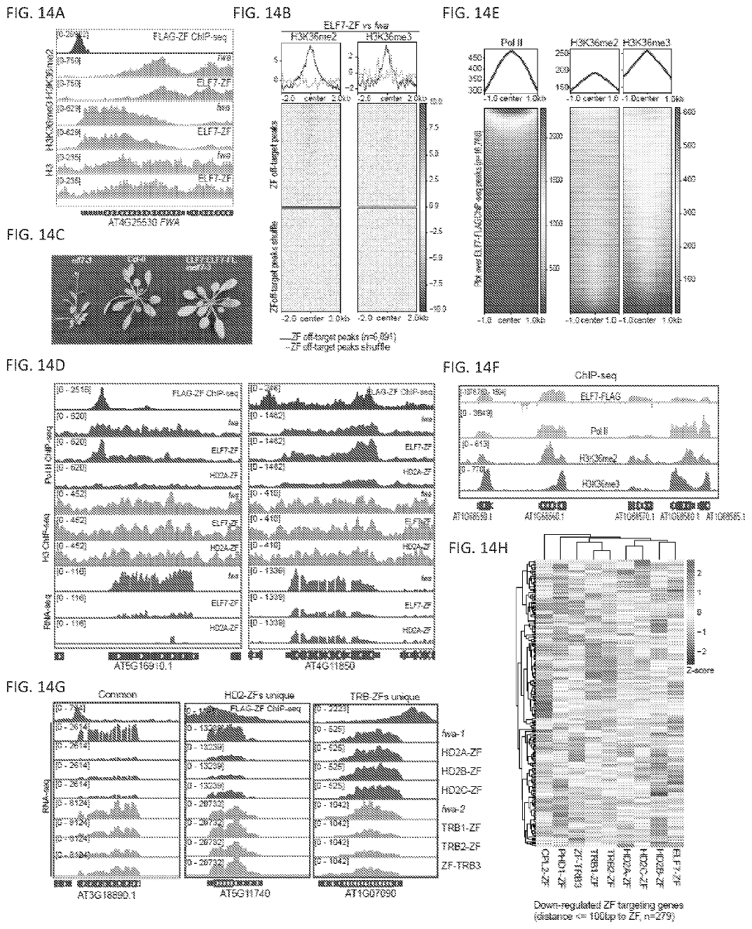

A ZF fusion with ELF7 also caused silencing of FWA without affecting DNA methylation (FIG. 1). ELF7 encodes an RNA Polymerase II-Associated Factor 1 (PAF1) homolog, which is a subunit of the PAF1 complex (PAF1C). PAF1C is a conserved protein complex in eukaryotes that collaborates with RNA polymerase II during transcription initiation and elongation (see e.g., Antosz, Plant Cell Rep., 2017; Tomson, B B A, 2013). In Arabidopsis, mutation of PAF1C subunit VIP3 caused a redistribution of histone H3K4me3 and H3K36me2 in certain genes (see e.g., Oh, Nat. Comm., 2008). Therefore, H3K4me3, H3K36me2, and H3K36me3 ChIP-seq was performed to see whether changes in these epigenetic marks might explain ELF7-ZF triggered FWA suppression. Some reduction of H3K4me3 at FWA in ELF7-ZF compared to fwa control plants was observed (FIG. 4F). However, unlike TRB-ZFs and JMJ14-ZF, H3K4me3 signal was largely unaffected near ZF off-target sites (FIG. 4G). Considering that ELF7-ZF did trigger gene silencing at ZF off-target sites (FIG. 1E), it seemed unlikely that H3K4me3 reduction was the relevant mechanism. In addition, signals of both H3K36me2 and H3K36me3 were slightly decreased at the FWA locus (FIG. 14A), while at the same time somewhat increased over ZF off-target sites (FIG. 14B), making it unlikely that changes in H3K36me2 or H3K36me3 levels were the direct cause of ELF7-ZF mediated gene silencing.

To explore other possible mechanisms by which ELF7-ZF may be silencing FWA, pELF7:ELF-FLAG transgenic lines in the elf7-3 mutant background were generated to perform IP-MS to identify ELF7 interacting proteins. The pELF7:ELF-FLAG transgenic lines successfully complemented the early flowering phenotype of elf7-3 mutant (see e.g., He, Genes Dev., 2004) (FIG. 14C). Consistent with previous work (see e.g., Antosz, Plant Cell., 2017) the ELF7 IP-MS data included peptides corresponding to all of the subunits of the PAF1 complex, as well as Pol II subunits and transcription factors, consistent with a role of ELF7 in Pol II transcription. Since ELF7 is a Pol II interacting protein, it was hypothesized that ELF7-ZF might interact with Pol II at the FWA promoter region, retaining it there, and inhibiting transcriptional elongation. To test this hypothesis, Pol II ChIP-seq was performed in ELF7-ZF transgenic lines, as well as untransformed fwa and HD2A-ZF as controls. As expected, Pol II occupancy at FWA transcribed regions was significantly reduced in ELF7-ZF, as well as in HD2A-ZF (FIG. 7A), consistent with the silencing of FWA expression in these lines (FIG. 1B). However, a very prominent Pol II peak at the FWA promoter in the ELF7-ZF line, but not in HD2A-ZF nor fwa plants, was observed (FIG. 7A). Moreover, Pol II enrichment was also observed at the ZF off-target binding sites in ELF7-ZF (FIG. 7B and FIG. 14D). Thus, Pol II appears to be tethered to the ZF binding sites via interaction with ELF7-ZF, which in turn appears to inhibit Pol II transcription elongation, leading to gene silencing.

To better understand the endogenous function of ELF7, we performed ChIP-seq in pELF7:ELF-FLAG transgenic lines. Consistent with its role in transcriptional elongation, ELF7 was exclusively distributed over gene body regions, with most ELF7 signals overlapping with both Pol II peaks and H3K36me2 or H3K36me3 peaks (FIGS. 14E-14F). Pol II ChIP-seq in elf7-3 was performed and a significant accumulation of Pol II at ELF7 enriched sites was found (FIGS. 7C-7D), suggesting that transcriptional elongation is impeded, resulting in a higher Pol II occupancy reflected in the ChIP-seq data. Together this data show that the Arabidopsis PAF1 complex is required for proper Pol II transcriptional elongation, as has been shown in yeast and animal systems (see e.g., Fischl, Mol. Cell, 2017; Hou, PNAS, 2019), and that tethering the ELF7 component of the complex to promoters represents a novel synthetic mechanism to induce gene silencing that is likely independent of changes of particular epigenetic marks.

### Example 9: Target Silencing by CPL2-ZF and Pol II CTD Ser5 Dephosphorylation

The ZF fusion with CPL2 caused strong silencing of FWA without affecting DNA methylation and led to silencing of many ZF off-target loci (FIG. 1). CPL2 is a well characterized phosphatase that specifically acts on serine 5 (Ser5) of the Pol II C-terminal domain (see e.g., Koiwa, PNAS, 2004), and represses transcription through inhibiting Pol II activity (see e.g., Qian et al., Nat. Comm., 2021; Zhang, Nat. Comm., 2020). Therefore, a Pol II Ser5 ChIP-seq in CPL2-ZF transgenic lines was performed. A reduced signal at FWA as well as ZF off-target sites that had preexisting Pol II Ser5 was observed (FIGS. 7E-7F), suggesting that CPL2-ZF indeed silenced target genes through Pol II CTD Ser5 dephosphorylation. The promoter tethering of CPL2 thus represents a new mechanism for targeted gene silencing.

### Example 10: Target Genes Vary Widely in their Sensitivity to Different Gene Silencing Approaches

The set of downregulated genes at ZF off-target sites were partially non-overlapping with each of the gene silencing approaches employed by the fusion proteins. For example, although there was high degree of overlap of the genes downregulated by TRB1-ZF, TRB2-ZF and ZF-TRB3, and also a high degree of overlap of genes downregulated by HD2A-ZF, HD2B-ZF and HD2C-ZF, there was much less overlap in the downregulated genes comparing those commonly downregulated by TRBs and HD2s (FIGS. 7G-7H and FIGS. 14G-S7H). This suggests that the set of genes that are sensitive to downregulation by targeted histone methylation changes are distinct from those sensitive to downregulation by targeted histone acetylation changes. These results suggest that the best gene silencing approach will greatly depend on the particular target gene of interest, highlighting the utility of gene silencing tools that work by different mechanisms. The genes susceptible to silencing are involved in a wide range of biological processes including development and disease resistance, suggesting that the tools described here could be useful for the engineering of many different traits.

### Example 11: Target Silencing by MBD2-ZF, ZF-SUVH7, SSRP1-ZF, SPT16-ZF, JMJ18-ZF, TRBIP1-ZF, TRBIP2-ZF and ASF1B-ZF

To measure the relative DNA methylation level over FWA promoter, BS-PCR-seq and McrBC-qPCR methods were used. McrBC is an endonuclease that can only digest the DNA sequence with cytosine methylation. FWA promoter region was highly methylated in Col-0, while the methylation was removed in the fwa background. Therefore, FWA promoter region can be largely digested by McrBC in Col-0 but not in fwa. If same amount of the undigested and McrBC digested DNA were used as templates for qPCR to amplify FWA promoter region, McrBC treated DNA usually generate much less or equal amount of PCR products in Col-0 and fwa, respectively. Thereby, if any ZF lines gained DNA methylation over FWA promoter region, the reduced PCR products should be observed in the McrBC digested vs undigested sample.

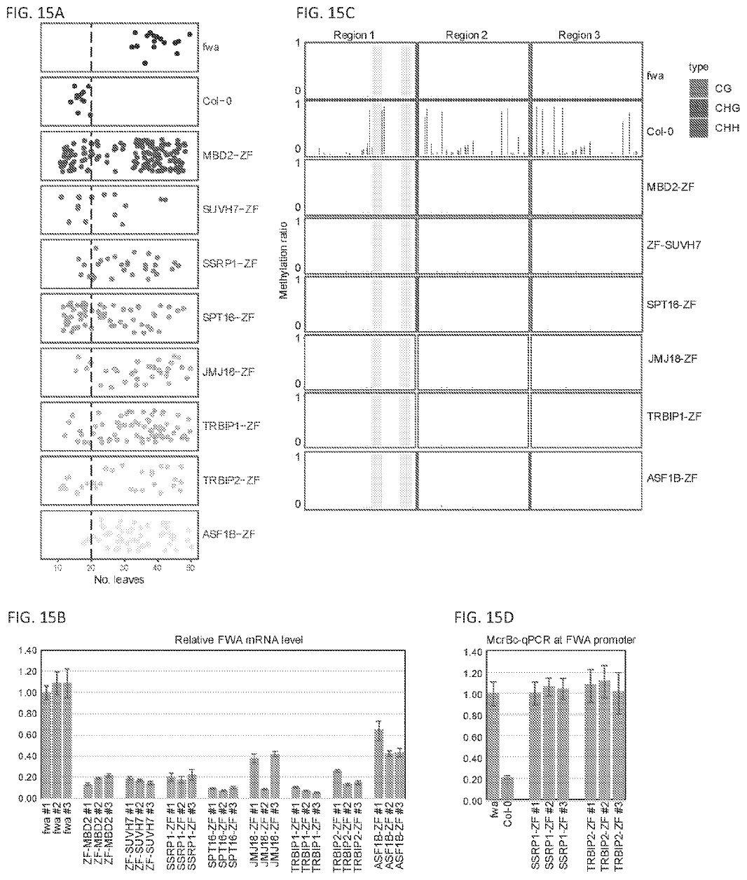

In addition to the proteins obtained from the screening above, other proteins of interest were constructed and tested. Among them, MBD2-ZF, ZF-SUVH7, SSRP1-ZF, SPT16-ZF, JMJ18-ZF, TRBIP1-ZF, TRBIP2-ZF and ASF1B-ZF were also capable of triggering early flowering phenotype and FWA suppression (FIGS. 15A-15B). BS-PCR-seq and McrBC-qPCR assays indicated that all these proteins didn't cause DNA methylation over FWA promoter region (FIGS. 15C-15D).

By constructing ZF fusions to target a collection of putative chromatin regulators to FWA, a variety of proteins capable of inducing gene silencing at FWA as well as at many other loci have been uncovered. To study the mechanism of action of each new component in the gene silencing arsenal, proteomic, genomic, and loss-of-function experiments were performed. Components of the well-known MOM1 complex were discovered to be able to induce de novo DNA methylation and gene silencing at targeted loci, a process that requires the MORC6 protein and the Pol V arm of the RdDM pathway. MOM1 complex components were also found to co-localize with sites of RdDM and to be required for the de novo silencing of FWA containing transgenes. This work highlights a new function for MOM1 complex components, which are known for silencing genes in heterochromatin downstream of DNA methylation (see e.g., Amedeo, Nature, 2000; Han, Plant Cell., 2016), and also provides additional mechanistic information about the RdDM pathway which is essential for controlling transposon proliferation in plant genomes (see e.g., Ito, Nature, 2011).

A number of gene silencing regulators that act in a DNA methylation-independent manner by altering local patterns of either histone acetylation or histone methylation were also described. The TRB factors were mechanistically interesting as they appear to act via both targeting the repressive H3K27me3 mark through PRC2, and by removing the activating H3K4me3 mark through the demethylase JMJ14. Finally, two factors that appear to act by directly interacting with Pol II, the ELF7 component of the elongation factor PAF1C and the CPL2 enzyme which dephosphorylates the RNA Pol II C-terminus, were found. Thus, these Examples provide evidence that the different effector proteins repressed target gene expression through diverse mechanisms including H3K27me3 deposition, H3K4me3 demethylation, H3K9, H3K14, H3K27 and H4K16 deacetylation, inhibition of Pol II transcriptional elongation, or Pol II dephosphorylation.

These results show that a number of different pathways can be harnessed for the development of synthetic biology tools to downregulate genes. From an engineering perspective, it will be crucial to have a range of tools available for the control of gene silencing. DNA methylation represents a strong and potentially heritable type of silencing, but only some genes will be amenable to this type of modification due to low densities of CG dinucleotides that are needed for silencing and heritability (see e.g., Gallego-Bartolome, Cell, 2019) or high levels of endogenous expression that can compete with methylation maintenance (see e.g., Papikian, Nat. Comm., 2019). Furthermore, the data showed that certain genes were more amenable to particular gene silencing approaches, meaning that having a wider array of silencing tools expands the range of genes that can be successfully targeted. In addition, it is likely that highly expressed genes may require a combination of silencing mechanisms for successful silencing. These findings lay a comprehensive foundation for more detailed mechanistic understanding of gene silencing pathways and provide an array of new tools for targeted gene silencing. In conclusion, this work provides mechanistic detail for an array of key plant gene silencing pathways, and describes a collection of new tools that should be useful both in basic research and crop improvement.

### Example 12: ZF Transgenic Lines do not Silence their Own Endogenous Genes

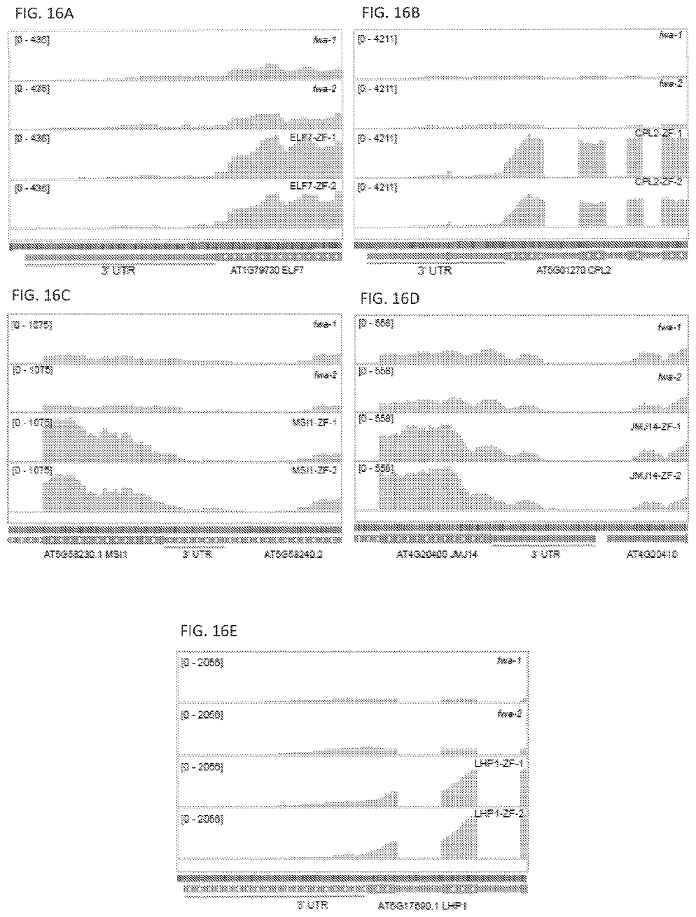

This Example describes additional data related to the data and information provided in FIG. 16.

FIGS. 16A-16E shows that the endogenous genes are not silenced in the respective ZF transgenic lines. 3′ UTRs of each ZF line were not included in the transgene.

### Example 13: EYFP-ZF Serves as a Negative Control

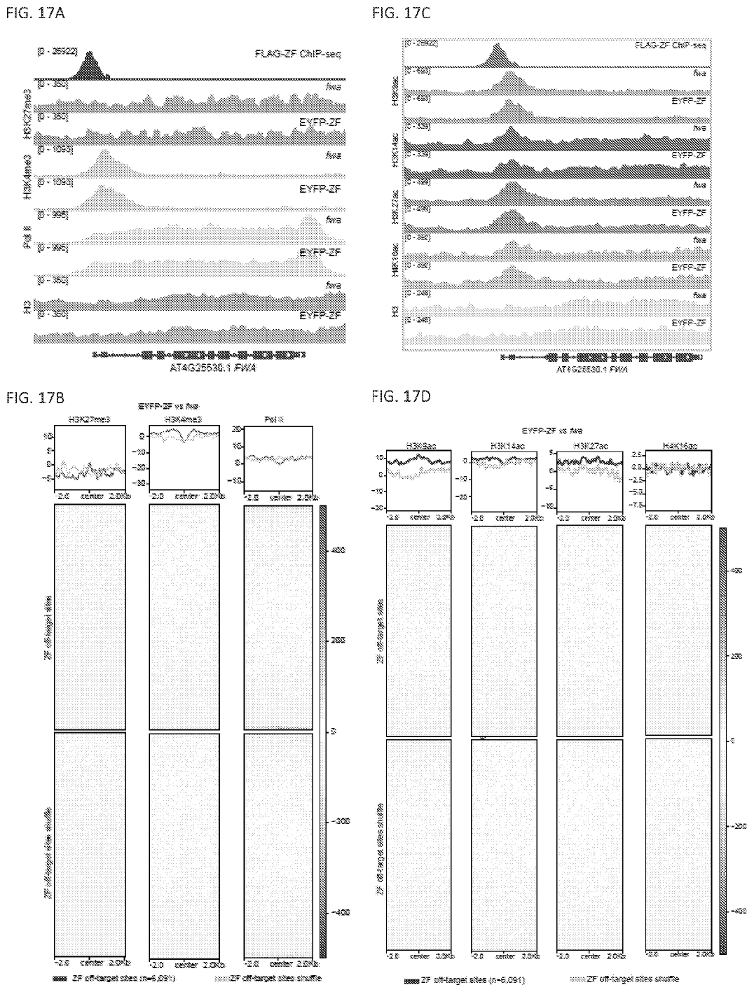

This Example describes additional data related to the data and information provided in FIG. 17.

EYFP-ZF was added as another negative control. Leaf count, RNA-seq, BS-PCR, histone ChIP-seq, etc were performed in fwa and EYFP-ZF transgenic lines to confirm that EYFP-ZF behaved similarly to fwa. H3K27me3, H3K4me3, and Pol II ChIP-seq levels were not changed in EYFP-ZF compared to fwa (FIGS. 17A-17B). Histone acetylation levels were not reduced in EYFP-ZF T2 transgenic lines compared to fwa (FIGS. 17C-17D).

### Example 14: ZF Transgenic Lines are Able to Target Pre-Existing H3K4Me3 and Histone Acetylation

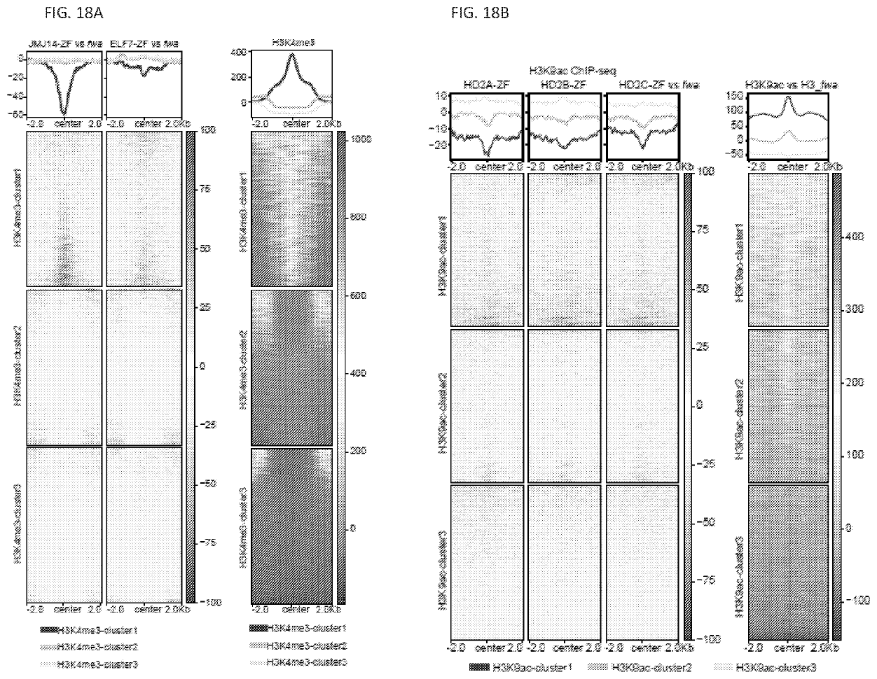

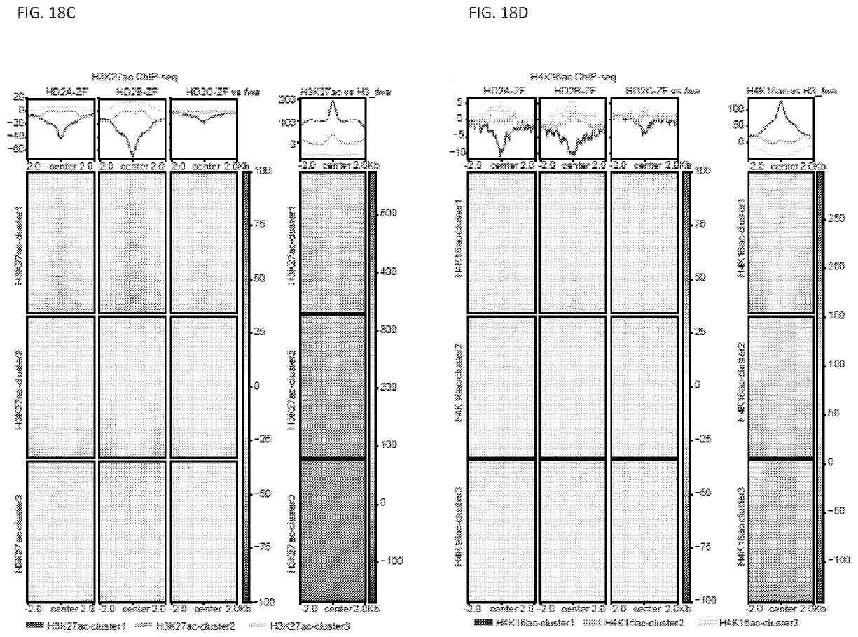

This Example describes additional data related to the data and information provided in FIG. 18.

The removal of H3K4me3 and histone acetylation ChIP-seq levels in were mainly over the regions with high levels of pre-existing H3K4me and histone acetylation ChIP-seq signal, respectively. The removal of H3K4me3 in JMJ14-ZF and ELF7-ZF transgenic lines mainly occurred over ZF off-target sites with pre-existing high level of H3K4me3 (FIG. 18A). Histone deacetylation in HD2A-ZF, HD2B-ZF, and HD2C-ZF mainly occurred over ZF off-target sites with pre-existing high level of histone acetylation (FIGS. 18B-18D).

### Example 15: Target Gene Silencing by Histone HDA6-ZF, MSI1-ZF, and Histone Deacetylation

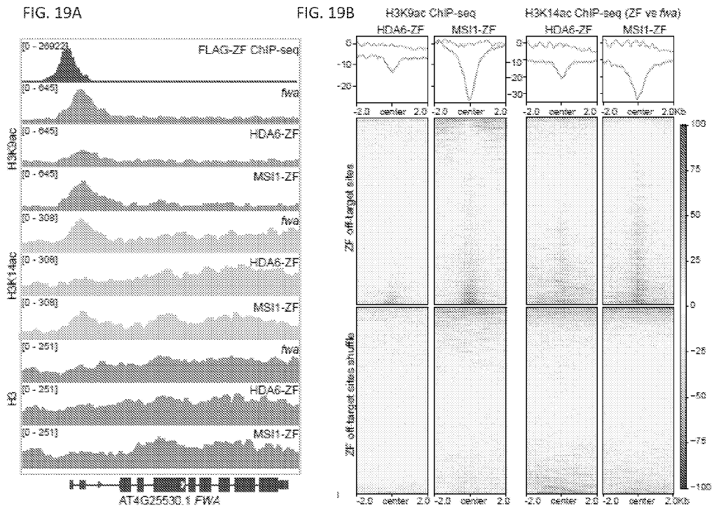

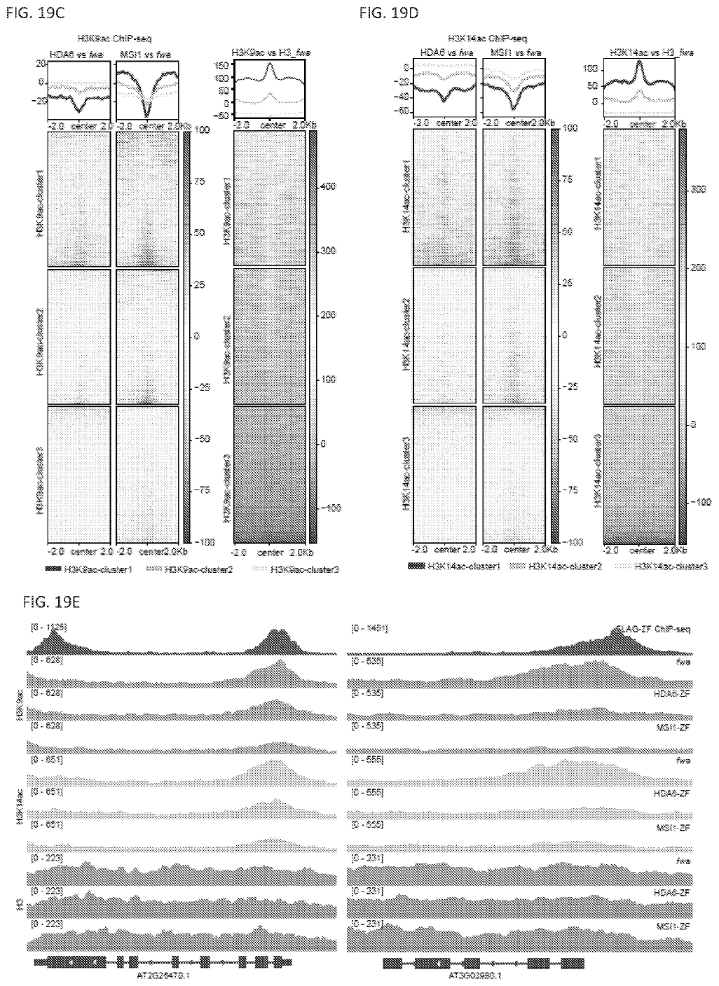

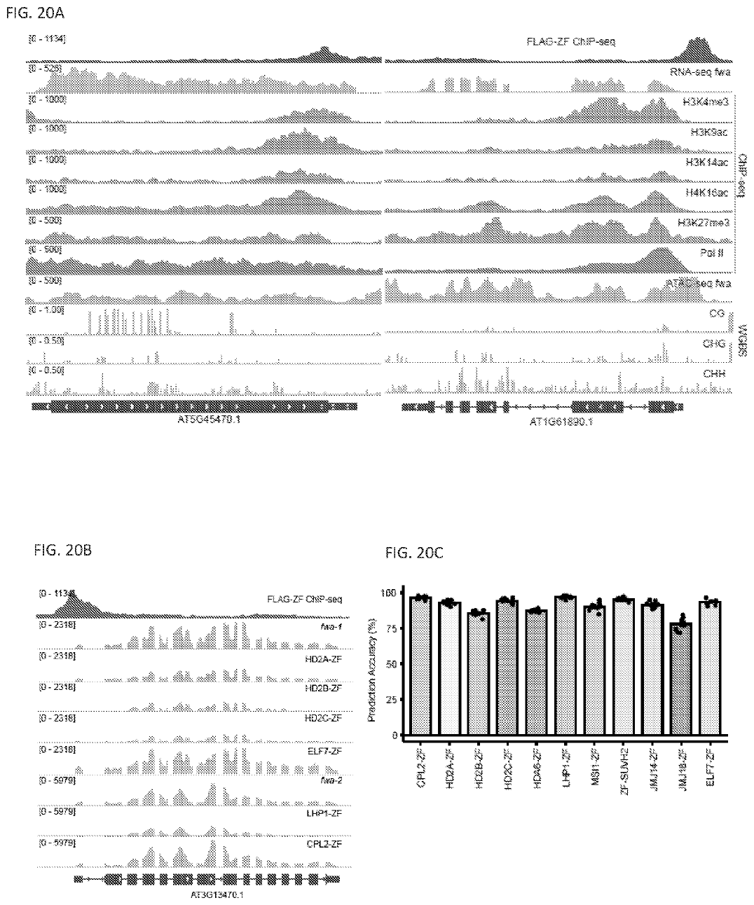

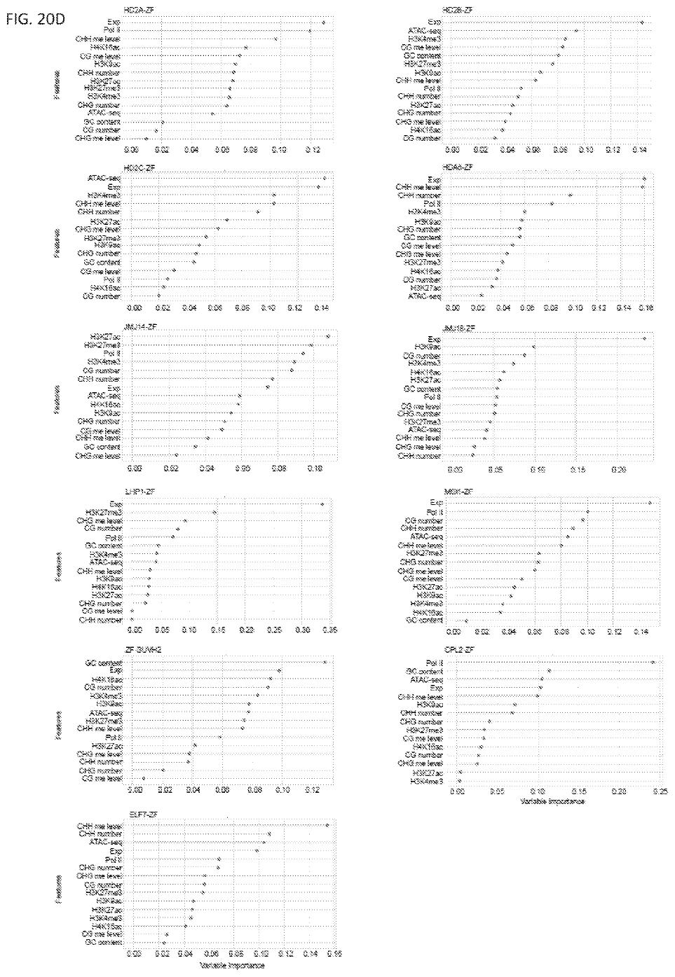

This Example describes additional data related to the data and information provided in FIG. 19.

MSI1-ZF and HDA6-ZF also triggered histone deacetylation (FIGS. 19A-19D), leading to target gene silencing (FIG. 19E) due to the interaction between MSI1, HDA6, and HDA19.

### Example 16: A New Machine Learning Model to Predict and Provide Importance of Various Chromatin Features

This Example describes additional data related to the data and information provided in FIG. 20.

A new machine learning model was built using the Decision Tree Classifier method, which not only predicts with a high accuracy, but also provides the importance of various chromatin features. The efficacy of different effector proteins was predicted using a machine learning model built by the Decision Tree Classifier method. Expression level, ATAC-seq signal, H3K4me3 ChIP-seq signal, CG methylation level, GC content, H3K27me3 ChIP-seq signal, H3K9ac ChIP-seq signal, CHH methylation level, RNA Polymerase II ChIP-seq signal, CHH number, H3K27ac ChIP-seq signal, CHG number, CHG methylation number, H4K16ac ChIP-seq signal, and CG number all contribute to the modeling construction by machine learning of each ZF transgenic line (FIG. 20).

### Example 17: Additional Data

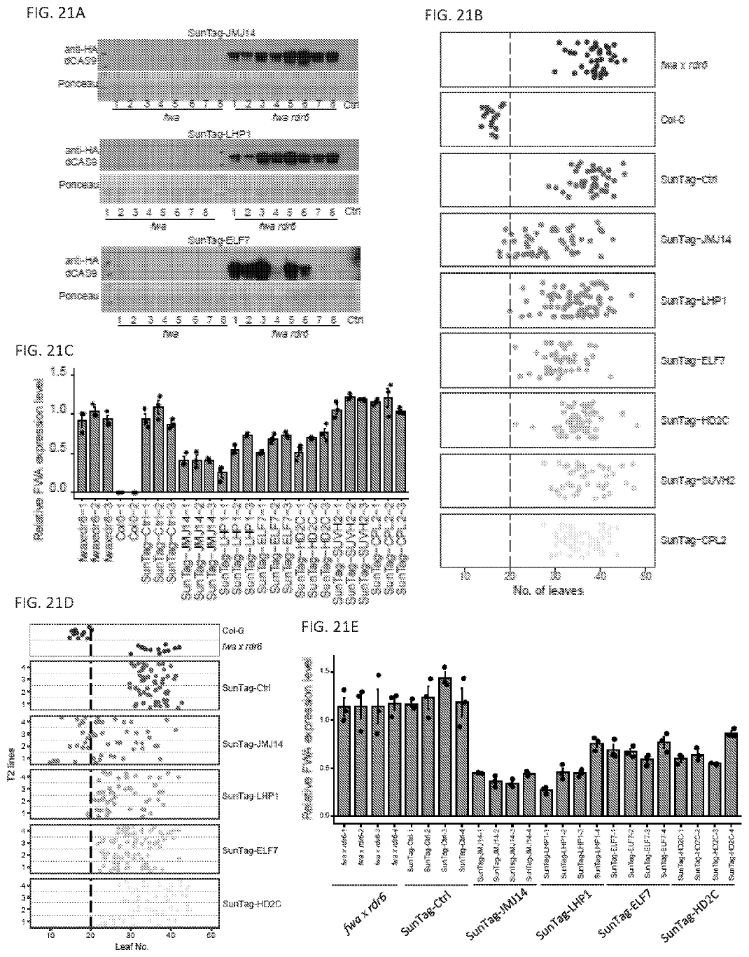

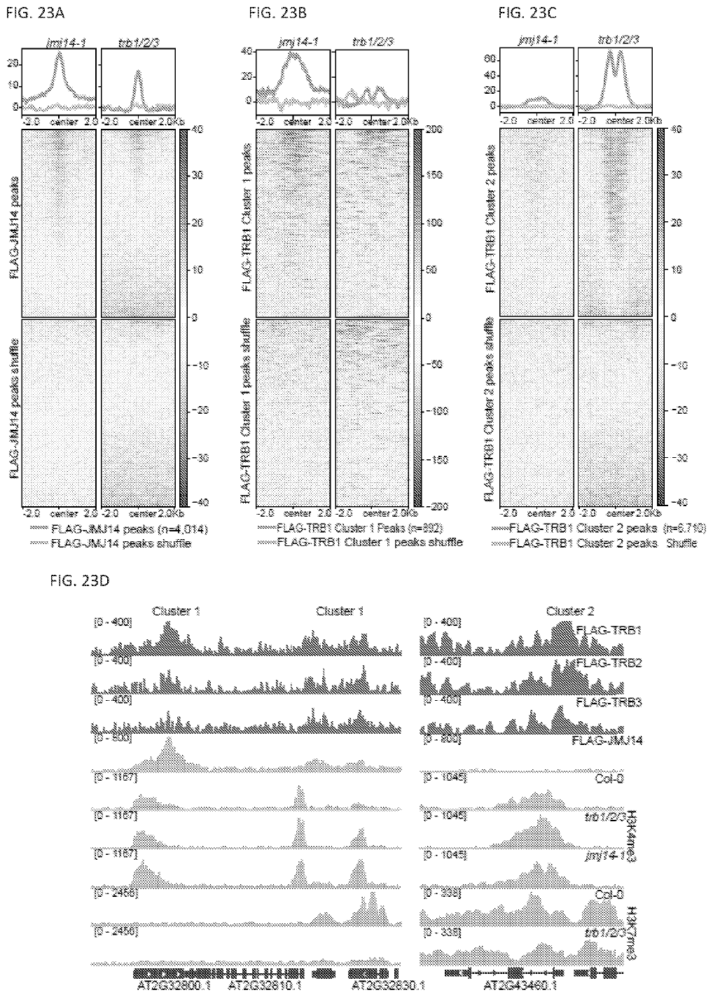

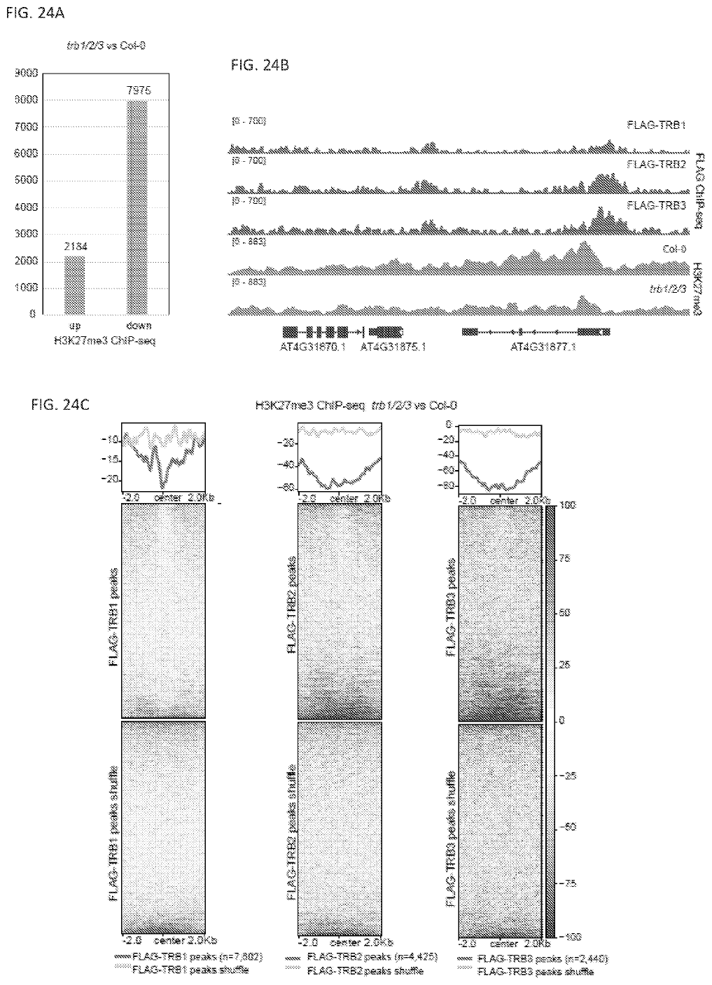

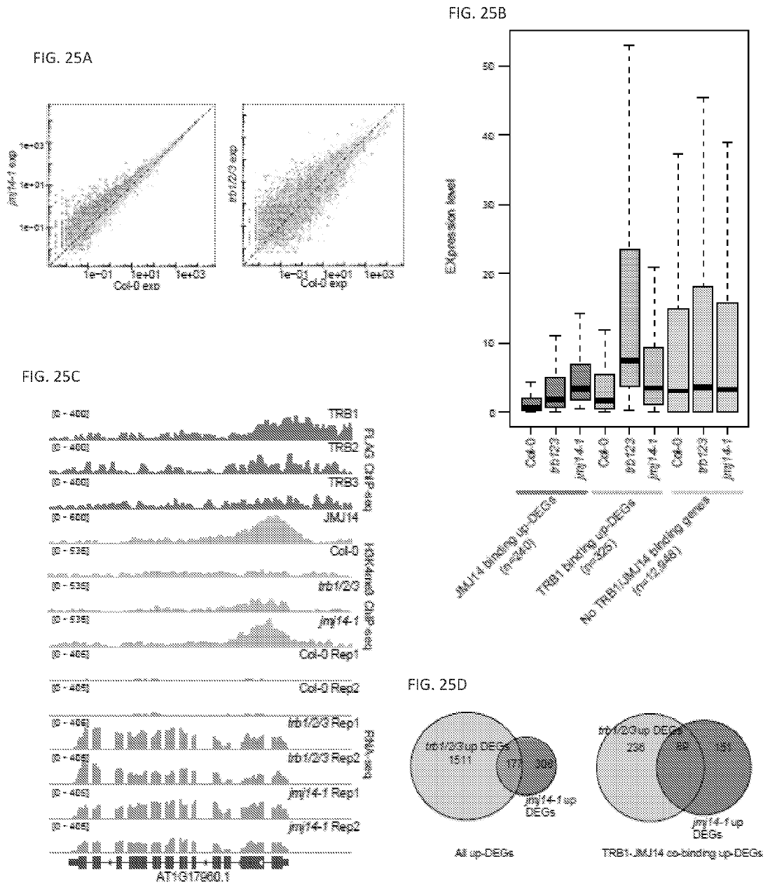

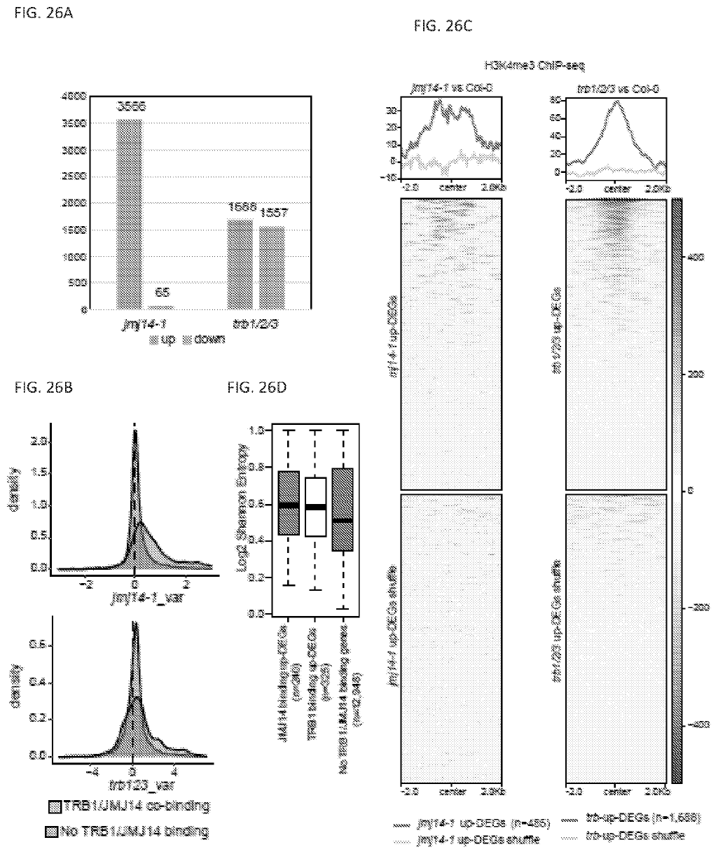

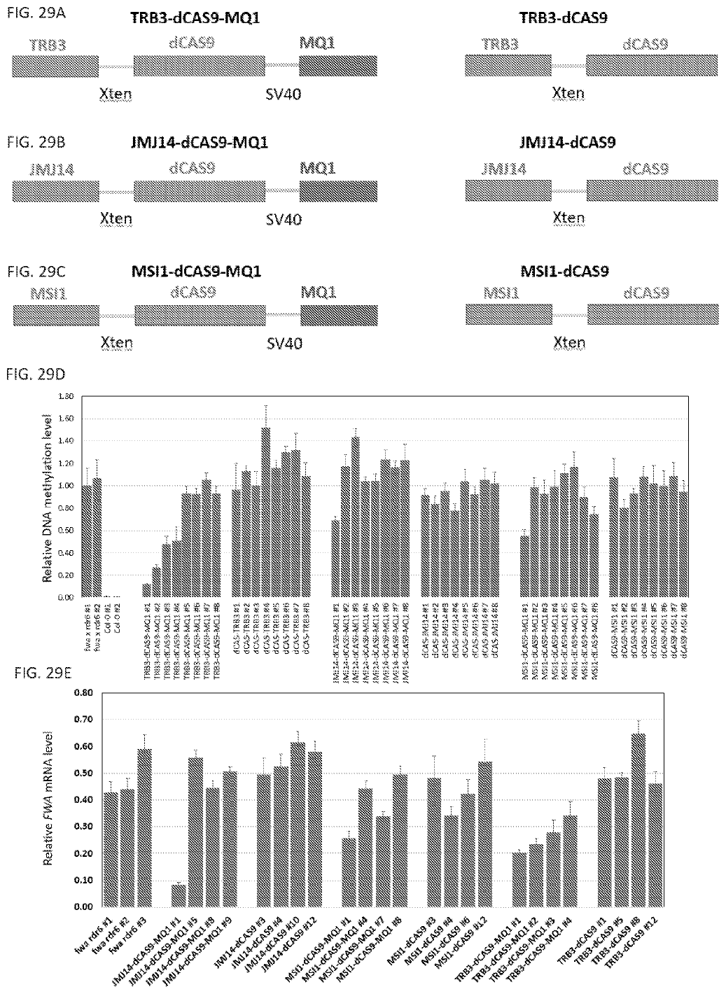

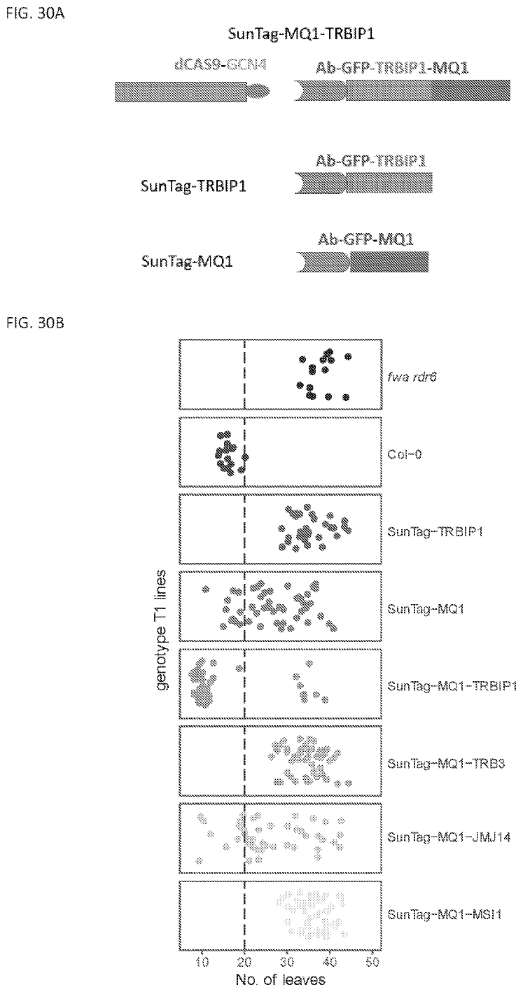

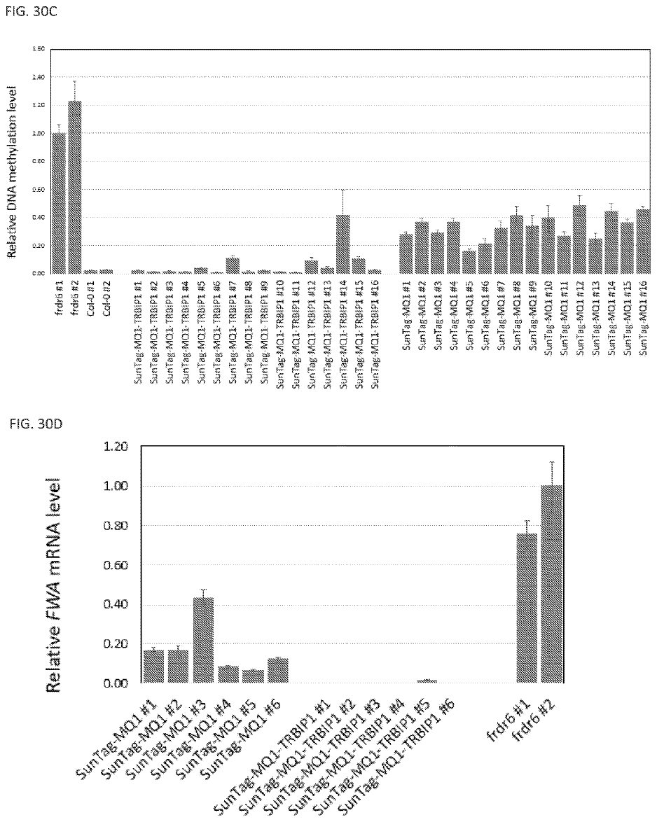

This Example describes additional data related to the data and information provided in FIGS. 21-30.

Some effector proteins, such as JMJ14, LHP1, HD2C, and ELF7, also triggered gene silencing in SunTag system, T1 lines (FIG. 21).

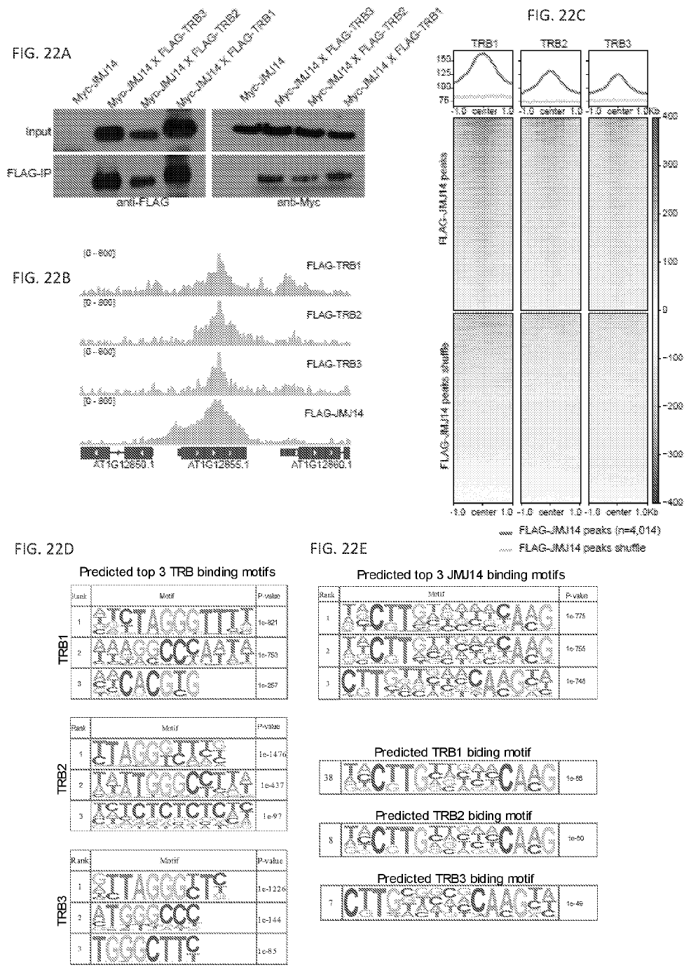

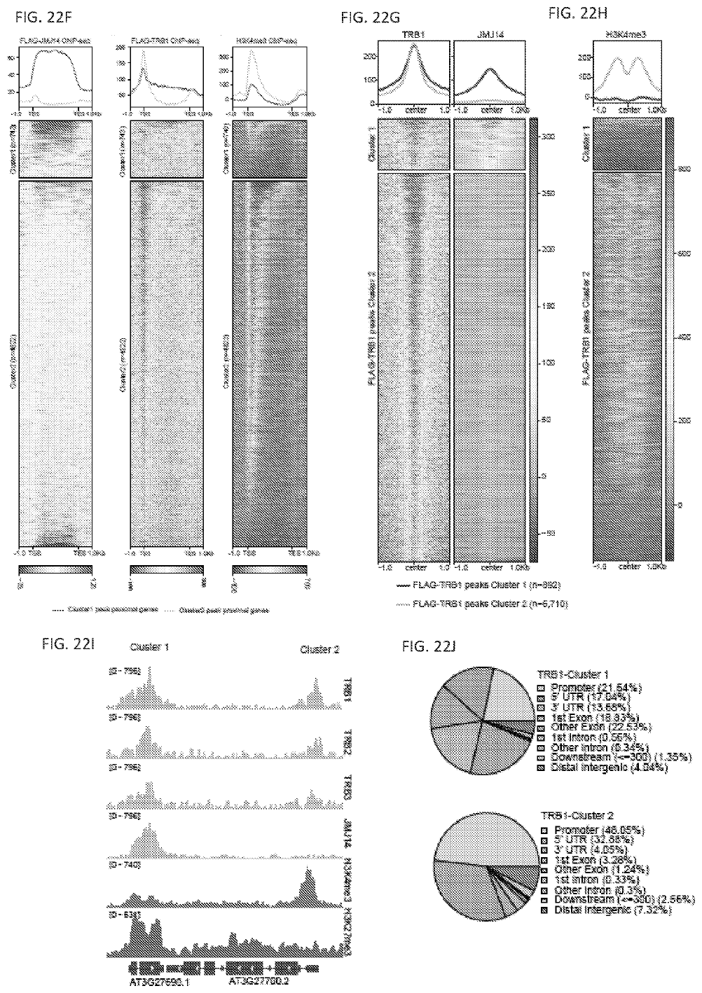

TRB proteins interacted and colocalized with JMJ14 over gene body regions. The up-regulated DEGs in trb1/2/3 mutant and jmj14-1 mutant were highly overlapped. H3K4me3 ChIP-seq signals were up-regulated in both trb1/2/3 mutant and jmj14-1 mutant over JMJ14 and TRB co-targeting regions. H3K27me3 ChIP-seq signals were reduced in trb1/2/3 mutant. The results here can be used to support the conclusion that TRB proteins can silence target genes through a combination of H3K4me3 removal and H3K27me3 deposition (FIGS. 22-26).

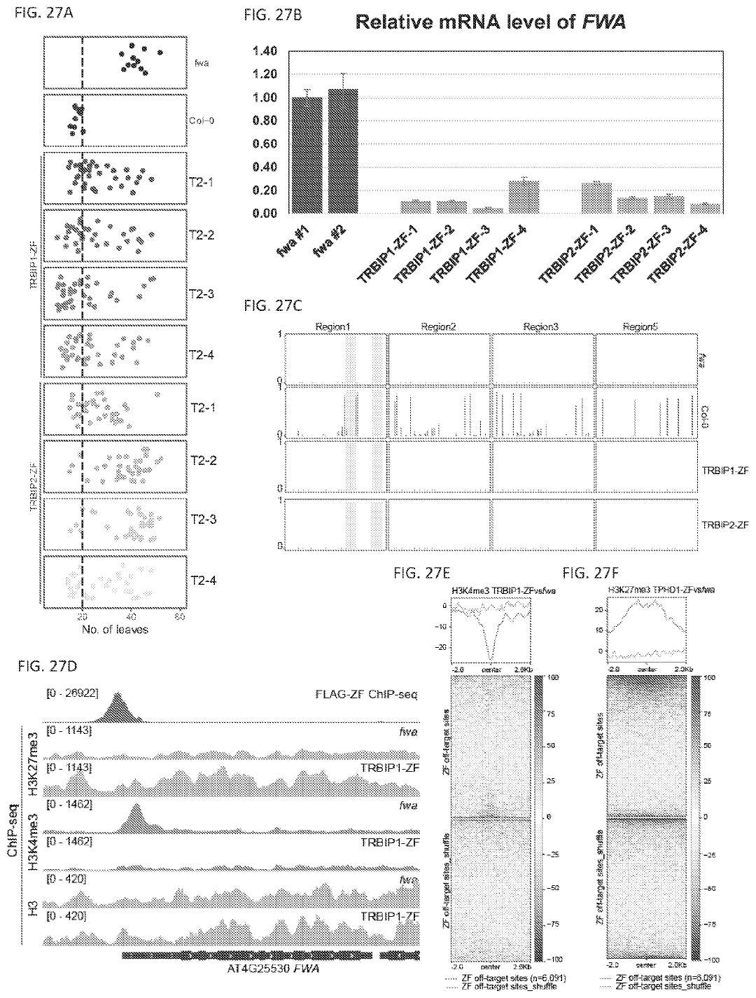

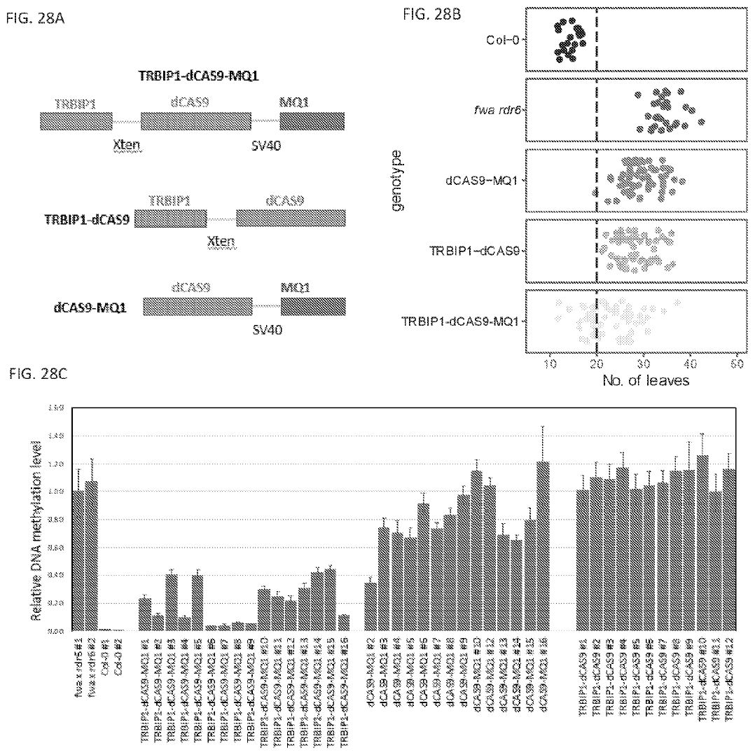

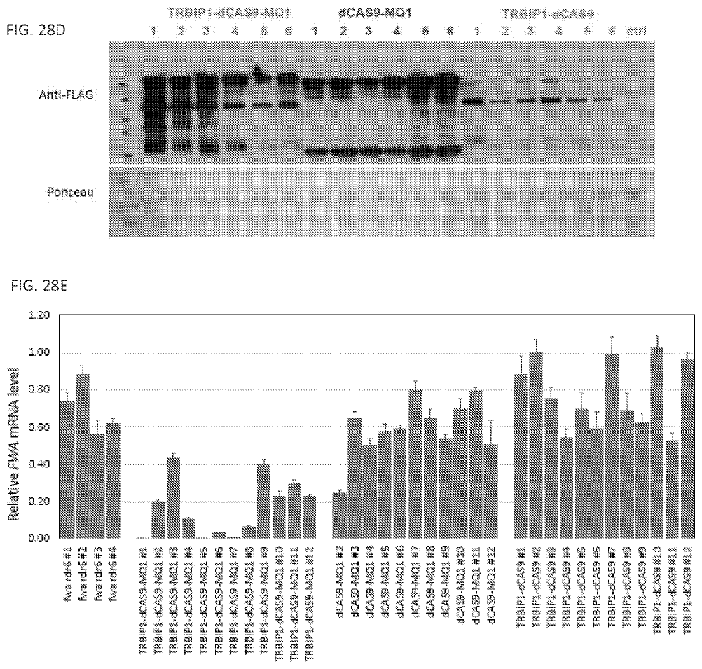

TRBIP1 and 2 were identified from TRB IP-MS, and TRBIP1 and TRBIP2 IP-MS also pulled down JMJ14, PRC2 related proteins, HDACs, and other proteins. TRBIP1-ZF and TRBIP2-ZF lines also triggered gene silencing over the FWA locus and zinc finger off-target sites, and the silencing was DNA methylation independent. TRBIP1/2-ZF triggered early flowering phenotype and FWA silencing, but without deposition of DNA methylation. ChIP-seq results also suggested that TRBIP1 and TRBIP2 might induce target gene silencing through a combination of H3K4me3 removal and H3K27me3 deposition. TRBIP1-ZF triggered H3K4me3 removal and H3K27me3 deposition at the FWA locus and zinc finger off-target loci (FIG. 27).

Since TRBIP1-ZF silenced target gene with the highest efficiency among all the non-DNA methylation related effector proteins, co-targeting of each of TRBIP1, TRB3, JMJ14, and MSI1, with DNA methyltransferase MQ1 was assayed, and shown to trigger DNA methylation over target sites in CRISPR-dCas9 system. Two methods were used for co-targeting: first, straight fusion of TRBIP1, dCas9, and MQ1 as a single expression protein; and second, fusion of TRBIP1 with MQ1 as an effector protein following antibody-GFP in the SunTag system. Both methods were found to trigger a synergistic effect on establishment of DNA methylation and target gene silencing. Co-targeting of TRBIP1 with MQ1 by straight fusion method triggered synergistic earlier flowering phenotype than dCas9-MQ1 and TRBIP1-dCas9. TRBIP1-dCas9-MQ1 triggered a higher efficiency and stronger level of DNA methylation than dCas9-MQ1. The amino acid sequence of the TRBIP1-dCas9-MQ1 fusion polypeptide is set forth in SEQ ID NO: 217. TRBIP1-dCas9-MQ1 triggered a higher efficiency and stronger level of FWA gene silencing than dCas9-MQ1 and TRBIP1-dCas9. TRB3-dCas9-MQ1 but not JMJ14-dCas9-MQ1 or MSI1-dCas9-MQ1 also triggered synergistic establishment of DNA methylation. TRB3-dCas9-MQ1 but not JMJ14-dCas9-MQ1 or MSI1-dCas9-MQ1 also triggered synergistic FWA gene silencing. Co-targeting of TRBIP1 with MQ1 by SunTag method triggered synergistic earlier flowering phenotype than SunTag-MQ1 and SunTag-TRBIP1. SunTag-MQ1-TRBIP1 also triggered synergistic establishment of DNA methylation. SunTag-MQ1-TRBIP1 triggered a higher level of FWA gene silencing than SunTag-MQ1 (FIGS. 28-30). For the SunTag-TRBIP1-MQ1 assays, the amino acid sequence of scFv-sfGFP-TRBIP1-MQ1 is set forth in SEQ ID NO: 218, and the amino acid sequence of dCAS9-GCN4×10 is set forth in SEQ ID NO: 219.

TABLE 22
TRB proteins IP-MS.

| | | TRB IP-MS | | | | | | | | X-link-IP-MS | | | |
|---|---|---|---|---|---|---|---|---|---|---|---|---|---|
| | | | | FLAG- | FLAG- | FLAG- | FLAG- | FLAG- | FLAG- | | FLAG- | FLAG- | FLAG- |
| | | Col- | Col- | TRB1- | TRB1- | TRB2- | TRB2- | TRB3- | TRB3- | Col- | TRB1- | TRB2- | TRB3- |
| Protein | | 0-1 | 0-2 | 1-IP | 2-IP | 1-IP | 2-IP | 1-IP | 2-IP | 0 | IP | IP | IP |
| AT1G49950.3 TRB1 | | 0 | 0 | 189 | 181 | 0 | 4 | 0 | 0 | 0 | 72 | 10 | 9 |
| AT5G67580.1 TRB2 | | 0 | 0 | 0 | 0 | 117 | 117 | 12 | 11 | 0 | 2 | 126 | 11 |
| AT3G49850.1 TRB3 | | 0 | 0 | 0 | 0 | 0 | 0 | 134 | 144 | 0 | 0 | 4 | 78 |
| AT4G20400.1 JMJ14 | JMJ14 | 0 | 0 | 34 | 35 | 3 | 4 | 13 | 12 | 0 | 77 | 78 | 72 |
| AT3G10490.2 NAC052 | | 0 | 0 | 103 | 103 | 11 | 22 | 24 | 28 | 0 | 55 | 55 | 59 |
| AT3G10480.3 NAC050 | | 0 | 0 | 33 | 35 | 1 | 1 | 5 | 4 | 0 | 40 | 39 | 39 |
| AT5G17690.1 LHP1 | PRC2 | 0 | 0 | 0 | 1 | 15 | 15 | 15 | 19 | 0 | 0 | 17 | 18 |
| AT5G11530.1 EMF1 | | 0 | 0 | 3 | 2 | 55 | 56 | 81 | 83 | 0 | 11 | 90 | 104 |
| AT5G51230.1 EMF2 | | 0 | 0 | 0 | 0 | 10 | 15 | 23 | 23 | 0 | 0 | 27 | 29 |
| AT3G20740.1 FIE | | 0 | 0 | 1 | 3 | 30 | 28 | 34 | 41 | 0 | 5 | 35 | 34 |
| AT2G23380.1 CLF | | 0 | 0 | 0 | 0 | 5 | 5 | 15 | 19 | 0 | 0 | 15 | 19 |
| AT4G02020.1 SWN | | 0 | 0 | 1 | 0 | 11 | 12 | 16 | 24 | 0 | 2 | 33 | 36 |
| AT5G58230.1 MSI1 | | 0 | 1 | 11 | 12 | 26 | 21 | 37 | 35 | 0 | 12 | 30 | 31 |
| AT1G22950.1 ICU11 | | 0 | 0 | 75 | 79 | 53 | 50 | 79 | 73 | 0 | 27 | 33 | 37 |
| AT3G18210.1 CP2 | | 0 | 0 | 13 | 17 | 4 | 5 | 38 | 31 | 0 | 11 | 17 | 12 |
| AT4G35510.1 TRBIP1 | | 0 | 0 | 5 | 11 | 22 | 22 | 24 | 38 | 0 | 0 | 16 | 17 |
| AT2G17540.2 TRBIP2 | | 0 | 0 | 18 | 22 | 10 | 10 | 16 | 20 | 0 | 0 | 7 | 3 |

TABLE 23
TRBIP proteins IP-MS

| | Protein IDs | WT-1 | WT-2 | TRBIP1-1 | TRBIP1-2 | TRBIP2-1 | TRBIP2-2 |
|---|---|---|---|---|---|---|---|
| | AT4G35510.1 TRBIP1 | 0 | 0 | 180 | 111 | 1 | 0 |
| | AT2G17540.2 TRBIP2 | 0 | 0 | 0 | 0 | 95 | 34 |
| | AT1G49950.3 TRB1 | 0 | 0 | 39 | 15 | 26 | 5 |
| | AT5G67580.1 TRB2 | 0 | 0 | 66 | 36 | 43 | 3 |
| | AT3G49850.1 TRB3 | 0 | 0 | 31 | 19 | 8 | 6 |
| PRC2 | | | | | | | |
| | AT5G11530.1 EMF1 | 0 | 0 | 36 | 7 | 4 | 0 |
| | AT1G22950.1 ICU11 | 0 | 0 | 32 | 19 | 16 | 4 |
| | AT3G20740.1 FIE | 0 | 0 | 22 | 3 | 1 | 0 |
| | AT5G58230.1 MSI1 | 0 | 0 | 20 | 9 | 3 | 0 |
| | AT4G02020.1 SWN | 0 | 0 | 15 | 6 | 3 | 0 |
| | AT5G17690.1 LHP1 | 0 | 0 | 14 | 8 | 2 | 1 |
| | AT5G51230.1 EMF2 | 0 | 0 | 10 | 3 | 0 | 0 |
| | AT2G23380.1 CLF | 0 | 0 | 7 | 0 | 0 | 0 |
| JMJ14 | | | | | | | |
| | AT4G20400.1 JMJ14 | 0 | 0 | 7 | 0 | 3 | 0 |
| | AT3G10490.2 NAC052 | 0 | 0 | 10 | 2 | 4 | 0 |
| | AT3G10480.3 NAC050 | 0 | 0 | 2 | 0 | 1 | 0 |
| HDACS | | | | | | | |
| | AT5G08450.1 HDC1 | 0 | 0 | 8 | 2 | 2 | 0 |
| | AT4G38130.1 HDA19 | 0 | 0 | 4 | 3 | 4 | 1 |
| | AT5G63110.1 HDA6 | 0 | 0 | 7 | 5 | 10 | 3 |

### REFERENCES

1. Amedeo, P., Habu, Y., Afsar, K., Mittelsten Scheid, O., and Paszkowski, J. (2000). Disruption of the plant gene MOM releases transcriptional silencing of methylated genes. Nature 405, 203-206.
2. Antosz, W., Pfab, A., Ehrnsberger, H. F., Holzinger, P., Kollen, K., Mortensen, S. A., Bruckmann, A., Schubert, T., Langst, G., Griesenbeck, J., et al. (2017). The Composition of the Arabidopsis RNA Polymerase II Transcript Elongation Complex Reveals the Interplay between Elongation and mRNA Processing Factors. The Plant cell 29, 854-870.
3. Blevins, T., Podicheti, R., Mishra, V., Marasco, M., Wang, J., Rusch, D., Tang, H., and Pikaard, C. S. (2015). Identification of Pol IV and RDR2-dependent precursors of 24 nt siRNAs guiding de novo DNA methylation in Arabidopsis. eLife 4, e09591.
4. Bloomer, R. H., Hutchison, C. E., Baurle, I., Walker, J., Fang, X., Perera, P., Velanis, C. N., Gumus, S., Spanos, C., Rappsilber, J., et al. (2020). The Arabidopsis epigenetic regulator ICU11 as an accessory protein of Polycomb Repressive Complex 2. Proceedings of the National Academy of Sciences of the United States of America 117, 16660-16666.
5. Bond, D. M., and Baulcombe, D. C. (2015). Epigenetic transitions leading to heritable, RNA-mediated de novo silencing in Arabidopsis thaliana. Proceedings of the National Academy of Sciences of the United States of America 112, 917-922.
6. Caikovski, M., Yokthongwattana, C., Habu, Y., Nishimura, T., Mathieu, O., and Paszkowski, J. (2008). Divergent evolution of CHD3 proteins resulted in MOM1 refining epigenetic control in vascular plants. PLoS genetics 4, e1000165.
7. Cao, X., and Jacobsen, S. E. (2002). Role of the Arabidopsis DRM methyltransferases in de novo DNA methylation and gene silencing. Current biology: CB 12, 1138-1144.
8. Catoni, M., Tsang, J. M., Greco, A. P., and Zabet, N. R. (2018). DMRcaller: a versatile R/Bioconductor package for detection and visualization of differentially methylated regions in CpG and non-CpG contexts. Nucleic acids research 46, e114.
9. Chen, L. T., Luo, M., Wang, Y. Y., and Wu, K. (2010). Involvement of Arabidopsis histone deacetylase HDA6 in ABA and salt stress response. Journal of experimental botany 61, 3345-3353.
10. Chen, X., Lu, L., Qian, S., Scalf, M., Smith, L. M., and Zhong, X. (2018). Canonical and Noncanonical Actions of Arabidopsis Histone Deacetylases in Ribosomal RNA Processing. The Plant cell 30, 134-152.
11. Colville, A., Alhattab, R., Hu, M., Labbe, H., Xing, T., and Miki, B. (2011). Role of HD2 genes in seed germination and early seedling growth in Arabidopsis. Plant cell reports 30, 1969-1979.
12. Deleris, A., Greenberg, M. V., Ausin, I., Law, R. W., Moissiard, G., Schubert, D., and Jacobsen, S. E. (2010). Involvement of a Jumonji-C domain-containing histone demethylase in DRM2-mediated maintenance of DNA methylation. EMBO reports 11, 950-955.
13. Derkacheva, M., Steinbach, Y., Wildhaber, T., Mozgova, I., Mahrez, W., Nanni, P., Bischof, S., Gruissem, W., and Hennig, L. (2013). Arabidopsis MSI1 connects LHP1 to PRC2 complexes. The EMBO journal 32, 2073-2085.
14. Du, J., Johnson, L. M., Jacobsen, S. E., and Patel, D. J. (2015). DNA methylation pathways and their crosstalk with histone methylation. Nature reviews Molecular cell biology 16, 519-532.
15. Duan, C. G., Wang, X., Zhang, L., Xiong, X., Zhang, Z., Tang, K., Pan, L., Hsu, C. C., Xu, H., Tao, W. A., et al. (2017). A protein complex regulates RNA processing of intronic heterochromatin-containing genes in Arabidopsis. Proceedings of the National Academy of Sciences of the United States of America 114, E7377-E7384.
16. Earley, K., Lawrence, R. J., Pontes, O., Reuther, R., Enciso, A. J., Silva, M., Neves, N., Gross, M., Viegas, W., and Pikaard, C. S. (2006). Erasure of histone acetylation by Arabidopsis HDA6 mediates large-scale gene silencing in nucleolar dominance. Genes & development 20, 1283-1293.
17. Feng, S., Jacobsen, S. E., and Reik, W. (2010). Epigenetic reprogramming in plant and animal development. Science 330, 622-627.
18. Fischl, H., Howe, F. S., Furger, A., and Mellor, J. (2017). Paf1 Has Distinct Roles in Transcription Elongation and Differential Transcript Fate. Molecular cell 65, 685-698 e688.
19. Gallego-Bartolome, J., Liu, W., Kuo, P. H., Feng, S., Ghoshal, B., Gardiner, J., Zhao, J. M., Park, S. Y., Chory, J., and Jacobsen, S. E. (2019). Co-targeting RNA Polymerases IV and V Promotes Efficient De Novo DNA Methylation in Arabidopsis. Cell 176, 1068-1082 e1019.
20. Gao, Z., Liu, H. L., Daxinger, L., Pontes, O., He, X., Qian, W., Lin, H., Xie, M., Lorkovic, Z. J., Zhang, S., et al. (2010). An RNA polymerase II- and AGO4-associated protein acts in RNA-directed DNA methylation. Nature 465, 106-109.
21. Geisler, S. J., and Paro, R. (2015). Trithorax and Polycomb group-dependent regulation: a tale of opposing activities. Development 142, 2876-2887.
22. Grabherr, M. G., Haas, B. J., Yassour, M., Levin, J. Z., Thompson, D. A., Amit, I., Adiconis, X., Fan, L., Raychowdhury, R., Zeng, Q., et al. (2011). Full-length transcriptome assembly from RNA-Seq data without a reference genome. Nat Biotechnol 29, 644-652.
23. Greenberg, M. V., Deleris, A., Hale, C. J., Liu, A., Feng, S., and Jacobsen, S. E. (2013). Interplay between active chromatin marks and RNA-directed DNA methylation in Arabidopsis thaliana. PLoS genetics 9, e1003946.
24. Guo, Y., Xue, Z., Yuan, R., Li, J. J., Pastor, W. A., and Liu, W. (2021). RAD: a web application to identify region associated differentially expressed genes. Bioinformatics.
25. Habu, Y., Mathieu, O., Tariq, M., Probst, A. V., Smathajitt, C., Zhu, T., and Paszkowski, J. (2006). Epigenetic regulation of transcription in intermediate heterochromatin. EMBO reports 7, 1279-1284.
26. Han, Y. F., Zhao, Q. Y., Dang, L. L., Luo, Y. X., Chen, S. S., Shao, C. R., Huang, H. W., Li, Y. Q., Li, L., Cai, T., et al. (2016). The SUMO E3 Ligase-Like Proteins PIAL1 and PIAL2 Interact with MOM1 and Form a Novel Complex Required for Transcriptional Silencing. The Plant cell 28, 1215-1229.
27. Harris, C. J., Husmann, D., Liu, W., Kasmi, F. E., Wang, H., Papikian, A., Pastor, W. A., Moissiard, G., Vashisht, A. A., Dangl, J. L., et al. (2016). Arabidopsis AtMORC4 and AtMORC7 Form Nuclear Bodies and Repress a Large Number of Protein-Coding Genes. PLoS genetics 12, e1005998.
28. He, Y., Doyle, M. R., and Amasino, R. M. (2004). PAF1-complex-mediated histone methylation of FLOWERING LOCUS C chromatin is required for the vernalization-responsive, winter-annual habit in Arabidopsis. Genes & development 18, 2774-2784.
29. Hollender, C., and Liu, Z. (2008). Histone deacetylase genes in Arabidopsis development. Journal of integrative plant biology 50, 875-885.
30. Hou, L., Wang, Y., Liu, Y., Zhang, N., Shamovsky, I., Nudler, E., Tian, B., and Dynlacht, B. D. (2019). Paf1C regulates RNA polymerase II progression by modulating elongation rate. Proceedings of the National Academy of Sciences of the United States of America 116, 14583-14592.
31. Ito, H., Gaubert, H., Bucher, E., Mirouze, M., Vaillant, I., and Paszkowski, J. (2011). An siRNA pathway prevents transgenerational retrotransposition in plants subjected to stress. Nature 472, 115-119.
32. Jiang, D., Wang, Y., Wang, Y., and He, Y. (2008). Repression of FLOWERING LOCUS C and FLOWERING LOCUS T by the Arabidopsis Polycomb repressive complex 2 components. PloS one 3, e3404.
33. Jing, Y., Sun, H., Yuan, W., Wang, Y., Li, Q., Liu, Y., Li, Y., and Qian, W. (2016). SUVH2 and SUVH9 Couple Two Essential Steps for Transcriptional Gene Silencing in Arabidopsis. Molecular plant 9, 1156-1167.
34. Johnson, L. M., Du, J., Hale, C. J., Bischof, S., Feng, S., Chodavarapu, R. K., Zhong, X., Marson, G., Pellegrini, M., Segal, D. J., et al. (2014). SRA- and SET-domain-containing proteins link RNA polymerase V occupancy to DNA methylation. Nature 507, 124-128.
35. Kanno, T., Mette, M. F., Kreil, D. P., Aufsatz, W., Matzke, M., and Matzke, A. J. (2004). Involvement of putative SNF2 chromatin remodeling protein DRD1 in RNA-directed DNA methylation. Current biology: CB 14, 801-805.
36. Kinoshita, T., Miura, A., Choi, Y., Kinoshita, Y., Cao, X., Jacobsen, S. E., Fischer, R. L., and Kakutani, T. (2004). One-way control of FWA imprinting in Arabidopsis endosperm by DNA methylation. Science 303, 521-523.
37. Koiwa, H., Hausmann, S., Bang, W. Y., Ueda, A., Kondo, N., Hiraguri, A., Fukuhara, T., Bahk, J. D., Yun, D. J., Bressan, R. A., et al. (2004). Arabidopsis C-terminal domain phosphatase-like 1 and 2 are essential Ser-5-specific C-terminal domain phosphatases. Proceedings of the National Academy of Sciences of the United States of America 101, 14539-14544.
38. Kuchar, M., and Fajkus, J. (2004). Interactions of putative telomere-binding proteins in Arabidopsis thaliana: identification of functional TRF2 homolog in plants. FEBS letters 578, 311-315.
39. Langmead, B., and Salzberg, S. L. (2012). Fast gapped-read alignment with Bowtie 2. Nat Methods 9, 357-359.
40. Langmead, B., Trapnell, C., Pop, M., and Salzberg, S. L. (2009). Ultrafast and memory-efficient alignment of short
41. Law, J. A., and Jacobsen, S. E. (2010). Establishing, maintaining and modifying DNA methylation patterns in plants and animals. Nature reviews Genetics 11, 204-220.
42. Lawrence, R. J., Earley, K., Pontes, O., Silva, M., Chen, Z. J., Neves, N., Viegas, W., and Pikaard, C. S. (2004). A concerted DNA methylation/histone methylation switch regulates rRNA gene dosage control and nucleolar dominance. Molecular cell 13, 599-609.
43. Li, B., and Dewey, C. N. (2011). RSEM: accurate transcript quantification from RNA-Seq data with or without a reference genome. BMC Bioinformatics 12, 323.
44. Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
45. Li, H., Torres-Garcia, J., Latrasse, D., Benhamed, M., Schilderink, S., Zhou, W., Kulikova, O., Hirt, H., and Bisseling, T. (2017). Plant-Specific Histone Deacetylases HDT1/2 Regulate GIBBERELLIN 2-OXIDASE2 Expression to Control Arabidopsis Root Meristem Cell Number. The Plant cell 29, 2183-2196.
46. Lin, J., Hung, F. Y., Ye, C., Hong, L., Shih, Y. H., Wu, K., and Li, Q. Q. (2020). HDA6-dependent histone deacetylation regulates mRNA polyadenylation in Arabidopsis. Genome research 30, 1407-1417.
47. Liu, W., Duttke, S. H., Hetzel, J., Groth, M., Feng, S., Gallego-Bartolome, J., Zhong, Z., Kuo, H. Y., Wang, Z., Zhai, J., et al. (2018). RNA-directed DNA methylation involves co-transcriptional small-RNA-guided slicing of polymerase V transcripts in Arabidopsis. Nature plants 4, 181-188.
48. Liu, Z. W., Shao, C. R., Zhang, C. J., Zhou, J. X., Zhang, S. W., Li, L., Chen, S., Huang, H. W., Cai, T., and He, X. J. (2014). The SET domain proteins SUVH2 and SUVH9 are required for Pol V occupancy at RNA-directed DNA methylation loci. PLoS genetics 10, e1003948.
49. Liu, Z. W., Zhou, J. X., Huang, H. W., Li, Y. Q., Shao, C. R., Li, L., Cai, T., Chen, S., and He, X. J. (2016). Two Components of the RNA-Directed DNA Methylation Pathway Associate with MORC6 and Silence Loci Targeted by MORC6 in Arabidopsis. PLoS genetics 12, e1006026.
50. Lu, F., Cui, X., Zhang, S., Liu, C., and Cao, X. (2010). JMJ14 is an H3K4 demethylase regulating flowering time in Arabidopsis. Cell research 20, 387-390.
51. Luo, M., Wang, Y. Y., Liu, X., Yang, S., and Wu, K. (2012). HD2 proteins interact with RPD3-type histone deacetylases. Plant signaling & behavior 7, 608-610.
52. Matzke, M. A., and Mosher, R. A. (2014). RNA-directed DNA methylation: an epigenetic pathway of increasing complexity. Nature reviews Genetics 15, 394-408.
53. Moissiard, G., Cokus, S. J., Cary, J., Feng, S., Billi, A. C., Stroud, H., Husmann, D., Zhan, Y., Lajoie, B. R., McCord, R. P., et al. (2012). MORC family ATPases required for heterochromatin condensation and gene silencing. Science 336, 1448-1451.
54. Mozgova, I., and Hennig, L. (2015). The polycomb group protein regulatory network. Annual review of plant biology 66, 269-296.
55. Mylne, J. S., Barrett, L., Tessadori, F., Mesnage, S., Johnson, L., Bernatavichute, Y. V., Jacobsen, S. E., Fransz, P., and Dean, C. (2006). LHP1, the Arabidopsis homologue of HETEROCHROMATIN PROTEIN1, is required for epigenetic silencing of FLC. Proceedings of the National Academy of Sciences of the United States of America 103, 5012-5017.
56. Ning, Y. Q., Ma, Z. Y., Huang, H. W., Mo, H., Zhao, T. T., Li, L., Cai, T., Chen, S., Ma, L., and He, X. J. (2015). Two novel NAC transcription factors regulate gene expression and flowering time by associating with the histone demethylase JMJ14. Nucleic acids research 43, 1469-1484.
57. Nishimura, T., Molinard, G., Petty, T. J., Broger, L., Gabus, C., Halazonetis, T. D., Thore, S., and Paszkowski, J. (2012). Structural basis of transcriptional gene silencing mediated by Arabidopsis MOM1. PLoS genetics 8, e1002484.
58. Oh, S., Park, S., and van Nocker, S. (2008). Genic and global functions for Paf1C in chromatin modification and gene expression in Arabidopsis. PLoS genetics 4, e1000077.
59. Papikian, A., Liu, W., Gallego-Bartolome, J., and Jacobsen, S. E. (2019). Site-specific manipulation of Arabidopsis loci using CRISPR-Cas9 SunTag systems. Nature communications 10, 729.
60. Piunti, A., and Shilatifard, A. (2016). Epigenetic balance of gene expression by Polycomb and COMPASS families. Science 352, aad9780.
61. Pruneda-Paz, J. L., Breton, G., Nagel, D. H., Kang, S. E., Bonaldi, K., Doherty, C. J., Ravelo, S., Galli, M., Ecker, J. R., and Kay, S. A. (2014). A genome-scale resource for the functional characterization of Arabidopsis transcription factors. Cell reports 8, 622-632.
62. Qian, F., Zhao, Q. Y., Zhang, T. N., Li, Y. L., Su, Y. N., Li, L., Sui, J. H., Chen, S., and He, X. J. (2021). A histone H3K27me3 reader cooperates with a family of PHD finger-containing proteins to regulate flowering time in Arabidopsis. Journal of integrative plant biology.
63. Qian, S., Lv, X., Scheid, R. N., Lu, L., Yang, Z., Chen, W., Liu, R., Boersma, M. D., Denu, J. M., Zhong, X., et al. (2018). Dual recognition of H3K4me3 and H3K27me3 by a plant histone reader SHL. Nature communications 9, 2425.
64. Ramirez, F., Ryan, D. P., Gruning, B., Bhardwaj, V., Kilpert, F., Richter, A. S., Heyne, S., Dundar, F., and Manke, T. (2016). deepTools2: a next generation web server for deep-sequencing data analysis. Nucleic acids research 44, W160-165.
65. Rothbart, S. B., and Strahl, B. D. (2014). Interpreting the language of histone and DNA modifications. Biochimica et biophysica acta 1839, 627-643.
66. Schrumpfova, P. P., Vychodilova, I., Dvorackova, M., Majerska, J., Dokladal, L., Schorova, S., and Fajkus, J. (2014). Telomere repeat binding proteins are functional components of Arabidopsis telomeres and interact with telomerase. The Plant journal: for cell and molecular biology 77, 770-781.
67. Segal, D. J., Dreier, B., Beerli, R. R., and Barbas, C. F., 3rd (1999). Toward controlling gene expression at will: selection and design of zinc finger domains recognizing each of the 5′-GNN-3′ DNA target sequences. Proceedings of the National Academy of Sciences of the United States of America 96, 2758-2763.
68. Soppe, W. J., Jacobsen, S. E., Alonso-Blanco, C., Jackson, J. P., Kakutani, T., Koornneef, M., and Peeters, A. J. (2000). The late flowering phenotype of fwa mutants is caused by gain-of-function epigenetic alleles of a homeodomain gene. Molecular cell 6, 791-802.
69. Stroud, H., Greenberg, M. V., Feng, S., Bernatavichute, Y. V., and Jacobsen, S. E. (2013). Comprehensive analysis of silencing mutants reveals complex regulation of the Arabidopsis methylome. Cell 152, 352-364.
70. To, T. K., Kim, J. M., Matsui, A., Kurihara, Y., Morosawa, T., Ishida, J., Tanaka, M., Endo, T., Kakutani, T., Toyoda, T., et al. (2011). Arabidopsis HDA6 regulates locus-directed heterochromatin silencing in cooperation with MET1. PLoS genetics 7, e1002055.
71. Tomanov, K., Zeschmann, A., Hermkes, R., Eifler, K., Ziba, I., Grieco, M., Novatchkova, M., Hofmann, K., Hesse, H., and Bachmair, A. (2014). Arabidopsis PIAL1 and 2 promote SUMO chain formation as E4-type SUMO ligases and are involved in stress responses and sulfur metabolism. The Plant cell 26, 4547-4560.
72. Tomson, B. N., and Arndt, K. M. (2013). The many roles of the conserved eukaryotic Paf1 complex in regulating transcription, histone modifications, and disease states. Biochimica et biophysica acta 1829, 116-126.
73. Voigt, P., Tee, W. W., and Reinberg, D. (2013). A double take on bivalent promoters. Genes & development 27, 1318-1338.
74. Wang, Q., Zuo, Z., Wang, X., Gu, L., Yoshizumi, T., Yang, Z., Yang, L., Liu, Q., Liu, W., Han, Y. J., et al. (2016). Photoactivation and inactivation of Arabidopsis cryptochrome 2. Science 354, 343-347.
75. Xi, Y., and Li, W. (2009). BSMAP: whole genome bisulfite sequence MAPping program. BMC Bioinformatics 10, 232.
76. Wongpalee, S. P., Liu, S., Gallego-Bartolome, J., Leitner, A., Aebersold, R., Liu, W., Yen, L., Nohales, M. A., Kuo, P. H., Vashisht, A. A., et al. (2019). CryoEM structures of Arabidopsis DDR complexes involved in RNA-directed DNA methylation. Nature communications 10, 3916.
77. Xue, Y., Zhong, Z., Harris, C. J., Gallego-Bartolome, J., Wang, M., Picard, C., Cao, X., Hua, S., Kwok, I., Feng, S., et al. (2021). Arabidopsis MORC proteins function in the efficient establishment of RNA directed DNA methylation. Nature communications 12, 4292.
78. Yamada, K., Lim, J., Dale, J. M., Chen, H., Shinn, P., Palm, C. J., Southwick, A. M., Wu, H. C., Kim, C., Nguyen, M., et al. (2003). Empirical analysis of transcriptional activity in the Arabidopsis genome. Science 302, 842-846.
79. Yan, L., Wei, S., Wu, Y., Hu, R., Li, H., Yang, W., and Xie, Q. (2015). High-Efficiency Genome Editing in Arabidopsis Using YAO Promoter-Driven CRISPR/Cas9 System. Molecular plant 8, 1820-1823.
80. Yang, Z., Qian, S., Scheid, R. N., Lu, L., Chen, X., Liu, R., Du, X., Lv, X., Boersma, M. D., Scalf, M., et al. (2018). EBS is a bivalent histone reader that regulates floral phase transition in Arabidopsis. Nature genetics 50, 1247-1253.
81. Yu, C. W., Liu, X., Luo, M., Chen, C., Lin, X., Tian, G., Lu, Q., Cui, Y., and Wu, K. (2011). HISTONE DEACETYLASE6 interacts with FLOWERING LOCUS D and regulates flowering in Arabidopsis. Plant physiology 156, 173-184.
82. Zhai, J., Bischof, S., Wang, H., Feng, S., Lee, T. F., Teng, C., Chen, X., Park, S. Y., Liu, L., Gallego-Bartolome, J., et al. (2015). A One Precursor One siRNA Model for Pol IV-Dependent siRNA Biogenesis. Cell 163, 445-455.
83. Zhang, S., Zhou, B., Kang, Y., Cui, X., Liu, A., Deleris, A., Greenberg, M. V., Cui, X., Qiu, Q., Lu, F., et al. (2015). C-terminal domains of a histone demethylase interact with a pair of transcription factors and mediate specific chromatin association. Cell discovery 1.
84. Zhang, X., Germann, S., Blus, B. J., Khorasanizadeh, S., Gaudin, V., and Jacobsen, S. E. (2007). The Arabidopsis LHP1 protein colocalizes with histone H3 Lys27 trimethylation. Nature structural & molecular biology 14, 869-871.
85. Zhang, Y., Harris, C. J., Liu, Q., Liu, W., Ausin, I., Long, Y., Xiao, L., Feng, L., Chen, X., Xie, Y., et al. (2018). Large-scale comparative epigenomics reveals hierarchical regulation of non-CG methylation in Arabidopsis. Proceedings of the National Academy of Sciences of the United States of America 115, E1069-E1074.
86. Zhang, Y. Z., Yuan, J., Zhang, L., Chen, C., Wang, Y., Zhang, G., Peng, L., Xie, S. S., Jiang, J., Zhu, J. K., et al. (2020). Coupling of H3K27me3 recognition with transcriptional repression through the BAH-PHD-CPL2 complex in Arabidopsis. Nature communications 11, 6212.
87. Zhong, X., Du, J., Hale, C. J., Gallego-Bartolome, J., Feng, S., Vashisht, A. A., Chory, J., Wohlschlegel, J. A., Patel, D. J., and Jacobsen, S. E. (2014). Molecular mechanism of action of plant DRM de novo DNA methyltransferases. Cell 157, 1050-1060.
88. Zhou, Y., Hartwig, B., James, G. V., Schneeberger, K., and Turck, F. (2016). Complementary Activities of TELOMERE REPEAT BINDING Proteins and Polycomb Group Complexes in Transcriptional Regulation of Target Genes. The Plant cell 28, 87-101.
89. Zhou, Y., Wang, Y., Krause, K., Yang, T., Dongus, J. A., Zhang, Y., and Turck, F. (2018). Telobox motifs recruit CLF/SWN-PRC2 for H3K27me3 deposition via TRB factors in Arabidopsis. Nature genetics 50, 638-644.
90. Zilberman, D., Cao, X., and Jacobsen, S. E. (2003). ARGONAUTE4 control of locus-specific siRNA accumulation and DNA and histone methylation. Science 299, 716-719.
91. Antosz, W., Pfab, A., Ehrnsberger, H. F., Holzinger, P., Kollen, K., Mortensen, S. A., Bruckmann, A., Schubert, T., Langst, G., Griesenbeck, J., et al. (2017). The Composition of the Arabidopsis RNA Polymerase II Transcript Elongation Complex Reveals the Interplay between Elongation and mRNA Processing Factors. The Plant cell 29, 854-870.
92. Bloomer, R. H., Hutchison, C. E., Baurle, I., Walker, J., Fang, X., Perera, P., Velanis, C. N., Gumus, S., Spanos, C., Rappsilber, J., et al. (2020). The Arabidopsis epigenetic regulator ICU11 as an accessory protein of Polycomb Repressive Complex 2. Proceedings of the National Academy of Sciences of the United States of America 117, 16660-16666.
93. Chen, X., Lu, L., Qian, S., Scalf, M., Smith, L. M., and Zhong, X. (2018). Canonical and Noncanonical Actions of Arabidopsis Histone Deacetylases in Ribosomal RNA Processing. The Plant cell 30, 134-152.
94. Derkacheva, M., Steinbach, Y., Wildhaber, T., Mozgova, I., Mahrez, W., Nanni, P., Bischof, S., Gruissem, W., and Hennig, L. (2013). Arabidopsis MSI1 connects LHP1 to PRC2 complexes. The EMBO journal 32, 2073-2085.
95. Frost, J. M., Kim, M. Y., Park, G. T., Hsieh, P. H., Nakamura, M., Lin, S. J. H., Yoo, H., Choi, J., Ikeda, Y., Kinoshita, T., et al. (2018). FACT complex is required for DNA demethylation at heterochromatin during reproduction in Arabidopsis. Proceedings of the National Academy of Sciences of the United States of America 115, E4720-E4729.
96. Han, Y. F., Zhao, Q. Y., Dang, L. L., Luo, Y. X., Chen, S. S., Shao, C. R., Huang, H. W., Li, Y. Q., Li, L., Cai, T., et al. (2016). The SUMO E3 Ligase-Like Proteins PIAL1 and PIAL2 Interact with MOM1 and Form a Novel Complex Required for Transcriptional Silencing. The Plant cell 28, 1215-1229.
97. Harris, C. J., Scheibe, M., Wongpalee, S. P., Liu, W., Cornett, E. M., Vaughan, R. M., Li, X., Chen, W., Xue, Y., Zhong, Z., et al. (2018). A DNA methylation reader complex that enhances gene transcription. Science 362, 1182-1186.
98. He, Y., Doyle, M. R., and Amasino, R. M. (2004). PAF1-complex-mediated histone methylation of FLOWERING LOCUS C chromatin is required for the vernalization-responsive, winter-annual habit in Arabidopsis. Genes & development 18, 2774-2784.
99. Hennig, L., Bouveret, R., and Gruissem, W. (2005). MSI1-like proteins: an escort service for chromatin assembly and remodeling complexes. Trends Cell Biol 15, 295-302.
100. Jackson, J. P., Lindroth, A. M., Cao, X., and Jacobsen, S. E. (2002). Control of CpNpG DNA methylation by the KRYPTONITE histone H3 methyltransferase. Nature 416, 556-560.
101. Lario, L. D., Ramirez-Parra, E., Gutierrez, C., Spampinato, C. P., and Casati, P. (2013). ANTI-SILENCING FUNCTION1 proteins are involved in ultraviolet-induced DNA damage repair and are cell cycle regulated by E2F transcription factors in Arabidopsis. Plant physiology 162, 1164-1177.
102. Lawrence, R. J., Earley, K., Pontes, O., Silva, M., Chen, Z. J., Neves, N., Viegas, W., and Pikaard, C. S. (2004). A concerted DNA methylation/histone methylation switch regulates rRNA gene dosage control and nucleolar dominance. Molecular cell 13, 599-609.
103. Luo, M., Wang, Y. Y., Liu, X., Yang, S., Lu, Q., Cui, Y., and Wu, K. (2012a). HD2C interacts with HDA6 and is involved in ABA and salt stress response in Arabidopsis. Journal of experimental botany 63, 3297-3306.
104. Luo, M., Wang, Y. Y., Liu, X., Yang, S., and Wu, K. (2012b). HD2 proteins interact with RPD3-type histone deacetylases. Plant signaling & behavior 7, 608-610.
105. Qian, F., Zhao, Q. Y., Zhang, T. N., Li, Y. L., Su, Y. N., Li, L., Sui, J. H., Chen, S., and He, X. J. (2021). A histone H3K27me3 reader cooperates with a family of PHD finger-containing proteins to regulate flowering time in Arabidopsis. Journal of integrative plant biology 63, 787-802.
106. Schrumpfova, P. P., Vychodilova, I., Dvorackova, M., Majerska, J., Dokladal, L., Schorova, S., and Fajkus, J. (2014). Telomere repeat binding proteins are functional components of Arabidopsis telomeres and interact with telomerase. The Plant journal: for cell and molecular biology 77, 770-781.
107. Tan, L. M., Zhang, C. J., Hou, X. M., Shao, C. R., Lu, Y. J., Zhou, J. X., Li, Y. Q., Li, L., Chen, S., and He, X. J. (2018). The PEAT protein complexes are required for histone deacetylation and heterochromatin silencing. The EMBO journal 37.
108. Tomanov, K., Zeschmann, A., Hermkes, R., Eifler, K., Ziba, I., Grieco, M., Novatchkova, M., Hofmann, K., Hesse, H., and Bachmair, A. (2014). Arabidopsis PIAL1 and 2 promote SUMO chain formation as E4-type SUMO ligases and are involved in stress responses and sulfur metabolism. The Plant cell 26, 4547-4560.
109. Turck, F., Roudier, F., Farrona, S., Martin-Magniette, M. L., Guillaume, E., Buisine, N., Gagnot, S., Martienssen, R. A., Coupland, G., and Colot, V. (2007). Arabidopsis TFL2/LHP1 specifically associates with genes marked by trimethylation of histone H3 lysine 27. PLoS genetics 3, e86.
110. Wu, K., Tian, L., Zhou, C., Brown, D., and Miki, B. (2003). Repression of gene expression by Arabidopsis HD2 histone deacetylases. The Plant journal: for cell and molecular biology 34, 241-247.
111. Yang, H., Han, Z., Cao, Y., Fan, D., Li, H., Mo, H., Feng, Y., Liu, L., Wang, Z., Yue, Y., et al. (2012). A companion cell-dominant and developmentally regulated H3K4 demethylase controls flowering time in Arabidopsis via the repression of FLC expression. PLoS genetics 8, e1002664.
112. Yano, R., Takebayashi, Y., Nambara, E., Kamiya, Y., and Seo, M. (2013). Combining association mapping and transcriptomics identify HD2B histone deacetylase as a genetic factor associated with seed dormancy in Arabidopsis thaliana. The Plant journal: for cell and molecular biology 74, 815-828.
113. Zhang, Y. Z., Yuan, J., Zhang, L., Chen, C., Wang, Y., Zhang, G., Peng, L., Xie, S. S., Jiang, J., Zhu, J. K., et al. (2020). Coupling of H3K27me3 recognition with transcriptional repression through the BAH-PHD-CPL2 complex in Arabidopsis. Nature communications 11, 6212.
114. Zhou, C., Labbe, H., Sridha, S., Wang, L., Tian, L., Latoszek-Green, M., Yang, Z., Brown, D., Miki, B., and Wu, K. (2004). Expression and function of HD2-type histone deacetylases in Arabidopsis development. The Plant journal: for cell and molecular biology 38, 715-724.
115. Zhou, Y., Wang, Y., Krause, K., Yang, T., Dongus, J. A., Zhang, Y., and Turck, F. (2018). Telobox motifs recruit CLF/SWN-PRC2 for H3K27me3 deposition via TRB factors in Arabidopsis. Nature genetics 50, 638-644.

What is claimed is:

1. A method for producing a plant with reduced expression of a target nucleic acid, comprising:
(a) providing a plant comprising a recombinant polypeptide comprising a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide, and;
(b) growing the plant under conditions whereby the recombinant polypeptide is expressed and targeted to the target nucleic acid, thereby reducing expression of the target nucleic acid to produce the plant with reduced expression of the target nucleic acid.

2. The method of claim 1, wherein the transcriptional repressor polypeptide comprises an amino acid sequence having at least 80%, at least 85%, at least 90%, at least 95%, or 100% amino acid identity to any one of SEQ ID NO: 1-210.

3. The method of any one of claims 1-2, wherein the targeting domain comprises a DNA-binding domain.

4. The method of claim 3, wherein the DNA binding domain comprises a zinc finger.

5. The method of any one of claims 1-4, wherein expression of the target nucleic acid is reduced by at least 50% as compared to a corresponding control nucleic acid.

6. The method of any one of claims 1-5, wherein the recombinant polypeptide is encoded by a recombinant nucleic acid.

7. The method of any one of claim 1-6, wherein the transcriptional repressor polypeptide is a TRBIP1 polypeptide.

8. The method of claim 7, wherein the TRBIP1 polypeptide comprises an amino acid sequence having at least 80% identity to SEQ ID NO: 181.

9. The method of any one of claims 1-8, wherein the plant further comprises a recombinant DNA methyltransferase polypeptide which is capable of being targeted to the target nucleic acid.

10. The method of claim 9, wherein the DNA methyltransferase polypeptide is an MQ1 polypeptide.

11. The method of claim 10, wherein the MQ1 polypeptide comprises an amino acid sequence having at least 80% identity to SEQ ID NO: 212.

12. The method of any one of claims 1-11, further comprising:
(c) crossing the plant with reduced expression of the target nucleic acid to a second plant to produce one or more F1 plants.

13. The method of claim 12, further comprising:
(d) selecting from the one or more F1 plants an F1 plant that (i) lacks the recombinant polypeptide, and (ii) has reduced expression of the target nucleic acid.

14. A recombinant nucleic acid comprising a plant promoter and which encodes a recombinant polypeptide comprising a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide.

15. The recombinant nucleic acid of claim 14, wherein the transcriptional repressor polypeptide comprises an amino acid sequence having at least 80%, at least 85%, at least 90%, at least 95%, or 100% amino acid identity to any one of SEQ ID NO: 1-210.

16. The recombinant nucleic acid of any one of claims 14-15, wherein the targeting domain comprises a DNA-binding domain.

17. The recombinant nucleic acid of claim 16, wherein the DNA binding domain comprises a zinc finger.

18. An expression vector comprising the recombinant nucleic acid of any one of claims 14-17.

19. A plant cell comprising the expression vector of claim 18.

20. A plant comprising the plant cell of claim 19.

21. A plant cell comprising a recombinant polypeptide comprising a transcriptional repressor polypeptide and a targeting domain, wherein the transcriptional repressor polypeptide is selected from the group consisting of: a PHD1 polypeptide, a PIAL1 polypeptide, a PIAL2 polypeptide, a TRB1 polypeptide, a TRB2 polypeptide, a TRB3 polypeptide, a MSI1 polypeptide, a LHP1 polypeptide, a HD2A polypeptide, a HD2B polypeptide, a HD2C polypeptide, an ELF7 polypeptide, a CPL2 polypeptide, a MBD2 polypeptide, a SUVH7 polypeptide, a SSRP1 polypeptide, a SPT16 polypeptide, a JMJ18 polypeptide, a TRBIP1 polypeptide, a TRBIP2 polypeptide, and an ASF1B polypeptide, wherein the plant cell comprises a nucleic acid with reduced expression as compared to a corresponding control nucleic acid.

22. The plant cell of claim 21, wherein the transcriptional repressor polypeptide comprises an amino acid sequence having at least 80%, at least 85%, at least 90%, at least 95%, or 100% amino acid identity to any one of SEQ ID NO: 1-210.

23. The plant cell of any one of claims 21-22, wherein the targeting domain comprises a DNA-binding domain.

24. The plant cell of any one of claims 21-23, wherein the DNA binding domain comprises a zinc finger.

25. The plant cell of any one of claims 21-24, wherein expression of the target nucleic acid is reduced by at least 50% as compared to a corresponding control nucleic acid.

26. The plant cell of any one of claims 21-25, wherein the recombinant polypeptide is encoded by a recombinant nucleic acid.

27. A plant comprising the plant cell of any one of claims 21-26.


SEQUENCE LISTING (219 sequences)

SEQ ID NO: 1 - protein, 431 aa, Arabidopsis thaliana
MSLHVKGPVCQTCGDIGFEEALVFCDSCMFESIHRYCLGITPIPFTEYITWICEDCDNSESDSDCNEVDQTVKKKKKSRNQPLVVLAEDNVLQEPLEGSKREESSCSRKPHELTCLDGNGESVLEAADSSSVPDHSSCTSKRKEVDQTANLGHILERSEKKKKKKKKKKSINHSPPVLAVEDHELQGTTNVVEPVEVSSSSPTKETMESKRQESSDSRKPHELTCLVGDSETANSSSVPEHNSCAVKKRKLSSGNKQIQLADGSSSCRVAESNMPLTLTSRNYRAQPIKIPIWRGLMSVKGGNSCTMDGIVAHVSSLACPKVHETASSLKGRLSAEILPRLEVWPKTFLKNGGPKDESVALFFFPSSESNDEKVFDSLVDKMKKNDSAMRCVLNDAELLLFTSYMLPKDSWTFNSKYYLWGVFKPRQTSRY

SEQ ID NO: 2 - protein, 343 aa, Glycine max
MEAVCLHCGDRGFPETLVFCTECKAYALHRYCLKGPVVFTDAVTWFCEDCATKLGVPPALDQSTPISSVSRNNCIQRVKKHNKQRKKKLKKKQKKGKANSSLVAKTKEVLSGCHISPEPEHPQCSNRGEECEMKDECGPAPRDVANFDVGEEESVARDLANSDEGFKSVPVSQGATDNDSGCVELDGHVYAHPTIDPIWRGSMYFCNETNGTVNGVLAHMSNLACSKVAEETGHFPEVLHAELLPRDKVWPESFKSRRPTDQDIALFIFPDTEGSEKDFDKLVEDIMINEHVIRIVAKKAELLIFHSIELPIQYWRFEAKYYLWGVFRRKQTSEETNDAVYRD

SEQ ID NO: 3 - protein, 602 aa, Zea mays
MARTSKNSNPRYVLASKCASQPPTMWLENSLNTSSGFKCSKLSIVRNALPSKTPKASVSWKSKDDSNAIPRHLEQSHCKKTSRATRTHFKAMPNLIHKAKNVDTSNLNDGTSGSSWRGQSDSYKGEDINIGKECTKTSELLKSREASRKSNILVVDRKDPNKDEPDKVKGIQDEVMTKEEGVKGHTCEGRATCWSCLKQANTTKTNDAPNLGSLLVGNEHMQGLMSNLRDNSSHLFEKNHSRHQRDDPCEYRETNLKFPNRIQSVGGNGKTNDVTSRRKRLLLEEKASAHVQNDLTYGPMKRRRYIEANEDEEEQNDLTYGPMKRRRYIEANEDEEDYVGDHHPVHIEDATTELTPQDSISKHCVERQCYCCSKPIDIPRWSGTFKIDGKEHISLAGHLSSKSCEKVWKLSSLPKVVQVTKVPRMAVWPKIFKASEPSGDNIGLYFFPPEMRHDEELDQLVNEVMDKDLILRAIIGEAEMLIFPSVLLPERYQTFQTKHYLWAAFKAKEDKGDAIVEEEVEKGNCVSNQPDKVQSDNASFPSNHGLDRPSMEAPPQSSSSVFGIVVRQAPNLEPEVKRFIQEMERKGALVTVMRGEAIGGGP

SEQ ID NO: 4 - protein, 902 aa, Oryza sativa
  source 1..902: note = Japonica Group
MATTPRLLQPSSEPRGLRRRAAHRRQVEPEPRRPGERAAVGERRRIQVSRSRGGGSVSTAEKPEGRSPQRSRAAPQHQAAISANMITVCEVCGVLGYKNLLLSCKNCNGAAVHRYCLDKVDFDGTVDWSCDECHPRHGKGTNRRSLEVTLDDKTVVGKQPENQSSLDTNHDKPGMNGGNDDYVSDDLMLERNKERFQLHDEANNDIHLKSMSANVPRSSTLHENSVARNVSSSANTGLPMDSNCVPSAHIDIGNPRDSSVRLILTGENRRESSMLLDGACSGSLSKDSSKEKIDREANSSHMEPSDAVKNFCKDNPMKRRKLMLLDDDDDDVGVELSDTVQNVVKDNPSKRRKLILPDDDDVEVELCNTVQSIAKDNPGKTIQLIVLDDDDDKQEDAENLNHLSLEFEGPIEKHKIHIGYATGQRCLEDDEHGLLDSLDHRSLENTCPTKKRRRYICPSDDEEEEVIKGSTTTGCAPNDVANTASQHVDAKDHHLQSRMAFASDFTKHQYYIYSEPMGEPAWSGIFMTDSNVPIMLAAHLSTKACQRVSEFARSLQPVVEVIKLPRLKAWPERWDKSGPTDDSIGLFFFPHSMRPNEELDKLVKEVIESDVVLKAVLGTVELLIFPSILLPEQYHEFQGKYYLWGVCKARKHNPDTAVLVEEQCGLVSASEEGSSDKESYVMKHVEDRLPADCNPEAREGDIKTALGEGCFSPDSCLSSNKASPVKGGSPCFMQPGLGDKPHEPGVADQKEDEQDFTSLPRRNDQNATNPPIDSLPSATRLFGFVTARSERCQQLIQEMVKEGALLFSVPEDMTINRSTISKSNGVGAAQAPDSGCQHVQERCEPIEFVPIDHNDPDSASEACLDLFPVRHEQIGLTSQVDVKEVELDLSLGAFRRAPSELP

SEQ ID NO: 5 - protein, 826 aa, Sorghum bicolor
MNALCEVCGAIGYEHLLLCCTDCKGGHTHQYCLDKVLFDATLEDWFCDECKQWRNEGSHSRSLEKVSSERPSNHARFDSTSQQAIIKRPESARVVGTWGQQKRKSTVVGANLKKKHSSRAKSLRKKCIRNKSNMRGKGINRRGIRSKRSSNFTEQLNLSIGQVGSALLSKGVKQPTPVIVNALGYTLGDTGQSHVLKPLEGLAHKSMQTPQSLTTDQVYSTSSLEISSKAKFALEASATEVEISDTVQNLRKDSPKKRRRLILTGEDEEEEEEEEEEEEEEEEEEEEEEEEKAEDVQQENVNHQPLKCNEPMVKHQINTEYYVEEAVQTGDINDQCLIKGRRMKQRRRYIVENEDEEDTEGAECALNNASKWPSNDSAKMASQTPVATDHSHQSRRSESERVDRHYHICLQPLDEPVWSGVFNIDNEVFLKLDAHLSNKACQRVHELSGLLQPVVEVKTLSRFQVWPERWISSGPTDESIGLFFLPHSSMQDEELTRLNRIIKSDDALQITIGIAELLIFPSVLLPEQYHLFQRKHYLWGVFRQRKDILDNGVHVEVRDGSAHATGEGQHQEYNLLDQQDEALYELSDKETFAVKHVVDVEDQLQVERDPEVQNDIMKVATREGITSLSSSWSPATGSSKAGSNSPVQPRTERKLYTPGDAEQQEDFTSSPQWDASSIKQSTAAIPAEHGNKQAHPDSEPSTAKLFGFIAVRTPRSQQLIQEMVSEGAVLFPVPEEIATTDSVTGSGTGVVSSALNPDAKCEAPQAFDFVSVGHGELGVDSEACLELFPVRQEQIGWAPRAEVSGEVDLNLSLGKRLPAPSSPPLL

SEQ ID NO: 6 - protein, 388 aa, Cucumis sativus
MLHSDTHPFKMESLHVVPLTLPTADLRPSNKPIEHFSSLTVILNVDICQKCGDRGFVEALNFCNECQTYPIHRYCLDELPKYLDEYVPWLCEDCEAAILPTINSEVKLKKKKQVKQLKKKKKKKKKQKKIKGNDDCDKSSYSLQLPEAHCSENKNESTPGRLGEPVLEDGAVRPPITHTYKRKGKKGSVLTGANHGQVATSSDMCNSTRLNCYVAQPIVDPVWRGTLKFWNRSSGKVCVLVAHMSSLACSKVYEEAKMLPVLLSAELFRRCDVWPRGFQKLGPTDQNIALYFFPDGESQKAFDLLVNAMMSQDLAMKAVLKNAELLVFTSSMLPMRYWRFQTKYYLWGVFRGKQTAKPRNVVASEERTFAESTCMQGPISPDQYTSSC

SEQ ID NO: 7 - protein, 439 aa, Vitis vinifera
MCEGCAPRISKESPLSTPSSVPARTGDRLRLKKIVQVSHCRTRLSYKNEIPGSVTKTVVQRREHLRSAQATECRSRLKEKKSKPSLVTKAEVQSSEHSLSHQPCDAPCSKIHEKDQTLGKHGQSVSDDAGNFIEEAEFVKTNASEITTGEPSNMLEIICFESEQPMVDTNLSETYEKYQELGKQRKLVHEDQGNTREEAEPAKDTVSELAIIGPSNTPELDPSNVPEFSCFVHAQPVVDPIWRGSFSICNENFDSVNGLAAHLSSKACLKVCQQASLLPALLCPTMLPKFDVWPKSFQISQPSDDNIALYFFPENERDEKVFDRLVLDMVSHELAMQTVVENAELLVFASTELPLRHWRFHGKLYLWGVFRGKQVSSTSCQVDDRHLIPNSSKESVTSSNLANDAGIRKEKDTSMVTTWDSRSPLSPLSNSGSHGSGSL

SEQ ID NO: 8 - protein, 1380 aa, Gossypium hirsutum
MRGVFMVPPKTVKRLVRRSLSQKVHTKAESGTCNVCSAPCSSCMHLSISQMGSKKNEFSDETDSVAVATQYSINDDKTNDSLQNTHSEASNLLSVNSSHDSYSENIESKATTRPSDASEDVGIQRNFSNKYDGSKGIEGHDDSSLLASRASGANTAFSYCNKDLDSNNSSRSSVSVCSLGSGKVLSSQKVDLSELPSVKEVDDSKVSLRIQSPYSHSQSDKSTVGGSSEISTKIHLKSEADTDRKALDKADKSLNEDEHDESNELVELPGKLESPLQAASGDESCESVATELDVKVCDICGDAGREDLLAICSKCADGAEHTYCMREMLQKVPVGDWLCEECKLAEETESQKQGLDSEGKKAKKLSSTTQNLGKRHAENLEDASASKRQAVEQKMGSPKSLSPSKASALSREGSFKNMDKGKVRPSPQLSLGCHSGNDMLETTCSPASGPRLQSPKGTLLKSNSFNALNSKPKVKLVDEVALQKEKIFRKHASLNSKEEASRVMGKSMSFKSTNSGRLSTGESKFKMLSSKYAYVQDLKGLKQVKEQISLERKNLSKLDRSSSTVSTPKVDQKLTTRTDTISHSSASNSRETKAVRSDGKPSTLSRSTTNLVRNGVENAVISASGVSTTNGGISSEQKLNQVSLKEEPSSSTSWTVERQPSNNNVIISDGLSWSLDLTNQSERMGHTAEYCSVSQASGADESAPRNFKEEINKGNKLKAAIEAAICLRPRICERTSQDPSSVSVKAKNMISVEGTHESQTNVQNQTSIGNMKLLNAHCTDALSVVSSVGNAPPLVAESAVSEMSAIPEHECIWQGAFQVHKMGKPPDFCGGIQAHLSTLASPKVIEVVNTFPLEVPLNEVPRLSTWPTQFHNSGPKEDNIALYFFAKDLESYEKYYKVLLDTMVKNDLALKGNFEGVEFLIFPSNKLPESCQRWNTLSFLWGVFKGRRANCSDTSKSAYVPDASMVHLEREKSTDVPQPVENESAACHSSCNVVVTSAVEKTCISTERVSDSKVSSFGKTYVGIKAKLEEQDGNIDNKYLSRIATNSIKVHPDMKSTSPLDDGKVPDCRLDTELKPCHQATETNIRSFEVKKEEMQMPVEEDYLSLKDRPTGKQEAVIVGKIDGEYVKTRESRDDGYADVNTYSKRDVSSWQLNHRKRPYLDFTEAVPEISTESSQRMPWSEVRRVSIDRGSDNKKLKTGFTGIYKYNGARDEGTFSDSTASDRHGLGSGSCVKERRCNIACEEKGVPKDMGSGERFFPMGSDRVREIRMDWREELLVKDENRAGDASPNLELALGAEMRPPNKGILPFFVGTADKSENLDKVTGKEEADDVSASLSLSLSFPFPETEGNVKSVSKTEQLLPESHGVNTSLLLFGGFPNK

SEQ ID NO: 9 - protein, 572 aa, Citrus sinensis
MVGTSISPNYGVLWLCFHRKSDFEVTVCQQCGDKGFYEALIGCEKCQTTAVHIYCLPVLPASFEDDVLWYCEDCEPKVAKPSTVVNPSSVLGGRCDSDSENLEIVQATRSNLRLKKNAVDRLSKNERKGNDSGSLAKAEDQKSGNSSSYLLQVHHSNNHEKDKKCGRRGELDGRSIYEEAESVKTKTSLATGGDSNSNPQYSCKLLCNESDENYEKLGRQIGLDRSSFNDEAESFRTRTSQVVTNDSPYTRECSSNAHKVVVIEVHSMGNDKKGYQFRRQRELVKNSSDTATKSLKNSLVTTSNSFSIPKHSSNVSGKSIFEVQCKEDAVKDLSPIRWSGLNGGSSTEESESSKKETSLIASSRPSNIPEHCYVPAQPIIEPIWKGNLRLSSDKYGSIEGLVAHLSTSACSKVFQEAKLLPQLLCPELLPRSNSWPKSFHKWGPSDDNIALYLFPNNERSEKVYDSLVHDMISHDVALRAVVKNAELLLFTSNLLPFQFWRFQSKVYLWGVFRAKQASQSQMLAVDSVNAISISKPNGLVKALTQYPQSPGSPLSNSGSHGSGSVHSCQSML

SEQ ID NO: 10 - protein, 451 aa, Populus trichocarpa
MVTICQKCGDRGFDAALIFCDECQAYAVHCYCLDVLPATFDEYVVWLCYHCESKAVKLSSLDRPNSPISTESDSDSLKIIQLKKKNPLKRLEGKPKEMVFDCSLTNSDLLRPQISSDFQLVEVDCCEDDGKDQKLGSQNGLHEDSVPEVAEYLESKNPVSPLPDLQLVDVDCSQNDEKDQKLGGKNSLEESGFQETEPLRNKNSQLVVCDVQPLQIHCREDGGGSQKVGRQNDLNEGNFVEEDELDKTKICHVDAPYFAEQSSIRVLPIRDPIWRGSMSIFQNNYGAPGGIVAHLSSIACSRASEEAKGLSGLLSPELLPRSGVWPKSFRKLGPAADHIGLYFFPDNERNEIVFDSLVNDMISQDLAMRVVIENAELLIYTSRILPMDCWRFQSKFYLWGVFRPKKPSAPDVVPGEQQGLTKAITWERRSPVSPLSNGSYGSGLMCPSQDS

SEQ ID NO: 11 - protein, 829 aa, Arabidopsis thaliana
MVIPATSRFGFRAEFNTKEFQASCISLANEIDAAIGRNEVPGNIQELALILNNVCRRKCDDYQTRAVVMALMISVKSACQLGWFPERETQELLAIIDLMWNGFSCPENVTSCVNSPVTLISQVIERFYPCVKLGHILVSFEAKPESKMMMKDFHISKKMPHSPKQKVGLFVVRTEDISRSNCIVHPQGVSFLLNGKGIDKRVNISMESGPQLPTNVTALLNLGANLLQAIGCFGGSYLIAIAFMDVIPLPNKPLLKDYVHPEVVGSNSDCDIIEGPSRISLSCPISRTRIKLPVKGHVCKHLQCFDFWNYVNMNTRRHHGAARIILEEVGRNAADVVISADGTWMVETENDEDVELVPETTHDHGDPNSFINLGPTVKNPARDENEMETSTQVEEHNPCLSEIQGPSNDTHRPASDYTMLNQSHTSTNTLPQLPRTLNAFDGQQFVNLPQVINTRDSPASQALPMTFSPTPSPQDILATNAANFGTSMPAAQSSQFQGSHVTSLGNCEGRTSDLMARWNHIYGRVQTQFPPAPLSHHHYSMQNQSPSPAQQRPVPSYIAHPQTFHVNYGENADQRWMPSSIAHPQTLPVNYGGNTNQRPIPSSIAHPQTLPVNYRGNTDHRSTPYSITHLQTLLNYGGNADQRPMPSSITNLQTLPATYGGYAHQRPMSSSITHPRTSPVNYGGTPDQRPMPSSITHPQTLPVSYGGTTDQILNPGGAMGQFSSREFMNLTPANTENWRPQSRMRGSVAPGTGYDHMIIHPTRPVHPQAQTPPAPLSTSYDGADEIQAFIGHPSYPVSNNETQAGTSSLPVAEGLGYSGSFWSMPPETW

SEQ ID NO: 12 - protein, 730 aa, Glycine max
MQKFYPKFKLGPILASIEAQPGYGASVVDFHITKSEVLKDKIFLLVAQTDNIETSACLINPQQVNFLLNGKGVLNRTNVQMDPGPQVPTNVTGMLKFGTNLLQAVGQFNGRYVVLVAYMSFTPFLEDPVLQDYLQPAVTSVDSDSDIIEGASQISLNCPISFTRIKTPVKGHSCKHFQCFDFDNFINMNSKRPSWRCPHCIQNVCYADIRLDRNMVEVLKNVGENITEVIVLANGSWKAVLEKDHDVDKMQKKARNCEKEQTQPQESTCPPGTVDLTKDDDGLDTVGSCDIVERKPTPASIHSQFVTPNSTSLGMNSTGVNQNVATQIDDFWPGVCFVRSRSDTPTVGNSELPVLPDTVSPTFSQESAGHDNNPVVNSAMHNQFLGPNNLQMQMNHMNSVNEYGRSSSAPRHIHRTPVAVQALPVQSQALGPQQNSITNLNSSLLPSNSSATPHISLSNPTSVDTLNAILSDTERQQHFSRTPMNLPQVSGVNSPAFQHHTATQNRGPLINTSAPTQPQNQYRANAFSEFRNLHLQQALNLRPPPPRSSNAQWPRIQQGVPQSGNFQAAARGASVAAGQGSSHARNVPTSGATTHSHQARGMVANQPARPSVLVQNQSTVAGTPFHGLTTEQRGNTAQSVSRPEELFSSQSEQNWAPTGRMRGSLDLSQLNDESIAQRIITPTQGQNSRPPGPQPIRRTGISSLQPATTQQDVLIANNRNANAHNRSSSR

SEQ ID NO: 13 - protein, 860 aa, Zea mays
MTNAAGTAPHPTPPTPLQQQQHQPPKDQQQKALALNAFRLKGIGDRIRFHLKGMSVPPVAELAHLIYAFARGIDFAVSAGDIPLTAGDIPEILRKVYELRKETIIQSSLMILAISCKNACSNKWFQPADSRDILRMVNELSGNFCTSTGQAASDSTVLEIISQIMPRYYPRLKFERLITSIEAKVGYDILMADFFIERSLSRDEKIRLIVVQKENLDASSCVSSPPHVSFLVNGKGVDKRTNVSMEQGPQLPTDITKMLKYGANIIQGVGYFNANYIIAVAVVNDLTSFSAPKLDDYAQPITVYPADSDVLEGPSRVSLNCPISFRRIKTPIKGRLCKHYQCFDYDNYMEMNSRKPNWRCPYCNTSSSFTDLRIDQKMMKILEETGDDVTDVLVFADGSWKVVPAQDEKSDRHRGDTIQQAGDAVETGSPSRDVIDLINGNDDGDMQMDWASAPEDSKPVLNSQDLSVSDYLTDFPMTAQTEDLNRGDGNNGGSNMASTSRQNLLLPPTSGLGSSSLGTLESILPQNVLHPVITDAVSPSLETSSASGMQHVSQETHCGTVQLQPQIGPVHGSEVRRLPIPRNPRREPIGVQGLTVPPQNPGSSTRLQPNILNCPPPIPLSSPSSSTYQAHQVKNPDSVIASRNSGSGPLPRTPSVAAASLHLQSTARDIRNTSSHLPSRVVGLPAPHLMGARPPPGISVQAGGANAYRTMQQTPTMDPLRHTRMSMNQTALAAAGQTTAAAYVRPTQADIQNHLFPVQQSQAPRSQQVPRTTTPPLQRAPPHLQPPSVPPTVPSTPQAGPSHGLPPELPVDDSWRPTGQMRGSLTGNAYSSAIRHYRGHSEQQQGEPRPPNTSGARRPQ

SEQ ID NO: 14 - protein, 872 aa, Oryza sativa
  source 1..872: note = Japonica Group
MASAAPLPPTPPPSQQQQPQPQGKEQQQQMAVAMNARRLVMIGDRLRTHFRGGGGTVLEPPDLAHLVYAFARGIDFALSSGDVPTVASEIPSILKKVYLVGKDQFLQSSVMVLMISCKNACSEKWFQPTDCTEILRMANELSGKFCTPVSQPDNDSTVIQIISTIMPRYYPQLKFERLVTSLEAKVGYDVLMADFFIHKNVPREEKINLIVVQKEDLNASSCIANPPHVSFLVNGKGVDKRTNVSMETGPQFPTDITRMLKYGANIIQAIGYFNANYIIAVAFLNKLESFDAPNLNDYAQPVAADPPDSDLLEGPSRVSLKCPISFRRIKTPIKGRLCKHYQCFDYDNYMELNLRKPTWRCPFCNTPSNFTDLRIDQKMVKILQETGEDTIDVLVFADGSWKAISTNDERSDRHSSDVIQQSRDTMDTDATADDVIDLINEDNDGDVPMSFTSASEDVKPFLNCQDLSVADYLSDLPMNTVSQAEDLYAGGASRGNNERGNATSTSGQNSSLPSTGGLGSSSFGTLESILPHNILHPVITDAVSPSLDTSNSVVLRQHVAQGTRSDIVPSQPRIDPQLRLEIARPPIPRNVAREPTGIQALPVQPQRVRPNIYNCPPPFPQSSPASAYQVHQVTNADSVITAMSTGIGSLSRAPDAAPLLQHQSTQQEIRATQNYHQGQFIGLTAPQNFMGTRPPPGVPGQAIGANAHGAPPAQQSHHVHRLVSNLMNQLGQATVAQPSTAPQVLPSQPGGTSAVNPQIRGHLFPAQQRSQAMRPQAVPRPTISQAPPRAQSPFLPATARPPSTPPPIGTSDDLQELPVDESWRPTGQMRGSLTGEAYSVAIGRYNPSVNIAGQQTSHVTSQARPAGPDARR

SEQ ID NO: 15 - protein, 859 aa, Sorghum bicolor
MATAAATAPHPTPPTPLQQQQHQQDKDQKQQKALSMNAIRLKAIGDRLKAHLRGMNVPPIAEFAHLVYAFARGIDFAVSAGDIPQMAGDIPDILRKVYDLRREMFIQSSLMVLVISCKNACSNNWFQPADSRDIFRMANELSGNFCTSTGQAASDSTVLEIISQIMPRYYPRLKFERLITSIEAKVGYDILMADFFIERNLSRDEKIRLIVVQKENLDASSCVSSPPHVSFLVNGRGVDRRTNVSMEPGPQFPTDITKMLKYGANIIQAVGYFNANYIIAVAFVNNLTSFSAPKLDDYAQPITVYPADSDVLEGPSRVSLNCPISIRRIKTPIKGRLCKHYQCFDYDNYMDMNSRKPNWRCPYCNTSSSFTDLRIDQKMMKILEETGDDVTDVLVFADGSWKAAPAQDEKSDRHRGDAIQQTGDSTETDSPSSDVIDLINGNDDGDMQMDWVSAPEDTKPLLNSQDLSVSDYLTDLPMAVQTGDLYRGDGNNGGSNMAFTSRQNLLLPPTSGLGSSSFGTLESILPQNVLRPVITDAVSPSLETSTSTSGMQHVSQETHCGTVQLQAQIGPVHGSELRRLPIPRNPRREPVGVQALAVPPQNPGSSTRLQPNILNCPPPIPLSSPSSSTYQTHQVTNPDSVIAPMNSGSGPLPRTAASLHLQSTAREIRNTSSHLPSRLVGLPAPHLMGARPPPGISGQAGGANAYRPIQQTPTLDQLRHTRMNMNQTALAAAAQTTSAAHVRPTQADIQSHLFPTQQSQAPRSQPVPRAASPSPLQRAPPHLQPPSVPPTVPSTPQVGSPHGLPPELPVDESWRPTGQMRGSLTGNAYSSAIRHYRGRSEQQQGQPRPPSTSGARRPQ

SEQ ID NO: 16 - protein, 834 aa, Cucumis sativus
MGASSQHDTNLKKIISYIDGLTLLINHVAQIDLANLCSLCFSISRSIDYAIANNAVPSKAHSLPSLVKQLCQLKHSHRSKAALMVLMLTIKNACKVRWFSEKDAEELQRLANEIGNDFFGDTNIGQANSLTTITTVMERYFPCLKLGQIVASLEVKPGYGVYALDFNISRTVQYASQEKLRLFVIQKDNTETSACIISPPQVNFLVNGRGINGRINTHMDTGPQLPTNITHMLKLGSNLLQAVGSFNGHYVLAIAITGTAPSPDSSVLHDHIQPIVSTLDSDSDIIEGPSRISLNCPISYTRIKIPVKGCSCKHLQCFDFDNFIDINSRRPSWRCPHCNQYICFLDIRVDRNMLKVIREVAENVTEVIISVDGSWKAILENDNGDGRSLNDSLNHQNERAQEESAASPDVLDHTEVGDDMDIFNSEIEDRKPCLGNKNQRVSSSLDMSSGMNMNSFSQNLSAVMDDDIWSRIDGVLISTAGLDAPMVNSTYPPGFTGHANFPSPAFYNQNNVQIQVSNSNENNQYGRVTSISRPVSRTPVAVQALPAQSHAAGQQYSSRTPIISSPQVGQSIPINRDGLNALSRDLERRQQFSRHHGDSHHSTNLASFHHPQTVQNRDPQDRSFTTGQSIQTSSGARPSPGLLADFQNPHLQQALNMRMPHLQNQNSSSVRTSLSFSRPMSQVGGGYGGSTYTTVTPNSQHARMLAASQRVEMMRQSPPMSLHNQTSRSAHSLQTTPDGLRRPSGDLRNVGVSQSVTMAAGSVDLSAEQNWQPAGRMRGSLSGRVYSDAYGVIIQPTQAAQSARPPSNLTPTQPIAPSTQAQWSSGLDSHVSRT

SEQ ID NO: 17 - protein, 922 aa, Vitis vinifera
MTGATISLPTSVSNIAGVETTSTSYSASLANSFRLNAVLERLAMHVRSGHRILDGQRSTEEFHNLCLSLARGIDYSLANGEVPARVQDLPLLLKQICQRRNDLFLLGGIMVLMVSVKNACKVGWFTEKDTEELLTLVNEIGSNFCNLGDNNTEPISFHPTISKIMTRFYPRMEMGQILASREVKPGYGTFLVDFHISKSTKFSSQEKIRLFVAQTDNIETSSCIITPPQVNFLLNGKGVERRTNVFMDSGPQIPTNVTPMLKYGTNLLQAVGQFNGHYILAIAFMAVISSPDNPVLQDYVQPAVSMLHSDNEIVEGPSRISLNCPISRTRIKVPVKGHSCKHLQCFDFGNFVEINSRRPSWRCPHCNQYVCYTDIRIDQNMVKVLKEVGENVADVIISADGSWKAILESNDHVDQPRVGTLNSQQKGPDLQGSTSFSNASPNVWDLTEGDDEMNAFDACEIEDRKPFQSNIQGHSITTKQTMAPELNNATEVNQNAVSRVQDGFCSGILLSTYGSSTHSARSDAQFIGGTSQPSPANFLLPPVLTDAISPALNRGTEDIRGNTHLTTSTLHDQLPIPDSLQLQQAQFGHSIVSNEYGRFPTIPRHITRTPIAVQALPAQTQTSGPHHRSRTTLISMVPNGPNTVGSDMERPQQFSRSIFNPVQISDISASALQHHSMSQNWNQQVAGHPTTSQRPGPGAYRTSSGLPTEPQTLQQQQSPQARTHSNLLRSSAHHHSRSQVQQGGAQGRATHAVGTGISQNAQPMVAAQRAAQMTRMPLPVQNQTSRTGSAFPVNANGGRSTAGEQRGNIEGMVQAVSRPESLVDLASEQNWRPTGLMRGSLVGRAYNSALNQLVIQPTQPTQSTRPPTPITSPPPGFPPHLQALLTNIRTPLVPQAPNYPMTQPASTTGGSGILPERSLGLH

SEQ ID NO: 18 - protein, 833 aa, Gossypium hirsutum
MTAVPPAASGQPISASVVNSFRVAAVAERLATHTQPGRQPQSSEFFSLCLSLARGIDYAIANNEVPAKAQELPLLLKQICQHRNDLFLQAAIMVLMISVKNACKMSWFSDGESRELLTLANEVGSCFCIPGVINNELDGSLATILEVMSRFYPLMKMGQILASLEAKPGYGALVVDFHISKNMTYSPQEKIRLFVAQKDNVETSACIISPQLVSFLLNGKGVERRTNVSVDMGPQMPTNVTAMLKYGTNLLQAVGQFSGHYLIVVAFMSMESSSPDASTLPDYVQSGDFAPDSDSDLIEGPSRISLKCPISRTRIKTPVKGHACKHLQCFDFNNYVNINSRRPSWRCPHCNQHVCYTNIRIDQNMVKVLKEVAEDVSDVIISADGSWKAVMENDDDVDELHGNTLNCQKDGSELPESATGVPMVLDLTQTVDAMETIETEDRKPPVATLQSHRSGTSTSRTDTQVGGTESTRNFTVSPVFSDAVSPAPNRADAHGNANLATPGIQNQVATANNLPLHPSQVTNSMSNHEYGSLQNIPRHVSRSSIAVQALPAMSQTQTPTQQRSSNSMNTMNTTSSARIPHQMQSRIQQERSFAPARPVQQVGAAAPSQLPGPYRPPGFRAEYQNPHLQQALNTRLSQPRSPSPGLIRSPSPILRAQAQQGAAQVGVGYTAGNVNSNPTRFMAASQRTTQMARQPPMVAVQTQTPRAASYPGNVDGSRASAVEQRLNIGGVAPAASRPDTSADLASEQNWRPTGRMRGSLTGRVYSESLSQMMIQPTQSTQAARPQTNITSPPSVPPHLQAFLANSRNPVTPQVRNNATTETTATNGGSGPAR

SEQ ID NO: 19 - protein, 870 aa, Citrus sinensis
MGAVQQQLLQQLKASAANSQRVELAAQRLAAYVLLPDHQNVREFFSLCLALSRGIDYAVANNEVPPKAQELPSLLKQICQRKNDPVLQAAIMVLMFSVKSACRIEWFSVEEAQELIALADEIGSGFLGPSINGNLVSTVSTIMTRFYPLLKMGQILASLEVEPGYGAFMIDFHISKNMIQSTEEKIRLFVAQTDKTETSACVISPQHVNFILNGKGIERRTNVFMDPGPQLPTNVSPMLKYGTNLLQAVGQFNGHYIIIVAVMSTASSLESSKLQDYVQSGITMQDSDSDLIEGPSRISLNCPISYKRINTPVKGHSCRHHQCFDFSNYVHINSRRPSWRCPHCNQHVCYTDIRVDQNMVKVLREVGENVADVIISADGSWKAIMEADDNVDQAHDRILSSEKEGCEHQESAAVANSNPVILDLTKNDDEIDAMSTGEIEDVKPDLHSQPVSTNLTMPSELISTVQADQNFVTTDDDFWAGILYPDGSASSDARSDAQTVGGVSAPSSTSFMVSPVLTDAISPAFNREVDALGYTHLTTPVMQSLCSAPNNLQIQQTQLMNPSVNYEYGRSAVARHLNRTPMAVQALPAASHGFSDMEQQQRISRSHMNTVLGSDIASSPLQHQSAAQAVGLQASSALSGAYRVSSGLSTNNHNLHQQHQALNPRMPPLMSQSPSAAQSSSPYSLTPQQGSVQVGSGHPAINESRQHARLMAVAQRPLSRPQMTRQPPTVPVQVQTPSAGPRYPTTSVGVRGSVGDQRENVAGSMQSVMIDNPTDFPLEQNWRPTGRMRGSLSGRAYSDALSHMMILPTQPVAQPARPQLSPPPHLSVPNQLQALLGNSNTRFPQLQTNPVTDPGSRGSGTRPERSHGMH

SEQ ID NO: 20 - protein, 913 aa, Populus trichocarpa
MVGMMAGTMMPPPQVAEGVTIRAAGQQMSASLANSFRVHAVAERLSLHIQPGFDSNPTEFFGLCLSLARGIDFAVANNETLLKAQELPFLLKQVCQRKNDVFLQAAIMVLMASVKNACMVGWFQEKETQELVTLATEIGKVFCTPGDINAGTTDSLSIIPTIMSRFYPLMKMGQIIVSLEVKPGFGAHVIDFHISKTTRNSTEDKIWLFVAQTDNTETSTCIVTPQEVNFLLNGKGVERRTNVIMDTGPQMPTNVTGMLKYGTNLLQAVGQFKGHYVIAVAFMSVEPKPETPVLQDYVHPCAAELDPDSDIIEGPSRISLNCPISYTRIRTPVKGHSCRHLQCFDFSNFVDINTRRPSWRCPHCNQHVCYTDIRVDQNMVKVLREVGDHCSDVIISADGSLKAISESDNKVDQTQERTLHCEKGMPEQVESMTSTRALPMVMDLTVDDDEINGEDNIDAEDRKPFLATLQNHPVDTNPIPTMPSQLINANAPSRNFSTLADEFWSSPYWSSSASDAQMVNGFSEPSTTTFMTSPVITDSVSPALNCDVGGYGNTTTSSVMHNQLSASIYLQSLQQNFVNSVANGEYGTLPPIYHVDRSPIAVQALPARPQTPAPQQRSRTPNPAISSGASLSSHGTLPEAANGLSPVSGNMDRQQQFARSLNTNSSSSQNWNMQDHPFMHGQSAQQQAVTLPSSSQLAGAHRASSPNLLYQQPLRVPQSRSHSPNVVRSSLPLAPAQTQQGAAQVGVGNSAGATNSQQSRLMVAAQLAAQRARQPPSVPVQIQTSGAGASYLTSADGIRAPATEQRGNAGGALPAVSGTEGLVDLASEQNWRPTGRMRGSLSGRAYSAALKEFMVQPTQQTQTPRPPPNLPPSQSSMPPHLQFLFARNAQVPQAQSSPVTGSAISNGSSSILP

SEQ ID NO: 21 - protein, 760 aa, Arabidopsis thaliana
MSTAAAARPVAGTGLREKTAASLVNSFRLASVTQRLRYHIQDGAKVDPKEFQICCISFAKGIDFAIANNDIPKKVEEFPWLLKQLCRHGTDVYTKTALMVLMISVKHACHLGWFSDSESQELIALADEIRTCFGSSGSTSPGIKSPGSTFSQIMERFYPFVKLGHVLVSFEVKAGYTMLAHDFYISKNMPHSLQEKIRLFVAQTDNIDTSACISNPPEVSFLLNGKGVEKRVNIAMDTGPQLPTNVTAQLKYGTNLLQVMGNFKGNYIIIIAFTGLVVPPEKPVLKDYLQSGVIEASPDSDIIEGPSRVSLSCPISRKRIKLPVKGQLCKHLQCFDFSNYVHINMRNPTWRCPHCNQPVCYPDIRLDQNMAKILKDVEHNAADVIIDAGGTWKVTKNTGETPEPVREIIHDLEDPMSLLNSGPVVFDLTGDDDAELEVFGDNKVEDRKPCMSDAQGQSNNNNTNKHPSNDDYSSIFDISDVIALDPEILSALGNTAPQPHQASNTGTGQQYSNLSQIPMSIDPMPVPVPFSQTPSPRDRPATTSTVFTIPNPSPQYSQVHASPVTPTGTYLGRTTSPRWNQTYQSQAPPMTTPYTSRKVSVPVTSQSPANVSSFVQSQHVPRVLSQPNNYGVRGLTSSHASTSRQHPSGPTVQSVSRLSDLVDVDLTVPDTSNWRPRMRGSLVPGSHSTALDHMIIRPSQQSQTSTRLNSSQPVQTPSVQTSQAQSPFTTAAYRTETVLGNRNHPVPAPPGIVRPTGPTS

SEQ ID NO: 22 - protein, 876 aa, Glycine max
MNNSLPALTSDTSPSVVNLFRINKVADRLSWIAQPGNRGEPYEFYNLCLSLSRGIDYALANGETPPKAHELPLLVKQICQLKNDECSQAAMMVLMISIKNACEIGWFQTKESEELVTIADEIRKVYSSLGTINVGPRSCSTAISTIMQKFYPKFKLGPILASIEAQPGYGASVVDFHITKSEVLKDKIFLLVAQTDNIETSACLINPQQVNFLLNGKGVLNRTNVQMDPGPQVPTNVTGMLKFGTNLLQAVGQFNGRYVVLVAYMSFTPFLEDPVLQDYLQPAVTSVDSDSDIIEGASQISLNCPISFTRIKTPVKGHSCKHFQCFDFDNFINMNSKRPSWRCPHCIQNVCYADIRLDRNMVEVLKNVGENITEVIVLANGSWKAVLEKDHDVDKMQKKARNCEKEQTQPQESTCPPGTVDLTKDDDGLDTVGSCDIVERKPTPASIHSQFVTPNSTSLGMNSTGVNQNVATQIDDFWPGVCFVRSRSDTPTVGNSELPVLPDTVSPTFSQESAGHDNNPVVNSAMHNQFLGPNNLQMQMNHMNSVNEYGRSSSAPRHIHRTPVAVQALPVQSQALGPQQNSITNLNSSLLPSNSSATPHISLSNPTSVDTLNAILSDTERQQHFSRTPMNLPQVSGVNSPAFQHHTATQNRGPLINTSAPTQPQNQYRANAFSEFRNLHLQQALNLRPPPPRSSNAQWPRIQQGVPQSGNFQAAARGASVAAGQGSSHARNVPTSGATTHSHQARGMVANQPARPSVLVQNQSTVAGTPFHGLTTEQRGNTAQSVSRPEELFSSQSEQNWAPTGRMRGSLDLSQLNDESIAQRIITPTQGQNSRPPGPQPIRRTGISSLQPATTQQDVLIANNRNANAHNRSSSR

SEQ ID NO: 23 - protein, 860 aa, Zea mays; identical to SEQ ID NO: 13

SEQ ID NO: 24 - protein, 872 aa, Oryza sativa; identical to SEQ ID NO: 14
  source 1..872: note = Japonica Group

SEQ ID NO: 25 - protein, 859 aa, Sorghum bicolor; identical to SEQ ID NO: 15

SEQ ID NO: 26 - protein, 857 aa, Cucumis sativus
MGASSQHDTNLKKIISYIDGLTLLINHVAQIDLANLCSLCFSISRSIDYAIANNAVPSKAHSLPSLVKQLCQLKHSHRSKAALMVLMLTIKNACKVRWFSEKDAEELQRLANEIGNDFFGDTNIGQANSLTTITTVMERYFPCLKLGQIVASLEVKPGYGVYALDFNISRTVQYASQEKLRLFVIQKDNTETSACIISPPQVNFLVNGRGINGRINTHMDTGPQLPTNITHMLKLGSNLLQAVGSFNGHYVLAIAITGTAPSPDSSVLHDHIQPIVSTLDSDSDIIEGPSRISLNCPISYTRIKIPVKGCSCKHLQCFDFDNFIDINSRRPSWRCPHCNQYICFLDIRVDRNMLKVIREVAENVTEVIISVDGSWKAILENDNGDGRSLNDSLNHQNERAQEESAASPDVLDHTEVGDDMDIFNSEIEDRKPCLGNKNQRVSSSLDMSSGMNMNSFSQNLSAVMDDDIWSRIDGVLISTAGLDAPMVNSTYPPGFTGTMQSAVLTDAVQPVLNHGVGVSGHANFPSPAFYNQNNVQIQVSNSNENNQYGRVTSISRPVSRTPVAVQALPAQSHAAGQQYSSRTPIISSPQVGQSIPINRDGLNALSRDLERRQQFSRHHGDSHHSTNLASFHHPQTVQNRDPQDRSFTTGQSIQTSSGARPSPGLLADFQNPHLQQALNMRMPHLQNQNSSSVRTSLSFSRPMSQVGGGYGGSTYTTVTPNSQHARMLAASQRVEMMRQSPPMSLHNQTSRSAHSLQTTPDGLRRPSGDLRNVGVSQSVTMAAGSVDLSAEQNWQPAGRMRGSLSGRVYSDAYGVIIQPTQAAQSARPPSNLTPTQPIAPSTQAQWSSGLDSHVSRT

SEQ ID NO: 27 - protein, 898 aa, Vitis vinifera
MTGATISLPTSVSNIAGVETTSTSYSASLANSFRLNAVLERLAMHVRSGHRILDGQRSTEEFHNLCLSLARGIDYSLANGEVPARVQDLPLLLKQICQRRNDLFLLGGIMVLMVSVKNACKVGWFTEKDTEELLTLVNEIGSNFCNLGDNNTEPISFHPTISKIMTRFYPRMEMGQILASREVKRLFVAQTDNIETSSCIITPPQVNFLLNGKGVERRTNVFMDSGPQIPTNVTPMLKYGTNLLQAVGQFNGHYILAIAFMAVISSPDNPVLQDYVQPAVSMLHSDNEIVEGPSRISLNCPISRTRIKVPVKGHSCKHLQCFDFGNFVEINSRRPSWRCPHCNQYVCYTDIRIDQNMVKVLKEVGENVADVIISADGSWKAILESNDHVDQPRVGTLNSQQKGPDLQGSTSFSNASPNVWDLTEGDDEMNAFDACEIEDRKPFQSNIQGHSITTKQTMAPELNNATEVNQNAVSRVQDGFCSGILLSTYGSSTHSARSDAQFIGGTSQPSPANFLLPPVLTDAISPALNRGTEDIRGNTHLTTSTLHDQLPIPDSLQLQQAQFGHSIVSNEYGRFPTIPRHITRTPIAVQALPAQTQTSGPHHRSRTTLISMVPNGPNTVGSDMERPQQFSRSIFNPVQISDISASALQHHSMSQNWNQQVAGHPTTSQRPGPGAYRTSSGLPTEPQTLQQQQSPQARTHSNLLRSSAHHHSRSQVQQGGAQGRATHAVGTGISQNAQPMVAAQRAAQMTRMPLPVQNQTSRTGSAFPVNANGGRSTAGEQRGNIEGMVQAVSRPESLVDLASEQNWRPTGLMRGSLVGRAYNSALNQLVIQPTQPTQSTRPPTPITSPPPGFPPHLQALLTNIRTPLVPQAPNYPMTQPASTTGGSGILPERSLGLH

SEQ ID NO: 28 - protein, 834 aa, Gossypium hirsutum
MTAVPPVASGQPISASVVNSFRVAAVAERLATHTQPGRQPQSSEFFSLCLSLARGIDYAIANNEVPAKAQELPLLLKQICQHRNDLFLQAAIMVLMISVKNACKMSWFSDGESRELLTLANEVGSCFCIPGVINNELDGSLSTILEVMSRFYPLMKMGQILASLEAKPGYGALVVDFHISKNMTHSPQEKIRLFVAQKDNVETSACIISPQLVSFLLNGKGVERRTNVLMDMGPQMPTNVTAMLKYGTNLLQAVGQFSGHYLIVVAFMGMEESSPDASTLPDYVQSGDFAPDSDSDLIEGPSRISLKCPISRTRIKTPVKGHACKHLQCFDFNNYVNINSRRPSWRCPHCNQHVCYTNIRVDQNMVKVLKEVAEDVSDVIISADGSWKAVMENDDDVDELHGNTLNCQKDGSERPESATGVPMVLDLTQTVDAMETIETEDRKPPVATLESHGSGTSTSRTDTQVGGTESTRNFTVSPVFSDAVSPALNRADAHGNANLATPGIQNQVATTNNLPLHPSQVTNSMSNHEYGSLQNIPRHVSRSSIAVQALPAMSQTQTPTQQRSSNSVNTKNTTSSARIPHQMQSRIQQERSFVPARPVQQVGAAAPSQLPGPYRPPGFRAEYQNPHLQQALNTRLSQPRSPSPGLIRSPSPILRAQAQQGAAQVGVGYTAGNVNSNPTRFMAASQRTTQMARQPPMVAVQTQTPRAASSYPGNVDGSRASAVEQRLNMGGVAPAASRPDTSADLASEQNWRPTGRMRGSLTGRVYSESLSQMMIQPTQSTQAARPQTNITSPPSVPPHLQAFLANSRNPVTPQMRNNATTETTATNGGSGPAR

SEQ ID NO: 29 - protein, 870 aa, Citrus sinensis; identical to SEQ ID NO: 19

SEQ ID NO: 30 - protein, 913 aa, Populus trichocarpa; identical to SEQ ID NO: 20

SEQ ID NO: 31 - protein, 300 aa, Arabidopsis thaliana
MGAPKQKWTQEEESALKSGVIKHGPGKWRTILKDPEFSGVLYLRSNVDLKDKWRNMSVMANGWGSREKSRLAVKRTFSLPKQEENSLALTNSLQSDEENVDATSGLQVSSNPPPRRPNVRLDSLIMEAIATLKEPGGCNKTTIGAYIEDQYHAPPDFKRLLSTKLKYLTSCGKLVKVKRKYRIPNSTPLSSHRRKGLGVFGGKQRTSSLPSPKTDIDEVNFQTRSQIDTEIARMKSMNVHEAAAVAAQAVAEAEAAMAEAEEAAKEAEAAEAEAEAAQAFAEEASKTLKGRNICKMMIRA

SEQ ID NO: 32 - protein, 299 aa, Glycine max
MGAPKQKWTAEEEQALKAGVVKHGVGKWRTILKDPEFSGVLYLRSNVDLKDKWRNLSVMANGWSSREKSRLSVRRVHQVPRQDENSMAITPVVPSDEEIVDVKPLQVSRDIVHIPGPKRSNLSLDKLIMEAITSLKENGGSNKTAIAAFIEDQYWALPGLKSMLSAKLKFLTASGKLIKVNRKYRIAPIAAYSDRRRNSSMLYLKGRQKASMKIDRDETNILTRSQIDLELEKIRSMTPQEAAAFAARAVAEAEAAIAEAEEAAKEAEAAEADAEAAQAFADATTKSAKGRKSPKRIHT

SEQ ID NO: 33 - protein, 299 aa, Zea mays
MGAPKHRWTPEEEAALKAGVAKHGPGKWRTILRDSDFSALLRLRSNVDLKDKWRNLSVTAGGYGSREKARMALKKGRRVVPKLTAEPMDVDVKNMDDAHDTAIDVEPLAMAFESLPTEESPDKSVARLDDLILEAIRKLKEPSGSSKAAIAAYIEDQYWPPADFQRLLSTKLKALVNSGKLIKVNQKYRIAPSPPPSGRIGTKVSSAEGMKAENNNAKRLTKHQVIAELEKMKGMTKEEAAAFAAKAVAEAEVAIAEAEEAARVAEAAENDAEAAKAFLDAVTLSMRNRNAASMMLRAC

SEQ ID NO: 34 - protein, 300 aa, Oryza sativa
  source 1..300: note = Japonica Group
MGAPKQRWTAEEEAALKAGVAKHGTGKWRTILRDPEFTALLRLRSNVDLKDKWRNLSVTAGGYGSRERARVALKGGKRGPKALAEPMDADEKNPDIDDNAIIDAQPLAVVVEPMQLESTPEKEKSVARLDDLILEAIKKLKEPSGSNRTTISSYIEEQYWPPEDFQRLLSTKLKALVATGKLIKVNQKYRIAPSSNSSGGKSIKVYSTGEMNIENNNVRQLSKPQVDAELDKMKSMSKEEAASFAARAVAEAEAAIAEAEEAARAAEAAEAEADAAKAFLDAVVTTMQNRNHASAMLRAC

SEQ ID NO: 35 - protein, 302 aa, Sorghum bicolor
MGAPKQRWTSEEEAALRAGVARHGVGNWRMILNDPELGSTLRYRSNVDLKDKWRNMNVIVTSSSARDRGRTSTRRTRAAPKNSDQSLPMSTVTSDVDDEIVDVNPIASVVPVESWNTSNSKKSHSRLDNIIMEAIKNLNEPTGSHRTTIANYIEEQYWPPSDFDHLLSAKLKDLATSGKLLKVNRKYRIAPSSPRLEGRSPKMMLLEDVQGEPLKLGSDASRTLTRSQIDAELVRMATMTAEAAAAAAAHAVAEAEAIMAEAEAAAREAEAAEAEARAAQAFAEAAVLTLKNRNAAKLMAQA

SEQ ID NO: 36 - protein, 297 aa, Cucumis sativus
MGAPKQKWTSEEEAALKAGVVKHGAGKWRTILKDPEFSSVLYLRSNVDLKDKWRNMSVMANGWGSREKARLALKRLHAPRKDENAVGPSVAAQSEDELAEAKSVSLSSDIKQITGPKRSNVRLDNLIIEAITTLREPGGSNKTKITSYIEDQYWAPPDFKRLLSSKLKFLTASRKLVKVKRKYRLPSVAASERRSSMLLLEDQQKASVRADKDDMCILAKAQIDLELAKMRTMTSQEAAAAAARAVAEAEAAIAEAEEAAREAEAAEADAEAAQSFAEAAMKTLKGRNLPKMQMIRV

SEQ ID NO: 37 - protein, 300 aa, Vitis vinifera
MGAPKQKWTPEEEAALKAGVVKHGAGKWRTILKDPEFSGVLFLRSNVDLKDKWRNMSVMANGWGSREKARLALRKVPSAPKAEENPLSLGTAVQSDDETVDTKALALPSNPVQITGSKRSFSRLDNLILEAITNLKEPGGSNKTTIATYIEDQYRAPPNFKRLLSAKLKFFTTNGKLIKVKRKYRIAHTPAFSERRRNSSMPFLEGRQGFSPKVDKDDIHILTKAQVDFELAQMRGMTAEEAAIVAARAVAEAEAALAEAEEAAREADAAEADAEAAQAFAAAAMKTLKGRSNPKTNGHP

SEQ ID NO: 38 - protein, 300 aa, Gossypium hirsutum
MGAPKQKWTPEEEAALKAGVIKHGAGKWRTILKDPEFSGVLYLRSNVDLKDKWRNMSVMANGWGSRDKARLAVKRTSSFPKQEESAVDLAVAPSDEEIVDVKSVPVSSATLQIPSAAKRSIVRLDNLIMEAITTLKEPGGSNKTNIAAYIEEQYWAPPDFKRLLSAKLKYLTACGRLIKVKRRYRIAPALSFSDRRRNHPMLFSEGRQRVSPRFDRDDLKIITKSQIDLELARMRKMTPQEAAAAAARAVAEAEAAIAEAEEAAREAEVAEADAEAAQAFAEAAMKTLKGRNNQKVMVRA

SEQ ID NO: 39 - protein, 303 aa, Citrus sinensis
MGAPKQKWTQEEEAALKAGVIKHGAGKWRTILKDPEFSGVLYLRSNVDLKDKWRNMSVMSNGWGSRDKSRLAVKRIPHIAKSEENSLALSSTVVQSDEEEIVDAKPLAVSGATPQIAAAPKRSIVRLDNLIMEAITTLKEPGGSNKTTIAAYIEEKYWAPPDFKRLLSAKLKYLAGTGKLVKIKRKYRIAPTLPFQDRRRNSMLLLEGRQRISHKVNNDDINVLTKSQIDLELAKLRSMTPQEAAAAAARAVAEAEAAVAEAEEAAREADVAEADAEVAQAFAAAAMKTLKGRNNSQKMMIRA

SEQ ID NO: 40 - protein, 316 aa, Populus trichocarpa
MSLEEPDLFVWGVYLLISLMGAPKQKWTAEEEAALKAGVIKHGAGKWRTILKDPEFSGVLYLRSNVDLKDKWRNMSVMVNGYGSREKPKLAPKRVHHVHRQDENLMSVGTVQSDEDIPDAKALPVSSAALPVVNPKRTTVRLDNLIMEAITSLKEPGGSHKTAIASYIEEQYWPPNDFKRILSAKLKYLAANGKLIKVKRKYRIAPTSAFAERRVSPLFFEGRQRISPKIERDDFNILTMSQIDLDLGKMRTMTAEQAAEVAARAVAEAEAAIAVAEEAAREAETAEADAEAAQAFAEAAMKTLKGRTAPKVTVRA

SEQ ID NO: 41 - protein, 299 aa, Arabidopsis thaliana
MGAPKQKWTPEEEAALKAGVLKHGTGKWRTILSDSEFSLILKSRSNVDLKDKWRNISVTALWGSRKKAKLALKRTPPGTKQDDNNTALTIVALTNDDERAKPTSPGGSGGGSPRTCASKRSITSLDKIIFEAITNLRELRGSDRTSIFLYIEENFKTPPNMKRHVAVRLKHLSSNGTLVKIKHKYRFSSNFIPAGARQKAPQLFLEGNNKKDPTKPEENGANSLTKFRVDGELYMIKGMTAQEAAEAAARAVAEAEFAITEAEQAAKEAERAEAEAEAAQIFAKAAMKALKFRIRNHPW

SEQ ID NO: 42 - protein, 298 aa, Glycine max
MGAPKQKWTAEEEAALKAGVVKHGAGKWRTILTDPEFSSILRMRSNVDLKDKWRNINVTAIWGSRQKAKLALKKNLLPSTKIDNNHLALSTVVQRDKEVANPKPLAVSSGTSPNSKEKISKLQNFQLDNLILESIIKLKEPRGSDQAAIAAYIEDQYCSTPTLRKLLSTKLKHMVASGKLMKVKHKYRIATNLTISEKRRCSSLLLLEGRPKDSPKAEKTGVNILSKSEIDAELSKMKGVTPQEAAAAAAKAVAEAEAAIAEAEAAAREADAAEAEAEAARVFAKAAMKALKCKTLRI

SEQ ID NO: 43 - protein, 299 aa, Zea mays
MGAPKQRWTPEEEAALKAGVAKHGPGKWRTILRDSDFSALLRLRSNVDLKDKWRNLSVTAGGYGSREKARMALKKGRRVVPKLTAEPMDVDVKDMDDAHDTAIDVEPLAMAFESLPTEESPDKSVARLDDLILEAIRKLKEPSGPSKAAIAAYIEDQYWPPADFQRLLSTKLKALVNSGKLIKVNQKYRIAPSPPPSGRIGTKVSSAEGMKAENNNAKRLTKHQVIAELEKMKGMTKEEAAAFAAKAVAEAEVAIAEAEEAARVAEAAENDAEAAKAFLDAVTLSMRNRNAASMMLRAC

SEQ ID NO: 44 - protein, 300 aa, Oryza sativa; identical to SEQ ID NO: 34
  source 1..300: note = Japonica Group

SEQ ID NO: 45 - protein, 299 aa, Sorghum bicolor
MGAPKQRWTPEEEAALKAGVAKHGPGKWRTILRDSDFSELLRLRSNVDLKDKWRNLSVTAGGYGSREKARMALKKGKRVVPKLTAEPMDIDGKDMDNAHDAAIEAEPLAMALEPLAIEESPDKSVARLDDLIFEAIRKLNEPSGSNKAAIAAYIEEQYWPPADFQRLLSTKLKSLVNSGKLIKVNQKFRIAQSSPPLGGISTKVSSAEGMNTGKNNAKRLTKPQVVAELEKMKGMTKEEAAAFAAKAVAEAEVAIAEAEEAARVAEAAENDAEAAKAFLDAVTLSMRSRNAASMMLRAC

SEQ ID NO: 46 - protein, 308 aa, Cucumis sativus
MGAPKQKWTAEEEAALKAGVIKHGAGKWRTILTDPEFSSILHQRSNVDLKDKWRNINVTAIWGSRQKAKLALKKNSMAIKHHDNLVPVSTVLPNEEIVDAKPLAISNGTSRSNGPKEPLARLDKLISEAINNLKEPRGSDRAAIAMYIEEHYWPPSNLKKLLSTKLKHMTANGKLIKVKHKYRIAPNSPLPGRRNTPLLLLEDKQTDSSKTEKSEVKIITKSQVDSELSKMKVMTAEEAAIAAARAVAEAEAAIAEAERAAREAEQAEAEAETAQVFAEAAMKALECRTFPNRSPIQKVLLQDNKTLQ

SEQ ID NO: 47 - protein, 300 aa, Vitis vinifera; identical to SEQ ID NO: 37

SEQ ID NO: 48 - protein, 290 aa, Gossypium hirsutum
MGAPKQKWTAEEEAALKAGVLKHGTGKWRNILSDPEFSSVLRSRSNVDLKDKWRNINATAIWGSRQKAKLAFKRNQLAAKHDDNPMALITVPPREEVVDAKPLAVSSGTPRATGSKKLISRLDNILLEAITSLKEPAGSDRASIAVYIEEKYATPPNLKKLLATKLKLLVANGTLVKVKHKYKVAPRSTISEARKSPLLLLEGRPKESSKAKKKGINILTKTQVNADLLKMRSMTAEEAAAAAAQAVAEAEVAIAEAEKAAREADVAEAEAEAATIFAKAAEKALKSRML

SEQ ID NO: 49 - protein, 322 aa, Citrus sinensis
MGAPKQKWTAEEEAALKAGVRKHGTGKWRTILSDPEFSAILHSRSNVDLKDKWRNINVTAIWGSRQKAKLALKKTSLTPKLDGRTPVIPKHDGRTPVTPKQDGRTQMTPKHDDNTIALSTAPQNDEVVDAKPLAISSGTMQASGSKEPVARLDKLILEAISNLKEPNGSDRASIASYIEDQYWAPANLRKLLATKLKFLSANGTLIKAKHKYRITPNTPVSVPKRSPKLLLEGRQKGSPKAQKKEINILTKSLVDADLSRMRGMTAQEAAAAAAQAIAEAEVAIAEAEEAAREAERAEAEAEAAQVFAKAAIKALKCRALHT

SEQ ID NO: 50 - protein, 292 aa, Populus trichocarpa
MGAPKQKWTAEEEAALKAGVLKHGTGKWRTILMDPDFSAVLRLRSNVDLKDKWRNINVTAIWGSRQKAKLALKRSPLTPKREENGKALSVVVQSNEEVVDAKPLAMASGTPRNGGPKDLLARLDNLILEAITTLKEPSGSDRASIALYIQEKYWAPMNLRKLLGGKLKHLTANGKLIKVKHKYMIASSSTVSEGRRNHPLPEGKQKEMLKLEKSKNKILTKAQVDQDLLKIRGMTALEAAAAAAAAVAEAETAIAEAEAAAREAEKAEAEAEAAQVFAKAAIKAFKHRACHP

SEQ ID NO: 51 - protein, 295 aa, Arabidopsis thaliana
MGAPKLKWTPEEETALKAGVLKHGTGKWRTILSDPVYSTILKSRSNVDLKDKWRNISVTALWGSRKKAKLALKRTPLSGSRQDDNATAITIVSLANGDVGGQQIDAPSPPAGSCEPPRPSTSVDKIILEAITSLKRPFGPDGKSILMYIEENFKMQPDMKRLVTSRLKYLTNVGTLVKKKHKYRISQNYMAEGEGQRSPQLLLEGNKENTPKPEENGVKNLTKSQVGGEVMIMGMTEKEAAAAAARAVAEAEFAMAEAEEAAREADKAEAEAEAAHIFAKAAMKAVKYRMHSQTR

SEQ ID NO: 52 - protein, 298 aa, Glycine max; identical to SEQ ID NO: 42

SEQ ID NO: 53 - protein, 296 aa, Zea mays
MGVPKQRWTPEEEAALKAGVAKHGPGKWRTILRDSDFSALLRLRSNVDLKDKWRNLSVTAGGYGSREKARMALKKGRRVVPKLTAEPMDVDEKDMDNAHDTVIDVEPLAMAFEPLPFLESPDKSVARLDDLIVEAIRKLNEPSGSNKAVISGYIEDQYWPPADFQYLLSTKLKSLVNSGKLIKVNQKYRIAPSSSLGGISTKVSSSEGMNTENNNVKRLTKPQVVAELEKMKGMTREEAAAFAAKAVAEAEVAMAEAEEAARVAEAAENDAEAAKAFLDAVILSMRNRNAASMTLL

SEQ ID NO: 54 - protein, 300 aa, Oryza sativa; identical to SEQ ID NO: 34
  source 1..300: note = Japonica Group

SEQ ID NO: 55 - protein, 299 aa, Sorghum bicolor; identical to SEQ ID NO: 45

SEQ ID NO: 56 - protein, 308 aa, Cucumis sativus; identical to SEQ ID NO: 46

SEQ ID NO: 57 - protein, 300 aa, Vitis vinifera; identical to SEQ ID NO: 37

SEQ ID NO: 58 - protein, 290 aa, Gossypium hirsutum; identical to SEQ ID NO: 48

SEQ ID NO: 59 - protein, 357 aa, Citrus sinensis
MGAPKQKWTAEEEAALKAGVRKHGTGKWRTILSDPEFSAILHSRSNVDLKDKWRNINVTAIWGSRQKAKLALKKTSLTPKLDGRTPVIPKHDGRTPVTPKQDGRTQMTPKHDDNTIALSTAPQNDEVVDAKPLAISSGTMQASGSKEPVARLDKLILEAISNLKEPNGSDRASIASYIEDQYWAPANLRKLLATKLKFLSANGTLIKAKHKYRITPNTPVSVPKRSPKLLLEGRQKGSPKAQKKEINILTKSLVDADLSRMRGMTAQEAAAAAAQAIAEAEVAIAEAEEAAREAERAEAEAEAAQVFAKAAIKALKYDLEIEQRICREFLLPSIIFEHAAISTSAMSIIMLRASCKS

SEQ ID NO: 60 - protein, 292 aa, Populus trichocarpa; identical to SEQ ID NO: 50

SEQ ID NO: 61 - protein, 424 aa, Arabidopsis thaliana
MGKDEEEMRGEIEERLINEEYKIWKKNTPFLYDLVITHALEWPSLTVEWLPDREEPSGKDYSVQKMILGTHTSESEPNYLMLAQVQLPLDDTESEARQYDDDRSEFGGFGCATGKVQIIQQINHDGEVNRARYMPQNPFIIATKTVNAEVYVFDYSKHPSKPPLDGACNPDLKLRGHSSEGYGLSWSKFKQGHLLSGSDDAQICLWDINATPKNKSLDAQQIFKAHEGVVEDVAWHLRHEYLFGSVGDDQYLLIWDLRSPSASKPVQSVVAHSMEVNCLAFNPFNEWVVATGSTDKTVKLFDLRKLSTALHTFDSHKEEVFQVGWNPKNETILASCCLGRRLMVWDLSRIDEEQTVEDAEDGPPELLFIHGGHTSKISDFSWNPCEDWVISSVAEDNILQIWQMAENIYHDEDDAPGEEPSKAS

SEQ ID NO: 62 - protein, 425 aa, Glycine max
MGKEEEEMRGEIEERLINEEYKIWKKNSPFLYDLVITHALEWPSLTVEWLPDRHEPPGKDYSLQKVILGTHTSENEPNYLMLAQVQLPLDDAENDARHYDDDRPDIGGFGCANGKVQIIQQINHEGEVNRARYMPQNPFIIATKTVSAEVYVFDYSKHPSKPPLDGACNPDLRLRGHNTEGYGLSWSKFKQGHLLSGSDDAQICLWDINGTPKNKSLEAMQIFKVHEGVVEDVAWHLRHEYLFGSVGDDQYLLIWDLRTPAASKPVQSVVAHQSEVNCLAFNPFNEWVVATGSTDKTVKLFDLRKINTPLHIFDSHKEEVFQVGWNPKNETILASCCLGRRLMVWDLSRIDEEQSPEDAEDGPPELLFIHGGHTSKISDFSWNPCEDWVVASVAEDNILQIWQMAENIYHDEDDLPEESTKAAAS

SEQ ID NO: 63 - protein, 431 aa, Zea mays
MPKAGGGAADEEEFRAEVEERLINEEYKIWKKNTPFLYDLVITHALEWPSLTVQWLPDRTEPPGKDHSVQKMILGTHTSDNEPNYLMLAQVQLPLDDAEADARHYDDDHADIGGFGAASGKVQIVQQINHDGEVNRARYMPQNSFIIATKTVSAEVYVFDYSKHPSKPPLDGACNPDLRLKGHNSEGYGLSWSIFKEGHLLSGSDDAQICLWDIKANSRNKSLDALQIFKHHDGVVEDVAWHLRHEYLFGSVGDDYHLLIWDLRSPAPTKPVQSVVAHQGEVNCLAFNPFNEWVVATGSTDKTVKLFDLRKIDTSLHTFDCHKEEVFQVGWSPKNETVLASCCLGRRLMVWDLSRIDQEQTPEDAEDGPPELMFIHGGHTSKISDFSWNPCEDWVVASVAEDNILQIWQMAENIYHDEDDLPISDEPAKTS

SEQ ID NO: 64 - protein, 428 aa, Oryza sativa
  source 1..428: note = Japonica Group
MPKATAAEEEEFRAEVEERLINEEYKIWKKNTPFLYDLVITHALEWPSLTVQWLPDRAEPAGKDHSVQKMVLGTHTSDNEPNYLMLAQVQLPLDDAEADARHYDDDHAEIGGFGAASGKVQIVQQINHDGEVNRARYMPQNSFIIATKTVSAEVYVFDYSKHPSKPPLDGACNPDLRLKGHNSEGYGLSWSIFKEGHLLSGSDDAQICLWDIKANSKNKTLDALQIFKYHDGVVEDVAWHLRHEYLFGSVGDDHNLLIWDLRSPVSTKPVQSVAAHQGEVNCLAFNPFNEWVVATGSTDKTVKLFDLRKIDTSLHTFDCHKEEVFQVGWSPKNETILASCCLGRRLMVWDLSRIDQEQTPEDAEDGPPELLFIHGGHTSKISDFSWNPCEDWVIASVAEDNILQIWQMAENIYHDEDDVPTDDPAKAP

SEQ ID NO: 65 - protein, 431 aa, Sorghum bicolor
MPKAGGGAADEEEFRAEVEERLINEEYKIWKKNTPFLYDLVITHALEWPSLTVQWLPDRTEPPGKDHSVQKMILGTHTSDNEPNYLMLAQVQLPLDDAEADARHYDDEHTDIGGFGAASGKVQIVQQINHDGEVNRARYMPQNSFIIATKTVSAEVYVFDYSKHPSKPPLDGACNPDLRLKGHNSEGYGLSWSIFKEGHLLSGSDDAQICLWDIKANSKNKSLDALQIFKHHDGVVEDVAWHLRHEYLFGSVGDDHHLLIWDLRSPAPTKPVQSVVAHQGEVNCLAFNPFNEWVVATGSTDKTVKLFDLRKIDTSLHTFHNHKEEVFQVGWSPKNETILASCCLGRRLMIWDLSRIDQEQTPEDAEDGPPELMFIHGGHTSKISDFSWNPCEDWVIASVAEDNILQIWQMAENIYHDEDDLPISDEPAKTS

SEQ ID NO: 66 - protein, 423 aa, Cucumis sativus
MGKDDEEMRGEMEERLINEEYKIWKKNTPFLYDLVITHALEWPSLTVEWLPDREEPPGKDYSVQKMILGTHTSENEPNYLMLAQVQLPLEDSENDARHYDDDRADAGGFGCANGKVQIIQQINHDGEVNRARYMPQNPFIIATKTVSAEVFVFDYSKHPSKPPLDGTCNPDLRLRGHNTEGYGLSWSKFKQGHLLSGSDDAQICLWDINATPKNKTLEAMQIFKVHEGVVEDVAWHLRHEYLFGSVGDDQYLLVWDLRTPSANKPVQSVVAHQSEVNCLAFNPFNEWVVATGSTDKTVKLFDLRKISTALHTFDCHKEEVFQVGWNPKNETILASCCLGRRLMVWDLSRIDEEQTPEDAEDGPPELLFIHGGHTSKISDFSWNPCEDWVVASVAEDNILQIWQMAENIYHDEDDLPEEPPKAS

SEQ ID NO: 67 - protein, 424 aa, Vitis vinifera
MGKDEEEMRGEIEERLINEEYKIWKKNTPFLYDLVITHALEWPSLTVEWLPDREEPPGKDYSVQKMILGTHTSENEPNYLMLAQVQLPLEDAENDARQYDDDRFDVGGFGCANGKVQIIQQINHDGEVNRARYMPQNSFIIATKTVSAEVYVFDYSKHPSKPPLDGACSPDLRLRGHSTEGYGLSWSQFKQGHLLSGSDDAQICLWDINATPKNKALEAQQIFKVHEGVVEDVAWHLRHEYLFGSVGDDQYLLIWDLRTPSVSKPIQSVIAHQSEVNCLAFNPFNEWVVATGSTDKTVKLFDLRKINTALHTFDCHKEEVFQVGWNPKNETILASCCLGRRLMVWDLSRIDEEQTPEDAEDGPPELLFIHGGHTSKISDFSWNPCEDWVIASVAEDNILQIWQMAENIYHDEDDLPGEESTKAS

SEQ ID NO: 68 - protein, 424 aa, Gossypium hirsutum
MGKDEEEMRGEIEERLINEEYKIWKKNTPFLYDLVITHALEWPSLTVEWLPDRDEPAGKDYSVQKMILGTHTSENEPNYLMLAQVQLPLQDSENDARQYDHDRSDLGGFGCASGKVQIIQQINHEGEVNRARYMPQNSFIIATKTVSAEVFVFDYSKHPSKPPLDGACSPDLRLRGHSTEGYGLSWSKFKEGHLLSGSDDAQICLWDINATPKNKALDAMQIFKVHEGVVEDVAWHLRHEYLFGSVGDDQYLLIWDLRAPSVTKPIQSVVAHQSEVNCLAFNPFNEWVVATGSTDKTVKLFDLRKISTALHTFDSHKEEVFQVGWNPKNETILASCCLGRRLMVWDLSRIDEEQTPEDAEDGPPELLFIHGGHTSKISDFSWNPCEDWVIASVAEDNILQIWQMAENIYHDEDDVPGDESAKGS

SEQ ID NO: 69 - protein, 424 aa, Citrus sinensis
MGKDEEEMRGEIEERLINEEYKIWKKNTPFLYDLVITHALEWPSLTVEWLPDREEPPGKDYSVQKMILGTHTSENEPNYLMLAQVQLPLDDSENDARHYDDDRSDFGGFGCANGKVQIIQQINHDGEVNRARYMPQNPFLIATKTVSAEVYVFDYSKHPSKPPLDGACSPDLRLRGHSTEGYGLSWSKFKEGHLLSGSDDAQICLWDINAAPKNKSLEAMQIFKVHEGVVEDVAWHLRHEYLFGSVGDDQYLLIWDLRTPSVSKPVQSVVAHQSEVNCLAFNPFNEWILATGSTDKTVKLFDLRKISTALHTFDSHKEEVFQVGWNPKNETILASCCLGRRLMVWDLSRIDEEQTPEDAEDGPPELLFIHGGHTSKISDFSWNPCEDWVISSVAEDNILQIWQMAENIYHDEDDLPGDESAKAS

SEQ ID NO: 70 - protein, 424 aa, Populus trichocarpa
MGKDEDEMRGEIEERLINEEYKIWKKNTPFLYDLVITHALEWPSLTVEWLPDREEPPGKDYSVQKMILGTHTSENEPNYLMLAQVQLPLDDAENDARHYDDDRSDFGGFGAANGKVQIIQQINHDGEVNRARYMPQNPFMIATKTVSAEVYVFDYSKHPSKPPLDGACTPDLRLRGHSTEGYGLSWSKFKEGYLLSGSDDAQICLWDINTTPKNKSLDAMQIFKVHEGVVEDVAWHLRHEHLFGSVGDDQYLLIWDLRTPSVTKPVHSVVAHQSEVNCLAFNPFNEWVVATGSTDKTVKLFDIRKINTALHTFNCHKEEVFQVGWNPKNETILASCCLGRRLMVWDLSRIDDEQTPEDAEDGPPELLFIHGGHTSKISDFSWNPCEDWVVASVAEDNILQIWQMAENIYHDEDDIPADESTKDS

SEQ ID NO: 71 - protein, 445 aa, Arabidopsis thaliana
MKGASGAVKKKPQVLNEAGEAETAVETVGESRKISGDGGFGSDDGGGGGGGGSGESILREIGDDRPTEDGDEEEEEDEDEDDGGDEEDEEGEGEGGQEERPKLDEGFYEIEAIRRKRVRKGKVQYLIKWRGWPETANTWEPLENLQSIADVIDAFEGSLKPGKPGRKRKRKYAGPHSQMKKKQRLTSTSHDATEKSDSSTSLNNSSLPDIPDPLDLSGSSLLNRDVEAKNAYVSNQVEANSGSVGMARQVRLIDNEKEYDPTLNELRGPVNNSNGAGCSQGGGIGSEGDNVRPNGLLKVYPKELDKNSRFIGAKRRKSGSVKRFKQDGSTSNNHTAPTDQNLTPDLTTLDSFGRIARMGNEYPGVMENCNLSQKTKIEELDITKILKPMSFTASVSDNVQEVLVTFLALRSDGKEALVDNRFLKAHNPHLLIEFYEQHLKYNRTP

SEQ ID NO: 72 - protein, 448 aa, Glycine max
MKGGGGKRKASTSEAPNDVVVVVAAASAPSDDADLGVGDVGGVKVQNFEGNEGTQLRNTEEQEEEPHHVEGDEGGEDEEDYAEGEEEYEEENGGAVAGGQQGFPALGENFFEVEAIRRKRLRKGEVQYLIKWNGWPETANTWEPLENLESVPDVVEAFEESLKSGKHRKRKRKHVVHHTQPKKRLERSTTPYSLRRFSTSTAENHTQSAPPLNDPSLPDIPAFPHTVLFSDDVGNGAEGSSLRKATPSNANRSANGSEQNIKRNEENDYDPVLSELKAMTSNGNDADRLAIRIPEAKGSGPSGSNGQMDAKSKGACMETSESDRCRGSKRRKCGSVKRFKKELYANEPANAENPVSMPVGTAEPERTRDAGSGGNTNHARPASNIVNIVKPVGYSASVASGMQDVLVTFVASKSDGTEVMVDNKYLKAFNPLLLINFYEQHLRYSPTL

SEQ ID NO: 73 - protein, 346 aa, Zea mays
MRRNRKDSGYPTAAPGEEDEAEELTEEGDEEEEEQTEEGCGEEDEVEAETPAQEAVEPPKLAEGYFEIEAIRRRRLRKGQLQYLVKWRGWPESANTWEPLENLKACSDIVDAFNKRSRSPRSCGKRKRKTPTTPTSDPNPSRGKRGRPPRSEARSMHSIHASEVKKLPCRTSSRRANNNSNKTSPGGLGAAVNLLGQRIVQEGSSGVVSVGFPSQEAPLSVSLTDQQDAQHPANGSLKMDNEIRAITPQGGQVTGAKKRKSGCVRRFKQDEVTHEQGDIRDRTSDKPGNETVDSTEGDPGDKNKGEDSGNQIHMPKIIKIIKPVRYFATVMDGVQQVAITFKALRS

SEQ ID NO: 74 - protein, 415 aa, Oryza sativa
  source 1..415: note = Japonica Group
MARGKNHPDGEEEEAPAAAGEEEAPVEMDEEGEMEEEEEEEQGEGEGEERDEGEEEEEWEDAEEVEGGEESEQAAAEEEDSPLVVVEAEAAAPVVDGSPPKLAEGYYEIEDIRRRRLRKGKLQYLVKWRGWPESANTWEPLENLSACSDIIDAFEMRLQSPRPGRKRKRKITTTPVAGSNPSHGKRGRPRLDAKSHTRAPAPEPKQLPCRTSCRRATNCSSKTVAGLDASGSVVRNQLAQNIVQEGSSSVISRTPCQELPLSIRLTDQQNEHHLVNGSSNSENLVKVPPSQGGQVTGAKKRKSGNVRRFEQNKPTQGQGECGALVVAEDVGSTEGETGDKKKTEGCPNRVHITKIIKPVRFAAAVNNDVQQVSITFKALRSDGQEVMVDDKELKANNPLLLISYYEQCLRYNPTS

SEQ ID NO: 75 - protein, 383 aa, Sorghum bicolor
MGRKRNESGYLTAALEEREEAEEQTEEGDEEEGEEQTEEGSCGEDQAEAEAETLAQEAVEPPKLAEGFYEIEAIRRRRLRRGQLQYLVKWRGWPESANTWEPLENLKACSDIVDAFNKRSRSPRSSRKRKRKTATTPTSDPNPSRGKRGRPPRSEARSMQGIHASEVKKLPCRTSSRRANNHSNKTSPGGLDASVNLLGQRVVKEGSSGVVSVGFLSQGAPLSVSLTDQQDVQHPANGSLKVDNSTRATPPQGGQVTGAKKRKSGCVRRFKQDEVTQEQGDIRDRTGDKPGNETVDSTEGETGDKNKGEDSGNQIHMPKIIKIIKPVRYFATVMDGVQQVAITFKALRSDGEEVFVDDKQLKAKEPLVLINYYEQHLRYNPTS

SEQ ID NO: 76 - protein, 454 aa, Cucumis sativus
MKFKGGVGRKKSSSPSMEVAVGSIQDAMDSGEVHGGNSDYANVNINNKPINGSEPSNSHLTETHQDQNLEEVDDDDGEGDEEDEQDGDEAAFASQRTNLDDGFYEIEAIRRKRVRKGQLQYLIKWRGWPETANTWEPLENLHTCSDFIEAFEQSLMTGKQRKRKRKHGVVHTQTKKRQHQQRGSFSAYNVTDVEISVVDQRLPSAPLNMSSLTNPHAHSQSLVYNHEGEKNGDVTAIERGKQTDIDNMGRKATQRSEWKKDEHEYDPKLSELKATVLTNIAITDKHVINFQDSRATENNGSAAGLSKGAFVEPVTDNRCTGARRRKSGSVRRFRHDSTLSALPRSQNAELTLAVVESGARVEPIGVENSGYHGESLSRNNKTDDARNEMSITKIIKPLGYSASVSNNMQDVLVTFVAMRSDGTEVVVDNKFLKAINPLLLINFYEQHLRYTTRS

SEQ ID NO: 77 - protein, 424 aa, Vitis vinifera
MKTKRKTEAQTTPFHGPNLHPHPHPEHNDDGGTQHEEDETTPYQEAQQDEEVEEEEEEEEEEVEEGEGEGEGEGEGEGEGEGGEQSERPKLDDGFYEIEAIRRRRVRKGQLQYLIKWRGWPENANTWEPLENLQACSDVIDAFEDRLRSKRNRSSRKRKRKSGGSAQTLPKKKQQRSGASNVRGVHFRINDEHLLSTHVNTLGHADIEGVNGAVNNVEMTKESNENGSGDVSLQIPQREEDNDSDPKLIELRGSNEGNANKFKIQFQEVKALEGDCPMNGHSKVERVEMDQSSRRTGARRRKSGSVKRFTKDLAPCEPNDIQNTATRIQIGSCGRVESPEFPGNDSGLNNKFDGAINMSAITEIIKPISYSASISNDAQDVSVTFMAMRSDGKEVMVDNKFLKANNPLLLINFYEQHLRYSPTT

SEQ ID NO: 78 - protein, 436 aa, Gossypium hirsutum
MKGGRKRVSQSEGGGRGGSVKEKKKGGTEEEIVGTEGLGEKKEEEEEQEEEEDDDDDDDDEEEEEEEEEGKEGENGERKEDERTKLDDGFFEIEAIRRKRVRKGQLQYLIKWRGWPETANTWEPLENLQSCSDVIDAFEESLRSGKHSRKRKRKYGGPHTQSKKKQPCSSGSTYNATGLDLGVVDKTPLVPFDNSGIADLSASSPVIVLAREGERNGNSSNVRRAKRVKDNSSTNGSKKVDETKDENDYDTKLSELKGALSSKGGNTDKLAIRFQEGKASEGDGPVNGLQKVDRGESVQSDRRTGAKRRKAGSVKRFTQDPASSGPNLTQNATNVHVGYAITDAEMEIVNLGLAADGSSHRSPIDNSLNVPVITKILKPVGFSASVSANTQDVSVTFLALRSDGKEVMVDNQYLKANNPLLLISFYEQHLKYSPPS

SEQ ID NO: 79 - protein, 440 aa, Citrus sinensis
MKVKGGGKRKVVNPALNDAEEANDDIENQENINYDSIDLIDVEYQENQEQEKENEKEKEKGKEKVGEEKNNEEEEEEEEEEEEGEEEDEDAVPQEERPKLDEGFFEIEAIRRKRVRKGQLQYLIKWRGWPENANTWEPLENLQSCSDVIDAFEESLRSGKSSRKRKRKGGGSSSLPKKKQARTFSAPYYVTGGVGQSLPADPLINAGLIDLSPSTQSIGSGHVGGNVGNVNNLRTAKQTNDNRLANGSKQIDGRNEEAEYDPKLSELKGMISNNEANADKLALHFQEARVSEGNGLTNGLSKADQVEPLHSNRRTGARRRKPSSVKRFKQDLASTKVIVTQDSTPGIAVGCDSADEQLGIGNSSHKSKHEGPINASAIVKILKPIDFSASVSDNMQDVVVTFMAVRSDGKEVMVDNKYLKANNPLLLINFYEQHLKYSTQ

SEQ ID NO: 80 - protein, 435 aa, Populus trichocarpa
MKGKRKATANPVLTDAAEGSSSNLFETQVREGNVDEEEERIWAYNGENREETEGEEEEDDDDEEEEEEEEDSKGEQERDNLFDEERTKLDEGFFEIEAIRRKRVRKGQLQYLIKWRGWPETANTWEPLENLQSCADVIDAFEESLQSGKSFRKRKRKHGGPHTQSKKKQSRSSTSYNVLDVEISIADKHLPSAPLSSSLLGDLPSPLQFISSDPGGESNGEANNVKTFKQIDENGSMNGSKHIFPRKEDNEYDPKLSELRGTIPNIDVNTNNLAIHFQEEKTLEGNGLANGLPKVDYDPVQNSQRTGARKRKSGSVKRFKKDSVKPAFLHNSSANFSVGSTGGVAQLGIENPSLTWGNSSHMPTAENTINALAITKILKPTGFSASVSDNVQDVLITFRALRSDGQEVTVDNRFLKANNPHLLINFYEQHLKYST

SEQ ID NO: 81 - protein, 245 aa, Arabidopsis thaliana
MEFWGIEVKSGKPVTVTPEEGILIHVSQASLGECKNKKGEFVPLHVKVGNQNLVLGTLSTENIPQLFCDLVFDKEFELSHTWGKGSVYFVGYKTPNIEPQGYSEEEEEEEEEVPAGNAAKAVAKPKAKPAEVKPAVDDEEDESDSDGMDEDDSDGEDSEEEEPTPKKPASSKKRANETTPKAPVSAKKAKVAVTPQKTDEKKKGGKAANQSPKSASQVSCGSCKKTFNSGNALESHNKAKHAAAK

SEQ ID NO: 82 - protein, 295 aa, Glycine max
MEFWGVEVKVGQTVTVDPMDPVDSYIHISQVALGEAKKDKPNEPVVLYLKVGEQKIVLGTLSRDGIPHLSLDLVLDSDSELSHTSKSASVFFCGYKVLTGNDNASDFSDSSEEDEELALEGQDNGKPELKAEGAKVTKPSKSIPKIGAPAKAADPKKDEDDDSDDESDDDLAGEDESGSSDEMDDDSNSEEESDGDDEETPAKKVDQGKKRPNESAAKTPISAKKAKTATPEKTDGKKSVHVATPHPSKKGGKTPNSTKGQTPNSAGQLSCASCKKSFTNEAGLQQHKKAKHGGQ

SEQ ID NO: 83 - protein, 301 aa, Zea mays
MEFWGLEVKPGSTVKCEPGHGFILHVSQAALGESKKSDSALMYVKVDDKKLAIGTLSIDKYPQIQFDLVFNKEFELSHTSKTTSVFFSGYKVEQPIEGDEMDLDSEDEEEELNIPVIKENADGKEEQKNQEKAVAATASKSSLGLEKKSKDDSDDSDEDESDDSDEDDSDDSDEGEGLSPDEGDDDSSDEDDTSDDDEEETPTPKKPEAGKKRGAENALKTPLSDKKAKVATPPAQKTGGKKGATHVATPHPAKGKTPANNDKSTEKSPKSGGSVPCKSCSKTFNSEMALQAHSKAKHGAK

SEQ ID NO: 84 - protein, 274 aa, Oryza sativa
  source 1..274: note = Japonica Group
MEFWGLEVKPGQTVKCEPEDERFLHLSQAALGESKKGSDNAVMYVKTDDQKLVIGTLSADKFPQIQFDLVFDKEFELSHTSKTAKVEDEEEEEKIIPAPRANGKVEGKENEQKKQGKTDSSASKSKAAVNDDDDDDDSDEDDSEDEDLSPEDDDDDSSEDDSSEDDEDESDEEDTPKKPETGKRKVAEIVLKTPSSDKKAKIATPSGQKTGDKKGVHVATPHPAKQASKTPVNDKSKEKSPKSGGGSISCKSCSKTFNSEMALQSHSKAKHPAK

SEQ ID NO: 85 - protein, 277 aa, Sorghum bicolor
MEFWGKEVKPGATVSCEVGNDLVIHLSQAALGEPKKASENVIVSVKIDDKKLVLGTLSVEKHPQISCDLVFDKDFELSHNSKTASVFFCGYKSPVPQFESYSGEDSSDEELETDHQIPRNEISAAKVPVKGGIKVESSSSDDDDDDFTDSEMSDEDDSSDEDDVSTGDDSSDDSGSEEQTPTPKKTDVVVGKKRAIQAEAPSGKKAKSEQSAQKTGDKKVSTHSAKQSSKTPDKSTKTPTADKKSPKSGSHACKSCNKSFSSATGLESHQKAKKHEA

SEQ ID NO: 86 - protein, 293 aa, Cucumis sativus
MEFWGVEVKAGQSLIVKPGNEKLIHLSQATLGEIKKEKANENVTIFLKIGDQKLVLGILSGEKFPQLSFDLVFEKEFELSHNGKSGSVYCAGYQAYGDDHEQDGYLDSDSASEDEELPLVSAPNGKPSPKGVKPGLVESNDAKKASLKKPLKPSKDDDEDSEDDESDGDEDSDDESDEEMLDGDNSDEEDDDSESDEETPKKVESAKKRLNESATKTTPVPAKKAKLATPEKTDSKKGGHTATPHPAKKTGKTPAAKLETPKSGGQFSCKSCDRSFGSDVALQSHSKAKHGGK

SEQ ID NO: 87 - protein, 320 aa, Vitis vinifera
MEFWGVEVKAGEPLEVVPGDDMLVHISQASLGEIKKDKGNELVCLYANIGGQKLVLGTLSAEKFPQISFDLVFEKKFELSHNWKNGSVYFCGYKAANSVESESSDSSSDSDSEFEIDLPHNTTVDNGKPQQLKQEKPESAAAKQVKIVEPNKDAKSPKENDDDSDKDSDESMDDEDESSEDEDMVNDRSASSEDEDDDENEDNEDDKNEISKKKETPKKAGPSKKRPTESVSKTPVPDKKAKFVTPQKTDGKKSDDKKSGGHVATPYPSKQAGKTPANTEKPKQQVQKSGGSFSCKSCNRSFNSENALQSHTKAKHTAAK

SEQ ID NO: 88 - protein, 286 aa, Gossypium hirsutum
MEFWGIEVKAGQPIKTDPGASHVIHLSQATLGESKNKAESVPLYVNVNGKKLVLGTLSHQNCPQLCFDLVFEQGFELSHNWKNGSVYFLGYKAFVPEEGSDEEFDSEEESSEDEELPVAAENGKAKTDAKTAKANAGKPDAVKQAVKIAEPSNNKKNEDDDDDSDSEDGSGSDDEDDSEDEDEEMSVDESSDEDEETPKKVESSKKRPAEAATPVSAKKAKPAATPQKTDGKKGGHTATPHPSKQAGKTSGKSPKSGGQFSCGSCSKSFGSEGGLESHKKAKHGGK

SEQ ID NO: 89 - protein, 298 aa, Citrus sinensis
MEFWGVEVKAGQPLKVKPNFDNIIHLSQASLGESKKEKGHDSVPLFVKFGDKKLVLGTLIPDNIPQLSFDLVFEKEFELSHNWKNGSIYFCGYQTPLPMDSSDEFDMGDSDEEEQEELPSITAVAENGKAGQKAEKAKVATPKANLAAKPAKPIKDVKPEEDGDDESDDSDDEMVDGDDSDAISGEDDSDDDDEDDSEDDEDSDEDEETPKKPEVGKKRPTESATKTPAPAKKAKTPMSDGKKGVHVATPHPSKGVKTPNAKAQTPKSGGQVSCSSCSKTFNSENALQSHTKAKHSAK

SEQ ID NO: 90 - protein, 275 aa, Populus trichocarpa
MSSMEFWGVEVKAGEPLKVEPKDFCMIHLSQAALGESKKGNESVPLFVKFDEKKLVLGTLSPENIPQLSFDLVFEKEFELSHNWKKGSVFFCGYQVIPDDDASDFSGDEEDFEFAKADIMKAATAKDKAAKPEKPKATLVEPSIDDDEDDSDEDDSDEDGDSEEGMSLDEDSDDESDSEDEETPKKAETSKKRATDSATKTPVSSKKAKTATPLKTDAKKAGQVTPHPAKGKAAANGNSSKSPKSGGNFSCKSCDKAFGTDGALKSHSQAKHGAK

SEQ ID NO: 91 - protein, 306 aa, Arabidopsis thaliana
MEFWGVAVTPKNATKVTPEEDSLVHISQASLDCTVKSGESVVLSVTVGGAKLVIGTLSQDKFPQISFDLVFDKEFELSHSGTKANVHFIGYKSPNIEQDDFTSSDDEDVPEAVPAPAPTAVTANGNAGAAVVKADTKPKAKPAEVKPAEEKPESDEEDESDDEDESEEDDDSEKGMDVDEDDSDDDEEEDSEDEEEEETPKKPEPINKKRPNESVSKTPVSGKKAKPAAAPASTPQKTEEKKKGGHTATPHPAKKGGKSPVNANQSPKSGGQSSGGNNNKKPFNSGKQFGGSNNKGSNKGKGKGRA

SEQ ID NO: 92 - protein, 306 aa, Glycine max
MEFWGAEVKVGESVKVDPAEFEACIHLSQAALGEAKKDKPEPVVLYMKVGEQKFVLGTLSREKIPQISLELVLEKEFELSHSSKSASVHFCGYKAYYDADNSDEDEFTDSDEDDEDVPLINTENGKPETKAEDLKVPESKKAVAKASGSAKQVKVVEPKKDNEEDSDDDSDDDDFGSSDEEMEDADSDSDDESGDEDDDEETPPKKVDLGKKRPNESASKTPVSSKKAKNATPEKTDGKKGGHTATPHPAKKGGKTPNREAKAKTPNSEAKAKTPKSGGLSCKTCSKSFTNESGLQQHNKAKHGGQ

SEQ ID NO: 93 - protein, 285 aa, Zea mays
MEFWGEEVKPGATVSCKVGDGLVIHLSQAALGEPKKASENAIVSVKIDDKKLVLGTLSVEKHPQISCDLVFDKDFELSHNSKTASVFFCGYKSPVPLFESDSGEDSSDEEVEPDLIPMQNNEIKISTAKVPVKVGIQNADEDETSSGDDDDFTDSDSEMSEEDESSDEDEVSSDTDTSDDSGSEEQTPTPKKTEVVVGKKRAIEAETPSGKKAKSEQSAQKTGDKVSTSHPAKQSSKTPADKSTKTPTADKKSPKSGSHACKSCSKSFGSASALESHQKAKKHEA

SEQ ID NO: 94 - protein, 297 aa, Oryza sativa
MEFWGLEVKPGQTVKCEPEDERFLHLSQAALGESKKGSDNAVMYVKTDDQKLVIGTLSADKFPQIQFDLVFDKEFELSHTSKTASVFFSGYKVSQPAEEDEMDFDSEEVEDEEEEEKIIPAPRANGKVEGKENEQKKQGKTDSSASKSKAAVNDDDDDDDSAEDDSQDEDLSPEDDDDDSSEDDSSEDDEDESDEEDTPKKPETGKRKVAEIVLKTPSSDKKAKIATPSGQKTGDKKGVHVATPHPAKQASKTPVNDKSKEKSPKSGGGSISCKSCSKTFNSEMALQSHSKAKHPAK

SEQ ID NO: 95 - protein, 299 aa, Sorghum bicolor
MEFWGLEVKPGSTVKCEPGHGFILHLSQAALGESKKSDNALMYVKVDDQKLAIGTLSIDKFPQVQFDLVFDKEFELSHTSKTTSVFFSGYKVEQPMEEDEMDLDSEDEEELDVPVIKENGKADGKEQKSQEKAVAAASKSSLVSKKSKDDDDSDEDETDDSDSDDTDDSDEGEGLSPEEGDDDDSSDEDDTSDDEEEETPTPKKKPEAGKKRPAENSLTPLSDKKAKVATPSAQKTGGKKGAVHVATPHPAKGKTPANNDKSKEKSPKSGGSVPCKSCTKTFNSEMALQAHSKAKHGAK

SEQ ID NO: 96 - protein, 293 aa, Cucumis sativus; identical to SEQ ID NO: 86

SEQ ID NO: 97 - protein, 300 aa, Vitis vinifera
MEFWGVEVKAGESFKVKSEDDKILHLSQAALGESKKEKGNESVPLFLKIDQQKLVLGTLLPANIPQLSFDLVFDKEFELSHNWKNGSVFFMGYKSVLPDEEEFSEFDDSDSEEDLPVNAIENGKPEPKVEQAKAVPTNANAGKAKVKVEEPPKDAKDEEDDDDDSDEDMVDEDSDEDSDEDIMSDAEDGSDEGSESEDEETPKKKVEPGKKRPTESATKTPVPAKKAKLVSPQKTDGKKGGAHTATPHPNKKAGKTPASGDKGKGQSPKSGGQVSCKSCSKTFNSENALQSHSKAKHGAS

SEQ ID NO: 98 - protein, 295 aa, Gossypium hirsutum
MEFWGIEVKSGQPIKADPGANYVIHLSQASLGESKNKAESVPLYVNVDGKKLILGTLSHQSCPQLSFDLVFDEEFELSHNWKNGSVYFLGYKTFIPEEGDDFGMSSEEESEEEEEEELPVAAAAAAVNGKAKPDGKTAKANAGKPDAVKQSGKIAEPSSNKKSEDEDDDDESEDESGSDEEDDSDDDDESDEMSMDESSDDEDEETPKKVESSKKRPAEAATPASAKKAKSAVTPQKTDGKKGGHTATPHPAKQSGKNSAKSPKSGGQFSCGSCNKSFGSEGGLESHKKAKHGGK

SEQ ID NO: 99 - protein, 298 aa, Citrus sinensis; identical to SEQ ID NO: 89

SEQ ID NO: 100 - protein, 305 aa, Populus trichocarpa
MEFWGVEVKGGEPLRVESGDGFILHLSQACLGEAKKDKGNESVCLFVNFDDQKLVLGTLSHEKIPQIPFDLVFEKDFELSHNLKNGSVFFSGYKVAQPESDSDEFISDGEEDLPVPVDNGKEEAKQPKPENENAAKPDSKQKVKIVEPNKDGKPKPENDDSSDEEDDSSDDGESSDDQAMMMANDEDESSEDEDDESDEDDDDSDDGDVKPPKKAEVGKKRSAESASKNPVPDKKAKFVTPQKTDLKKAGVHIATPHPSKQAAKTSANSQRKEQAQKSFSCNSCNRSFGSENALQSHSKAKHSAA

SEQ ID NO: 101 - protein, 294 aa, Arabidopsis thaliana
MEFWGVEVKNGKPLHLDPGLDRLVHISQVALGESKNNVTEPIQLYVTVGSDKLLIGTLSHEKFPQLSTEIVLERNFALSHTWKNGSVFFSGYKVDASDPEPEDLIDDQLEAAGFKAAPKSAAKQVNFQLPNEDVKAKQDDDADGSEEDSSDDDDSENSGDEEEEKVTAESDSEEDDSSDDEEDDSSEEETPKKPEEPKKRSAEPNSSKNPASNKKAKFVTPQKTDSKKPHVHVATPHPSKQAGKNSGGGSTGETSKQQQTPKSAGAFGCKSCTRTFTSEMGLQSHTKAKHSAAA

SEQ ID NO: 102 - protein, 288 aa, Glycine max
MEFWGVEVKSGESLKVDPGDDKIIHLSNACLGDVTKDKEGLPVALYVKFGNQKLVLGTLSSEAFPQISYDLVFEKEFELSHNWKHGSVFFTGFKAQSQSESDNDEDSDDFDEDIPVSAANGKPEIEVKNGIKPDANEAKQKKIANGEDEDSSESESDDDSSEDQLTANGDIESSEGDDDSDDEEEGDDESDEETPKKIEASNKRGIDSSKKTPVPVKKTKFVTPQKTDSKNVVHVATPHPSKQTGKATANNKQQTPKSGGDYSCKPCNRSFKTEDALSSHNKAKHSTK

SEQ ID NO: 103 - protein, 271 aa, Zea mays
MEFWGLEVKPGSTVKCEPGHGFILHVSQAALGESKKSDSALMYVKVDDKKLAIGTLSIDKYPQIQFDLVFNKEFELSHTSKTTSVFFSGKADGKEEQKNQEKAVAATASKSSLGLEKKSKDDSDDSDEDESDDSDEDDSDDSDEGEGLSPDEGDDDSSDEDDTSDDDEEETPTPKKPEAGKKRGAENALKTPLSDKKAKVATPPAQKTGGKKGATHVATPHPAKGKTPANNDKSTEKSPKSGGSVPCKSCSKTFNSEMALQAHSKAKHGAK

SEQ ID NO: 104 - protein, 297 aa, Oryza sativa
  source 1..297: note = Japonica Group
MEFWGLEVKPGQTVKCEPEDERFLHLSQAALGESKKGSDNAVMYVKTDDQKLVIGTLSADKFPQIQFDLVFDKEFELSHTSKTASVFFSGYKVSQPAEEDEMDFDSEEVEDEEEEEKIIPAPRANGKVEGKENEQKKQGKTDSSASKSKAAVNDDDDDDDSDEDDSEDEDLSPEDDDDDSSEDDSSEDDEDESDEEDTPKKPETGKRKVAEIVLKTPSSDKKAKIATPSGQKTGDKKGVHVATPHPAKQASKTPVNDKSKEKSPKSGGGSISCKSCSKTFNSEMALQSHSKAKHPAK

SEQ ID NO: 105 - protein, 478 aa, Sorghum bicolor
MGGSGGGAGQRSGAHLTHAGAEGENAYWRRRDQYREANESGGLVGAGWQEARQPAGKPEAGGARAAVPSSPVCECVAVSSSAPREAGRSTRFGCSHQPRNAGSPLGQSRIRIPKPTLGITLLLVSGTGTAPSASLTSRSQKPRTLSSREAPRRREAAGRRSRTPSRALLPRRAMPMVNGIFEYWGVVVRPGATVKCDPGEFYCHVSQIALQDSKGNEDVRVFVKADGKKFLIGTLSVDKYPQYTTSLVFEKEFELLHTSKTSTISALGYKFSETSTEEGDESNEEVPQAIPLYPNADDDKSKESKCGVEKPAATESSKSKVSLEEAKVPDKHKADVGGDDNDESDEDVDSEDGESGDDESSSDEDDGESSNEDDDEDSPKSTKRNNRPAETPLKTPPGKKARITTPSMGKKSVSDDAKRSNHVHVATPYPSSKQVKTARSIIDNSKQSTGYACKSCSKTFYSSVGLETHCKVKHSACK

SEQ ID NO: 106 - protein, 318 aa, Cucumis sativus
MEFWGAEVKVGEPLLVSPGEEKILHLSQACLGESKSKGSEPVVLYVKVGNQKLVLGTLSSEKFPQVSFDLVFDKEFELSHNWKNGSVHFTGYTSLLPEEEESTYTDSEEDFPLAVTDNGKPEPEPEPKKDAKPDTSGTKKKVQIVEPSKVASNSNIDSKKNKDSSDEDDDEDEESTDNENENGGEEDDSDDDENESSGVDSEDSDEDSDDSLEEDEQTPKKAEASKKRPLDSANKTPAPDKKAKLVTPQKTDGKKGVAHVATPHPSKKAGKMAAANDKNKQQTPKSANAAFSCKTCNRTFGSETALQSHTKAKHTADK

SEQ ID NO: 107 - protein, 320 aa, Vitis vinifera; identical to SEQ ID NO: 87

SEQ ID NO: 108 - protein, 286 aa, Gossypium hirsutum; identical to SEQ ID NO: 88

SEQ ID NO: 109 - protein, 298 aa, Citrus sinensis; identical to SEQ ID NO: 89

SEQ ID NO: 110 - protein, 305 aa, Populus trichocarpa
MEFWGVEVKGGEPLRVESGDGFILHLSQACLGEAKKDKGNESVCLFVNFDDQKLVLGTLSHEKIPQIPFDLVFEKDFELSHNLKNGSVFFSGYKVAQPESDSDEFISDGEEDLPVPVDNGKEEAKQPKPENENAAKPDSKQKVKIVEPNKDGKPKPENDDSSDEEDDSSDDGESSDDQAMMMANDEDESSEDEDDESDEDDDDSDDGDVKTPKKAEVGKKRSAESASKNPVPDKKAKFVTPQKTDLKKAGVHIATPHPSKQAAKTSANSQRKEQAQKSFSCNSCNRSFGSENALQSHSKAKHSAA

SEQ ID NO: 111 - protein, 589 aa, Arabidopsis thaliana
MASYRPPYPPLPQPPSQNSLAPPPPPPSLPPPVPPPPPSHQPYSYPPPPPPPPHAYYQQGPHYPQFNQLQAPPPPPPPSAPPPLVPDPPRHQGPNDHEKGASKQVGRRERAKPDPSKHHHRSHLPHSKKIETEEERRLRKKRELEKQRQDEKHRQQMKNSHKSQMPKGHTEEKKPTPLLTTDRVENRLKKPTTFICKLKFRNELPDPSAQLKLMTIKRDKDQFTKYTITSLEKLWKPKIFVEPDLGIPLDLLDLSVYNPPKVKAPLAPEDEELLRDDDAVTPIKKDGIRRKERPTDKGMSWLVKTQYISSINNESARQSLTEKQAKELREMKGGINILHNLNNRERQIKDIEASFEACKSRPVHATNKNLQPVEVLPLLPYFDRYDEQFVVANFDGAPIADSEFFGKLDPSIRDAHESRAILKSYVVAGSDTANPEKFLAYMVPSLDELSKDIHDENEEISYTWVREYLWDVQPNANDPGTYLVSFDNGTASYLPLPMRLNLRKKRAREGRSSDEIEHFPVPSRVTVRRRSTVSVIEHKDSGVYSSRVGASSSKMRRLEDEGGLGRSWKHEPEQDANQYSDGNEDDYSE

SEQ ID NO: 112 - protein, 659 aa, Glycine max
MASYRPFPPQSSQIPNPIPGGNHQYNNNQNQNWGAYGDPSTSSFPQIPPNSNYHQNQQHHVPYAPPNPHHPHYPYPPPPPPPPPEASYQPPPPPPPAPMYYPSNNQYSNQPPPPPPPLSPPPPPPPVSPPPPPAVTQNNEERHFKEPSTSGRREYEHSNHGIAHKQHKQQPPVPVKKMNNGPPGRAETDEEKRLRKKREFEKQRQEEKHRQQLKESQNTVLQKTHMLSSGKGHGMIAGSRMGERRSTPLLGAERVENRLKKPTTFLCKLKFRNELPDPSAQPKLMASKKDKDQYAKYTITSLEKMYKPKLFVEPDLGIPLDLLDLSVYNPPSVRPPLAPEDKELLRDDEAVTPIKKDGIKRKERPTDKGVAWLVKTQYISPLSMESTKQSLTEKQAKELREMKGGRGILDNLNSRERQIREIEASFEAAKSDPVHATNKDLYPVEVMPLLPDFDRYDDQFVVAAFDNAPTADSEMHAKMDKSVRDAFESKAVMKSYVATSSDPANPEKFLAYMVPAPGELSKDIYDENEDVSYSWIREYHWDVRGDDADDPATFLVAFDESEARYLPLPTKLVLRKKRAKEGRSGDEVEQCPVPARVTVRRRSSVAAIERKDSGVYTSSKGNSSKRGGLEMDDGLEDQHRGAPHQDNYQSSGAEDYMSD

SEQ ID NO: 113 - protein, 621 aa, Zea mays
MASYRPYAPPPPQGGYPPQMNSYAPPPQQAPYSRMPAPTYHAGPPPPPPPGPPPPHQPQFNFGPGPPQQLPPPPPPQMYYQPPPPPYGGNSNPPPPPSAPPPPPSPPPAASPPPPPPPAQPPSAHALLPPKGPQPKATLLRAETEEERRARKKREFEKQRVDDRKQHQMMRQTQATILQKTQQVRTAQQQPQSRHHHHHPPGGSRAGATGSRPATAVNAERFENRLKKPTTFLCKHKFRNELPDPSAQLKWLPLNKDKDRYTKYRITSLEKNYIPKMIVPEDIGIPLDLLDMSVYNPPDVQPPMAPEDEELLRDDQVLTPIKQEGTRKRERPTDKGVSWLVKTQYISPLSADAAKTSLTEKQAKERRESRGGRNAFLDNLNDREKQIKAIEESFRAAKSRPVHQTKRGMQAEWVMPLLPDFDRYEEPFVMVNFDGDPTADSEQYNKLERPVRDECESRAVMKSFSVSGSDPSKQEKFLAYMAPAPHELTRDLDDENDDIQYSWLREYHWEVRGDDKDDPTTYLVTFDKKDGAKYLPLPTKLVLQKKKAKEGRSGDEIEHFPVPSRITVSKTAHGGTMERGESSGMHANSKPRRSHVDDDLDEHPKRTRVEDIDQYSEEYSE

SEQ ID NO: 114 - protein, 632 aa, Oryza sativa
  source 1..632: note = Japonica Group
MASYRPYAPHPPPPQGGFPPQPPPMNPYGPPPPQQPAYGHMPPPQGAPPPFLAPPPPPPPGPPPPHQPQFNFGPGPPQQQQPPPPPQMYYQPPPPPPPYGVNSSQPPPPPPPPPSPPPSAPPPPPPPPTQPPPREAQLAPPPPREQQSKSALPRAETEEERRARKKREYEKQRAEDRKNQQMMRQSQATVLQKTQQVRAAQQPQSRHHQQPSGGSRPAVTATRPAAAPNAERFENRLKKPTTFLCKHKFRNELPDPSSQLKWLPLNKDKDRYTKYRITSLEKNYIPKMIVPEDLGIPLDLLDMSVYNTPPVQPPMAPEDEELLRDDEVLTPVKKDGIRKKERPTDKGMSWLVKTQYISPLSTDAAKMSITEKQAKERRESREGRNTFLENINDREKQIKAIEDSFRAAKSRPVHQTKRGMEAEWVLPLLPDFDRYDDQFVMVNFDGDPTADSEQYNKLERSERDECESRAVMKSFLVNGSDPAKQEKFLAYMVPSPHELSKDLDDETEDIQYSWLREYHWEVRGDDKDDPTTYLVTFDDDGAKYLPLPTKLVLQKKKAKEGRSGDEIEHFPVPSRITVSRTAHGGMMEHGESSSMHENLKRQRSSVDDDLYDHPKHSRVEDMDQYSGDEYSD

SEQ ID NO: 115 - protein, 640 aa, Sorghum bicolor
MASYRPYAPPQQHPPPPPPQGGYPPQMNPYAPPPQQAPYNRMPAPPYHAGPPPPPPPGPPPPHQPQFNFGPGPPQQPPPPPQMYYQPPPPPYGGTSNPPPPPPSVPPPPPSPPPAAPPPPPPPPAQPPSVQAPLPPKEQPPKATLPRVETEEERRARKKREFEKQRVEDRKQQQMMRQTQAAILQKTQQRAAQQQPQSRHHHHQPPSGSRAPATGSRAVVTGSRAVATGSRPASAPNAERFENRLKKPTTFLCKHKFRNELPDPSAQLKWLPLNKDKDRYTKYRITSLEKNYIPKMIVPDDLGIPLDLLDMSVYNPPDVQPRMAPEDEELLRDDEVLTPIKQEGIRKRERPTDQGVSWLVKTQYISPLSTDAAKMSLTEKQAKERRESREGRNAFLDNLNDREKQIKAIEESFKAAKSRPVHQTKRGMQAEWVMPLLPDFDRYEEPFVMVNFDGDPTADSEQYNKLERSVRDECESRAVMKSFSVSGSDPTKQEKFLAYMAPAPHELTRDLDDDDDIQYSWLREYHWDVRGDDKDDPTTYLVTFDKEEGAKYLPLPTKLVLQKKKAKEGRSGDEIEHFPVPSRITVSKTAHGGTMERGESSGLHVNSKPRRSHVDDDLDEHPKRSRVEDIDQYSGEEYSE

SEQ ID NO: 116 - protein, 706 aa, Cucumis sativus
MASYRPYPPQSSFGSAPAQNSIPPPSAQSASVSSQQRGGATTQYNQNWGTYAGDASAPPAPSSSYPQNYNNQLHQTSNYHHQQYGPPRTQHPPPPPPPPHQSYPYAPQPPPPPPPDSSYPPPPPPPATSQPPNLYYPSSQYSQGNQNQQSMQPPPPPSSPPPSSSXPPPPPPNSPPPPSASQQKAEGTNMGAHERDKGVPKDPSYGRRDRENSNHDKHQKHSGPPMPPKKANGPSGRMETDDEKRLRKKREFEKQRQDERHRHHLKESQNTILQKTQMLSTGKVHGSIVGSRMGERKATPFLSGERIENRLKKPTTFLCKLKFRNELPDTSAQPKLMSLRKEKDHYTRYTITSLEKTYKPQLYVEPDLGIPLDLLDLSVYNPSSVRMPLAPEDEELLRDDVLKTPVKKDGGIKRKERPTDKGVAWLVKTQYISPLSIESAKQSLTEKQAKELREMKGGRNILENLNNRERQIKEIEASFEACKSRPIHATNKNLYPVEVLPLLPDFDRYDDPFVVVAFDSAPTADSETFNKLDQSIRDAHESQAIMKSYMATSSDPSKPEKFLAYMVPSPDELSKDIYDEQEDVSYSWVREYHWDVRGDNVDDPTTYLVSFDDAEARYVPLPTKLVLRKKRAKEGRSSDEVEHFPAPARVTVRRRPTVATLEVKDPGIYSNSKRGSDIEDGIGRSHKHDRHQDMDQFSGAEDEMSD

SEQ ID NO: 117 - protein, 702 aa, Vitis vinifera
MASYRPFPPQSSFPPPPNQNPVPQQPPPQPHPPQQRGNQYPQNWGYPNSNYHHGGYGSSASSSQHYGGPSRTQHPPPPPHQQQQYPYPPPPLDSTYPPPPPPPASMPQPPQGPMYYPSSQYSQFSHQPMQPPPPPPPSSPPPSSLIPPPPPPASPPPPPSSVPPQGQNKEPAPDGGSHGRDKGAPKDLRGAGRREPGHSNQGPSGKQQKPPVPPAPVKKSNGPPGRVETEEERRLRKKREFEKQRQEEKQKHQLKESQNTVLQKTQMLSSGKGHGSVVGGSRMGERRTTPFLSGDRIENRLRKPTTFLCKLKFRNELPDPTAQPKLMALKTDKDRFTKYTITSLEKMHKPQLFVEPDLGIPLDLLDLSVYNPPSVRRPLDPEDEELLRDDESVTPVKKEGIKKKERPTDKGVSWLVKTQYISPLSTESTKQSLTEKQAKELRETKGGRNILENFNSRERKIQNIEAAFAASKITPVHSTNKSLKPVEILPLLPDFARYDDSFVVASFDSAPTADSEIYSKLDKTVRDSHESQAILKSYMATGSDPSKPEKFLAYMAPSPDELSKDIYDENEDTSYSWVREYHWDVRGDDADDPTTYLVSFNKTDARYLPLPTKLLLRKKRAKEGRSSDEVEHFPVPSKVTVRQRPNVAAIELKDEEVYSSSKRGVSSSKRGVDMEDGLGRSYKGVQDQHMDQSSGAEDEMSD

SEQ ID NO: 118 - protein, 676 aa, Gossypium hirsutum
MASYRPFPPPQSFPPPPQNQNPLPPQQQQQQQYGQNYNHMNPNSNYGYPPRPPQQPPPPPPPQQQYSYPPPPPPPDSSYPPPPPPPLPSMPQNTINSQPPMYYPPSQYSSQYGSQAMQPPPPPPPPASPGSSIPPPPPPPSSPPPPPPPKENVGKTGLNERNKGGNRDYSGSGRREHGHSNHGVGGREQKPMVPPVKKPNGPPGRVETEEERRLRKKREFEKHRQEEKQRQQIKESQKTQMMTSGKAHGSIVGSRMGDKRATLFLSGERIENRLKKPTTFLCKLKFRNELPDPSAQPKLMSLKKDKDRFTKYTITSLEKMYKPKLFVEPDLGIPLDLLDLSVYNPPSIRLPLAPEDEELLRDDAAITPVKRDGIRRKERPTDKGVSWLVKTQYISPLSMESTKQSLTEKQAKELRELKGGRNLLVNLNNRERQIKEIEASFEASKLRPVHATNKNLEPVDVMPLLPDFDRYDDQFVMVAFDNAPTADSEIFSKLHGSVRDEHESRAIMKSYQAPSSDPVNPEKFLAYMVPSLGELSKDMYDEHEDITYSWVREYHWDVRGDNANDPTTYLVSFDEGEARYVPLPTKLNLRKKRAREGRSGDEIEHFPVPARITVRRRPTVAAIELHEPEVYSNSRGGISSSKMARLDAEDGLGRPHKLSRHQDIDQYSGAEDDLSD

SEQ ID NO: 119 - protein, 570 aa, Citrus sinensis
MYYSSNQYNQNSMYPPMQPPLPPPPPSSPPPSSSIPPPPPPGSPPPPPPKDVEGRDTGTSDHDKRASKEVSRHDPGHSKQHRPPVPPPGVKKVNGGSGRVETEEERRVRKKREYEKHRQEEKHRLQMKESQNVVMQKSQMVASGKGGHGSMAGSRMGDRRAAPLLSGERIENRLKKPTTFLCKLKFRNELPEPSAQPKLMALKKDKDRFTRYTFSSLEKNYKPQLHVEPDLGIPLDLLDLSVYNPPSVRPPLDPEDEELLRDDEVVTPVKKDGIKRKERPTDKGVSWLVKTQYISPLSMESARQSLTEKQAKELREMKGGRSILENLNDRERQIKEIEASFEACKLRPIHATNKNLQPVEILPLLPDFERYDDQFVAATFDGAPTADSEIYSKMDKSVRDAHESRAIMKSYVATGSDSANPEKFLAYMVPSVNELSKDMYDENEDVSFSWVREYHWDVRGDDADDPTTYLVSFDDDEARYVPLPTKLNLRKKRAIEGRSNDEVEHFPIPSSIAVRRRANVTAIELKEQGGNSSSSKMGRVDSQEDLERSHNGSRHQDPYQSSGAEDDMYD

SEQ ID NO: 120 - protein, 665 aa, Populus trichocarpa
MASYRPFTAPPQPPQPPQQQQQRPNQYTQSYNPQLAPPPQNYQNYSQHYPQPPRPAPPPHQQYPYQRPPPPESSYPPPPPPPAPLQQQPPPPPPPNMYYPTSHYALQSLQQPIQPPPPPPPPSSPPPSSSIPPPPPPSSPLPPPPKDSAFERDKGVSQEKREHDHPNHGKHQQQQSQLPLVVKKANGHPGRVETEEERRLRKKREFEKQRQEENRRQQLKESQNSALLKNHVISSQKGHGSIVGSRLGDRVATPLLGGERAENRLKKPTTFMCKLKFRNELPDPSAQPKLMPLKREKDRFTKYTITSLEKMYKPQLYVEPDLGIPLDLLDLSVYNPPSVRPLLAPEDEELLHDDESVTPVKRDGIKRKERPTDKGVSWLVKTQYISPLSMESAKLSLTEKQAKELREMKGGCKLLDNLNKRERQIKEIQASFASNKLPPVHATNKNLKPVEILPLLPDFDRYGDKFVTVAFDGAPTADAENYRKFDPSDRDAYESWAIMKACVASGSDPANPEKFLAYTVPSPDELSKDMYDENEDILYSWIREYHWDVRGDDVDDPSTFLVSFDEAEARYLPLPTKISLRKKRAREGRSGDEIEHFPIPSRVTVRKRAVAATIEQRDSGAISNSRGNNSRMERFEDEDGLGRLQRVALDEDLHHSSGAEDEMSE

SEQ ID NO: 121 - protein, 774 aa, Arabidopsis thaliana
MNRLGHKSVVYHGDLRLGELDVNHVSSSHEFRFPNDEIRIHHLSPAGERCPPLAILQTIASFAVRCKLESSAPVKSQELMHLHAVCFHELKTAVVMLGDEEIHLVAMPSKEKKFPCFWCFSVPSGLYDSCLRMLNTRCLSIVFDLDETLIVANTMKSFEDRIEALKSWISREMDPVRINGMSAELKRYMDDRMLLKQYIDNDYAFDNGVLLKAQPEEVRPTSDGQEKVCRPVIRLPEKNTVLTRIKPEIRDTSVLVKLRPAWEELRSYLTAKTRKRFEVYVCTMAERDYALEMWRLLDPEAHLISLKELRDRIVCVKPDAKKSLLSVFNGGICHPKMAMVIDDRMKVWEDKDQPRVHVVSAYLPYYAPQAETALVVPHLCVARNVACNVRGYFFKEFDESLMSSISLVYYEDDVENLPPSPDVSNYVVIEDPGFASNGNINAPPINEGMCGGEVERRLNQAAAADHSTLPATSNAEQKPETPKPQIAVIPNNASTATAAALLPSHKPSLLGAPRRDGFTFSDGGRPLMMRPGVDIRNQNFNQPPILAKIPMQPPSSSMHSPGGWLVDDENRPSFPGRPSGLYPSQFPHGTPGSAPVGPFAHPSHLRSEEVAMDDDLKRQNPSRQTTEGGISQNHLVSNGREHHTDGGKSNGGQSHLFVSALQEIGRRCGSKVEFRTVISTNKELQFSVEVLFTGEKIGIGMAKTKKDAHQQAAENALRSLAEKYVAHVAPLARETEKGPENDNGFLWESSEDVSNKGLEEEAPKENISELVLRK

SEQ ID NO: 122 - protein, 836 aa, Glycine max
MSRLGFKHEVYDGDKHVGELDVIPLSSSSTTTPFHNSFRFPNNEIRIHHFSAKSERCPPLSILQTVAAFNVRCKLDSSVATEQKELIAIHASCFYEMKTAVVVVNDEEIHLVSMPSKRKKFPCFWCFAVPLGLYDACLAMLNLRCLAIVFDLDETLIVANTMKSFEDRIEALRGWLLRETDPLRVQGMSSELKRYLEDRLLLKQYAESDTVVDNGKVYKVQMEEAPPLSGSHEKLVRPVVRLQERNIVLTRINPEIRDTSVLVRLRPAWEDLRSYLTAKGRKRFEVYVCTMAERDYALEIWRLLDPGAHLIGSKQVLNRVICVKSGSRKSLLNVFQDGVCHPKMAMVIDDRSKVWEDKDQPRVHVVPAFTPYYAPQAETANAVPVLCVARNVACNVRGCFFKEFDESLLQRIAEIFFEDDIGLLPLPPDVSNYLMSEDVPNGNANAPISEGINGAEVERRLSQPDDKFSVDLVTRPMTNSVEFRHETSQPTAGIISNVTGPASSRTLIPSQKPGLLGPPVKHDGNSVDRDYDMRKGLLGMRHGPDIRGQISAEPPLISRPPNQTSPSLIQPFGGGLVEDDIASRTQTNSWPSASFKESNVIKFDKHQAQQKPFSHSVIGSSPNVLPPQASQVKTEEATSVSDLQRHIAPSKSQLSSEDGISQNHATSNSKDFQNEAGKVNFLPSLSIQVLQEIGRRCNSKVEFKTILSTSKDLQFSVEVLFTGEKIGVGMARTRKDAQQQAAENALRSLAEKYVAHVEPQCRAVDKDFDKLSLGRDNGFLWDVVNLESSELQPEDGVPRENASEALDAETRSSTPNAINQQMDKRVSSPRSKRLKE

SEQ ID NO: 123 - protein, 906 aa, Zea mays
MFKSMVYFVNISIGEVEVWPKGQASAGLAVAAWARDIRVDRLSPPSERCPPLAVMHTVAVAARCLVMESRPPVAADVASLPLVDMHAACLRDNKTAVVALGDEELHLVAMTSRRNLTNHACFWGYKLPFGLYNSCLTMLNLRCLGIVFDLDETLVVANTSRSFEDRIDALQRKLSNETDPQRRNGMLSEIKRYQDDKSILKQYIEGDQVYDDGKVYKAQPEIVPPLSDNQQTMTRPVIRLQEKDIILTRINPLIRDTSVLVRLRPAWEDLRSYLIARGRKRFEVYVCTMAERDYALEMWRLLDPDSRLINSVRLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNDISSAPDVGNYLITEDENVALVNGNRDSLPFDGMSDAEIERRMKEANAQAFHQTATNFVMPVAPAQNFVSSSVAPLAPPLSMMPPPFSQPVVQPGFSDPLQGSPAREEGEVPESELDPDTRRRLLILQHGQDTRDPTPPHPAIPPVQVPVPPVQPHGNWFPTEDGINPSNLSRGSAGFTVESDSMPYEKKQPPHPSFFHGGDSPMSSDRFGYQNQRFPSQLPHTEDHHMLQNHAPPKYRSFSGEELATRHVPSSQRNHIESGRHFSQYAGTSAGVLEGIAVKCGSKVEYRSTLCDTAELQFSIEVWIVGEKFGEGIGRTRREAQRQAAEMSLRNLANKYLSSDPNKLTDMKQDAFGSNRNIFGYSGNTRDDMLPLSSTSEESRFMKMEENNSRKTGDSVTALKELVQIGRQILGKGVGLTWEEAKLQAADEALGTLRSMLGQLGHRRSGSPRSLAPNFNKRFKPDFPRTVQRVPYGTYSRIEGHAP

SEQ ID NO: 124 - protein, 748 aa, Oryza sativa
  source 1..748: note = Japonica Group
MATPRAPPQAAVPGGGAGATAGGVTMRMFHGDVFLGEAEVFPMKQGAEGSLPFPSNEIRISHLSPTSERCPPLAILQTIAPFSVRCKLQAKLMPPHPSLHRLYLTCFNEYKSAVVVVGDEELHLVAMPSKVEKVPCFWCCSVRSGIYAASVGMLNLRCLAIVFDLDETLIVANTMKSFEDRIEMLSRRMDIEDDPVRIAGMSAEIKRYIEDRELLKEFIDTDTVTDNGRIVGTQKEEVLPISGGQERVLRPVIRLPERNAILTRINPEVRDTSVFVKLRPAWEDLRSYLTAKGRKRFEVYVCTMAERDYALEMWRLLDPEANLISSHNLAERVVCVKSGSKKCLQNVFKDKGCHPKMAMVIDDRLQVWDEKDQPRVHVVPAYTPYYAPQAEMANAVPVLCVARNVACNVRGGFFREFDENLLRKVFELMYENELLDLPYAPDVGDYLVCEDTNFAPNNKDVAPIPEGMSGAEVGKRLNGLAYPRDQKQIPSSTRLSDDDGVALRGIPGGTNIQSNGGSLATTPSLFVTVLQEIGRLCESRVEFRSTVSSGKNMQFSVEVLFSNEKIGIGIGKTRDEAQVLAAEKALQNLESNYLSFMAPVAGVLNKDVNKPPGSGNGFLEDITLSEDISMEEPSGSTLKEQDHSKALDRLSSVISLIRELCLEDQHVVFRDQVRDSGSALNGEYHFQAELGGQILGRGIDLNKDFAKLQAAEEALKTLKTTTDPQIKKHLRPMQQLNFPLYPTEVCSM

SEQ ID NO: 125 - protein, 934 aa, Sorghum bicolor
MFKSMVYYGNTSIGEVEVWPKGEASAGLAVAAWAREIRVDRLSPPSERCPPLAVMHTVAVGARCLVMESRPPVAADVAPMPLVAMHAACLRDNKTAVVPLGDGELHLVAMTSRRNLTNHACFWGYKLPFGLYNSCLTMLNLRCLGIVFDLDETLIVANTSRSFEDRIDGLQRKLSNETDPQRRNGMLSEIKRYQDDKSILKQYIEGDQVYDDGKVYKAQPEIVPPLSDNQQPMTRPVIRLQDKNIILTRINPLIRDTSVLVRLRPAWEDLRSYLIARGRKRFEVYVCTMAERDYALEMWRLLDPDSRLINSVQLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNDISSAPDVGNYLITEDENAALVNGNRDSLPFDGMADAEVERRMKEANAQAFHQTAGNFVMPVAPAQNFVSSSVAPLAPPLGVMPPTFSQPVVQPGFSDSLQGSPAREEGEVPESELDPDTRRRLLILQHGQDIRDPTPPLPAIPPVQVPVPPVQPHGNWFPTEDGLNPSNLNRGSAGFTVESDPMLYEKKQPPHPSFFHGGDSPMSSDRFGYQNQRFPSQLPHTEDHHMLQNHAPPKYRSFSGEELAARHVPSSQRNNQIESGRHFAQYAGTSAGILDGIALKCGSKVEYRSTLCDTAELQFSIEVWIVGEKVGEGIGRTRREAQHKAAEMSLRNLANKYLSSDPNKLTDMKENGFSGNRNVFGYSGNTRDDMLPLSSTSEESRFMKMENNSRKTGGSVAALKELCTVEGYNLVFQERPSPADGLVGKESYAQVEVGGQILGKGVGLTWEEAKLQAADEALGTLRSMLGQLAHKRSGSPRSLAPNFNKRFKPDFPRTVQRVPYGTYSRIEGHVP

SEQ ID NO: 126 - protein, 829 aa, Cucumis sativus
MSRLGFKSVVFHGDVRLGELDAIPATDQNFQFPNNEIRIHRISPPSERCPPLSVLQTISSFSLRCKLQSSLPVEQPHLIQLHYSCFHELKTAIVLVGDEEIHLVAMPSKQKNFPCFWCFAVPCGLYLSCLGMLNLRCLAIVFDLDETLIVANTMKSFEDRIEALRIWIAREADPLRISGMSAELKRYVEDRLLLKQYIEHDTVNDNGRIFKAQLEEVPPLNGNCEKVVRPIIRLLDKNLVLTRINPEIRDTSVLVRLRPAWEDLRSYLTARGRKRFEVYVCTMAERDYALEMWRLLDPEAHLIATKQLLERVVCVKSGSKKSLLNVFQSGSCHPKMAMVIDDRSKVWEDKDQPRVHVVPAFTPYYAPQAETANAVPVLCVARNVACNVRGCYFKEYDESLLRRILEISYEDDVVDLPPAPDVSSYMMSEEAGFVPNETVNAPICEGMNGAEVERRLNQPDDKHVIDMANNPTTSQTDARADTSQLPTPLNPNITGPKSSRTLLPSQKPGLLGAPVRRDISSDHDMKRGLLAMKPSIDLRTQTFGDPPILSRMSPQISASGLQVLGASLVDEDSSQGHQNRRPSGLIPESDASKSDKHRSHQNLFSNNSQALTGVAAISQNHASNNNKEHQTEAGIVSIPPPSLYIGVLQEIGRRCSSKVEFKTVVSTSKDLQFSVEVLFTGEKIGVGLGKTRKDAQQQAAENALHNLADKYAAHIKPLMGPMDADLDKLSIGVENGFLWDTAAPAVNELPKEEDVWHKENSSEGCDEPESTNSDSTNHQQPQHVQKRQSSPRPFPSKRSKEDQPHGPQHHHHQLPPSRQQKNGHSNSIT

SEQ ID NO: 127 - protein, 856 aa, Vitis vinifera
MSRLGFKSLVYHGDLFLGELDAIPVKDQSFQFPNNEIRVHHISPISERCPPLSILHTISSFSVRCKLESSSPIQQPNLINLHASCFHELKTAVVLIGDEELHLVAMPSKQKKFPCFWCYSVPCGLYSSSLWMLNLRCLAIVFDLDETLIVANTMKSFEDRIDALRGWIARESDQVRISGMSAELKRYIDDRALLKQYTENDLVMDNGKILKVQSEEVAPLSDSHERVVRPVIRFPERNIVLTRINPEIRDTSVLVRLRPAWEDLRSYLIAKGRKRFEVYVCTMAERDYALEMWRLLDPEAHLIGSKQLLDRVVCVKSGSRKSLLNVFQNGSCHPKMAMVIDDRLKVWEDKDQPRVHVVPPFTPYYAPQAETTNPVPILCVARNVACNVRAGFFKEFDENILRQISELFYEDEVVNLPSAPDVSNYLMSEDAGFVPNGNANVPIAEGMHGAEVERRLNQPDEKHIVDSAASPIANSYEFRSETLQPPALTVQNVVGPTSSRLLMPSQKPSLLGAPIKRDFSSFESDADMKRRLLIMKHGQDVRNQSLGDPPILSRLPQISTSSLHPQGVWLVEDDSNRGHLNNRASGLVQEADVLKPDKQRGHQIPFGHNTPGSTPVSLLPHLPQLKNDEVSAANERQKKNLPPASQPSEVGVSQNQASTTGREQTEAGKVNMMPPHLSIGVLQEIGRRCSSKVEFRSVVSTSKDLQFSVEVLFTGEKIGVGMGKTRKDAQQQAAENALHSLADKYVAYTTPHSGAVDKDFDKLSLSNENGFLWDTTSAGSSELLMEDGFPKESISEAGEMAHGTTSSSVVNQQVQKRANSPRLPQSIPSKRSKEELMRGSQSLSSSWPQKNGHTIS

SEQ ID NO: 128 - protein, 865 aa, Gossypium hirsutum
MSRLGFKSMVYHGDFFLGELNTIPITDSNFQFPNNEIRIHHISPTSERCIPLSILHTISSFPVRCKLESPFPVEQPHLIHLHASCFYEFKTAVVLVGDEEVHLVAMPSKQKKFPCFWCFSVSTGLYNSCLGMLNLRCLAIVFDLDETLIVANTMKSFEDRIEVLRGWIARESDPIRLSGMSAELRRYIDDRLLLKQYAESDCVVDNGKMFKVQMEEVPPLSDGHEKVVRPVIRLQDRNIVLTRINPEIRDTSVLVRLRPAWDELRSYLTAKGRKRFEVYVCTMAERDYALEMWRLLDPGAHLIGSKQLLDRVVCVKSGSRKSLLNVFRDGKCHPQMAMVIDDRSKVWEDKDQPRVHVVPPFAPYYAPQAETANAVPVLCVARNVACNARGLFFKEFDDNVLRKMSEVFYEDEVVNLPLAPDVSNYLMSEEASFASNGNNGAPICEGMNGAEVERRMNQSEEKHVLDSSTRPVTSNPELRSETSQPPVTDIVGPASSVAPLPSQKPSILGAPGLLSNPMMLGASVRRDNNGSEGDYDMKRRALGIKQSLDLRNQSSIQPPLLPKFPIQTSSSSVVPQGGWLVEEDINESHLNDRPSGTTQESDVIKSDKLRGYQNPFPHTAPGSVSTGLPSYASQVKIEEARTGLDTPKQNVLPTAHLSEIGGTQNHLPSITRELQSEGGKMNLLPSHLSIGVLHEIGRRCGSKVEFRSVVSTSKDLQFSVEVLFTGEKIGVGMGKTRRDAQQQAAELALHNLAEKYVAYIAPRSGAVDRDFNNLSLGTENGFLWDVNPASNEAIKEGFPKDNTSEAAEEPGSNSSSIANQPVEKRANSPRLSESMPSKRSKEGVLRRLGSSLSSSRQPKNEHTIS

SEQ ID NO: 129 - protein, 771 aa, Citrus sinensis
MSRLGYKNIVYHGDVCLGELDTILVSDENFQFPNNEIRIRHISPSSERCIPLSILHTISSFSLRCKLESSAPVEQPHLINLHASCFYEFKTAVVVIGDEEIHLVAMPSKQKKFPCFWCYSVSSGLYNSCLGMLNLRCLAIVFDLDETLIVANTMKSFEDRIEALRSWIAREPDQIRASGMSAELKRYMDDRTLLKQYTENDCVMDNGKVFKVQQEEVPPPSENYERIVRPVIRIPERNLVLTRINPENRDTSVLVRLRPAWEDLRSYLIAKGRKRFEVYVCTMAERDYALEMWRLLDPEGHLIGSKQLLDRVVCVKSGSRKSLLNVFQRGLCHPKMAMVIDDRCKVWEDKDQPRVHVVPAFTPYYAPQAETANAVPVLCVARNVACNVRGCFFKEFDENLLRSISEVFYEDEAVNLPAAPDVSNYLMSEDANFAPNGSTNAPMSEGLNGLEVERRLNQSDEKYVVDSGLPSMKNSSDLKSETSLLPVAVASNATVPATVVPSQKPGLLGAPIRRDNSSMKHGFDLRNQNSAQPPLPKLHGQGGWIVEEEVNRVLPNNRPVSIATGLPSHASQAKGEEAIMAHDLHKQNLPPASQPPEIGVSQNHVSSNSREFQTEGGKTNLLPSYLSIGVLQEIGKRCSSKVEFRSVVSTSKDLQFSVEVLFTGEKIGVGMGKTRKDAQQQAAENALHYLAEKYVAYITPRSGAMDRDFDKLSLENENGFLWDTIISESNEGLGEDGLRKESTPEASEVEPGSTYASLGNQQVQKRPNVPK

SEQ ID NO: 130 - protein, 830 aa, Populus trichocarpa
MSPLGFKSLVYHGEVCLGELDVIPVTEKGFLFPNNEIRIHHISQQSERCPPLSILQTISPFSVRCKLESSSPMEQSHLINLHASCFHEVKTAVVLIGDEEIHLVAMPSKQKKFPCFWCFAVPVGLYDSCLRMLNMRCLSIVFDLDETLIVANTMKLFEDRIETLRGWIGREQDPMRVSGMIAEMKRYIDDRLLLKQYIENDVVMDNGKMYKVQLEDVLRSSDGHERVVRPVIRLPEKNIVLTRINPEIRDTSVLVRLRPAWEDLRSYLTAKGRKRFEVYVCTMAERDYALEMWRLLDPEAHLIASKQLSDRVVCVKSGSKKSLLNVFQDGMCHPKMAMVIDDRLKVWEDMDQPRVHVVPAFTPYYAPQAETANAVPVLCVARNVACNVRGCFFKEFDEILIRRISEVFYEDEVKSLPPTPDVSNYMMTEDSGFVPNGNNIAPFSEGMSGIEAERRLHQSDGKTVMESAPHSITNSAELRTEISQPNVAIIPNVAGPTLSATLLPSQKPSLLGAPVRRDLRNQNSGQPPLLSRIPAAIPLSSLQPQGGWLVEEDTSRAQSNNRPSATAQELDSLKSDKLRGLQNPLAHGASASAPAPSSLVSHSSELKVEEAIAGNDMHKQIVPAGEAGMSQNHVSSSSREFQAEAGKLNLLPSHLSIGVLQEIGRRCRSKVEFKSVVSTSKDLQFSVEVLFTGEKIGVGMGKTRKDAQQQAAENALRSLAEKYAAYVSPNSGAVDGDFDKLSIGNENGFVWDISSPESSDLVREDGSAKERPSEVADAELLNTSNMVNQQQQKRANSPRSPQSIPSKRSKEELCGSQSLPFRQSKNESHV

SEQ ID NO: 131 - protein, 272 aa, Arabidopsis thaliana
MSMSQSRAVQRSSSPNEDRGENQLVVYDLKGNDDTEEEVLPVQSQPLSSRTQCPSIGAFTVQCASCFKWRLMPSMQKYEEIREQLLENPFFCDTAREWKPDISCDVPADIYQDGTRLWAIDKPNISRPPAGWQRLLRIRGEGGTRFADVYYVAPSGKKLRSTVEVQKYLNDNSEYIGEGVKLSQFSFQIPKPLQDDYVRKRPARLLDSIDNTNTPVAKEANPLAWISPDDHISLQLGTPTESGLNNSHYQPSKKKKTSTLSIFGSNDELADR

SEQ ID NO: 132 - protein, 309 aa, Glycine max
MHRGKFSLTPKNEVKDLTGSSLTSYKHSSLLDPIDVSSSSDDENDLPNDDVSNQLVLYDPVANGNNAIELAPDPLQCEHPLLPRSKPSHSVPRVLPSVGAFTVQCASCFKWRLIPTKEKYEEIREHILEQPFVCQKAREWRPHVSCDDPEDISQDGSRVWAIDKPNIAQPPAGWERLLRIRAEGSSKFADIYYIAPSGKRLRSMVEIQKFLMEHPEYTRDGVTLSQFSFQIPRPLQENYVRKRPARLTSSYEVSEPVEHEQVSPLAWVDPEGHGRRLGLPAPPFMESHDIDPISISRPSKRQATHKGAL

SEQ ID NO: 133 - protein, 297 aa, Zea mays
MESSKSPQSSKNSRIVVPSDSNRSRFDNDGFSSETASNQMVVFNSEAADELGENRLQKSVITKGISPSIGAFTVQCAKCFKWRLIPTKEKYEEIRERIIQEPFVCKRAREWRPDITCNDPEDISQDGSRLWAIDKPNIAQPPHGWERQIRIRGEGGTKFADVYYTSPTGRKLRSLVEVDRFLQENPEHVAQGVTLAQFSFQIPRPLRQDYVKKKPKLINASDEASTITSKSSEPEEVNPIAWAVPTKHEGDASEEASFADETLASEVVLTRKRKIGSSLSVEPNHLSDELEPKLEDA

SEQ ID NO: 134 - protein, 315 aa, Oryza sativa
  source 1..315: note = Japonica Group
MGNSKTPQPSKKSRIMLSDTDGHQLDNDEFSSESASNQMVLFNPETVAKGQDELGENHSPSLQKSANNPNRGMPSIGAFTVQCAKCFKWRLIPTKEKYEEIRECIIQEPFECERAREWRPDVTCNDPEDISQDGSRLWAIDKPNIALPPPGWERQIRIRGEGGTKFADVYYTSPTGRKLRSLVEIDRYLLENPDYVAQGVTLTQFSFQIPRPLRQDYVKKRPKIVNPNDEASVVTTKSVKPEEVSPIAWAAPSVHQEGEAGERASHADEPPEAEELELTRKRKAESPLFEEAHSNHVSDEPKTKLEDTQNGGPSA

SEQ ID NO: 135 - protein, 308 aa, Sorghum bicolor
MESSKSPQSSKNSQITVPSDSNGPRFDNDGFASETASNQMVVFNSEAGDKEQDEPGENRLQKSVITRGISPSIGAFTVQCAKCFKWRLIPTKEKYEEIRERIIQEPFVCKRACEWKPGVTCNDPEDISQDGSRLWAIDKPNIAQPPRGWERQIRIRGEGGTKFADVYYTSPTGRKLRSLVEIDRFLQENPEYVAQGVTLAQFSFQIPRPLRQDYVKKKPKLINPSDEASTITSKSFQPEEVNPIAWAVPTAHEGDASEEASLADETLPSEVVLTRKRKAESSSSVEPNHLSDELEPKLADAQNGETSN

SEQ ID NO: 136 - protein, 342 aa, Cucumis sativus
MVGGEENDLNEPSLSNDQETPRQDVIAVSSSSSSLSSSSSENELHETRNENVQSQSNDNPSRELVIYDPSADDGHANDPVSTPVKQQPSPVLGHLTPNSSRVLPSVGAFTVQCAGCFKWRLIPTKEKYEEIREHLLEKPFYCETARGWRPDISCDDPADITQDGSRLWAIDKPNIAQPPPGWQRLLHIRGEGSTKFADVYYTAPSGKKLRSMVDIQKYLIEHPEYQTEGVSMTRFSFQIPKPLREDYVRKRRPNMNASCDASKLESGEVRPISWARPDDSTALQLGKPGLTELLSTSPVNNPVTRPAKKVKRALAQEMRRSSLTSTEPPAIKLERCFDQYEK

SEQ ID NO: 137 - protein, 354 aa, Vitis vinifera
MRHPGKIYRKANKEENDLTGSSLPRYLDALQVPIDLSSSEEEEEDFYAEEDDDNHSYENTSNQLVVYDPVANSATEVEPVPDPIQYRLPSFTGYSTTNAPSRVLPSVGAFTVQCANCFKWRLIPTKEKYEEIREHILEQPFVCETAREWRPNVSCDDPADISQDGSRLWAIDKPNIAQPPAGWQRLLRIRGEGSTKFADVYYVAPSGKRLRSMVEVQKYLLEHPEYMVHGVTLSQFSFQIPKPLQENYVRKRPARVNAGSYDDTTSSGMSRPLEPGEVNPLSWAGPAECIDLQLRTPGLSAPYFEAPVFDPVSRPTKQARRTLPRDMYDSNMVYNQQKVKVEEPNQSRAGDYDP

SEQ ID NO: 138 - protein, 340 aa, Gossypium hirsutum
MQSPPEKAFVMLKREATDLAGSSFTSHVDSTFQDAVNAAASSASSSSSDDDSQVTQNEDDLYNGNTSKQLVLYDPAVSSTVGCTPPGPIQCRPPVGPRFSSSKLLPSVGAFTVQCANCFKWRLIPTKEKYEEIREHILENPFVCETAREWRPDISCDDPTDISQDGSRLWAIDKPNIAQPPPGWQRLLRIRGEGSTKFADIYYQAPTGKRLRSMVEVQKYLIEHPEYATEGVNLSRFSFQIPKPLQEDYVRKRPARQTASHDNEVNPLAWAGPEDTEVQQGRLALPPPSVEASVSDSPYRPAKHARRTPSEHMYPSNQVPNKHGSKVEMGSELRNTNYGD

SEQ ID NO: 139 - protein, 340 aa, Citrus sinensis
MQSHSGKASVRVEKEANEFLGSSLLSNLDESLHEPIDVPSSTSSSASEGQSDETVEGDDQINGDASKQLVLYDPTANGTSEIEPVPESFQYNRAPFGRYSGPNTQSRVLPSVGAFTVQCADCFKWRLIPTKEKYEEIREHVLENPFTCEKAREWRPDVSCDDPTDISQDGSRLWAIDKPNIAQPPPGWQRLLRIRGEGSTKFADVYYEAPSGKKLRSMVEIQKYLLEHPEYARAGVKMSQFSFQIPKPLQENYVRKRVSKAHTSHDTPKALEPRAVSPLSWVSPDNFTDLQLGRPALPAPPVEAPISDPNPRPAKQARRAPKQMYSGSPISSDHKFRAEE

SEQ ID NO: 140 - protein, 348 aa, Populus trichocarpa
MQSHPGETFHVLKKEGDDCTGSRYTGHLNSSLQEPIEVSSSSNEDNSDDTWDIDDQSIEDSSKQLVLYDPLANDAGEIEPVPQPILSHHPFRRYSDLNVPSRVLPSVGAFTVQCAKCFKWRLIPTKQKYEELREHILEEPFFCETAREWRPDISCDDPTDIDQDGSRLWAIDKPNIAQPPPGWQRLLRIRGEGSTKFADVYYQAPSGKRLRSMVEIQKYLIEHPEYMRDGLTLTRFSFQIPKPLQENYVRKKRPRLSASCDDARPLEPGEANPLKWVGPGDCTELQLGRPAILPPPLIQSSAYLPFYWPVKKKARTPSKQSHRTNPVCNLDEPKVEEPDQSRNSGCDL

SEQ ID NO: 141 - protein, 693 aa, Arabidopsis thaliana
MDKSIPIKAIPVACVRPDLVDDVTKNTSTIPTMVSPVLTNMPSATSPLLMVPPLRTIWPSNKEWYDGDAGPSSTGPIKREASDNTNDTAHNTFAPPPEMVIPLITIRPSDDSSNYSCDAGAGPSTGPVKRGRGRPKGSKNSTPTEPKKPKVYDPNSLKVTSRGNFDSEITEAETETGNQEIVDSVMMRFDAVRRRLCQINHPEDILTTASGNCTKMGVKTNTRRRIGAVPGIHVGDIFYYWGEMCLVGLHKSNYGGIDFFTAAESAVEGHAAMCVVTAGQYDGETEGLDTLIYSGQGGTDVYGNARDQEMKGGNLALEASVSKGNDVRVVRGVIHPHENNQKIYIYDGMYLVSKFWTVTGKSGFKEFRFKLVRKPNQPPAYAIWKTVENLRNHDLIDSRQGFILEDLSFGAELLRVPLVNEVDEDDKTIPEDFDYIPSQCHSGMMTHEFHFDRQSLGCQNCRHQPCMHQNCTCVQRNGDLLPYHNNILVCRKPLIYECGGSCPCPDHCPTRLVQTGLKLHLEVFKTRNCGWGLRSWDPIRAGTFICEFAGLRKTKEEVEEDDDYLFDTSKIYQRFRWNYEPELLLEDSWEQVSEFINLPTQVLISAKEKGNVGRFMNHSCSPNVFWQPIEYENRGDVYLLIGLFAMKHIPPMTELTYDYGVSCVERSEEDEVLLYKGKKTCLCGSVKCRGSFT

SEQ ID NO: 142 - protein, 673 aa, Glycine max
MEEGSCQNSVPGSGFVDKTRIVEAKPLRSLAPVLPKSLQSSLSGRYPSVFPPFVLFEEPQESQPSPAPMPAPIRSYRDPLDEEEAPHGANGETSSPMEGLNGCNVDDGQKRVVLPMNSCKSSQKRSKKTKESPFDLSSSVGGISMAKREDGDREVVNLVLMTYDSLRRRLCQIEDAKELNTTMLIKRADLRASNAMTGKAFRTNMRRRVGAVPGVEIGDIFFLRMEMCLVGLHGQSMSGIDYMTIKDELQEETVALSIVSSGVYDNDAEDNDVLIYSGQGENFNKKDKHVIDQKLQRGNLALDRSSRRHNEVRVIRGLRDAANRNAKIYVYDGLYKIQDSWIERGKSGGGVFKYKFVRLSGQPSAFAVWKSVQKWKMGSSTSSRTGLILADLSTGVESIPVSLVNEVDNEKGPSFFTYFHSLKDPKPFSLLQSSHGCNCNKTCVPGDLSCSCIQRNEGDFPYTANGVLVSRKPLVHECGPLCKCSPNCKNRVSQTGLKHQMEVFKTKDRGWGLRSLDPIRAGTFICEYAGEVIDVAKVNKNRGYDDEYVFDTSRIYDPFKWNYEPSLLEEISSNVSCEDYDIPSPLIISSKKFGNVARYMNHSCSPNVFWQPVLYAENNQSFLHIAFFALRHIPPMTELTYDYGCSSHADHSSAPKGRKKCLCGSSKCRGSFG

SEQ ID NO: 143 - protein, 857 aa, Zea mays
MAQQMASVHLNDTTVVDAKPLRTLTPMFPAPLGLHTFTPQNSPSVVCVTPFGPYAGGTELGMPAVPPMFAAPVPAAGAEPCQRQPHTANMNGGDTELGMPAVPPMFAAPAPAAGAEPNQGQPHTANMNGTAVNSSVTSLQTPPSATTQESGKRKRGRPKRVPDTTVSSAPSAPLAPTISPVPSLPVVTPVPSAPQELNTIVSLTPSSDAPQESGKRKRGRPKRIQDVPVMVPLTSQGDITSVIQTLPGPSVHESGTRRGRPKRLQDSSDIVTPIDSKDSESSPQPPIAAPAESGKRKRGRPKRIDGSVTPSSHSVFSIDDDAVDTMKRGRPRKIDTKLLQLPSLFSDDPRQTADNVLMMFDALRRRLMQMDDVKQAAKQQPNLKAGSIMNNAELRVNKDKRIGEVPGVEVGDMFYFRIEMCLVGLNSQSMAGIDYMSAKFGNEEDPVAISVVSAGVYDNTGDDPDVLVYTGHGMSGKDDQKLERGNLALERSLHRGNPIRVIRSVKDLTCPTGKIYIYDGLYKIKEAWVEKGKSGFNVFKHKLLREPGQPDGIAVWKKTEKWRENPSSRDHVILLDISYGVESNPVCLVNEVDDEKGPSRFTYTTNLTYGNSLSSMRKMQGCKCISVCLPGDNNCSCTHRNAGDLPYSASGILVSRMPVLYECGDSCTCSQNCRNRVVQKGTQIRFEVFKTGDRGWGLRSWDPIRAGTFICEYAGEIIDINRVNGEDDYIFETSPLEQNLRWNYAPELLGEPSLSDSNETPKQLPIVISAKRTGNIARFMNHSCSPNVFWQPVLYDHGDEGYPHIAFFAIKHIPPMTELTYDYGQNQRNSIQMGTHSGFRKSKSCLCWSPKCRGSFG

SEQ ID NO: 144 - protein, 813 aa, Oryza sativa
  source 1..813: note = Japonica Group
MAGTRQTTSVPMDNAAVVDAKPLRTLTPMFPAALGLHTFTAKENSSSIVCITPFGPYAGGTEQAMPASIPPMFASPAAPADPNQRQPYAVHLNGAAPANGTANNTGVIPDLQIAVAGTVESAKRKRGRPKRVQDSSVPSAHLVPSAPGGNITAVQTPPSATTDESGKKKRGRPKRVQDVPVLSTPSAPQVDSTVFQTPASAVNESVTRKRGRPRRVQDGADTSAPPIQSKYNEPVLQTPSAVTLPEDGKRKRGRPKRVPDGALIPLSHSGVSIDDDSGEIITGKRGRPRKIDVNLLNLPSLFSDDPRESVDNVLMMFDALRRRLMQLDEVKQGAKQQHNLKAGSIMMSAELRANKNKRIGEVPGVEVGDMFYFRIEMCLVGLNSQSMSGIDYMSAKFGNEEDPVAISIVSAGVYENTEDDPDVLVYTGQGMSGKDDQKLERGNLALERSLHRGNQIRVVRSVRDLTCPTGKIYIYDGLYKIREAWVEKGKTGFNVFKHKLLREPGQPDGIAVWKKTEKWRENPSSRDHVILRDISYGAESKPVCLVNEVDDEKGPSHFNYTTKLNYRNSLSSMRKMQGCNCASVCLPGDNNCSCTHRNAGDLPYSASGILVSRMPMLYECNDSCTCSHNCRNRVVQKGSQIHFEVFKTGDRGWGLRSWDPIRAGTFICEYAGEVIDRNSIIGEDDYIFETPSSEQNLRWNYAPELLGEPSLSDSSETPKQLPIIISAKRTGNIARFMNHSCSPNVFWQPVLYDHGDEGYPHIAFFAIKHIPPMTELTYDYGQSQGNVQLGINSGCRKSKNCLCWSRKCRGSFG

SEQ ID NO: 145 - protein, 830 aa, Sorghum bicolor
MAQQMASVPLNDAAVVDAKPLRTLTPMFPAPLGLHTFTHQNSPSVVCVTPFGPYAGGTELGKPAVPPMFAAPTPAAEAEPSQRQLDTANMNGAAHANGTAVNSLVTPLQTPPSAATQESGKRKRGRPKRVLDTTVASVPSAPLATTIPPVPSLPVVTPVPSAPQEGNTIVSLTPSSDAPQESGKRKRGRPKRVQDVPVMAPPTSQGDSTPVIQTLPGPSVHEFGTRKRGRPKRLQDSSDIATPIDSKDLPISSPQPLSAAAPAESGKRKRGRPKRVLDGSATPSSHSGFSIDGDAVDTMKRGRPRKIDTNLLQLPSLSSDDPRQTADNVLMMFDALRRRLMQMDDVKQVAKQQPNLKAGSIMINAELRVNKNKRIGEVPGVEVGDMFYFRIEMCLVGLNSQSMAGIDYMSAKFGNEEDPVAISVVSAGVYDNTEDDPDILVYTGQGMSGKDDQKLERGNLALERSLHRGNPIRVIRSVKDMTCPTGKIYIYDGLYKIKEAWVEKGKSGFNVFKHKLLREPGQPDGIAVWKKTEKWRENPSSRDHVILLDISYGVESNPVCLVNEVDDEQGPSHFTYTTKLTYGNSLNSMRKMQGCKCISVCLPGDNSCSCTHRNAGDLPYSASGILVSRMPVLYECGDSCTCSYNCRNRVVQKGTQIRFEVFKTGERGWGLRSWDPIRAGTFICEYAGEIIDRNSVTGEDDYIFETSPSEQNLRWNYAPELLGEPSLSDSNETPKRLPIVISAKRTGNIARFINHSCSPNVFWQPVLYDHGDEGYPHIAFFAIKHIPPMTELTYDYGQNHHPNIQMGTHSSFGKSKSCLCWSPKCRGSFG

SEQ ID NO: 146 - protein, 713 aa, Cucumis sativus
MEQQLDQDSIPVGSLDKSKVLNVKPLRQLVPVFPSAQNVSSFSTPQGAAPFVCAGPSGPFPPGVAPFYPFFFSPAEQNQHTPGGTTNTNASFGLNSPISTAVPISSFRTPTEGTSTQNTGSRKNTRSRAQLQDGYSDSQNDNSQYYGMGVNDGEDSSKVGRKNKAKKKTRNGQDINFTSDVDIDAMLNEMVSTYNLSVLDSNRQAHGTIEAVSCVLMVFDLLRRKISQVEESKEPMPGSIRRPDLKTGAFLMTKGIRTNINKRIGTVPGVEIGDIFFFRMELCLVGLHAPSMAGIDYMGLKVSQDEEPVAVSIVSSGGYEDDTNDTDVLIYSGQGGVNRKDKESIDQKLERGNLALEKSLHRGNDVRVIRGVRDFSNPTGKIYVYDGLYKIQESWVEKGKSGCNVFKYKLVRLPGQQEAFLNWKLVQQWKDGNVSRIGVIIPDLASGAESLPVSLVNDVDDEKGPAYFTYYAGLKYLKPVYSMEPSAGCNCAGGCLPGNINCLCMQKNGGYLPYSSNGVLASQQSMIYECGASCQCPPNCRNRVSQGGLKFRLEVFRTKGKGWGLRSWDPIRAGAFICQYAGEVIDSPKAKDSVRDNEDGYIFDATRSYPNLEVISGDSDGPPKLQFPLVISAKNAGNVARFMNHSCYPNVYWKPIIRENKGEHDVHIAFHAIRHIPPMMELTYDYGVIPPESADGRKINCLCGSLKCRGYFC

SEQ ID NO: 147 - protein, 716 aa, Vitis vinifera
MDGGSGHGFVPPSASFDKSRVLDIKPLRSLVPVFPNPPQAPPFVCSPPFGPFPPGFTPFYPFSVAQGPQSSPELNQHKTPTGATNHETPISASANLFRTPPHFPGVVNGDAETSREYGVQFLNENSNMGVKQDGFFDDPKRAAPHLRASNSSRKKAKKSKDVDISLTVDNEKGSSKNFVMRFDSLQLDDGNREMVNYVLMTFDALRRRLSQIEEAKESPGGGIKRADLKAANILMSKGVRTNMRKRIGVTPGVEVGDIFFFRMEMCLAGLHAQSMAGIDYMFVKGGLEEEPVAVSIVSSGGYDDDADDADVLIYSGQGGNVNRKDKQVADQKLERGNLALDRSFHRANEVRVIRGVKDVVNPLSKVYVYDGLYTIQESWTEKGKSGCNMFKYKLVRIPGQPGAFAHWKSIQKWKEGFSSRIGLILPDLTSGAESIPVSLVNDVDDEKGPAHFTYFPTLRYSKSFNLKHPSFGCNCQNACLPGDLNCSCIRKNGGDFPYTSNGILVARRPLVHECGPTCPCIPNCKNRMSQTGLKVRLEVFKTNNRGWGLRSWDPIRTGTFICEYAGEVLDKVKVYQERDEGESNEYLFDTTHVYDNAFKWNHEPGLLDEEPSAEPNEYYDIPSPLIISAKYVGNVARFMNHSCSPNVFWQPVLYEHNNESFLHIAFFAIKHIPPMTELTYDYGMLQSENYEVQSNHTPNGKKKCLCGSSNCRGYYG

SEQ ID NO: 148 - protein, 585 aa, Gossypium hirsutum
MPIRSFRAEPPPASNGENVQSSNKRKSVGPSFVKKKVKRSNDSELALAALTNFKPGISAAEKDDGNRELVENVLMRFEALRRKLSQMEDAKESHSDIFKRANLKAGNIMFTKGVRTNGKKRIGAVPGVEIGDIFFFRMELILIGLHSQSMAGIDFMPMKADIEGERVAISIVSSGGYEDNAEDPDVLVYTGQGGNASADKEASDQKLVRGNIALERSLHRANEVRVIRGFKDATHQTSKVYVYDGLYKVQESWMEKGKTGCNMFKYKLVRLPGQTGAFSTWKSIRKWKEDPSSRDGLILPDLTSGAESIPVSLVNEVDDEKGPAYFTYVSTVKYPKSFKLVQPSYGCNCRDACQAGNSNCSCIQKNGGDFPYITTGILACRMPMIFECGSSCPCFRNCKNRVLQTGFKVHFEVFKTRDKGWGLRSWDPIRAGTFICEYAGEVIEKIKEKADGDDGENNDYVFDTNRVYESFKWNHETESAEERSDTSEKFDIPSPLIISSKNSGNIARFMNHSCSPNVFWQPIMYEHNNEAFLHIAFFAKKHIPPMTELTYDYGIPRSDETESNNMEHGKKKCLCGSPKCRGYFY

SEQ ID NO: 149 - protein, 662 aa, Citrus sinensis
MDGTVPSAPLDKTKVLDVKPLRSLRPVLPSSPQAPPFVCAPPFGPFPPGFSPFYPFSTPEFTPDNNQNNNTQTPPTSFATPIRSFRSPDVNFVDGSNGDLGSSDGFLDGKKRRASSYKQKRPKNAQDSDFSVGISSFERDDGNRQVVNNVLMRFDALRRRISQIEDAKETSTGLIRRADLKASNILMSKGVRTNMRKRLGVVPGVEIGDIFFFRMEMCLIGLHSQSMAGIDYMITRSDLDEEPVAVSIISSGGYDDDAEDSDILIYSGQGGNANRKGEQAADQKLERGNLALERSLRRASEVRVIRGMKDAINQSSKVYVYDGLYMVQESWTEKGKSGCNIFKYKLVRIPGQPGAFALWKLIQRWKDGMSGRVGLILPDLSSGAEAIPIALINDVDDEKGPAYFTYLTTVKYSKSFRLTQPSFGCNCYSACGPGNPNCSCVQKNGGDFPYTANGVLVSRKPLIYECGPSCPCNRDCKNRVSQTGLKVRLDVFKTKDRGWGLRSLDPIRAGTFICEYAGEVVDKFKARQDGEGSNEDYVFDTTRTYDSFKWNYEPGLIEDDDPSDTTEEYDLPYPLVISAKNVGNVARFMNHSCSPNVFWQPIIFENNNESFVHVAFFAMRHVPPMTELTYDYGISKSDGGNYEPHRKKKCLCGTLKCRGYFG

SEQ ID NO: 150 - protein, 673 aa, Populus trichocarpa
MEGGGSGHNSTPRIDKTRVLDVKPLRTLAPVFPSSSEAPPFVSTSPFGPHSSGFAPFYPFSAPQATQATPDLNQHTHATPAAPLRSFRGTESNGDAFNGEAGSFDGSKGSAKRKTKSSLQKRARKSQDLDFTLSVENNFVVGVSLSERDDGNGEVVHSIRMRFDALRRRLSQLEDAKESPVGIIRRADLKAGNILMTKQVRTNMRKRIGAVPGVEIGDIFFFRIEMCLLGLHAPSMAGIDYMSLRNDLEEEPLAVSIVSSGYYEDNAEDKDVLIYSGQGGAANKDKGATDQKLERGNLALERSLRRGNEVRVIRGMKDSVNQASKVYVYDGLYRVQESWVEKAKSGCNIFKYKLVRIPGQPDAFGVWKSIEKWKEGLSSRAGLILPDLTSGAESTAVSLLNDVDEEKGPAYFTYVSTVKYSKSFKLTQPAYGCNCPNACQPGNLNCSCIRKNEGNFPYTANGVLVCRAPMIDECGPTCPCFPNCKNRVSQTGLKVRLEVFKTKDRGWGLRSWDPIRAGTFICEYAGEVVEKVSQPGEEGDGDDYVFDTSRVYESFRWNYEPGLVEEDSSIEAIEEPKVPSPLVISSRNVGNVARFMNHGCYPNVFWQPIMYEHNSESFIHIGFFAMRHIPPMTELTYDYGKSCVGEAEADGGSTPRGRRKCLCGAPRCRGYFA

SEQ ID NO: 151 - protein, 646 aa, Arabidopsis thaliana
MADGHSFNNISLSGRGGKNPGLLKINSGGIQWKKQGGGKAVEVDRSDIVSVSWTKVTKSNQLGVKTKDGLYYKFVGFRDQDVPSLSSFFQSSYGKTPDEKQLSVSGRNWGEVDLHGNTLTFLVGSKQAFEVSLADVSQTQLQGKNDVTLEFHVDDTAGANEKDSLMEISFHIPNSNTQFVGDENRPPSQVFNDTIVAMADVSPGVEDAVVTFESIAILTPRGRYNVELHLSFLRLQGQANDFKIQYSSVVRLFLLPKSNQPHTFVVISLDPPIRKGQTMYPHIVMQFETDTVVESELSISDELMNTKFKDKLERSYKGLIHEVFTTVLRWLSGAKITKPGKFRSSQDGFAVKSSLKAEDGVLYPLEKGFFFLPKPPTLILHDEIDYVEFERHAAGGANMHYFDLLIRLKTDHEHLFRNIQRNEYHNLYTFISSKGLKIMNLGGAGTADGVAAVLGDNDDDDAVDPHLTRIRNQAADESDEEDEDFVMGEDDDGGSPTDDSGGDDSDASEGGVGEIKEKSIKKEPKKEASSSKGLPPKRKTVAADEGSSKRKKPKKKKDPNAPKRAMSGFMFFSQMERDNIKKEHPGIAFGEVGKVLGDKWRQMSADDKEPYEAKAQVDKQRYKDEISDYKNPQPMNVDSGNDSDSN

SEQ ID NO: 152 - protein, 640 aa, Glycine max
MTDGHLFNNITLGGRGGTNPGQIKIYPGGIIWKRQGGGKLIEVDKSDIMGVTWMKVPRSNQLGVQIKDGLYYKFTGFRDQDVVSLTNFFQNTCGISVEEKQLSVSGRNWGEVDLNGNMLAFTVGSKQAFEVSLADVSQTQLQGKNDVILEFHVDDTTGANEKDSLMEISFHIPNSNTQFVGDENRPPAQVFRDKIMSMADVGAGGEDAIVTFEGIAILTPRGRYSVELHMSFLRLQGQANDFKIQYSSVVRLFLLPKSNQPHTFVIISLDPPIRKGQTLYPHIVMQFETDYVVESELAINEDLYNTKYKDKLDLSYKGLIHEVFTTILRGLSGAKVTKPGKFRSCQDGYAVKSSLKAEDGILYPLEKSFFFLPKPPTLILHEEIDYVEFERHAAGGSNMHYFDLLIRLKSEQEHLFRNIQRNEYHNLYEFISSKGLKILNLGDAQPTVGIKKVLENDDDDAVDPHLERIKNEAGGDESDEEDSDFVADKDDEGSPTDDSGADDSDATDSGDEKEKPAKKESKKDLPSKASTSKKKSKDDEDGKKRKQKKRKDPNAPKRAMSGFMFFSKLERENLKKTNPGISFTDVSRVLGEKWKKLSVEEKEPYEAKAREDKKRYKDEISGYKNPQPMNIDSGNESDSA

SEQ ID NO: 153 - protein, 639 aa, Zea mays
MTDGHHFNNILLGGRGGTNPGQFKVHSGGLAWKRQGGGKTIEIDKADVTAVTWMKVPRAYQLGVRIKAGLFYRFIGFREQDVSNLTNFIQKNMGVTPDEKQLSVSGQNWGGIDIDGNMLTFMVGSKQAFEVSLPDVAQTQMQGKTDVLLELHVDDTTGANEKDSLMDLSFHVPTSNTQFVGDESRPPAHILWETILKFADVGSSEEPVVTFEGIAILTPRGRYSVELHLSFLRLQGQANDFKIQYSSIVRLFLLPKSNNPHTFVVITLDPPIRKGQTLYPHIVIQFETEAVVERDLALSKELLVEKYKDRLEESYKGLIHEVFTKVLRGLSGAKVTRPGSFRSCQDGYAVKSSLKAEDGLLYPLEKGFFFLPKPPTLILHEEIEFVEFERHGAGGASISSHYFDLLVKLKNDQEHLFRNIQRNEYHNLFNFINGKNIKIMNLGGDGQGASGVVTDVLRDTDDDAVDPHLERIKNQAGDEESDEEDEDFVADKDDSGSPTDDSGDEESDASDSGGEKEKSSKKEASSSKPVQKRKHKGRDDEGQEKKKPKKKKDPNAPKRAMTPFMYFSMAERGNMKSSNPDLPTTEIAKKLGEMWQKMSGEEKQPYIQQAQVDKKRYEKESAVYRGEATVDVDSGNESD

SEQ ID NO: 154 - protein, 641 aa, Oryza sativa
  source 1..641: note = Japonica Group
MTDGHLFNNILLGGRAGSNPGQFKVYSGGLAWKRQGGGKTIEIEKSDLTSVTWMKVPRAYQLGVRTKDGLFYKFIGFREQDVSSLTNFMQKNMGLSPDEKQLSVSGQNWGGIDINGNMLTFMVGSKQAFEVSLADVSQTQMQGKTDVLLEFHVDDTTGGNEKDSLMDLSFHVPTSNTQFLGDENRTAAQVLWETIMGVADVDSSEEAVVTFEGIAILTPRGRYSVELHLSFLRLQGQANDFKIQYSSIVRLFLLPKSNNPHTFVVVTLDPPIRKGQTLYPHIVIQFETEAVVERNLALTKEVLAEKYKDRLEESYKGLIHEVFTKVLRGLSGAKVTRPGSFRSCQDGYAVKSSLKAEDGLLYPLEKGFFFLPKPPTLILHEEIEFVEFERHGAGGASISSHYFDLLVKLKNDQEHLFRNIQRSEYHNLFNFINGKHLKIMNLGDGQGATGGVTAVLRDTDDDAVDPHLERIKNQAGDEESDEEDEDFVADKDDSGSPTDDSGGEDSDASESGGEKEKLSKKEASSSKPPVKRKPKGRDEEGSDKRKPKKKKDPNAPKRAMTPFMYFSMAERGNMKNNNPDLPTTEIAKKLGEMWQKMTGEEKQPYIQQSQVDKKRYEKESAVYRGAAAMDVDSGSGGNESD

SEQ ID NO: 155 - protein, 639 aa, Sorghum bicolor
MTGGHHFNNILLGGRGGTNPGQFKVYSGGLAWKRQGGGKTIEIDKADVTAVTWMKVPRAYQLGVRIKDGLFYRFIGFREQDVSSLTNFIQKNMGVTPDEKQLSVSGHNWGGIDIDGNMLTFMVGSKQAFEVSLADVAQTQMQGKTDVLLELHVDDTTGANEKDSLMDLSFHVPTSNTQFVGDENRPPAHILWETILKFADVGSSEEPVVTFDGIAILTPRGRYSVELHLSFLRLQGQANDFKIQYSSIVRLFLLPKSNNPHTFVVITLDPPIRKGQTLYPHIVIQFETEAVVERDLALSKELLVDKYKDRLEESYKGLIHEVFTKVLRGLSGAKVTRPGSFRSCQDGYAVKSSLKAEDGLLYPLEKGFFFLPKPPTLILHEEIEFVEFERHGAGGASISFHYFDLLVKLKNDQEHLFRNIQRNEYHNLFNFINGKNIKIMNLGGDGQGASGVVTDVLRDTDDDAVDPHLERIKNQAGDDESDEEDEDFVADKDDSGSPTDDSGDEDSDASDSGGEKEKSSKKEASSSKPVQKRKPKARDDEGHEKKKAKKKKDPNAPKRAMTPFMYFSMAERGNMKSSNPDLPTTEIAKKLGEMWQKMSSEEKQPYIQQAQVDKKRYEKESAVYRGEATADVDSGNESD

SEQ ID NO: 156 - protein, 642 aa, Cucumis sativus
MADGQLYNNISLGGRGGTNPGQLKTDQRGIQWKKQGGGKAIEVDKADIVGVTWMKVPRSNQLGIRVKDGLYYKFIGFRDQDISSLTKFFQSNCGIAPEEKQLSVSGRNWGEVDLNGNMLTFLVGSKQAFEVSLADVAQTQLQGKNDVMLEFHVDDTTGANEKDSLMEISFHIPNTNTQFVGDESRPPAQVFRDKIMSMADVSAGIEEAVVTFEGIAILTPRGRYSVELHLSFLRLQGQANDFKIQYSSVVRLFLLPKSNQPHTFVVVTLDPPIRKGQTLYPHIVLQFETDYVVQSTLQIGDELFNTKYKDKLEPSYKGLIHEVFTTILRGLSGAKITRPGKFRSCQDGYAVKSSLKAEDGVLYPLEKSFFFLPKPPTLILHEEIDYVEFERHAAGGSNMHYFDLLIRLKTEQEHLFRNIQRNEYHNLFDFISGKGLKIMNLGDAQARDGVAAVLQEDDDDAVDPHLERIRNEAGGDESDEEDSDFVADKDDGGSPTDDSGGDDSDGSASGGEKEKPGKKEAKKDPSASKAPAKKKSREGADDGSKKKKQKKKKDPNAPKRAISGFMFFSKMERENIKKSNPGISFTELGRVLGDKWNKMSAEEKEPYESKARDDKKRYKEEISGYKNPQPMNIDSGNESDSA

SEQ ID NO: 157 - protein, 644 aa, Vitis vinifera
MSEGHLFNNISLGGRGGTNPGQLRVHPGGILWKKQGGGKAVEVDKSDIVGVTWMKVPRTNQLGVRVKDGLYYKFTGFREQDVTNLTNFFQHSCGLNPEEKQLSVSGRNWGEVDLNGNMLTFLVGSKQAFEVSLADVSQTQMQGKNDVILEFHVDDTTGANEKDSLMEISFHIPNSNTQFVGDENRPPAQVFRDKIMSMADVGAGGEEAVVTFEGIAILTPRGRYSVELHLSFLRLQGQANDFKIQYSSVVRLFLLPKSNQPHTFVVVTLDPPIRKGQTLYPHIVMQFETDYVVQSELSLSEELLNSKYKDKLEPSYKGLIHEVFTLILRGLSGAKVTKPGKFRSCQDGYAVKSSLKAEDGVLYPLEKSFFFLPKPPTLILHEEIDYVEFERHAAGGSNMHYFDLLIRLKTEQEHLFRNIQRNEYHNLFDFISGKGLKIMNLGDVQTADGVAAVLQNDDDDAVDPHLERIKNEAGGDESDEEDEDFVLDKDDGGSPTDDSGEEESDASESGGEKEKPSKKESKKEPSVSKASSSKKKPKDGDEDGSKKRKQKKKKDPNAPKRAMSGFMFFSQTERENIKKSTPGIAFTEVGRVLGDKWKKMTAEEKEPYEAKAQADKKRYRDEISGYKSNPQPMNVDSGNESDSE

SEQ ID NO: 158 - protein, 639 aa, Gossypium hirsutum
MTDGHLFNNISLGGRGGTNPGQLKIYSGGILWKKQGGGKAVEVDKTDILGVTWMKVPRTNQLGVRIKDGLYYKFTGFRDQDVASLTSFFQNNCGIAPEEKQLSVSGRNWGEVDLSGNMLAFMTGAKQAFEVSLADVSQTQLQGKNDVILEFHVDDTTGANEKDSLMEISFHIPNSNTQFVGDENRPPAQVFREKIMSVADVGAGVEEAVVTFEGIAILTPRGRYSVELHLSFLRLQGQANDFKIQYSSVVRLFLLPKSNQPHTFVVVTLDPPIRKGQTLYPHIVMQFETDYVVQSTLSINEDLLNTKYKDRLEPSYKGLIHEVFTTVMRGLSGAKVTKPGKFRSCQDGYAVKSSLKAEDGVLYPLEKSFFFLPKPPTLILHEEIDYVEFERHAAGGSNMHYFDLLIRLKTEQEHLFRNIQRNEYHNLFDFISSKGLKIMNLGDVRTTDGVAEILQNDDDDAVDPHLERIKNEAGGDESDEEDEDFVLDKDDGGSPTDDSGEEDSDASDSGDEKEKPAKRDPRKEAASSKATKKKSKDGQDDGKKKKRRKKDPNAPKRAMSGFMFFSQVERENIKKTNPGASFAEVSRILGEKWKKLSEEEKEPYEAKAKVDKRRYDVERNAYKNPQPMNIDSGNESDSE

SEQ ID NO: 159 - protein, 642 aa, Citrus sinensis
MTDGPSFNNISLGGRGGTNPGQLKIYLGKISWKKLGGGKAVEVDKVDIAGVTWMKVPRTNQLGVRTKDGLYYKFTGFRDQDVATLTNFFQSNFGISPEEKQLSVSGRNWGEVDLNGNMLTFMVGQKQAFEVSLADVSQTQLQGKNDVILEFHVDDTTGANEKDSLMEISFHIPNSNTQFVGDENHPPAQVFRDKIMSMADVGAGGEEAVVTFEGIAILTPRGRYSVELHLSFLRLQGQANDFKIQYSSVVRLFLLPKSNQPHTFVVVTLDPPIRKGQTLYPHIVLQFETDYVVQSELLMSEELLNTKYKDKLELSYKGLIHEVFTTILRGLSGAKITKPGKFRSAQDGYAVKSSLKAEDGVLYPLEKSFFFLPKPPTLILHEEIDYVEFERHAAGGSNMHYFDLLIRLKTEQEHLFRNIQRNEYHNLFDFISGKGLKIMNLGDMKTTDGVAAVLQEDDDDAVDPHLERIKNEAGGDESDEEDSDFVADKDDGGSPTDDSGEEDSDASESGGEKEKPAKKESKKESSSVKASTSKKKSRDGDEDGKKKKQKKKKDPNAPKRAMSGFIFFSQMERENIKKSNPGIAFTDVGRVLGERWKKMSVEEREPYESKARADKKRYKDEISGYKNPKPMDIDSGNESDSA

SEQ ID NO: 160 - protein, 639 aa, Populus trichocarpa
MADAHLFNNISLGGRGGTNPGQLKISSGGIQWKKQGGGKAVEVDRTDIVGVTWMKVPRTNQLSVLIKGGPWYKFTGFRDQDLSTLTNFFQSHGITPEEKQLSVSGRNWGEVDLNGNMLTFLVGSKQAFEVSLADVSQTQMQGKNDVILEFHVDDTTGANEKDSLMELSFHIPNNNTQYIGDENHPPAQVFRDLIVQRADVGAGGEEAVVTFEGIAILTPRGRYSVELHLSFLRLQGQANDFKIQYSSVVRLFLLPKFNQPHTFVVVTLDPPIRKGQTLYPHIVLQFDTDFVVQSNLSMSEDLLYTKYKDKLEPSYKGLIHEVFTTVMRGLSAAKVTRPGKFRSCQDGYAVKSSLKAEDGVLYPLEKSFFFLPKPPTLILHEEIDYVEFERHAAGGSNMHYFDLLIRLKTEQEHLFRNIQRNEYHNLFDFISGKGLKIMNLGDMQTTKGVAAVLQNDDDDAVDPHLARIRNEAGDDESDDEDEDFVAGKDDGGSPTDDSGDGESDASESGDEKENPTKKDFKREASSSKATTKRKSRDGEESQKKRKPKKKKDPNAPKRSKSAYMFFSQMERENVRKSNPGIVFGEIAKALADKWNAMSAEEKEPYEEMARNDKKRYKLQVNDYKNPQPMMVDSENESDS

SEQ ID NO: 161 - protein, 1074 aa, Arabidopsis thaliana
MADSRNGNARAPPSGVPPKAGNTYSIDVKNFISRARALYEHWKKHSADLWGSADALAIATPPASDDLRYLKSSALNIWLLGYEFPDTIMVFTKKQIHFLCSRNKASLLEVVKKPAHDELKLDVIMHVKPKGDDGTGLMDAIFRAIRDLSRGDGNDSQVVGHIAREAPEGKLLETWTERLKNANFQFVDITGGLSDLFAVKDDTEVMSVKKAAYLAYSVMKNVVVPNLESAIDEEKDVTHSALMDLTEKAILEPTKASVKLKPENVDICYPPIFQSGGKFDLKPSAASNDELLTYDPASIIICAVGARYNSYCSNVARTYLIDATSLQSKAYEVLLKAHEAAIDALRSGRKINTVYQAALSVVEKNAPEFVDKLTKSAGTGIGLEFRESGLNINAKNDKVLRPKMAFNVSLGFQNLECESESRSKNKKFSLLLADTVLVTDQKPELLTKCSKSVKDVAYSFKEDEEEEKPRKKARTSGSENYITKTALRSDDHVVSKEELRKQHQAELARQKNEETARRLAGDSSGAGDSRSTAKTSADVVAYKNVNDMPHKELMIQVDTRNEAVLLPIYGSLVPFHVATIRTVSGNQDTNRNCYIRIIFNVPGTPFNPHDSNSLKNQGAIYLKEVSFRTKDSRHSSEVTQQIKTLRRQVMARESERAERATLVTQEKLQLAGNKFKPLRLSELWIRPPFSGRKKIPGTLEAHANGFRYSTTRPDERVDVLFANIKHAFFQPAEKEMITLLHFHLHNHIMVGTKKTKDVQFYVEVMDVVQSLGGGRRSAYDPDEIDEEQRERDRKNKINMDFNHFANRVNDMWQLPQFASLDLEFDQPLRELGFHGVPHKTSAFIIPTSSCLVELIEYPFLVVSLSEIEIVNLERVGFGQKNFDMAIIFKDFKKDVLRVDSVPTSSLEGIKEWLDTTDIKYYESKLNLNWRQILKTITDDPQSFIDDGGWEFLNLDGSDSESGGSEESDKGYEPSDVEVESESEDEASESESLVESDDDEEEDSEQESEEEKGKTWDELEREATNADREHGVESDSEEERKRRKMKAFGKSRPGTSGGGGSSSMKNMPPSKRKHR

SEQ ID NO: 162 - protein, 1064 aa, Glycine max
MADHRNGSAQAANGKASAAGTAYSIDLNAFQTRLRSFYKHWDAHKTDLWGSSDAIAVACPPPSEDLRYLKSTALNLWLLGFEFPETIMVFSKKQIHILCSQKKASILESVKKTAREAVDADLVLHVKPRNDDGTALMDAIFRALSKSDGRDTPTVGYISREAPEGKLLEMWTEKLKNTKFQLNDVANGLSSLFAAKNNEELTSIKRAAYLTTSVMKNFVVPKLENVIDEEKKVSHSTLMEDTEKAILEPSKVNCKLKADNVDICYPPIFQSGGEFDLKPSAVSNDELLHYDSASVILCAIGARYKSYCSNIARTFLIDADPLQSRAYGVLLKAHEAVIGSLKPGNRLSATYQAAVSVVENEAPDLISYLTKSAGTGIGIEFRESGLNINAKNEQIVKEGMVFNVSLGFQNVQRESTKSKSKHFSLLLADTVIINKDKTEVVTSMSSKALKDIAYSFNEDEEEENPSAKADANGAEPLMSKTTLRSDNHEMSKEELRRQHQAELARQKNEETARRLAGGGNETGDNRSSSRTSAELVAYKNINDLPPPREMMIQIDQKSEAVLLPINGSMVPFHVAFIRTVSSQQDTNRNCYIRIIFNVPGTPFSPHDSNSMKFQGSIYLKEASFRSKDSRHISEVVQSIKTLRRQVVARESERAERATLVTQEKLQLANNRFKPIRLSDLWIRPAFGGRGRKIPGTLEAHVNGFRYSTTRQDERVDIMFGNIKHSFFQPAENEMITLLHFHLHNHIMVGNKKTKDVQFYVEVMDMVQNVGGGKRSAYDPDELEEEQRERDRKNKINVEFQTFVNRLNDLWGQPQFNGLDLEFDQPLRELGFPGVPHKSSVFIVPTSACLVELIETPFLVVTLSEIEIVNLERVGLGQKNFDMTVVFKDFKRDVLRIDSIPSTSLDGIKEWLDTTDIKYYESRLNLNWRQILKTITDDPQSFIEGGGWEFLNLEATDSESENSEESDKGYEPSDVEPESDSEDEASDSESLVESEDDGEEDSEEDSEEEKGKTWEELEREASNADREKGNESDSEEDRKRRKAKSFGKSRGAGLSSSMTKRPKLR

SEQ ID NO: 163 - protein, 1067 aa, Zea mays
MVALVNVSLLTNMADNGDAKGGSGAYAINIENFSKRLKVFYDHWKEHKSDLWGSSDAIAIATPPPSDDLRYLKSSALDIWLLGYEFPETIIVFMHKQIHVLSSQKKANLIGTLKKAANEAVGVDIVLHVKTKNSDGADLMDDIVHAARNQSKSDKPVVGHIAKEAPEGKLLETWIKKLSGSGLRLVDVTNGFSELFAVKDTTEITCVKKAAYLTSSVLKNFVIPKLEKVIDEEKEVSHSSLMDDAEKAILDPLKVKVKLKPDNVDICYPPVFQSGGKFDLKPGASSNDEYLYYDSASIIICAIGSKYSSYCSNVARTYLIDATPTQNKAYETLRKAHEAAIQQVKPGNQMSAVYQAAVAVIERDAPELLPNLTKSAGTGIGLEFRESGLNLNAKNDRRIKKGMVFNVSLGLHNIQAETTSEKTKQFSLLLADTVLVNERGHEVLTAPCSKAFKDVAYSFNEDDDAVAAEVKIKSKTIDVMPTKATLRSDNQEMSKEELRRQHQAELARQKNEETARRLAGVGTGSGDGRGPARASNELVAYKNVNDVPFVRDLVIQVDQKNEAVLLPIYGSMVPFHVSTVKSVTSHQDNRTCTIRIFFNVPGMPFSNDSKFNSQGAIYLKEITFRSKDPRHSSEVVQQIKTLRRQVASRESERAERATLVTQEKLQIGSNRMKMMRLSDVWIRPAFGGRGRKLTGNLEAHFNGFRYSTSRSDERVDIMFGNIKHAFFQPAEKEMITLLHFHLHNHIMVGNKKTKDVQFYVEVMDVVQTLGGSRRSALDPDEIEEEQRERDRKNRINMDFQNFVNKVNDHWSQPQFKGLDLEFDVPLRELGFHGVPYKASAFIIPTSTCLVELIETPFLVVSLSEIEIVNLERVGFGTKNFDMAIVFKDFKKDVLRIDSIPSASLDAIKEWLDTTDLKYYESRLNLNWRPILKTIIDDPQKFIDDGGWEFLNMEASDSETEDTEESDQGYVPSDAEPESESEDDDSDSESLVESDDDDEESDEDSEEEKGKTWEELEREASNADREHGAESDSEEERRRRKAKTFGKSRAPERSSFKGAPPSKKPKFR

SEQ ID NO: 164 - protein, 1056 aa, Oryza sativa
  source 1..1056: note = Japonica Group
MADNGNAKPGGGGSGAYTINLDNFSKRLKVFYDHWKEHNSDLWGSSNAIAIATPPPSEDLRYLKSSALDVWLLGYEFPETIIVFMHKQIHFLCSQKKANLIGTLKKAANDAVGADIVLHVKAKNDSGVGLMEDIVRAVCAQSKSDDPIVGHIAKEAPEGKLLEAWADKLSSSSVQLTDITNGFSELFAMKDTSEITCVKKASYLTSSVMKNFVVPKLEKVIDEERKVTHSSLMDETEKAILDPLKVKVKLKAENVDICYPPVFQSGGKFDLKPGASSNDDYLYYDSASVIICAIGARYGNYCSNMARTFLIDATPTQIKAYETLMKAHEAALEALKPGNRMSAVYQAAVDVIEKNAPELLRNLTKSAGTGIGLEFRESGLNLNPKNDRIIKAGMVFNVSLGLHNLQAEKKSEKTKQYSLLLADTCLVPLENLTASCSKLVKDVAYSFNDEDEVLPVKKVEVNAKEALPPTKATLRSDNQEMSKEELRRQHQAELARQKNEETARRLAGVGSGSGDGRGPSRSSNELVAYKNVNDVPYARELVIQVDQKNEAVLLPIYGSMVPFHVSTVKSVTSHQDNRTCTIRIFFNVPGMPFSNDSNLKSQGAIYLKEITFRSKDPRHSSEVVQQIKTLRRQVASRESERAERATLVTQEKLQLTSNRNKPVRLSDVWIRPAFGGRGRKLTGTLESHVNGFRYSTSRADERVDIMYGNVKHAFFQPAEKEMITLLHFHLHNHIMVGNKKTKDVQFYVEVMDVVQTLGGNRRSALDPDEIEEEQRERDRKNRINMDFQNFVNKVNDHWSQPQFKGLDLEFDVPLRELGFHGVPYKASAFIIPTSTCLVELIETPFLVVTLSEIEIVNLERVGFGTKNFDMAIVFKDFKKDVLRIDSIPSTSLDAIKEWLDTTDLKYYESRLNLNWRPILKTIIDDPQKFIDDGGWEFLNMEASDSETEETEESDQGYEPSDAEPESESEDEDSDSESLVESDEDDEDDSEEDSEEEKGKTWEELEREASNADRENGAESDSEEERRRRKVKTFSKSRPPPERSSFKGGPSKKPKFR

SEQ ID NO: 165 - protein, 1054 aa, Sorghum bicolor
MADNGNAKGGSGAYTINLENFSKRLKVFYDHWKEHKSDLWSSSDAIAIATPPPSDDLRYLKSSALDIWLLGYEFPETIIVFMHKQIHVLCSQKKANLIGTLKKAANESVGVDIVLHVKTKNGDGADLMDHIVQAARNQSKSDKPVVGHIAKEVPEGKLLETWTEKLSGSGVRLTDVTNGFSELFAVKDTTEVTCVKKAAYLTSSVLKNFVVPKLEKVIDEEKKVSHSSLMDDTEKAILDPLKVKVKLKPDNVDICYPPVFQSGGKFDLKPGASSNDEYLYYDSASVIICAIGSKYSSYCSNVARTYLIDATPKQSKAYETLLKAHEAAVQEVKPGNQMSAVYQAAVAVIEKDAPELLPNLTKSAGTGIGLEFRESGLNLNAKNDRKIKQGMVFNVSLGLHNVQAETTSEKTKQFSLLLADTVLVNERGHDILTAPCSKAVKDVAYSFNEDDEDVAEVKMESKTIDVMPTKATLRSDNQEMSKEELRRQHQAELARQKNEETARRLAGVGSGSGDGRGPARASNELVAYKNVNDVPFVRDLVIQVDQKNEAVLLPIYGSMVPFHVSTVKSVTSHQDNRTCTIRIFFNVPGMPFSNDSKLNSQGAIYLKEITFRSKDPRHSSEVVQQIKTLRRQVASRESERAERATLVTQEKLQIGNNRMKMMRLSDVWIRPAFGGRGRKLTGNLEAHFNGFRYSTSRSDERVDIMFGNIKHAFFQPAEKEMITLLHFHLHNHIMVGNKKTKDVQFYVEVMDVVQTLGGSRRSALDPDEIEEEQRERDRKNRINMDFQNFVNKVNDHWSQPQFKGLDLEFDVPLRELGFYGVPYKASAFIIPTSTCLVELIENPFLVVSLSEIEIVNLERVGFGTKNFDMAIVFKDFKKDVLRIDSIPSASLDAIKEWLDTTDLKYYESRLNLNWRPILKTIIDDPQKFIDDGGWEFLNMEASDSETEDTEESDQGYVPSDAEPESESEDDDSDSESLVESDDDDEESDEDSEEEKGKTWEELEREASNADREHGAESDSEEERRRRKAKTFSKSRAPERSSFKGAPPSKKPKFR

SEQ ID NO: 166 - protein, 1073 aa, Cucumis sativus
MADRRNGNSQPSHGKASGAGNTYDIDLVNFSTRLKSLYSHWGEHKSDMWSSSDVLTIGTPPASEDLRYLKSSALHIWLFGYEFPETVIVFTKKQIHFLCSQKKVSLLDVVKKSAFDAVGADVVMHVKAKNDDGSSLMDSIFRAIRAQSKADGMENPVVGYIAREAPEGKLLETWSGKLKNANFELVDITNGLSDLFACKDDTEIMNIKKAAFLTVSVMNRVVVPKMENVIDEEKKITHSSLMDETEKAILEPTKAGVKLKTENVDICYPPIFQSGGVFDLRPSAASNDELLHYDPASVIICAVGSRYKSYCSNIARTFLIDANTLQSKAYEVLLKAQEVAISMLRPGNKVNAAYTAALSVVKKESPELVPNLTKSAGTGIGLEFRESGLNLNAKNDRIVKAGMVFNVSLGFQKLTPTDKLQSSAGKTKNQNFSLLISDTVIVGKEKTEVLTAPSSKSFKDIAYSFNEDEEEEEKLKVKSEANGKEAVVSKTTLRSDNHEISKEELRRQHQAELARQKNEETARRLAGVGNGAGDNRSSMRTAADLVAYKSVNDLPPQRDLMIHIDQKNETVLLPIYGSMVPFHVATIRTVSSQQDTNRTCYIRIIFNVPGTPFSPHDANSLKFQGSIYLKEVSFRSKDPRHISEVVQLIKTLRRQVVARESERAERATLVTQEKLQLAGNRFKPIRLPELWIRPAFGGRGRKLPGTLEAHLNGFRYATTRSEERVDIMFGNVKHAFFQPAENEMITLLHFHLHNHIMVGNKKTKDVQFYVEVMDVVQTIGGGKRSAYDPDEIEEEQRERDRKNKINMDFQSFVNRVNDLWGQPQFGGLDLEFDQPLRELGFHGVPYKSSAFIVPTSTCLVELIETPFLVVTLGEIEIVNLERVGFGQKNFDMTIVFKDFKRDVLRIDSIPSTSLDGIKEWLDTTDIKYYESKLNLNWRQILKTITEDPQSFIDEGGWEFLNLEATDSESENSEESDKGYEPSDVEPESDSEEDDSDSASLVESEDEEEEDSDGDSEEEKGKTWEELEREASNADREKGDESDSEEERKRRKMKTFGKFRAGPSGNAPKRPKMR

SEQ ID NO: 167 - protein, 1071 aa, Vitis vinifera
MAEHRNGNAKPSDGKASGAASPYAINLDNFTKRLKTLYSHWKEHSSDLWGSSDALAIATPPASDDLRYLKSSALNIWLLGYEFPETIMVFMKKQIHFLCSQKKASLLEVVRKSAKEAVGVEVVMHVKAKSDDGTGLMDAIFRAVRANSISHDTPVVGHIGREAPEGKLLEMWTEKLKNADFQLSDITNGFSDLFAMKDSTELTNVKKAAFLTSSVMKHFVVPKLEKVIDEEKKVSHSSLMDDTEKAILEPARVKVKLKAENVDICYPPIFQSGGEFDLRPSASSNDENLYYDSTSVIICAIGSRYNSYCSNVARTFLIDANAMQSKAYEVLLKAHEAAIGALKPGNKVSAAYQAALAVVEKDAPELVSNLTKSAGTGIGLEFRESGLNLNAKNDRVLKPGMVFNVSLGFQNLQTDTNNPKTQKFSVLLADSVIVGEKGPEVVTSISSKAVKDVAYSFNEDDDEEEEERPKVKPEANGGEAVSSKATLRSDNQEMSKEELRRQHQAELARQKNEETARRLAGGGSGAGDNRGAVKATGDLIAYKNVNDLPPPKELMIQVDQKNEAILLPIYGSMVPFHVATVKSVSSQQDTNRTCYIRIIFNVPGTPFSPHDSNSMKFQGSIYLKEVSFRSKDPRHISEVVQMIKTLRRQVASRESERAERATLVTQEKLQLAGTRFKPIRLSDLWIRPSFGGRGRKLTGSLESHTNGFRYSTSRPDERVDIMYGNIKHAFFQPAEKEMITLLHFHLHNHIMVGNKKTKDVQFFVEVMDVVQTLGGGKRSAYDPDEIEEEQRERDRKNKINMDFQNFVNRVNDLWGQPQFKGLDLEFDQPLRELGFHGVPHKASAFIVPTSSCLVELIETPFLVITLSEIEIVNLERVGLGQKNFDMTIVFKDFKRDVLRIDSIPSTSLDGIKEWLDTTDLKYYESRLNLNWRPILKTITEDPEKFIEDGGWEFLNLEVSDSDSENSQESDQGYEPSDVQSDTGSEEEGDDSESLVESEDDVEEDSDGDSEEEQGKTWEELEREASNADREKGDESDSEEERKRRKMKAFGKARVPEKRSTRGSLPKRPKLR

SEQ ID NO: 168 - protein, 1065 aa, Gossypium hirsutum
MADHRGANGQAPNGTAPGTGSVYSINLDNFSKRLKALYSHWNEHKPELWGSSDVFAVATPPTSEDLRYLKSSALNVWLLGYEFPETIIVFTKKQIHFLCSQKKVSLLEVVKKSAKEAVGADVVMHVKAKSDDGIASMDSIFRSIRVQYKGNENDAPNFGYIAREAPEGKLLETWAEQLKSAGFQLTDVTNGLSDLFAVKDKEEVMNVKKAAYLSYNVMNNIVVPRLESVIDEEKKITHATLMDETEKAIVNPQLAKVKLKPENVDICYPPIFQSGGEFDLRPSAASNEENLYYDSASVILCAVGARYNSYCSNIARTFLIDATPLQSKAYEVLLKAHEAAIGMLKPGRKCSAAYQAALSIVEKEAPDWIPNLTKSAGTGIGLEFRESGLNLNGKNDRLVKAGMVFNVSVGFQNLQCESKKPKSSVFSLLLADTVIVGEQNTEVVTGKCSKAVKDVAYSFNEDEEEEEKSVKAEANGFEPFMSKTVLRSDNHEISKEELRRQHQAELARQKNEETARRLAGGPGNGDNRSVAKSSTDLIAYKNVNDLPPPRDFMIRIDQKNEAVLLPIYGSMVPFHVATIRTVSSQQDTNRNCFIRIIFNVPGTPFSPHDLNSSKNQGAIYLKEVSFRSKDPRHISEVVQQIKTLRRQVMARESEKAERATLVTQEKLQLAGNRFKPIRLSDLWIRPVFGGRGRKIPGTLEAHVNGFRYSTTRADERVDIMYGNIKHAFFQPAEKEMITLVHFHLHNHIMVGNKKTKDVQFYVEVMDVVQTLGGGKRSAYDPDEIEEEQRERDRKNKINMDFQSFVNRVNDLWGQPQFNGLDLEFDQPLRELGFHGVPYKASAFIVPTSSCLVELVETPFLVVTLSEIEIVNLERVGLGQKNFDMTIVFKDFKKDVLRIDSIPSTSLDGIKEWLDTTDIKYYESRLNLNWRQILKTITDDPQSFIENGGWEFLNLEASDSDSEQSEESDQGYEPSDVEPESESEDDDSDSESLVESEDEEEEESDDDSEEEKGKTWEELEREASNADREKGNESDSEEDRRRRKMKAFGKSRAPPSSSIPKRSKLR

SEQ ID NO: 169 - protein, 1065 aa, Citrus sinensis
MADNRNGNAQMANGTGGANAYSINLENFSTRLKALYSHWNKHKSDYWGSADVLAIATPPASEDLRYLKSSALNIWLLGYEFPETVMVFMKKQIHFLCSQKKASLLGMVKRSAKDVVGVDVVIHVKAKTDDGVELMDAIFNAVRSQSNVDSGDGPIVGSIARETPEGRLLETWADRLQNSGFQLSDMTNGLSELFAVKDQEEIMNVKKAGYLTYNVMNKIVVPKLENVIDEEKKVTHSLLMDEAEKAILEPTKAGVKLRAENVDICYPPIFQSGGAFDLRPSAASNDELLYYDSGSVIICAVGSRYNSYCSNIARSFLIDATPLQSKAYEVLLKAHEAAIGALKPGNKVSAAYQAALSVVEREAPELVPNLTKSAGTGIGLEFRESGLNLNAKNDRVVKAKMIFNVSIGFQNLQNQTNKPKNQMFSLLLADTVIVGENNPEVVTCKSSKAVKDVAYSFNEDEEEEEKPKVKAEANGTEALPSKTTLRSDNQEISKEELRRQHQAELARQKNEETGRRLAGGGSGAGDNRASAKTTTDLIAYKNVNDLPPPRDLMIQIDQKNEAVLFPIYGSMVPFHVATIRTVSSQQDTNRNCYIRIIFNVPGTPFNPHDTNSLKHQGAIYLKEVSFRSKDPRHIGEVVGAIKTLRRQVMARESERAERATLVTQEKLQLAGNRFKPIKLHDLWIRPVFGGRGRKIPGTLEAHLNGFRFATSRPEERVDIMFGNIKHAFFQPAEKEMITLVHFHLHNHIMVGNKKTKDVQFYVEVMDVVQTLGGGKRSAYDPDEIEEEQRERARKNKINMDFQSFVNRVNDLWGQPKFNGLDLEFDQPLRDLGFHGVPHKASAFIVPTSSCLVELIETPFLVVTLGEIEIVNLERVGLGQKNFDMTIVFKDFKKDVLRIDSIPSSSLDSIKEWLDTTDIKYYESRLNLNWRQILKTITDDPQSFIDDGGWEFLNLEASDSESENSEESDQGYEPSDMEVDSVTEDEDSDSESLVESEDEEEEDSEEDSEEEKGKTWAELEREATNADREKGDDSDSEEERKRRKGKTFGKSRGPPSGGFPKRTKLR

SEQ ID NO: 170 - protein, 1082 aa, Populus trichocarpa
MADQRNGTGQPSNAARNAYAIDVEKFKTRLKALYSNWNENKADLWGSSDVVAIATPPPSEDLRYLKSSALNIWLLGYEFPETVMVFMKKQIHFLCSQKKASLLEVVKKPAREVVGVDVVMHVKAKTDNGTGLMDAIFHAIYAQSSADGKDTPVVGHIAREAPEGIILETWAEKLKGEGFELADVTSGLSDLIAVKDADELINVKKAAFLTFSVMNNVVVPKLENVIDEEKNITHSALMDEAEKAILDPTRAKAKLKADNVDICYPPIFQSGGEFDLRPSAASNDEPLYYDSASVIIIAVGSRYNSYCSNVARTLMIDATPLQSKAYAVLLKAHEAAIGALKPGNKVSAAYQAALSVVEEEAPELVPNLSKSAGTGIGLEFRESGLNLNAKNDRVVKAKMVFNVSLGFQNLQNQIDNPKIRNFSLLLADTVIVGDQNPDVVTSKSSKAVKDVAYSFNEGEEEEQKPKARAEVNGGENLMSKTTLRSDNGEISKEELRRQHQAELARQKNEETARRLAGGGSAKGDNRAASKTSTDLVAYKNVNDIPPARDLMIQIDQKNEAVLLPIYGNMVPFHVSTIRTVSSQQDTNRTCYIRIIFNVPGAAFNPHDSNSLKHQGAIYLKEVSFRSKDPRHISEVVQLIKTLRRHVVARESERAERATLVTQEKLQLAGNRFKPIRLTDLWIRPVFTGRGRKLPGALEAHVNGFRFSTSRSEERVDIMFSNIKHAFFQPAEKEMITLLHFHLHNHIMVGNKKTKDVQFYVEVMDVVQTLGGGKRSAYDPDEIEEEQRERDRKNKINMDFQSFVNRVNDLWSQPQFSGLDLEFDQPLRELGFHGVPHKVTSFIVPTSSCLVELVETPFLVVTLGEIEIVNLERVGLGQKNFDMTIVFKDFKRDVLRIDSIPSTSLDGIKEWLDTTDIKYYESRLNLNWRQILKTITDDPQSFIDDGGWEFLNLEASDSDSDNSEDSDQGYIPSDAEPESESEDDVSDSESLVESEDDEEEEDEEDSEEEKGKTWEELEREASNADREKGDDSDSEEERNRRKVKTFGKSRPAPRPAPRPAPRPAPRPPPRTVPGSMPKRPKFR

SEQ ID NO: 171 - protein, 819 aa, Arabidopsis thaliana [sequence omitted]

SEQ ID NO: 172 - protein, 1058 aa, Glycine max
MKQLKLAAESHAKEDNPCRHKPEMENTLESSGSPRHRKISARWDPDEACQPIVDEAPVFYPTIEEFEDTLGYIAKIRPQAEPYGICRIVPPACWVPPCPLQEKDLWENAKFPTRIQQIDLLQNREPMRKKIRGRKRKRRKQSKMGMGMRTAKSGSEANVASEPEEKFGFQSGSDFTLKDFQQYANVFKDCYFGLNDANEYEKVSDSSHQQRWKPSVEEIEGEYWRIIEQPTDEVEVYYGADLETGSLGSGFPKTSSLTKNESDRYALSGWNLNNFPRLPGSALCFEGSDISGVVVPWLYVGMCFSSFCWHVEDHHLYSLNYLHWGDPKVWYGVAGSHAPGLEDAMRKHLPDLFEEQPNLLNELVTQLSPSILKSEGVPVHRTIQHSGEFVVTFPRAYHCGFNCGFNCAEAVNVAPVDWLVHGQNAAELYSLQCRKTSLSHDKLLFGCAQEAMHALAELTLHGKENLKYIKWRSACGKDGVLTKAVKTRITMEKERLDCLPTHLKMLRMDSKFDLFEERECFSCFYDLHLSAIGCKCSPDCYSCLKHSNLFCSCEMDNRFILFRYTMNELSTLVEALEGESHAIEVWANRNSGMVSANAEDACIYKQDVESAICQTQSYKEGKNSTCAGTNDKSNSTILSSSYSHISAELVHSEFHHETFSAPYGTKDCHKDNLNEKDLVMDNKVMVEKGGSVDLNIDVMSGEPENYFLHAADYHHNKGVPYVEKVSFAEARKEQDNMEPGADCIAAKEFSSCSRDVQNSCTLDGYKLFGVDLQMHSDSGEQLNSVSKIGDAETSNTSISLTNQSFLMQKFGISVEPVNLGSVICGKLWCSKHAIYPKGFKSRVKFFSILDPPRICNYLSEVYDAGFLGPIFKVTMEELPNEAFTNTSADKCWESVLDRLNHEIKRQRSQGEIELPSLELLQSINGHKMFGFLSPSIIQAIEAEDPNHQCVEYWNHKEVVSESSGSAIDDCKFSHGSSNSLGDAKTKLFDAGLIRQEQDSIIGSYDSFEEMKLVLQGFLKKASSDELSAMHKLFSSDAQFTKCRAEFVSLIEEIQKACA

SEQ ID NO: 173 - protein, 1239 aa, Zea mays
MLSAMMGIEGIAATVTEDPEPSIPPGFGPFAASASWGIQNDVKPADDHSSSVLALQSIDDDVEILEYLSSSVDHQSDTGCCISGSNTCRKSLRNRPPIDYSRFDQIADEDSDAEVADKGVNAVKHRQQFPKGVLRGCPECANCQKVIARWNPSGARRPVLDEAPVYYPTEEEFQDTLKYIEIIRPTAEPYGICRIVPPASWKPPCLLKEKNIWECSKFSTRVQKVDKLQNRKSPKKSRRGGMMKKRRKISETEENNHHQIGMQQNQERFGFEPGPEFTLQMFQKYADDFSDQYFMKDKCRDSPPSVEDIEGEYWRIVERPTEEIEVIYGADLETGTFGSGFPKLCPEMKSDVEDKYAQSGWNLNNLPRLQGSVLSFEGGDISGVLVPWLYVGMCFSSFCWHVEDHHLYSLNYMHWGAPKMWYGVPGKDAVNLEAAMRKHLPELFEEQPDLLHNLVTQFSPSLLKSEGVPVYRCVQHEGEFVLTFPRAYHAGFNCGFNCAEAVNVAPIDWLPVGQNAVDLYREQARKITISHDKLLLGAAREAIRAQWDILFLKRNSSVNLRWKSICGPDSTICKSLKARIEMELVQRQNISSPCQSRKMDSEFDSTDRECALCYYDLHLSASGCPCSPEKYACLVHAKQLCSCDWDKRFFLFRYDVNELNILADALGGKLSAIHRWGVSDLGLSLSSCVKREKVQDSKTVRRLTDGPRRSYMSQASTVSLIPSSVSTEQKKNEDKTLDLGCPGMNLPKISPEANNLRPSTEQIKSENLSQLKEPCVKNELSCPTSNGTSQEHKGGIGGHKLAAASMMVLSGQSFPANAGESVRNAHVLAVFKEGRDCTSSLTLREYHNRPVSMIDNGANMKLDLENIDNSHMLMSSPDFNATVCHSYKDQTFLTLETNTSVMTEKDSSQARNASQQFVSTALRTQNVSQEPLCTAIAPKQLIDPQVQKKSYGVFGSGSAHLGHLTVGNQQLNERWHQRQSDSLSSVEVRARGHSAMIVQPALENHSRNGVAQKGPRIANVVHRFKCSVEPIEIGAVLSGKLWSSSQAIFPKGFKSRVKYFSVVDPVQMTYYISEILDAGQQGPLFMVTVENCPGEIFINISPTKCWNMVRERLNMEIRRQLNMGRANLPTLQPPGSVDGHEMFGLLTPAIVQAIEARDRDYICTEYWRSRPHATIENRDNQNMSPQDPPLVALRGLFQRANCDELRALRSLLMSNRILGDNSRQQACQILDEEIAKQWH

SEQ ID NO: 174 - protein, 984 aa, Oryza sativa
  source 1..984: note = Japonica Group
MEIASAAAHQISPMVSSRDPGEEASAPPPPPPRRGEKRRMRGRTPSPEPASAPQDLCPSGACGDNVAGATTTNGKWHPHESYRPEIDDAPVFTPTEEEFKDPIRYITSIRPQAEKYGICRIVPPSSWRPPCSLKEKNFWECTEFNTRVQQVDKLQNREPTKKKSQPRVQKKRKRRKRLRFGMTHRRPSANTSEDCADADEKFGFQSGSDFTLDEFQKYADEFKQQYFGIKGSDEIPLSEIKKKKKNWQPSVDEIEGEYWRIVVCPTDEVEVDYGADLDTSMFSSGFSKLSSDSNRRDPYGLSCWNLNNLPRIPGSVLSFETEDISGVVVPWLYVGMCFSSFCWHVEDHFLYSMNYMHFGEPKVWYGVPGADAVKLEEAMRKNLPRLFEEQPDLLHELVTQLSPSVLKSEGVPVYRVVQNPGEFVLTLPRAYHSGFNCGFNCAEAVNVAPVDWLPHGQCAVELYREQRRKTSISHDKLLLKTANEAVRQLWMNLSDCKSEQGVYRWQDTCGKDGMLTSAIKTRVKMEKAARGGNMALRYKKMDGDYDSADRECFSCFYDLHLSAVSCQCSPNRFACLNHANILCSCEMDRKTALLRYTIEELHTLVAALEGDPTAVYQWGQNDLGLVCPSGSTQYKKMDLGENTEFPDSATNVNHGCSLGSQDQYHYDPAKPAGYQQEKGIQIASEKHDKNKMVVNLESPATASNPSRSKSDCSGSLSLNHSSELPSSRIQTGNSTLASITTEKLFGVDIKSNLAQSSDGQVSQLAKPSSSQTDEVSKPAIAKYTVELLDSGTMMIGKKWCNQQAIFPKGFKSRVTFHSVLDPTRTCCYISEVLDAGLLGPLFRVTVEGLPEVSFTHTSPMQCWDSVRDRVNEEIAKQISFGKSGLPDFLSCNSLNGLEMFGFLSSPIIKEIEALDPCHQCLDYWLSRVSSVGTELPSESVMAAMVNDSTNPPIKLLGIEINRRESEQSSSFNNSCVRRSHLAGC

SEQ ID NO: 175 - protein, 1249 aa, Sorghum bicolor
MMGTEGIAATVIEDPEPSIPPGFGPFAAPASWGIQNDVKPADAHSSSVLALQSIDDDVEILECLSSSVDRQSDTGCSISGSNTCRKSLRNRPPIDYSRFDQIAYEDSDAEVADKGASSVKHRQQFPKGVLRGCPECADCQKVIARWNPSGARRPVLDEAPVYYPTEEEFQDTLKYIESIRPTAEPYGICRIVPPASWKPPCLLKEKNIWECSKFSTRVQKVDKLQNRKSSKKSRRGGMMKKRRKISETEEINHNQIGMQQNQERFGFEPGPEFTLQMFQKYADDFSDQYFMKDKCRDSPPSVEDIEGEYWRIVERPTEEIEVIYGADLETGTFGSGFPKLCPEMKSDAEDKYAQSGWNLNNLPRLQGSVLSFEGGDISGVLVPWLYVGMCFSSFCWHVEDHHLYSLNYMHWGAPKMWYGVPGKDAVNLEAAMRKHLPELFEEQPDLLHNLVTQFSPSLLKSEGVPVYRCVQHEGEFVLTFPRAYHAGFNCGFNCAEAVNVAPIDWLPVGQNAVEVYREQARKITISHDKLLLGAAREAIRAQWDILFLKRNSADNLRWKSVCGPDSTICKTLKARIEMELAQRQNISSPCQSRKMDTEFDSTDRECALCYYDLHLSASGCPCSPEKYACLVHAKQLCSCDWNKRFFLFRYDVNELNILADALAGKLSAIHRWGVSDLGLSLSSCVKREKVQDSKTVRRLTDGPRRSYLSQASTVSLVPSSVSTEPKKNENKTLDLGCPGMNLLKISPEANNACPLTKQIKSENDSQLKEPCVKNELSCPTSNGTSRQHNGGIVGHKVAAPSLMVPSGQSLPANAVTKPLITSGESMRSTHVLAVLKESRETSSQTGDCTSSLTIREYHNRPLSMIDNGVNMKPDLENLDNSHRLMASSDFNATLCHSYKDQTFLTPETNTSVMTEKDSSQARTASQQFVNTASRTQNVSQEPFCSVTAPKHLIDPQVVKNSYGVFGSGSAHLGHPTVGNQQLNERWHQRQSDSLSSVEVRARGLSAMIVQPALENHSRNGVVQKGPRIANVVHRFKCSVEPIEIGVVLSGKLWSSSQAIFPKGFKSRVKYFSVVDPVQMTYYISEILDAGQQGPLFMVTVENCPGEIFINISPTKCWNMVRERLNMEIRRQINMGRANLPTLQPPGSVDGHEMFGLLTPAIVQAIEARDRDHICTEYWRSRSHVTTENRDNQNMPPQDPLLIALRGLFQRANCDELRALRSLLMSNRTLDDSSRQQACQILDEEIAKQW

SEQ ID NO: 176 - protein, 1064 aa, Cucumis sativus
MDRNKLEADSETKGDQSSKSSHKSNQTVERSGSPQHQKISARWDPNEACRPLVDEAPVFYPTVEEFEDTLGYIAKIRPQAESYGICRIVPPSSWNPPCVLKEKSKWENATFSTRIQQVDLLQNREPMKKKSRGRKRKRRRQSKAGTSARSTNLGVEATVTSESDEKFGFNSGSDFTLKDFQAYADHFRESYFGITKAQEDINFDIESSKRWEPSVEDIEGEYWRIVEKSNDEVEVYYGADIESATFCSGFPKASSLVTEGNLDPYVKSGWNLNNFPRLQGSVLCFEESDISGVLVPWLYVGMCFSSFCWHVEDHHLYSLNYMHWGDPKVWYGVPGSHASSLEAAMKKHLPDLFAEQPDLLHELVTQLSPSVLKSEGVPVYRVVQNSREFVLTFPRAYHAGFNCGFNCAEAVNVAPVDWLVHGQNAVELYSAQRHRTSLSHDKLLFGSAREATQALWEILVLEKKTPNNLNWKSVCGIDGDLTKVIKTRVKMEEERMNCLPTNMKLQKMESEIDCKSERECYACFYDLYLSSTSCKCSPDRFSCLKHASNFCSCQVDDRSVLFRYSINELHTLVEALEGGLDAIKEWASRYCKMEKDNESVAKVELDSGLNDKPSWSPQITDKLKRSDVPCSSSSHASSEVVQSESHRGSLSLNNSNLSSDSQNDNVNSEMLINKGKKVEQEGCIDLNVDIISEGNANCGPHISDTKTIIDLEETYPLVFEQKYICKAAHESELMDLDTDHVTTLPAHDYSSSVKDGVRICGSNASKLFGVDLSQSQSQLHSAFPYNNCSKVETSEHLDKRIPSWSSSHLKTFPFVEPLNIGTIMFGKPWHCEKAIFPKGFRSRVKFLSVLDPTSIVTYTSEVLDAGLLGPLFKVTLEESPGENFTNVSATKCWDMVVQRINREIERHNLRSGGRLPGQLLKEVDGLEMFGFLSPHVIQAIEALDPTHQCMEYWNHRKQQAIPANSGDNTFCESSALGLNFCWGETSATTFDINREEDETVTPTIGMERHHQNEVQVRSVLKGLLNKANPEELSVLQTIFCTDSQTTELRAEFASLIKEKQDKCR

SEQ ID NO: 177 - protein, 1086 aa, Vitis vinifera
MEQSSLEPEFQIKEDHSSKHALKNDSNIEYSGSPQNQKISARWNPTEACRPLIEEAPVFYPTVEEFQDTLNYIASIRPKAEPYGICRIVPPPSWVPPCPLREESIWKHLKFPTRMQQVDLLQNREPMRKKNRGRKRKRRRYSRMGTTRRHSRSEVSEANIVSDSDEKFGFHSGSDFTLEEFQKHADSFKEFYFGIKDAKDNLNSDGVECNKRWEPSVEDIEGEYWRIVEKPTDEVEVYYGADLETEAFVSGFPKASSLISENDSDQYVASGWNLNNFPRLPGSVLCFEQNDISGVLVPWLYVGMCFSSFCWHVEDHHLYSLNYLHWGDSKVWYGVPGSHASALENAMRKHLPDLFEEQPYLLNELVTQLSPSVLKSENVPVYRAIQNSGEFILTFPRAYHSGFNCGFNCAEAVNVAPVDWLSHGQSAVELYSEQCRKTSISHDKLLLASAQKAVQALRDPSVLGKEDQVNLSWKSVCGKDGTLTKAVKTRVQMEEERLDRLPIGWRLQKMERDFDLKNERECFSCFYDLHLSAASCECSPDQFACLKHASLICSCEPNRKFVLLRYTMDDLKTLVESLEGGLDAIEVWASEDLGLVSADKDACGAMLDQEREISGPIGCDQKESPPCSSRTQENLDINEPCSSSYHVSSEVVQSENQQGTFGFCVSHIRTDRHNDNLNKEGLTKGYESKVGQGFCIDLNLDTMSDEHVSGLQQVSYSCDSKATGNVAETFLSVCKEEKVNCADVPKQPDIVRLGGDCDSSVSYVLPNKHHFPYPVDNGNPCISDGSKLFGADILVSLPHSSTLPSSLPKTEILGSSDVKACATDQTCLIPKMNFCVEPMHFGTVLFGKPWCSKQAIFPKGFTSRVKFFSVCDPTQMCYYISEVLDAGLLGPLFKVTSEGCPSETFANVSPEKCWEMVLQKLQQEIIRHSSLGKQLLPSLECLQGVNGLEMFGFLSPPIIQVIEALDPNHQCLEYWNQKSRVKMENVNDMSASNSRKYPFGLSCSPGETKAKLFGFDLTKQDPDNSSIGRGDHSVGEDIKTTLQGFFKKANREELIMMYKVFCSEYTSAEWGVAFTTLTEEIRKTCK

SEQ ID NO: 178 - protein, 947 aa, Gossypium hirsutum
MKRDNNIEPSGSPRSRKVSARWDPNEACRPTIDDAPVFYPTVEEFEDTLGYVEKIREEAESFGICRIVPPPSWTPPCLLKEKDIWEHAKFSTRIQQVDLLQNREPMRKKTRSRKRKRKRQSKKGAARRCANSSVESTSASSEKFGFSSGSDFTLEEFQRYANEFKETYFQRDRDEDLKPGVVEYSKWEPSWEDIEGEYWRIVEQPTDEIEVYYGADLETGTFGSGFPKASSMLTGNDADKYAMSGWNLNNFSRLQGSVLSFEGCDISGVLVPWLYVGMCFSSFCWHVEDHHLYSLNYMHWGDPKVWYGVPQNHASSLEAAMRKHLPDLFEEQPGLLHDLVTQLSPSILKAEGVPIYRAVQYSGEFVLTFPRAYHSGFNCGFNCAEAVNVAPVDWLEHGQHAVELYSEQHRRTSLSHDKLLLGSARQAIQALWEIFFAGRETPGNLRWKHVCGKDGMLTKAVRMRVQMEEERVNCLPPHLPLRKMEKDFDLESERECFSCFYDLHLSACSCNCSPKQFACLKHVNNFCSCQMEDRFVLLRYTIDELQMLVKALEGGVDAVKVWACKDIGLLPGKDCDAYMPNLVQDSEAVKLEPSEPSGSWSSSRQMEEKANISSSSYGHVSEVLHQHGTKLKASPSTGGCENNAYNIGVLITENRVNLEQDACMKLNLDILTDYPASKSVNALDSPNNKSDVEIFLPSFNQEKICGFDEVRKPVLKRLKSDCSSSVSQESPNKYQHSTSCVHQDSDGFDGKKLFDVELHPGQSNTFWKTATVNSSDLNASIVARGDPLLISSVEPLSFGSVMLGKLWCSKKAIFPKGFRSRVKFFSVIDPTETSTYISEVLDAGPLGPLFKVTLEGCPTVSFSNVSVEKCWELVVQQLKQEILRSNLGERGVLPLQSLKRVNGLEMFGFLSPPIIQAVEALDPNHQCLEYWNHKTSSDKNEVNKDLD

SEQ ID NO: 179 - protein, 1048 aa, Citrus sinensis
MEQSKLAAESHIKEISARWDPAEACRPIIDEAPVFYPTVEEFEDTLGYIAKIRSKAESFGICRIVPPSSWTPPCPLKAKNIWENAKFSTRIQQIDLLQNREPMRKKIRSRKRKRRRQSRMGSTRRNANSSSEANAAETDEKFGFQSGPDLTLEGFQKYAQNFKECYFGMNDSKEDVKSDGFEHKRLEPSVVDIEGEYWRIIERPTDEVEVYYGADLETGAFASGFPKASSLGTESDLDQYAMSGWNLNNLPRLPGSVLAFEGSDISGVLVPWLYVGMCFSSFCWHVEDHHLYSLNYLHWGDPKIWYGVPGSHASTLEKAMRKHLPDLFEEQPDLLHELVTQLSPSVLKAEGVPVYHVVQHSGEFVLTFPRAYHSGFNCGFNCAEAVNVAPVDWLAHGQQAVELYSEQHRKTSLSHDKLLFGSVQAAIKALWELSVLQKKTPGNRKWKDACGKDGVLTKAIKTRVQMKKEGLQKLPSYFKLQKMEIDFDLKTERECFSCFYDLHLSAAGCKCSPDRFACLKHANIFCSCEIDHRFVILRYSTDELNTLVEALEGGLDALKELASKNFKWADCSDTDGGLVKMDMESEVFPMDCCEQKESSSSSPRVENIVEGNGPCCSRSHVSSEVVQSEPQRGTSGLSASHVSVNSHNEGNDETQVMNKKAKVKHEVCIDLNMDVIPDGNESKLLLSDSHGKEAIENLKAHLSACYQEKVLCSGTVKEQDTMQVRSDCNSSNSHKDPNKDQPSCSRVIEGTCSFDVKKLFGVDLSLPHQQSKLPLVDFLKTDTINGSNVRTSVTDQRFQKKLETCVEPINFGCVMCGKLWCSKQAIFPKGFRSRVNFYSVLNPEKVCNYISEVLDAGLLGPLFKVTLEECPSETFVNVSAQKCWEMVLQRLNQEIERQGGLHERGLPHPQSLQSIDGLEMFGFLSSPIIQAIEALDPNHLCMEYWNHKLLTFGKTTEVNKNSSSGLSCSEAETKSKIFGVALMDEDQNSPSGQNSVEEEAQLVLRGLFQKASPKELKVMQRILYSEGRSDEWRVALATLIEEIQKSCR

SEQ ID NO: 180 - protein, 1128 aa, Populus trichocarpa
MEQFKSPAYSHVKEDHSIKQSWRSDNTPKGPRSPQNQKVTARWDPVEACRPLIDDAPVFYPTVEEFEDTLGYISKIRAKAELYGICRIVPPPSWSPPCRLKEKDIWEHAKFSTRIQYVELLQNREPMRKKSKSRKRKRRFSRMGTTRRRKRRLTNSSSEGNVASETDETFGFHSGSDFTLEEFEKEAAYFKECYFGTKDLMDDGNETQKWEPSVEDIEGEYWRIVEKPTDEVKVLYGADLETATFGSGFPKASALMTEGDSDQYVVSGWNLNNLPRLPGSVLCFEGCDISGVLVPWLYVGMCFSSFCWHVEDHHLYSLNYLHWGDPKIWYGVPESHASNLEDAMRKHLPDLFEEQPDLLHGLVTQLSPSVLKAEGVPVYRVVQHSGEFVLTFPRAYHSGFNCGFNCAEAVNVAPVDWLAHGQHAVELYSEQRRKTSISHDKLLMGAAQEANHALRELLLLGKETPENLRWMSVCGKDGVLTAAVKTRVKMEEERIKSLPTNLKLQKMEKDFDLQKERECFSCFYDLHLSSASCKCSPERFACLQHASHFCSCEIDHRYVLLRYTMDELNTLVDGLEGESYGLKDCPDEQGLVSLGDNGTRVPELELKGEEFQTNYSKRKESPRCSKKTEEKLSTKGSCSFNSNTSSEVIQSESYHNSFPVMKNKDKVKQEGCIDLNIDVMSIDQESKHLLESDGCDNKAISYVKETHGSPCMQETPGSSDAAKEQDREQAVGDCEAKLQELSNKNDPSYPMFTQDTCASRNKLFGVDLSRSHSVRPAKSFKTEMNKGGLDVRPATNGSSDAAKEQDREQAVGDCEAKLQELSNKNDPSYPMFTQDTCASRNKLFGVDLSRSHAVRPAKSFKTEMNKGGLDVRPATNQSIPVKKLNPCVEPINVGSVMFGKFWCCKQAIFPKGFKSRVKFFNVHDPIKKCSYISEVRDAGPLGPLFKVSLEKFPGETLAADVSIQKCWEMVVQRLNDEIGRRNSLGERNLPPSQSINGIEMFGFLSPPIVQAIEALDPDHRCVEYWNHRLVNLRNTREAKQPPFGSSCCLTKMKEKIDINLLTQEPGSLFIGGHRSVDEDVQHVLRGLFKKASQEELKTMHRILHSDAQSAERREAFTTLMEEIQKTSR

SEQ ID NO: 181 - protein, 333 aa, Arabidopsis thaliana
METNPCDMNQLDSDSHLPPRKRLLAGFKKFNSNGINGSSPSDFASSSSTSNSNGSSSASTNVQTHLGNLLSSPFNNDQSPEELVEATRSAAALAVKAAKAARAIANEKALISAKAIAAAKRALELVDSFPKEAMADCKERSPRKNKQKKHVPVELLYSKGQLRDEEDDLARRLHRAIDNTSYPRVLRTSEENGQRYKKQKKNKSVVEGGSSSIIVTGSMKDIAGVVDSDSSYEGLEIARSNRDEADSMLMMEKSGEESNSLVKRRGRVKLKKLPLSICNSRNQENGTSSASPLPVAQPQEDGGAITVIAGSSSWKCQDIKAPECVKQNKAVRS

SEQ ID NO: 182 - protein, 375 aa, Glycine max
MENVCDVNHLDADVLLPPRKRLLAGLKKQSSDGDAAASPPLVVASCVTTVSEVASPSLTSYSSEFEARLKNLLGSHSDNPNLTPEEVVEASKAAAVAASKAAKAARAAAEEKAETAAKAVAAAKRALDLVASFSEEAVSSKERNLKKNKLKKHLPVHLLYKKYQPIENCGTDEELARKLHRAMNSSPRISKNSPNSDSKGSKHKKPKNSWSFEMTEVSDARMAFGQDCLSLNNGHAVVGKVDSEGSIQEVCSNKEDKKSQMEITDNGEAESSQSKEKNTEDLSSVGRKRGRVKLKKLPLSICTSKDRALPKEGVRARSAPLTEMNTGGNHAVGNIPLFPMEPSTDRVIPIEATTTCKCQELKAPACIKQNKAVQS

SEQ ID NO: 183 - protein, 380 aa, Setaria italica
MEPSDLNTHLPPRKRLLAGLRTAATSCDAAEPLPSPLASGDLAARLREMALAANASASSPEEMIEAARAAAEAATDAAAAARAAAAEKAAVAAKARAAARAAMEFLDSFSRTGTSRNGLQFKVKSRKKHVQVKMLYRPNGTLGDAPKPRRRKQSDEEIARNLHRAMNSSPRISHTGPPKRPRGKAGDGKDGAAPGEGNREGRDDACNGSSTHAPIEAGGGLSNGCCEEKSSETTVPLFKHGGPDDGGDDPSRHAAKSRGDVTDNGVGTGNLSAARKVKIKRKELLLNQHNNKETEEPKETEPSVQPIGQDEPKTNGNGTEKHGSPADAKAPGDGTASMKITSVWKFKKLKTSHCSSDSKVLHNVCSSPTAAETSASVKAD

SEQ ID NO: 184 - protein, 375 aa, Spinacia oleracea
MDANDCDTNHLDGDVRLPPRKRLLAGLKKQSCSESNSPCSQENTPSPIPSSSPSPSFSASPLSEFDARLNNLLKSYKNGSNMSPEEIAVAAGSAAASAAKAAEVARAAAEEKAAIAAKAVAAAKTALNAVASVSEETSSKDKYQKKNKLKKHVPVQLLYKNYQPVENCGTDEELARKLHRAINSSPRINRHSSGSDSKNHKHKKLKISPSSEKSGVVANGDVVEGGMDCEGSNEEPYAVKLDDKVLSKFTTKADEVEVEVDSGEAESSHSKAKTFTAASDDVCNNGRKRGRIKQKKLPLSICSSKDQSNPKEDPPILTNSLLSRARTDKSTARHVSLLSLERPGENVTPMEVAPTWKCQDFKVSQCINQDKIVQS

SEQ ID NO: 185 - protein, 359 aa, Sorghum bicolor
MEPPPSDLNTHLPPRKRLLAGLRTAVATTGDAAAAEPLPSPPASAGDLATRLREMALAANASASSPEEMIEAARAAAQAAADAAVAARAVAAEKAAVAAKARAAARAAMEFLDSFSSRGAGASRNGNGLQFKVKSRKKHVQVKMLYRPNGTLGLGEAPKPRRRKQSDEEVARNLHRVMNSSPRISHTGPPKRPRTITAGDAKDAVVAPGGGNGEGDTDACNGSSIHVPIEAGTGLPNGCSEGKSSERTVPLLNREGPDVDGQDSSRHAAKSSGDIADNGVWVRNLSAGQKVKIKRKELLLNQNGDTKTPGDGVTPMKITSVWKFKKLKTSHCSSDSKVLHKVCPPPPAAAETSASVKAD

SEQ ID NO: 186 - protein, 375 aa, Cucumis sativus
MEGNICDVNHLNSDVLLPPRKRLLAGLRKQGGDGDGTFNLPPVASSSCSPPPSPSYGFTSIEFNIRLNSLLSAHSNSNLSPEEIVQASRSAAAAAVKAAEAARAAAEEKAAIAARAVTVAKSAMDLVASISEEAAYKEINLRKNKLKKHVPVQLLYTKYQPLENTKTDEELARKLHRAINSSPRILKNSSGSDVRSHKHKKLKSSTSSEKIRVSNCGISQDLDPTTTCNGHAKPNEVDSECSFQEVYKLKPDEKTSKYEKSNPSLTDNGEETSQKEKMCDDISVTIKKRGRVKLKKLPLSICSFRDKTTLKEDMNNGSSPILTVQNRGSPTSEKVILHSVDSPTEGVMPIDSTSVWKCQEFKAPLSVKQNKVVQS

SEQ ID NO: 187 - protein, 369 aa, Vitis vinifera
MEANICDINHLDADVLLPPRKRLLAGLKKQSSDGDATLHPSPIAMTSNEFDARLNKLLSSHLKNPSLTPEEIVEASRSAAKAAAKAAEAARAAAEEKAAIAAKAMAAAKSALELIASVSEETSSKERYLKKNKSKKHVPVQLLYKKHQPVENYRTDEELARKLHRAMNSSPRISKNSSSSDWKNHKHKKPKIFPAVEKTRVPNGGMVLEGNSPSTCNGSSIAGDADSQNSAREVYTVKVDERALRSHRADQLETDNGEAGCSKEKNSEALDHLSTTGRKRGRIKLKKLPLSICTIRDRANPKEELKSRSAPMTEENIFKQTVKPTAGNIPLFSVGPSAGSVMPVEATTMWKCQEFKAPACVKQNKVVQS

SEQ ID NO: 188 - protein, 384 aa, Gossypium hirsutum
MEANICDINHLDADVLLPPRKRLLAGFKKQASNANGASDQPAVASSSSSPPSPSPSPSPSPSPSHSPSTSSSDVNDHLKNLLMSSHLYNPNLSPEEILEASRAAATSAAKAAEAARAAAEEKAAIAAKAVAAAKNALDMVAKFSEDTINKERHLKKNKLKKHVPVQLLYKKHEPIESPRTDEELARKLHRAINSSPRISKNSPTSAWRGHKHKRPKIMPTLEKTKASNGGIVLGGSPSSTCNGGTIAGEIDSGDSMEESVKAEAKGAKYEKSGKSELDNGEAESSHLKEKACDDVTPSKKRGRMKLKRLPLSICSFRDRVNPKEDMITKSSPLTEKNMDNSTAAVKTFYSLEPSDDGVISIEGMRKCQDFKAPACIKQNKVMQS

SEQ ID NO: 189 - protein, 379 aa, Citrus sinensis
MEANICDINHLDGDVLLPPRKRLLAGFKKQSSDGNGNCNDNDNGAVHQPDVPSSSSPYSPSSSEYSSEFNARLNNLLKSTMSSSNLSPEEIAELAESAAIAAVKAAEDARTAAEEKAATATRAITAAKSALDLVASFNEEGASKERHLKKNKLKKHVPVQLLYKKNQLKEDSKTDEELARRLHRVMNSSPRISKNLTSSDLKGHKHKKLKISPASEKIRVSYGGLVLGGNTVCTSSRHAVVGEVDFENEGIFREVYTVDEDEKVDQMQVGSGEAGVSHSKEKALEETSSPGRKRGRLKLKKLPLSYCTSRDRENTKEGMITRTSPLTDKNIGNPPGGNAVPLFSMEPSSDGVMPIEATPWKCQDFKAPECIKQNKVVQS

SEQ ID NO: 190 - protein, 385 aa, Populus trichocarpa
MEANICDISHLDADVLLPPRKRLLAGFKKQSSDGDGASRPPIVASSSSSASPPSPSPSPTPRSPSSNEFQTQLNILLSSHFNSSHNLSTEQIVEASKSAAEAAVKAAEAARAAAQEKAIIAAKAVSAARSALALVASFPEETASKDKFLKKNKLKKHVQVQLLYKKHQPVENFRDDEKLARKLHRVMNSSPRISKNSSSSDLKGGHRNKKPKSSPSSERTRVSNGSTMFGENLPSSCNGHAIAGELDSDESIQEAHTSIPDEKALKYEKTGQLDIDNWEAESSHLKEKNRGDSGSPSKKRGRLKLKKLPLSICNSKDQSNPKEGSLPKILPLTDKTMGNPTTRSKPLFPMEPSSGTLVQIEATSIWKCQEFKAPACIKQNKVVQS

SEQ ID NO: 191 - protein, 275 aa, Arabidopsis thaliana
MATNACLESDSLLPPRKRLLAGFKNQNSRISNESPSPPFASSSSSTTTSNGSSASAVVHTHHLDHLLNDQTRSPEELAQASKATAALAVKVAKAARATANEKAIIASKAVAAAKNALELFASFPAAETVSCKEPRLIRAINNSPRVLTDCSGHRNKKQKTLTSTMNDGNDVAGVVDSDDGTRVNGKLLLCDNTLKEKTEEESSSLGKRRGRVKLPLSMCAYKDQENGITLEENNLSVSKSEGYNGGVAQVIERPSWKCQDLKSPECVKQNKVVRS

SEQ ID NO: 192 - protein, 375 aa, Glycine max; identical to SEQ ID NO: 182

SEQ ID NO: 193 - protein, 383 aa, Theobroma cacao
MMEANICDINHLDADVLLPPRKRLLAGFKKQASNANGSSDQPTVASSSSSLPSPSPSPSPSPSPSTSSSDVNTHLNNLLSSHINNPNLSPEEILAASRVAAIAAAKAAEAARAAAEEKAAIAAKAVAAAKSALDLVATFSEETVSKDRYLKKNKLKKHVPVQLLYKKHQPIENNRTDEELAHRLHRAINSSPRISKNSPTSEWKGHKHKRPKSLPTLEKTKIYNGGIVLGGSQSSTCNGDTVAGEIDSEDSIQESVKAEAKGTKYEKSGQSELDNGEAESNQSKEKACEDVYSPGKRRGRVKLKKLPLSICSFRDRVNPKEETITKSSPLTEKNMGNPSAAVKPLFSLEPSTDGVISIEGTPIWKCQDYKAPACIKQNKVMQS

SEQ ID NO: 194 - protein, 375 aa, Spinacia oleracea; identical to SEQ ID NO: 184

SEQ ID NO: 195 - protein, 341 aa, Cajanus cajan
MENACDVNQLDADVLLPPRKRLLAGLKKQGSDSDATASASASPSPVAASCVTDVASPSSSSFSSEFEARLKHLLSCHSNNPNLTPEEIVEASKAAATVATKAAEAARAVAEEKAARAAKAVEAAKRALDLVASLSEEAAGKERNLKKNKTDEELARKLHRAMNSSPRISKNSPNSDSKGSKHKKPRSSSSLEISEGTESGVAFGQDCLSLNNEHAIAGKIDSEGSIQEACSNNREAESSMLKGKNCEDSSPIGKKRGRVKLKKLPLSICTSKDRAQPKEEIRARSSQMTGMNTGKNPVDSRPLLTVEPSTDRVMPIEATSMWKCQDFKAPACIKQNKAVQS

SEQ ID NO: 196 - protein, 375 aa, Cucumis sativus; identical to SEQ ID NO: 186

SEQ ID NO: 197 - protein, 369 aa, Vitis vinifera; identical to SEQ ID NO: 187

SEQ ID NO: 198 - protein, 339 aa, Gossypium hirsutum
MEANVCDINHLDADVLLPPRKRLLAGFKKQASNANGTSDQPSAASSTSSPPSPSPSSATSSTDFNIHLNNLLSSHVNDPNLSPEKIFEASRAASIAAAKAAEAARAAAEEKAAIAAKAIAAAKSALDLVATFSEDAGSKDRYLKRNKLKKHVPVQALYKKDQQPIENSRVDEDLVSRLNRAINSSPRISKDSLKSEWRGHQRKRPKSLPTFDNTKVSNGGIMLGGSPSSTCNGAMIAVEIDSDDLVEEHVKVEAKVANYKKSRPSMLDNGEAESGYLKEKANEDAYSFGKKRGRVKLKKLPLSICSYRDRVNLKEEMITKNMGNSTAVVKPVFFSGTLC

SEQ ID NO: 199 - protein, 379 aa, Citrus sinensis; identical to SEQ ID NO: 189

SEQ ID NO: 200 - protein, 385 aa, Populus trichocarpa; identical to SEQ ID NO: 190

SEQ ID NO: 201 - protein, 218 aa, Arabidopsis thaliana
MSSINITNVTVLDNPAPFVNPFQFEISYECLTSLKDDLEWKLIYVGSAEDETYDQVLESVLVGPVNVGNYRFVLQADSPDPLKIREEDIIGVTVLLLTCSYMDQEFIRVGYYVNNDYDDEQLREEPPTKVLIDKVQRNILTDKPRVTKFPINFHPENEQTLGDGPAPTEPFADSVVNGEAPVFLEQPQKLQEIEQFDDSDVNGEAIALLDQPQNLQET

SEQ ID NO: 202 - protein, 192 aa, Glycine max
MSAVNITNVTVLDNPASFLTPFQFEISYECLTALKDDLEWKLIYVGSAEDETYDQLLESVLVGPVNVGNYRFVLQADPPDPSKIREEDIIGVTVLLLTCSYLGQEFIHVGYYVNNDYDDEQLREEPPPKVLIDRVQRNILSDKPRVTKFPINFHPENNENEEQQPPPSEHPSETGEDPLAVVDRDPPDEKDS

SEQ ID NO: 203 - protein, 193 aa, Zea mays
MSAVNITNVAVLDNPTAFINPFQFEISYECLVPLDDDLEWKLIYVGSAEDENYDQQLESVLVGPVNVGTYRFVLQADPPDPSKIREEDIIGVTVLLLTCSYMGQEFMRVGYYVNNDYGDEQLREEPPAKVLIDRVQRNILADKPRVTKFPINFHPEPSTGTGRQQQQQEPQTASPEKHAGSGEGNGSKPEADQ

SEQ ID NO: 204 - protein, 188 aa, Oryza sativa
  source 1..188: note = Japonica Group
MSAVNITNVAVLDNPTAFLNPFQFEISYECLIPLDDDLEWKLIYVGSAEDENYDQQLESVLVGPVNVGTYRFVLQADPPDPSKIREEDIIGVTVLLLTCSYMGQEFMRVGYYVNNDYDDEQLREEPPAKLLIDRVQRNILADKPRVTKFPINFHPEPSTSAGQQQQEPQTASPENHTGGEGSKPAADQ

SEQ ID NO: 205 - protein, 193 aa, Sorghum bicolor
MSAVNITNVAVLDNPTAFLNPFQFEISYECLVPLDDDLEWKLIYVGSAEDENYDQQLESVLVGPVNVGTYRFVLQADPPDPSKIREEDIIGVTVLLLTCSYMGQEFMRVGYYVNNDYDDEQLREEPPAKVLIDRVQRNILADKPRVTKFPINFHPEPSTGTGQQQQEPQAASPENHTGNGHGNGDGSKPEADQ

SEQ ID NO: 206 - protein, 210 aa, Cucumis sativus
MSAVNITNVTVLDNPAAFLNPFQFEISYECLTSLKDDLEWKLTYVGSAEDETYDQLLESVLVGPINVGNYRFVLQADPPDPSKIREEDITGVTVLLLTCSYMGQEFVRVGYYVNNDYDDEQLREEPPPKVLIDRVQRNILADKPRVTKFPINFHPENSEHGAEQQPSSPHHSVEALNNEEEELQPPPSPSAADEPPLDKEAIDSGSQPSV

SEQ ID NO: 207 - protein, 186 aa, Vitis vinifera
MSAVNITNVTVLDNPAAFLNPFQFEISYECLIPLKDDLEWKLIYVGSAEDETYDQLLESVLVGPVNVGNYRFVFQADPPDPSKIREEDIIGVTVLLLTCSYLGQEFIRVGYYVNNDYDDEQLREEPPQKVLIDRVQRNILADKPRVTKFPINFHPENNEHGEQPPPSPEINENGEQPPPSHHDPSD

SEQ ID NO: 208 - protein, 190 aa, Gossypium hirsutum
MSAVNITNVTVLDNPASFLSAFQFEISYECLTPLKDDLEWKLIYVGSAEDETYDQLLESVLVGPVNVGNYRFVLQADPPDPSRIREEDIIGVTVLLLTCSYLGQEFIRVGYYVNNDYDDEQLREEPPPKVLIEKVQRNILSDKPRVTKFPINFHPENGGNEEPPPSDQPVENVGNEEVPASPNQKEEQDP

SEQ ID NO: 209 - protein, 190 aa, Citrus sinensis
MSAVNITNVTVLDNPAAFLNPFQFEISYECLTPLKDDLEWKLIYVGSAEDETYDQLLESVLVGPVNVGNYRFVLQADPPDPSKIREEDIIGVTVLLLTCSYIGQEFVRVGYYVNNDYDDEQLREEPPPKVLIDRVQRNILADKPRVTKFPINFHPENNEHGEDSPPDQATETNENGEEQLPSPDHPSHEQ

SEQ ID NO: 210 - protein, 193 aa, Populus trichocarpa
MSAVNLTNVTVLDNPAPFLSPFQFEISYECLTPLKDDLEWKLIYVGSAEDETYDQLLESVLVGPVNVGNYRFVLQADPPDPSKIRDEDIIGVTVLLLTCSYLGQEFVRVGYYVNNDYEDEQLREEPPPKVLIDKVQRNILSDKPRVTKFPINFYPENTEGAEEPLANDQPAETDGNEERLPASPHHSSDKEGP

SEQ ID NO: 211 - protein, 169 aa, synthetic construct
LEPYKCPECGKSFSQSGDLRRHQRTHTGEKPYKCPECGKSFSRSDHLTTHQRTHTGEKPYKCPECGKSFSQSSSLVRHQRTHTGEKPYKCPECGKSFSTSGNLVRHQRTHTGEKPYKCPECGKSFSQRANLRAHQRTHTGEKPYKCPECGKSFSRSDKLTEHQRTHTGL

SEQ ID NO: 212 - protein, 391 aa, synthetic construct
MARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQLGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPS

SEQ ID NO: 213 - protein, 9 aa, synthetic construct
SSGPPPGTG

SEQ ID NO: 214 - protein, 12 aa, synthetic construct
AEAAAKEAAAKA

SEQ ID NO: 215 - protein, 16 aa, synthetic construct
SGSETPGTSESATPES

SEQ ID NO: 216 - protein, 1407 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 217 - protein, 2217 aa, synthetic construct
METNPCDMNQLDSDSHLPPRKRLLAGFKKFNSNGINGSSPSDFASSSSTSNSNGSSSASTNVQTHLGNLLSSPFNNDQSPEELVEATRSAAALAVKAAKAARAIANEKALISAKAIAAAKRALELVDSFPKEAMADCKERSPRKNKQKKHVPVELLYSKGQLRDEEDDLARRLHRAIDNTSYPRVLRTSEENGQRYKKQKKNKSVVEGGSSSIIVTGSMKDIAGVVDSDSSYEGLEIARSNRDEADSMLMMEKSGEESNSLVKRRGRVKLKKLPLSICNSRNQENGTSSASPLPVAQPQEDGGAITVIAGSSSWKCQDIKAPECVKQNKAVRSGGPSSGAPPPSGGSPAGSPTSTEEGTSESATPESGPGTSTEPSEGSAPGSPAGSPTSTEEGTSTEPSEGSAPGTSTEPSEMDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQLGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPS

SEQ ID NO: 218 - protein, 1270 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGRTGGGGGMETNPCDMNQLDSDSHLPPRKRLLAGFKKFNSNGINGSSPSDFASSSSTSNSNGSSSASTNVQTHLGNLLSSPFNNDQSPEELVEATRSAAALAVKAAKAARAIANEKALISAKAIAAAKRALELVDSFPKEAMADCKERSPRKNKQKKHVPVELLYSKGQLRDEEDDLARRLHRAIDNTSYPRVLRTSEENGQRYKKQKKNKSVVEGGSSSIIVTGSMKDIAGVVDSDSSYEGLEIARSNRDEADSMLMMEKSGEESNSLVKRRGRVKLKKLPLSICNSRNQENGTSSASPLPVAQPQEDGGAITVIAGSSSWKCQDIKAPECVKQNKAVRSSRADPKKKRKVARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQLGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPS

SEQ ID NO: 219 - protein, 1841 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVEDAPKKKRKVDGIGSGSNGSSGSNGPTDAAEEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSG